(19) United States
(12) Patent Application Publication
(10) Pub. No.: US 2023/0124361 A1
(43) Pub. Date: Apr. 20, 2023

(54) SMALL MOLECULE STING ANTAGONISTS

(71) Applicant: CURADEV PHARMA PVT. LTD., Noida, Uttar Pradesh (IN)

(72) Inventors: Monali BANERJEE, Noida (IN); Sourav BASU, Noida (IN); Ritesh Kumar SHRIVASTAVA, Noida (IN); David Cameron PRYDE, Noida (IN); Sandip Kumar MIDDYA, Noida (IN); Rajib GHOSH, Noida (IN); Dharmendra B. YADAV, Noida (IN); Arjun SURYA, Noida (IN)

(21) Appl. No.: 17/798,552

(22) PCT Filed: Feb. 12, 2021
(86) PCT No.: PCT/IB2021/051154

§ 371 Date: Aug. 9, 2022

(30) Foreign Application Priority Data
Feb. 12, 2020 (GB) 2001884.2
Feb. 12, 2020 (IN) 202011006115

(51) Int. Cl. C07D 417/12; C07D 279/16; C07D 417/06; C07D 417/14; C07D 471/04; C07D 413/12; C07D 265/36; C07D 413/14; C07D 403/12; C07D 239/80; C07D 417/04

(52) U.S. Cl. CPC C07D 417/12; C07D 279/16; C07D 417/06; C07D 417/14; C07D 471/04; C07D 413/12; C07D 265/36; C07D 413/14; C07D 403/12; C07D 239/80; C07D 417/04

(57) ABSTRACT

The present invention relates to compounds of formula (I). The compounds maybe used to antagonise the Stimulator of Interferon Genes (STING) protein and may thereby treat liver fibrosis, fatty liver disease, non-alcoholic steatohepatitis (NASH), pulmonary fibrosis, lupus, sepsis, rheumatoid arthritis (RA), type I diabetes, STING-associated vasculopathy with onset in infancy (SAVI), Aicardi-Goutieres syndrome (AGS), familial chilblain lupus (FCL), systemic lupus erythematosus (SLE), retinal vasculopathy, neuroinflammation, systemic inflammatory response syndrome, pancreatitis, cardiovascular disease, renal fibrosis, stroke and age-related macular degeneration (AMD).

The present invention relates to small molecule antagonists of the Stimulator of Interferon Genes (STING) protein. Accordingly, the small molecule antagonists may be of use in the treatment of various inflammatory diseases such as fatty liver disease, pulmonary fibrosis, pancreatitis, lupus, and so on. The invention extends to the pharmaceutical compositions of the compounds per se, methods of making the compounds and methods of modulating the STING protein using these compounds.

STING (STimulator of INterferon Genes) is an innate signalling molecule that plays a crucial role in mediating an immune response to cytosolic DNA.

The human immune system has evolved to recognize and respond to different types of threats and pathogens to maintain a healthy host. The innate arm of the immune system is mainly responsible for a rapid initial inflammatory response to danger signals associated with cellular or tissue damage from bacteria, viruses and other infectious threats. The innate immune system responds to these damage-associated molecular patterns (DAMPs) or microbial product pathogen-associated molecular patterns (PAMPs) through an array of sentinel proteins called pattern recognition receptors (PRRs) to provide broad and lasting protection to the host against a wide range of threats (P. Broz et. al., Nat. Revs Immunol., 2013, 13, 551).

The PAMPs and DAMPs are often constituents or replication intermediates of intracellular pathogens. PRRs include Toll-like receptors (TLRs; activated by endosomal nucleic acids), C-type lectin receptors, retinoic acid inducible gene I (RIGI-like receptors; activated by cytosolic RNA), NOD-like receptors (NLRs) and also double stranded DNA sensors (Diebold et. al., Science, 2004, 303, 1529-1531; O. Takeuchi et. al., Cell, 2010, 140, 805; Pichlmair et. al., 2006, 314, 997). PRRs respond to DAMPs and PAMPs by up-regulating type-1 interferons and cytokines. Free cytosolic nucleic acids (DNA and RNA) are known PAMPs/DAMPs. The main sensor for cytosolic DNA is cGAS (cyclic GMP-AMP synthase). Upon recognition of cytosolic dsDNA, cGAS triggers formation of one specific isomer of the cyclic dinucleotide (CDN) cGAMP, c[G(2′,5′)pA(3′,5′)p] (Gao et. al., Cell, 2013, 153, 1094).

CDNs are second messenger signalling molecules produced by diverse bacteria and con sist of two ribonucleotides that are connected via phosphodiester bonds to make a cyclic structure. CDNs cyclo-di(GMP) (c-diGMP), cyclo-di(AMP) (c-diAMP) and hybrid cyclo-(AMP/GMP) (cGAMP) derivatives (A. Ablasser et. al., Nature, 2013, 498, 380) all bind strongly to the ER-transmembrane adaptor protein STING (D. L. Burdette et. al., Nature, 2011, 478, 515; H. Ishikawa, Nature, 2008, 455, 674).

STING recognises CDNs through its cytosolic carboxy-terminal domain, which forms a homodimer and adopts a V-shaped binding pocket to bind CDNs (Zhang et. al., Mol. Cell, 2013, 51, 226; G. N. Barber et. al., Nat. Immunol., 2011, 12, 929). Ligand-induced activation of STING triggers its relocation to the Golgi and a conformational change to facilitate binding to TBK1. TBK1 in turn signals through the transcription factors IRF-3, STAT6 and NF_{K}B to induce type-I interferons and other cytokines and interferon-stimulated genes (C. Greenhill, Nat. Revs., Endocrinol., 2018, 14, 192; Y. Li, H. L. Wilson, and E. Kiss-Toth, J. Inflamm., 2017, 14, 11). Following its activation, STING is rapidly degraded in the normal response.

Excessive activation of STING is associated with a range of monogenic autoinflammatory disorders referred to as interferonopathies (Y. J. Crow and N. Manel, Nat. Revs. Immunol., 2015, 15, 429-440). Loss of function mutations in the human DNAse Trex1 are associated with elevated levels of cGAMP and autoimmune diseases such as the rare but severe inflammatory disease Aicardi-Goutieres syndrome (AGS), familial chilblain lupus (FCL), systemic lupus erythematosus (SLE) and retinal vasculopathy (Y. Crow et. al., Hum. Mol. Gen., 2009, 18, R130).

Inhalation of silica particles can result in lung inflammation and pulmonary fibrosis, triggered by lung cell death and release of dsDNA products. Benmerzoug et. al. have reported that this increase in circulating dsDNA activates STING and via increased levels of CXCL10 and IFN signalling produces lung inflammation (S. Benmerzoug et. al., Nat. Comm., 2018, 4, 5226).

Increased cytosolic dsDNA was detected in fibroblast-like synoviocytes (FLS) taken from rheumatoid arthritis (RA) patients with the levels of dsDNA correlating with the severity of rheumatoid synovitis (J. Wang et. al., Int. Immunopharm., 2019, 26, 105791). These findings indicated that increased dsDNA promoted an inflammatory response via the STING pathway in RA FLS and led to increased expression of STING, suggesting that cytosolic DNA accumulation is an important factor in RA-related inflammation.

Patients with autosomal dominant gain of function mutations in STING have a pediatric autoinflammatory condition called SAVI (STING-associated vasculopathy with onset in infancy), manifest clinically as skin rash, vasculopathy, lupus-like syndromes and pulmonary fibrosis characterised by aberrant IFN production and systemic inflammation that are associated with high morbidity and mortality (N. Konig, et. al., Ann. Rheum., Dis., 2017, 26, 468). Characterised mutations in humans include V147L, N154S, V155M and G166E which are all located at the interfacial region between the trans-membrane domain and the ligand binding domain and result in ligand-independent constitutively activated protein. More recently, three other gain of function STING mutations C206Y, R281Q and R284S have been identified at a cluster region that is proposed to promote STING aggregation and disfavour complexation to the C-terminal tail region (H. Konno, et. al., Cell Rep. 2018, 23, 1112 and I. Melki, et. al., J Allergy Clin Immunol. 2017, 140(2), 543.

A recent report by Habtezion et al. has shown that in mice with acute pancreatitis, STING responds to acinar cell death by detecting DNA from necrotic cells and promotes acute pancreatic inflammation (A. Habtezion et. al., Gastroenterology, 2018, 154, 1822). STING-knockout mice had less severe acute pancreatitis (less edema, less inflammation) while administering a STING agonist resulted in more severe pancreatitis.

Luo et al. have also shown recently that levels of STING were increased in liver tissues from patients with non-alcoholic fatty liver disease and in mice with a high-fat diet induced hepatic steatosis. Once again, STING-knockout mice developed less severe liver fibrosis and a less acute inflammatory response (X. Luo et. al., Gastroenterology, 2018, 155, 1971).

Elevated cGAMP levels in the peripheral blood mononuclear cells of SLE patients was associated with higher disease scores (J. An et. al., Arthritis Rheum., 2017, 69, 800) suggesting a link between disease severity in lupus and activation of the STING pathway.

The kidney tubule cells of subjects with fibrosis have been shown to lack mitochondrial transcription factor A (TFAM). Mice lacking tubule TFAM developed severe mitochondrial loss and energy deficit caused by aberrant packaging of mitochondrial DNA and its translocation to the cytosol, where the STING pathway was activated (K. W. Chung, Cell Metab., 2019, 30, 1). The ensuing cytokine expression and inflammation led to renal fibrosis.

Bennion et. al. have demonstrated that the gain of function mutation N153S knock-in mice showed enhanced susceptibility to viral infection and responded to infection by a murine gamma herpesvirus γHV68 with severe autoinflammation and pulmonary fibrosis (B. Bennion et. al., J. Virol., 2019, 93, e01806).

Other conditions where excessive immune system activation may be linked to STING pathway activation include systemic inflammatory response syndrome (R. K. Boyapati et. al., F1000 Res., 2017, 6, 169), cardiovascular disease (K. R. King et. al., Nat. Med., 2017, 23, 1481), stroke (A. M. Jeffries et. al., Neurosci. Lett., 2017, 658, 53) and age-related macular degeneration (N. Kerur et. al., Nat. Med., 2018, 24, 50).

There is therefore a compelling body of evidence that blocking, inhibiting or antagonising the STING pathway could have therapeutic benefit in a number of conditions and disease states. There is therefore a pressing need for improved small molecule blockers of the STING pathway, and in particular for small molecule direct antagonists of the STING protein.

The present invention has arisen from the inventors work in attempting to identify STING protein modulators.

In a first aspect of the invention, there is provided a compound of formula (I):

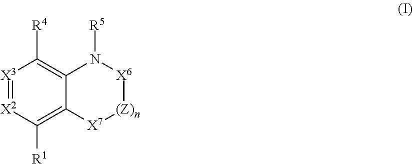

wherein X² is CR² or N;

X³ is CR³ or N;

X⁶ is C═O, C═S or CR⁷R⁸;

the or each Z is independently CR⁹R¹⁰ or NR⁹;

X⁷ is S, SO, SO₂, O, NR¹¹ or CR¹¹R¹²;

n is 0, 1 or 2;

R¹, R⁴, R⁸, R⁹, R¹⁰, R¹¹ and R¹² are each independently selected from the group consisting of H, halogen, OH, CN, COOR¹³, CONR¹³R¹⁴, NR¹³R¹⁴, NR¹³COR¹⁴, optionally substituted C₁-C₆ alkyl, optionally substituted C₁-C₆ alkylsulfonyl, optionally substituted mono or bicyclic C₃-C₆ cycloalkyl, optionally substituted C₂-C₆ alkenyl, optionally substituted C₂-C₆ alkynyl, optionally substituted C₁-C₆ alkoxy, optionally substituted C₁-C₆ alkoxycarbonyl group, mono or bicyclic optionally substituted C₆-C₁₂ aryl, mono or bicyclic optionally substituted 5 to 10 membered heteroaryl, optionally substituted mono or bicyclic 3 to 8 membered heterocycle, optionally substituted aryloxy, optionally substituted heteroaryloxy and optionally substituted heterocyclyloxy; one of R² and R³ is -L¹-L²-L³-L⁴-R¹⁵ and, when X² is CR² and X³ is CR³, the other of R² and R³ is selected from the group consisting of H, halogen, OH, CN, COOR¹³, CONR¹³R¹⁴, NR¹³R¹⁴, NR¹³COR¹⁴, optionally substituted C₁-C₆ alkyl, optionally substituted C₁-C₆ alkylsulfonyl, optionally substituted mono or bicyclic C₃-C₆ cycloalkyl, optionally substituted C₂-C₆ alkenyl, optionally substituted C₂-C₆ alkynyl, optionally substituted C₁-C₆ alkoxy, optionally substituted C₁-C₆ alkoxycarbonyl group, mono or bicyclic optionally substituted C₆-C₁₂ aryl, mono or bicyclic optionally substituted 5 to membered heteroaryl, optionally substituted mono or bicyclic 3 to 8 membered heterocycle, optionally substituted aryloxy, optionally substituted heteroaryloxy and optionally substituted heterocyclyloxy;

R⁵ and R⁷ are each independently selected from the group consisting of H, halogen, OH, CN, COOR¹³, CONR¹³R¹⁴, NR¹³R¹⁴, NR¹³COR¹⁴, optionally substituted C₁-C₆ alkyl, optionally substituted C₁-C₆ alkylsulfonyl, optionally substituted mono or bicyclic C₃-C₆ cycloalkyl, optionally substituted C₂-C₆ alkenyl, optionally substituted C₂-C₆ alkynyl, optionally substituted C₁-C₆ alkoxy, optionally substituted C₁-C₆ alkoxycarbonyl group, mono or bicyclic optionally substituted C₆-C₁₂ aryl, mono or bicyclic optionally substituted 5 to 10 membered heteroaryl, optionally substituted mono or bicyclic 3 to 8 membered heterocycle, optionally substituted aryloxy, optionally substituted heteroaryloxy, optionally substituted heterocyclyloxy and L⁵-L⁶-R¹⁶; wherein a maximum of one of R⁵ and R⁷ is -L⁵-L⁶-R¹⁶;

R¹³ and R¹⁴ are each independently selected from the group consisting of H, halogen, OH, CN, COOH, CONH₂, NH₂, NHCOH, optionally substituted C₁-C₆ alkyl, optionally substituted C₁-C₆ alkylsulfonyl, optionally substituted mono or bicyclic C₃-C₆ cycloalkyl, optionally substituted C₂-C₆ alkenyl, optionally substituted C₂-C₆ alkynyl, optionally substituted C₁-C₆ alkoxy, optionally substituted C₁-C₆ alkoxycarbonyl group, mono or bicyclic optionally substituted C₆-C₁₂ aryl, mono or bicyclic optionally substituted 5 to membered heteroaryl, optionally substituted mono or bicyclic 3 to 8 membered heterocycle, optionally substituted aryloxy, optionally substituted heteroaryloxy and optionally substituted heterocyclyloxy;

L¹ is absent or is NR¹⁷, O, an optionally substituted C₁-C₆ alkylene, an optionally substituted C₂-C₆ alkenylene, an optionally substituted C₂-C₆ alkynylene, an optionally substituted C₃-C₆ cycloalkylene, an optionally substituted C₆-C₁₂ arylene, an optionally substituted 5 to 10 membered heteroarylene or an optionally substituted 3 to 8 membered heterocyclylene;

L² is absent or is C═O, C═S, C═NR¹⁹ or SO₂;

L³ is absent or is NR¹⁸, O, an optionally substituted C₁-C₆ alkylene, an optionally substituted C₂-C₆ alkenylene, an optionally substituted C₂-C₆ alkynylene, an optionally substituted C₃-C₆ cycloalkylene, an optionally substituted C₆-C₁₂ arylene, an optionally substituted 5 to 10 membered heteroarylene or an optionally substituted 3 to 8 membered heterocyclylene;

L⁴ is absent or is an optionally substituted C₁-C₆ alkylene, an optionally substituted C₂-C₆ alkenylene, an optionally substituted C₂-C₆ alkynylene, an optionally substituted C₃-C₆ cycloalkylene, an optionally substituted C₆-C₁₂ arylene, an optionally substituted 5 to membered heteroarylene or an optionally substituted 3 to 8 membered heterocyclylene;

L⁵ is absent or an optionally substituted C₁-C₆ alkylene, an optionally substituted C₂-C₆ alkenylene, an optionally substituted C₂-C₆ alkynylene, O, S, S═O, SO₂ or NR¹⁹;

L⁶ is absent or an optionally substituted C₁-C₆ alkylene, an optionally substituted C₂-C₆ alkenylene, an optionally substituted C₂-C₆ alkynylene, O, S, S═O, SO₂ or NR¹⁹;

R¹⁵ is H, optionally substituted C₁-C₆ alkyl, optionally substituted C₂-C₆ alkenyl, optionally substituted C₂-C₆ alkynyl, optionally substituted mono or bicyclic C₃-C₆ cycloalkyl, mono or bicyclic optionally substituted C₆-C₁₂ aryl, mono or bicyclic optionally substituted 5 to 10 membered heteroaryl or optionally substituted mono or bicyclic 3 to 8 membered heterocycle;

R¹⁶ is H, optionally substituted C₂-C₆ alkenyl, optionally substituted C₂-C₆ alkynyl, optionally substituted mono or bicyclic C₃-C₆ cycloalkyl, mono or bicyclic optionally substituted C₆-C₁₂ aryl, mono or bicyclic optionally substituted 5 to 10 membered heteroaryl or optionally substituted mono or bicyclic 3 to 8 membered heterocycle; and

R¹⁷ to R¹⁹ are independently H, an optionally substituted C₁-C₆ alkyl, an optionally substituted C₂-C₆ alkenyl, an optionally substituted C₂-C₆ alkynyl or CN;

wherein, when X² is N, X³ is CR³; and

when L¹ is absent and L² is C═O, L³ is not NR¹⁸;

or a pharmaceutically acceptable complex, salt, solvate, tautomeric form or polymorphic form thereof.

The compounds of formula (I) may be used as medicaments.

Hence, in a second aspect, there is provided a compound of formula (I), or a pharmaceutically acceptable complex, salt, solvate, tautomeric form or polymorphic form thereof, for use as a medicament.

The inventors have found that compounds of formula (I) are useful in modulating the STimulator of INterferon Genes (STING) protein.

Hence, in a third aspect, there is provided a compound of formula (I), or a pharmaceutically acceptable complex, salt, solvate, tautomeric form or polymorphic form thereof, for use in modulating the STimulator of INterferon Genes (STING) protein.

Preferably, the compound of formula (I) is for use in inhibiting, or inactivating, the STING protein. The compound of formula (I) may be for use in inhibiting, or inactivating, STING functional activity as evidenced by a reduction of one or more biological effects selected from the group consisting of cellular interferon β production, cellular levels of interferon-stimulated genes, production of cytokines and phosphorylation of the transcription factors IRF-3 and NF-κB.

By inhibiting the STING protein, it is possible to treat, ameliorate or prevent liver fibrosis, fatty liver disease, pulmonary fibrosis, lupus, rheumatoid arthritis (RA), STING-associated vasculopathy with onset in infancy (SAVI), pancreatitis, cardiovascular disease, non-alcoholic fatty liver disease and renal fibrosis.

By inhibiting the STING protein, it is possible to treat, ameliorate or prevent liver fibrosis, fatty liver disease, non-alcoholic steatohepatitis (NASH), pulmonary fibrosis, lupus, rheumatoid arthritis (RA), STING-associated vasculopathy with onset in infancy (SAVI), Aicardi-Goutieres syndrome (AGS), familial chilblain lupus (FCL), systemic lupus erythematosus (SLE), retinal vasculopathy, neuroinflammation, systemic inflammatory response syndrome, pancreatitis, cardiovascular disease, renal fibrosis, stroke and age-related macular degeneration (AMD).

Accordingly, in a fourth aspect there is provided a compound of formula (I), or a pharmaceutically acceptable complex, salt, solvate, tautomeric form or polymorphic form thereof, for use in treating, ameliorating or preventing a disease selected from liver fibrosis, fatty liver disease, non-alcoholic steatohepatitis (NASH), pulmonary fibrosis, lupus, sepsis, rheumatoid arthritis (RA), type I diabetes, STING-associated vasculopathy with onset in infancy (SAVI), Aicardi-Goutieres syndrome (AGS), familial chilblain lupus (FCL), systemic lupus erythematosus (SLE), retinal vasculopathy, neuroinflammation, systemic inflammatory response syndrome, pancreatitis, cardiovascular disease, renal fibrosis, stroke and age-related macular degeneration (AMD).

In a fifth aspect, there is provided a method of modulating the STING protein in a subject, the method comprising administering, to a subject in need of such treatment, a therapeutically effective amount of a compound of formula (I), or a pharmaceutically acceptable complex, salt, solvate, tautomeric form or polymorphic form thereof. Preferably, the method comprises inhibiting the STING protein.

Preferably, the method is a method of inhibiting, or inactivating, the STING protein.

In a sixth aspect, there is provided a method of treating, ameliorating or preventing a disease selected from liver fibrosis, fatty liver disease, non-alcoholic steatohepatitis (NASH), pulmonary fibrosis, lupus, sepsis, rheumatoid arthritis (RA), type I diabetes, STING-associated vasculopathy with onset in infancy (SAVI), Aicardi-Goutieres syndrome (AGS), familial chilblain lupus (FCL), systemic lupus erythematosus (SLE), retinal vasculopathy, neuroinflammation, systemic inflammatory response syndrome, pancreatitis, cardiovascular disease, renal fibrosis, stroke and age-related macular degeneration (AMD); the method comprising administering, to a subject in need of such treatment, a therapeutically effective amount of a compound of formula (I), or a pharmaceutically acceptable complex, salt, solvate, tautomeric form or polymorphic form thereof.

It may be appreciated that the term “preventing” can mean “reducing the likelihood of”.

In one preferred embodiment, the disease is fibrosis. The fibrosis may be selected from the group consisting of liver fibrosis, pulmonary fibrosis or renal fibrosis. In some embodiments, the fibrosis patient may have upregulated STING expression and/or STING activity in a tissue compared to that of a healthy subject.

In an alternative preferred embodiment, the disease is fatty liver disease. The fatty liver disease may be non-alcoholic (or simple) fatty liver or non-alcoholic steatohepatitis (NASH).

The following definitions are used in connection with the compounds of the present invention unless the context indicates otherwise.

Throughout the description and the claims of this specification the word “comprise” and other forms of the word, such as “comprising” and “comprises,” means including but not limited to, and is not intended to exclude for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions.

“Optional” or “optionally” means that the subsequently described event, operation or circumstances can or cannot occur, and that the description includes instances where the event, operation or circumstance occurs and instances where it does not.

The term “alkyl” as used herein, unless otherwise specified, refers to a saturated straight or branched hydrocarbon. In certain embodiments, the alkyl group is a primary, secondary, or tertiary hydrocarbon. In certain embodiments, the alkyl group includes one to six carbon atoms, i.e. C₁-C₆ alkyl. C₁-C₆ alkyl includes for example methyl, ethyl, n-propyl (1-propyl) and isopropyl (2-propyl, 1-methylethyl), butyl, pentyl, hexyl, isobutyl, sec-butyl, tert-butyl, isopentyl, neopentyl and isohexyl. An alkyl group can be unsubstituted or substituted with one or more of halogen, OH, optionally substituted C₁-C₆ alkoxy, CN, oxo, C(O)R²⁰, COOR²⁰, OC(O)R²⁰, CONR²⁰R²¹, NR²OR²¹, NR²⁰C(O)R²¹, ═NOR²⁰, SR²⁰, SO₂R²⁰, OSO₂R²⁰, SO₂NR²⁰R²¹, OP(O)(OR²⁰)(OR²¹), optionally substituted C₆-C₁₂ aryl, optionally substituted 5 to 10 membered heteroaryl, optionally substituted C₃-C₆ cycloalkyl and optionally substituted 3 to 8 membered heterocycle. Accordingly, it will be appreciated that an optionally substituted C₁-C₆ alkyl may be an optionally substituted C₁-C₆ haloalkyl, i.e. a C₁-C₆ alkyl substituted with at least one halogen, and optionally further substituted with one or more of OH, optionally substituted C₁-C₆ alkoxy, CN, oxo, C(O)R²⁰, COOR²⁰, OC(O)R²⁰, CONR²⁰R²¹, NR²⁰R²¹, NR²⁰C(O)R²¹, ═NOR²⁰, SR²⁰, SO₂R²⁰, OSO₂R²⁰, SO₂NR²⁰R²¹, OP(O)(OR²⁰)(OR²¹), optionally substituted C₆-C₁₂ aryl, optionally substituted 5 to 10 membered heteroaryl, optionally substituted C₃-C₆ cycloalkyl and optionally substituted 3 to 8 membered heterocycle. The optionally substituted C₁-C₆ alkyl may be a polyfluoroalkyl, preferably a C₁-C₃ polyfluoroalkyl.

R²⁰ and R²¹ may each independently be selected from the group consisting of H, halogen, OH, CN, COOH, CONH₂, NH₂, NHCOH, optionally substituted C₁-C₆ alkyl, optionally substituted C₁-C₆ alkylsulfonyl, optionally substituted mono or bicyclic C₃-C₆ cycloalkyl, optionally substituted C₂-C₆ alkenyl, optionally substituted C₂-C₆ alkynyl, optionally substituted C₁-C₆ alkoxy, optionally substituted C₁-C₆ alkoxycarbonyl group, mono or bicyclic optionally substituted C₆-C₁₂ aryl, mono or bicyclic optionally substituted 5 to 10 membered heteroaryl, optionally substituted mono or bicyclic 3 to 8 membered heterocycle, optionally substituted aryloxy, optionally substituted heteroaryloxy and optionally substituted heterocyclyloxy. R²⁰ and R²¹ may each independently be selected from the group consisting of H and halogen.

The term “alkylene”, as used herein, unless otherwise specified, refers to a bivalent saturated straight or branched hydrocarbon. In certain embodiments, the alkylene group is a primary, secondary, or tertiary hydrocarbon. In certain embodiments, the alkylene group includes one to six carbon atoms, i.e. C₁-C₆ alkylene. C₁-C₆ alkylene includes for example methylene, ethylene, n-propylene and isopropylene, butylene, pentylene, hexylene, isobutylene, sec-butylene, tert-butylene, isopentylene, neopentylene, and isohexylene. An alkylene group can be unsubstituted or substituted with one or more of optionally substituted C₁-C₆ alkyl, halogen, OH, optionally substituted C₁-C₆ alkoxy, CN, oxo, C(O)R²⁰, COOR²⁰, OC(O)R²⁰, CONR²⁰R²¹, NR²OR²¹, NR²⁰C(O)R²¹, ═NOR²⁰, SR²⁰, SO₂R²⁰, OSO₂R²⁰, SO₂NR²OR²¹, OP(O)(OR²⁰)(OR²¹), optionally substituted C₆-C₁₂ aryl, optionally substituted 5 to 10 membered heteroaryl, optionally substituted C₃-C₆ cycloalkyl and optionally substituted 3 to 8 membered heterocycle. Accordingly, it will be appreciated that an optionally substituted C₁-C₆ alkylene may be an optionally substituted C₁-C₆ haloalkylene, i.e. a C₁-C₆ alkylene substituted with at least one halogen, and optionally further substituted with one or more of optionally substituted C₁-C₆ alkyl, OH, optionally substituted C₁-C₆ alkoxy, CN, oxo, C(O)R²⁰, COOR²⁰, OC(O)R²⁰, CONR²OR²¹, NR²⁰R²¹, NR²⁰C(O)R²¹, ═NOR²⁰, SR²⁰, SO₂R²⁰, OSO₂R²⁰, SO₂NR²⁰R²¹, OP(O)(OR²⁰)(OR²¹), optionally substituted C₆-C₁₂ aryl, optionally substituted 5 to 10 membered heteroaryl, optionally substituted C₃-C₆ cycloalkyl and optionally substituted 3 to 8 membered heterocycle. It will be appreciated that an optionally substituted C₁-C₆ alkylene may be an optionally substituted polyfluoroalkylene, preferably a C₁-C₃ polyfluoroalkylene. R²⁰ and R²¹ may be as defined above. R²⁰ and R²¹ may each independently be selected from the group consisting of H, halogen and optionally substituted C₁-C₆ alkyl.

The term “halo” or “halogen” includes fluoro (—F), chloro (—Cl), bromo (—Br) and iodo (—I).

The term “polyfluoroalkyl” may denote a C₁-C₃ alkyl group in which two or more hydrogen atoms are replaced by fluorine atoms. The term may include perfluoroalkyl groups, i.e. a C₁-C₃ alkyl group in which all the hydrogen atoms are replaced by fluorine atoms. Accordingly, the term C₁-C₃ polyfluoroalkyl includes, but is not limited to, difluoromethyl, trifluoromethyl, 2,2,2-trifluoroethyl, pentafluoroethyl, 3,3,3-trifluoropropyl, 2,2,3,3,3-pentafluoropropyl, and 2,2,2-trifluoro-1-(trifluoromethyl)ethyl.

“Alkoxy” refers to the group R²²—O—, where R²² is an optionally substituted C₁-C₆ alkyl group, an optionally substituted C₃-C₆ cycloalkyl group, an optionally substituted C₂-C₆ alkenyl or an optionally substituted C₂-C₆ alkynyl. Exemplary C₁-C₆ alkoxy groups include but are not limited to methoxy, ethoxy, n-propoxy (1-propoxy), n-butoxy and tert-butoxy. An alkoxy group can be unsubstituted or substituted with one or more of halogen, OH, CN, oxo, C(O)R²⁰, COOR²⁰, OC(O)R²⁰, CONR²OR²¹, NR²OR²¹, NR²⁰C(O)R²¹, ═NOR²⁰, SR²⁰, SO₂R²⁰, OSO₂R²⁰, SO₂NR²⁰R²¹, OP(O)(OR²⁰)(OR²¹), optionally substituted C₆-C₁₂ aryl, optionally substituted 5 to 10 membered heteroaryl, optionally substituted C₃-C₆ cycloalkyl and optionally substituted 3 to 8 membered heterocycle. R²⁰ and R²¹ may be as defined above. R²⁰ and R²¹ may each independently be selected from the group consisting of H, halogen and optionally substituted C₁-C₆ alkyl.

“Aryl” refers to an aromatic 6 to 12 membered hydrocarbon group. The term includes bicyclic groups where one of the rings is aromatic and the other is not. Examples of a C₆-C₁₂ aryl group include, but are not limited to, phenyl, α-naphthyl, β-naphthyl, biphenyl, tetrahydronaphthyl and indanyl. An aryl group can be unsubstituted or substituted with one or more of optionally substituted C₁-C₆ alkyl, optionally substituted C₂-C₆ alkenyl, optionally substituted C₂-C₆ alkynyl, optionally substituted C₁-C₆ alkoxy, halogen, OH, CN, oxo, C(O)R²⁰, COOR²⁰, OC(O)R²⁰, CONR²⁰R²¹, NR²⁰R²¹, NR²⁰C(O)R²¹, ═NOR²⁰, SR²⁰, SO₂R²⁰, OSO₂R²⁰, SO₂NR²⁰R²¹, OP(O)(OR²⁰)(OR²¹), optionally substituted C₆-C₁₂ aryl, optionally substituted 5 to 10 membered heteroaryl, optionally substituted C₃-C₆ cycloalkyl and optionally substituted 3 to 8 membered heterocycle. R²⁰ and R²¹ may be as defined above. R²⁰ and R²¹ may each independently be selected from the group consisting of H, halogen and optionally substituted C₁-C₆ alkyl.

“Arylene” refers to a bivalent aromatic 6 to 10 membered hydrocarbon group. An arylene group may be as defined above in relation the aryl group, but with a hydrogen atom removed therefrom to cause the group to be bivalent.

The term “bicycle” or “bicyclic” as used herein refers to a molecule that features two fused rings, which rings are a cycloalkyl, heterocyclyl, or heteroaryl. In one embodiment, the rings are fused across a bond between two atoms. The bicyclic moiety formed therefrom shares a bond between the rings. In another embodiment, the bicyclic moiety is formed by the fusion of two rings across a sequence of atoms of the rings to form a bridgehead. Similarly, a “bridge” is an unbranched chain of one or more atoms connecting two bridgeheads in a polycyclic compound. In another embodiment, the bicyclic molecule is a “spiro” or “spirocyclic” moiety. The spirocyclic group may be a C₃-C₆ cycloalkyl or a mono or bicyclic 3 to 8 membered heterocycle which is bound through a single carbon atom of the spirocyclic moiety to a single carbon atom of a carbocyclic or heterocyclic moiety. In one embodiment, the spirocyclic group is a cycloalkyl and is bound to another cycloalkyl. In another embodiment, the spirocyclic group is a cycloalkyl and is bound to a heterocyclyl. In a further embodiment, the spirocyclic group is a heterocyclyl and is bound to another heterocyclyl. In still another embodiment, the spirocyclic group is a heterocyclyl and is bound to a cycloalkyl. A spirocyclic group can be unsubstituted or substituted with one or more of optionally substituted C₁-C₆ alkyl, optionally substituted C₂-C₆ alkenyl, optionally substituted C₂-C₆ alkynyl, optionally substituted C₁-C₆ alkoxy, halogen, OH, CN, oxo, C(O)R²⁰, COOR²⁰, OC(O)R²⁰, CONR²OR²¹, NR²OR²¹, NR²⁰C(O)R²¹, ═NOR²⁰, SR²⁰, SO₂R²⁰, OSO₂R²⁰, SO₂NR²⁰R²¹, OP(O)(OR²⁰)(OR²¹), optionally substituted C₆-C₁₂ aryl, optionally substituted 5 to 10 membered heteroaryl, optionally substituted C₃-C₆ cycloalkyl and optionally substituted 3 to 8 membered heterocycle. R²⁰ and R²¹ may be as defined above. R²⁰ and R²¹ may each independently be selected from the group consisting of H, halogen and optionally substituted C₁-C₆ alkyl.

“Cycloalkyl” refers to a non-aromatic, saturated, partially saturated, monocyclic, bicyclic or polycyclic hydrocarbon 3 to 6 membered ring system. Representative examples of a C₃-C₆ cycloalkyl include, but are not limited to, cyclopropyl, cyclobutyl, cyclopentyl, cyclohexyl. A cycloalkyl group can be unsubstituted or substituted with one or more of optionally substituted C₁-C₆ alkyl, optionally substituted C₂-C₆ alkenyl, optionally substituted C₂-C₆ alkynyl, optionally substituted C₁-C₆ alkoxy, halogen, OH, CN, oxo, C(O)R²⁰, COOR²⁰, OC(O)R²⁰, CONR²OR²¹, NR²OR²¹, NR²⁰C(O)R²¹, ═NOR²⁰, SR²⁰, SO₂R²⁰, OSO₂R²⁰, SO₂NR²⁰R²¹, OP(O)(OR²⁰)(OR²¹), optionally substituted C₆-C₁₂ aryl, optionally substituted 5 to 10 membered heteroaryl, optionally substituted C₃-C₆ cycloalkyl and optionally substituted 3 to 8 membered heterocycle. R²⁰ and R²¹ may be as defined above. R²⁰ and R²¹ may each independently be selected from the group consisting of H, halogen and optionally substituted C₁-C₆ alkyl.

“Cycloalkylene” refers to a bivalent non-aromatic, saturated, partially saturated, monocyclic, bicyclic or polycyclic hydrocarbon 3 to 6 membered ring system. A cycloalkylene group may be as defined above in relation to the cycloalkyl group, but with a hydrogen atom removed therefrom to cause the group to be bivalent.

“Heteroaryl” refers to a monocyclic or bicyclic aromatic 5 to 10 membered ring system in which at least one ring atom is a heteroatom. The term includes bicyclic groups where one of the rings is aromatic and the other is not. The or each heteroatom may be independently selected from the group consisting of oxygen, sulfur and nitrogen. Examples of 5 to 10 membered heteroaryl groups include furan, thiophene, indole, azaindole, oxazole, thiazole, isoxazole, isothiazole, imidazole, N-methylimidazole, pyridine, pyrimidine, pyrazine, pyrrole, N-methylpyrrole, pyrazole, N-methylpyrazole, 1,3,4-oxadiazole, 1,2,4-triazole, 1-methyl-1,2,4-triazole, 1H-tetrazole, 1-methyltetrazole, benzoxazole, benzothiazole, benzofuran, benzisoxazole, benzimidazole, N-methylbenzimidazole, azabenzimidazole, indazole, quinazoline, quinoline, and isoquinoline. Bicyclic 5 to 10 membered heteroaryl groups include those where a phenyl, pyridine, pyrimidine, pyrazine or pyridazine ring is fused to a 5 or 6-membered monocyclic heteroaryl ring. A heteroaryl group can be unsubstituted or substituted with one or more of optionally substituted C₁-C₆ alkyl, optionally substituted C₂-C₆ alkenyl, optionally substituted C₂-C₆ alkynyl, optionally substituted C₁-C₆ alkoxy, halogen, OH, CN, oxo, C(O)R²⁰, COOR²⁰, OC(O)R²⁰, CONR²OR²¹, NR²⁰R²¹, NR²⁰C(O)R²¹, ═NOR²⁰, SR²⁰, SO₂R²⁰, OSO₂R²⁰, SO₂NR²⁰R²¹, OP(O)(OR²⁰)(OR²¹), optionally substituted C₆-C₁₂ aryl, optionally substituted 5 to 10 membered heteroaryl, optionally substituted C₃-C₆ cycloalkyl and optionally substituted 3 to 8 membered heterocycle. R²⁰ and R²¹ may be as defined above. R²⁰ and R²¹ may each independently be selected from the group consisting of H, halogen and optionally substituted C₁-C₆ alkyl.

“Heteroarylene” refers to a bivalent monocyclic or bicyclic aromatic 5 to 10 membered ring system in which at least one ring atom is a heteroatom. A heteroarylene group may be as defined above in relation to the heteroaryl group, but with a hydrogen atom removed therefrom to cause the group to be bivalent.

“Heterocycle” or “heterocyclyl” refers to 3 to 8 membered monocyclic, bicyclic or bridged molecules in which at least one ring atom is a heteroatom. The or each heteroatom may be independently selected from the group consisting of oxygen, sulfur and nitrogen. A heterocycle may be saturated or partially saturated. Exemplary 3 to 8 membered heterocycle groups include but are not limited to aziridine, oxirane, oxirene, thiirane, pyrroline, pyrrolidine, dihydrofuran, tetrahydrofuran, dihydrothiophene, tetrahydrothiophene, dithiolane, piperidine, 1,2,3,6-tetrahydropyridine-1-yl, tetrahydropyran, pyran, morpholine, piperazine, thiane, thiine, piperazine, azepane, diazepane and oxazine. A heterocycle group can be unsubstituted or substituted with one or more of optionally substituted C₁-C₆ alkyl, optionally substituted C₂-C₆ alkenyl, optionally substituted C₂-C₆ alkynyl, optionally substituted C₁-C₆ alkoxy, halogen, OH, CN, oxo, C(O)R²⁰, COOR²⁰, OC(O)R²⁰, CONR²OR²¹, NR²⁰R²¹, NR²⁰C(O)R²¹, ═NOR²⁰, SR²⁰, SO₂R²⁰, OSO₂R²⁰, SO₂NR²⁰R²¹, OP(O)(OR²⁰)(OR²¹), optionally substituted C₆-C₁₂ aryl, optionally substituted 5 to 10 membered heteroaryl, optionally substituted C₃-C₆ cycloalkyl and optionally substituted 3 to 8 membered heterocycle. R²⁰ and R²¹ may be as defined above. R²⁰ and R²¹ may each independently be selected from the group consisting of H, halogen and optionally substituted C₁-C₆ alkyl.

“Heterocyclylene” refers to a bivalent 3 to 8 membered monocyclic, bicyclic or bridged molecules in which at least one ring atom is a heteroatom. A heterocyclylene group may be as defined above in relation to the heterocycle group, but with a hydrogen atom removed therefrom to cause the group to be bivalent.

“Alkenyl” refers to an olefinically unsaturated hydrocarbon groups which can be unbranched or branched. In certain embodiments, the alkenyl group has 2 to 6 carbons, i.e. it is a C₂-C₆ alkenyl. C₂-C₆ alkenyl includes for example vinyl, allyl, propenyl, butenyl, pentenyl and hexenyl. An alkenyl group can be unsubstituted or substituted with one or more of optionally substituted C₂-C₆ alkynyl, optionally substituted C₁-C₆ alkoxy, halogen, OH, CN, oxo, C(O)R²⁰, COOR²⁰, OC(O)R²⁰, CONR²OR²¹, NR²OR²¹, NR²⁰C(O)R²¹, ═NOR²⁰, SR²⁰, SO₂R²⁰, OSO₂R²⁰, SO₂NR²OR²¹, OP(O)(OR²⁰)(OR²¹), optionally substituted C₆-C₁₂ aryl, optionally substituted 5 to 10 membered heteroaryl, optionally substituted C₃-C₆ cycloalkyl and optionally substituted 3 to 8 membered heterocycle. R²⁰ and R²¹ may be as defined above. R²⁰ and R²¹ may each independently be selected from the group consisting of H, halogen and optionally substituted C₁-C₆ alkyl.

“Alkynyl” refers to an acetylenically unsaturated hydrocarbon groups which can be unbranched or branched. In certain embodiments, the alkynyl group has 2 to 6 carbons, i.e. it is a C₂-C₆ alkynyl. C₂-C₆ alkynyl includes for example propargyl, propynyl, butynyl, pentynyl and hexynyl. An alkynyl group can be unsubstituted or substituted with one or more of optionally substituted C₂-C₆ alkenyl, optionally substituted C₁-C₆ alkoxy, halogen, OH, CN, oxo, C(O)R²⁰, COOR²⁰, OC(O)R²⁰, CONR²OR²¹, NR²⁰R²¹, NR²⁰C(O)R²¹, ═NOR²⁰, SR²⁰, SO₂R²⁰, OSO₂R²⁰, SO₂NR²OR²¹, OP(O)(OR²⁰)(OR²¹), optionally substituted C₆-C₁₂ aryl, optionally substituted 5 to 10 membered heteroaryl, optionally substituted C₃-C₆ cycloalkyl and optionally substituted 3 to 8 membered heterocycle. R²⁰ and R²¹ may be as defined above. R²⁰ and R²¹ may each independently be selected from the group consisting of H, halogen and optionally substituted C₁-C₆ alkyl.

The term “alkenylene”, as used herein, unless otherwise specified, refers to a bivalent olefinically unsaturated straight or branched hydrocarbon. An alkenylene group may be as defined above in relation the alkenyl group, but with a hydrogen atom removed therefrom to cause the group to be bivalent.

The term “alkynylene”, as used herein, unless otherwise specified, refers to a bivalent acetylenically unsaturated straight or branched hydrocarbon. An alkynylene group may be as defined above in relation the alkynyl group, but with a hydrogen atom removed therefrom to cause the group to be bivalent.

“Alkylsulfonyl” refers to the group alkyl-SO₂— where alkyl is an optionally substituted C₁-C₆ alkyl, and is as defined as above.

“Alkoxycarbonyl” refers to the group alkyl-O—C(O)—, where alkyl is an optionally substituted C₁-C₆ alkyl. An alkoxycarbonyl group can be unsubstituted or substituted with one or more of optionally substituted C₂-C₆ alkenyl, optionally substituted C₂-C₆ alkynyl, optionally substituted C₁-C₆ alkoxy, halogen, OH, CN, oxo, C(O)R²⁰, COOR²⁰, OC(O)R²⁰, CONR²OR²¹, NR²⁰R²¹, NR²⁰C(O)R²¹, ═NOR²⁰, SR²⁰, SO₂R²⁰, OSO₂R²⁰, SO₂NR²⁰R²¹, OP(O)(OR²⁰)(OR²¹), optionally substituted C₆-C₁₂ aryl, optionally substituted 5 to 10 membered heteroaryl, optionally substituted C₃-C₆ cycloalkyl and optionally substituted 3 to 8 membered heterocycle.

“Aryloxy” refers to the group Ar—O— where Ar is a mono or bicyclic optionally substituted C₆-C₁₂ aryl group, as defined above.

“Heteroaryloxy” refers to the group heteroaryl-O— where the heteroaryl is a mono or bicyclic optionally substituted 5 to 10 membered heteroaryl, and is as defined above.

“Heterocyclyloxy” refers to the group heterocycle-O— where heterocycle is an optionally substituted mono or bicyclic 3 to 8 membered heterocycle, and is as defined as above.

A complex of the compound of formula (I) may be understood to be a multi-component complex, wherein the drug and at least one other component are present in stoichiometric or non-stoichiometric amounts. The complex may be other than a salt or solvate. Complexes of this type include clathrates (drug-host inclusion complexes) and co-crystals. The latter are typically defined as crystalline complexes of neutral molecular constituents which are bound together through non-covalent interactions, but could also be a complex of a neutral molecule with a salt. Co-crystals may be prepared by melt crystallisation, by recrystallisation from solvents, or by physically grinding the components together—see Chem Commun, S, 1889-1896, by O. Almarsson and M. J. Zaworotko (2004), incorporated herein by reference. For a general review of multi-component complexes, see J Pharm Sci, 64 (8), 1269-1288, by Haleblian (August 1975), incorporated herein by reference.

The term “pharmaceutically acceptable salt” may be understood to refer to any salt of a compound provided herein which retains its biological properties and which is not toxic or otherwise undesirable for pharmaceutical use. Such salts may be derived from a variety of organic and inorganic counter-ions well known in the art. Such salts include, but are not limited to: (1) acid addition salts formed with organic or inorganic acids such as hydrochloric, hydrobromic, sulfuric, nitric, phosphoric, sulfamic, acetic, adepic, aspartic, trifluoroacetic, trichloroacetic, propionic, hexanoic, cyclopentylpropionic, glycolic, glutaric, pyruvic, lactic, malonic, succinic, sorbic, ascorbic, malic, maleic, fumaric, tartaric, citric, benzoic, 3-(4-hydroxybenzoyl)benzoic, picric, cinnamic, mandelic, phthalic, lauric, methanesulfonic, ethanesulfonic, 1,2-ethane-disulfonic, 2-hydroxyethanesulfonic, benzenesulfonic, 4-chlorobenzenesulfonic, 2-naphthalenesulfonic, 4-toluenesulfonic, camphoric, camphorsulfonic, 4-methylbicyclo[2.2.2]-oct-2-ene-1-carboxylic, glucoheptonic, 3-phenylpropionic, trimethylacetic, tert-butylacetic, lauryl sulfuric, gluconic, benzoic, glutamic, hydroxynaphthoic, salicylic, stearic, cyclohexylsulfamic, quinic, muconic acid and the like acids; or (2) base addition salts formed when an acidic proton present in the parent compound either (a) is replaced by a metal ion, e.g., an alkali metal ion, an alkaline earth ion or an aluminium ion, or alkali metal or alkaline earth metal hydroxides, such as sodium, potassium, calcium, magnesium, aluminium, lithium, zinc, and barium hydroxide, ammonia or (b) coordinates with an organic base, such as aliphatic, alicyclic, or aromatic organic amines, such as ammonia, methylamine, dimethylamine, diethylamine, picoline, ethanolamine, diethanolamine, triethanolamine, ethylenediamine, lysine, arginine, ornithine, choline, N,N′-dibenzylethylene-diamine, chloroprocaine, diethanolamine, procaine, N-benzylphenethylamine, N-methylglucamine piperazine, tris(hydroxymethyl)-aminomethane, tetramethylammonium hydroxide, and the like.

Pharmaceutically acceptable salts may include, sodium, potassium, calcium, magnesium, ammonium, tetraalkylammonium and the like, and when the compound contains a basic functionality, salts of non-toxic organic or inorganic acids, such as hydrohalides, e.g. hydrochloride, hydrobromide and hydroiodide, carbonate or bicarbonate, sulfate or bisulfate, borate, phosphate, hydrogen phosphate, dihydrogen phosphate, pyroglutamate, saccharate, stearate, sulfamate, nitrate, orotate, oxalate, palmitate, pamoate, acetate, trifluoroacetate, trichloroacetate, propionate, hexanoate, cyclopentylpropionate, glycolate, glutarate, pyruvate, lactate, malonate, succinate, tannate, tartrate, tosylate, sorbate, ascorbate, malate, maleate, fumarate, tartarate, camsylate, citrate, cyclamate, benzoate, isethionate, esylate, formate, 3-(4-hydroxybenzoyl)benzoate, picrate, cinnamate, mandelate, phthalate, laurate, methanesulfonate (mesylate), methylsulphate, naphthylate, 2-napsylate, nicotinate, ethanesulfonate, 1,2-ethane-disulfonate, 2-hydroxyethanesulfonate, benzenesulfonate (besylate), 4-chlorobenzenesulfonate, 2-naphthalenesulfonate, 4-toluenesulfonate, camphorate, camphorsulfonate, 4-methylbicyclo[2.2.2]-oct-2-ene-1-carboxylate, glucoheptonate, 3-phenylpropionate, trimethylacetate, tert-butylacetate, lauryl sulfate, gluceptate, gluconate, glucoronate, hexafluorophosphate, hibenzate, benzoate, glutamate, hydroxynaphthoate, salicylate, stearate, cyclohexylsulfamate, quinate, muconate, xinofoate and the like.

Hemisalts of acids and bases may also be formed, for example, hemisulphate salts.

The skilled person will appreciate that the aforementioned salts include ones wherein the counterion is optically active, for example D-lactate, or racemic, for example DL-tartrate.

For a review on suitable salts, see “Handbook of Pharmaceutical Salts: Properties, Selection, and Use” by Stahl and Wermuth (Wiley-VCH, Weinheim, Germany, 2002). Pharmaceutically acceptable salts of compounds of formula (I) may be prepared by one or more of three methods:
(i) by reacting the compound of formula (I) with the desired acid or base;
(ii) by removing an acid- or base-labile protecting group from a suitable precursor of the compound of formula (I) using the desired acid or base; or
(iii) by converting one salt of the compound of formula (I) to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionisation in the resulting salt may vary from completely ionised to almost non-ionised.

The term “solvate” may be understood to refer to a compound provided herein or a salt thereof, that further includes a stoichiometric or non-stoichiometric amount of solvent bound by non-covalent intermolecular forces. Where the solvent is water, the solvate is a hydrate. Pharmaceutically acceptable solvates in accordance with the invention include those wherein the solvent of crystallization may be isotopically substituted, e.g. D₂O, d₆-acetone and d₆-DMSO.

A currently accepted classification system for organic hydrates is one that defines isolated site, channel, or metal-ion coordinated hydrates—see Polymorphism in Pharmaceutical Solids by K. R. Morris (Ed. H. G. Brittain, Marcel Dekker, 1995), incorporated herein by reference. Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm.

The compounds of the invention may exist in a continuum of solid states ranging from fully amorphous to fully crystalline, including polymorphs of said crystalline material. The term ‘amorphous’ refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterised by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterised by a phase change, typically first order (‘melting point’).

The compounds of the invention may also exist in a mesomorphic state (mesophase or liquid crystal) when subjected to suitable conditions. The mesomorphic state is intermediate between the true crystalline state and the true liquid state (either melt or solution). Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphilic’ and consist of molecules which possess an ionic (such as —COO—Na⁺, —COO—K⁺, or —SO₃—Na⁺) or non-ionic (such as —N⁻N⁺(CH₃)₃) polar head group. For more information, see Crystals and the Polarizing Microscope by N. H. Hartshorne and A. Stuart, 4^{th} Edition (Edward Arnold, 1970), incorporated herein by reference.

Compounds of formula (I) may include one or more stereogenic centers and so may exist as optical isomers, such as enantiomers and diastereomers. All such isomers and mixtures thereof are included within the scope of the present invention.

It will be understood that the above compounds may exist as enantiomers and as diastereoisomeric pairs. These isomers also represent further embodiments of the invention.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of formula (I) contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from o to 50% by volume of isopropanol, typically from 2% to 20%, and from 0 to 5% by volume of an alkylamine, typically 0.1% diethylamine. Concentration of the eluate affords the enriched mixture.

Mixtures of stereoisomers may be separated by conventional techniques known to those skilled in the art; see, for example, “Stereochemistry of Organic Compounds” by E. L. Eliel and S. H. Wilen (Wiley, New York, 1994).

R¹ may be H, halogen, OH, CN, optionally substituted C₁-C₆ alkyl, optionally substituted C₂-C₆ alkenyl or optionally substituted C₂-C₆ alkynyl. R¹ may be H, halogen, OH, CN, C₁-C₃ alkyl, C₂-C₃ alkenyl or C₂-C₃ alkynyl. Preferably, R¹ is H.

X² may be CR².

X³ may be CR³.

In one embodiment X² is N and X³ is CR³. In this embodiment, R³ is -L¹-L²-L³-L⁴-R¹⁵.

In an alternative embodiment, X² is CR² and X³ is N. In this embodiment, R² is -L¹-L²-L³-L⁴-R¹⁵.

However, in a preferred embodiment, X² is CR² and X³ is CR³. In some embodiments, R² is -L¹-L²-L³-L⁴-R¹⁵. In alternative embodiments, R³ is -L¹-L²-L³-L⁴-R¹⁵. Accordingly, the compound may be a compound of Formula (Ia) or Formula (Ib):

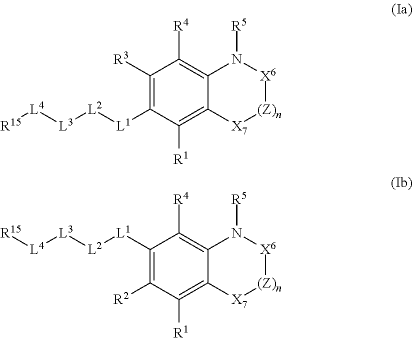

Preferably, one of R² and R³ is -L¹-L²-L³-L⁴-R¹⁵ and the other of R² and R³ is H, halogen, OH, CN, COOR¹³, CONR¹³R¹⁴, NR¹³R¹⁴, NR¹³COR¹⁴, optionally substituted C₁-C₆ alkyl, optionally substituted C₂-C₆ alkenyl or optionally substituted C₂-C₆ alkynyl, and R¹³ and R¹⁴ are each independently selected from the group consisting of H, optionally substituted C₁-C₃ alkyl, optionally substituted C₂-C₃ alkenyl and optionally substituted C₂—C alkynyl. More preferably, one of R² and R³ is -L¹-L²-L³-L⁴-R¹⁵ and the other of R² and R³ is H, halogen, OH, CN, CONR¹³R¹⁴, NR¹³R¹⁴, C₁-C₃ alkyl, C₂-C₃ alkenyl or C₂-C₃ alkynyl, and R¹³ and R¹⁴ are each independently selected from the group consisting of H, C₁-C₃ alkyl, C₂-C₃ alkenyl and C₂—C alkynyl. Preferably, one of R² and R³ is -L¹-L²-L³-L⁴-R¹⁵ and the other of R² and R³ is H, bromine or CONH₂. In a preferred embodiment, one of R² and R³ is -L¹-L²-L³-L⁴-R¹⁵ and the other of R² and R³ is H.

Preferably at least one of L¹ to L⁴ is present.

In some embodiments, Li is absent or is NR¹⁷. L² may be C═O, C═S, C═NR¹⁹ or SO₂. L³ may be absent or is NR¹⁸. Accordingly, in some embodiments, -L¹-L²-L³- may be

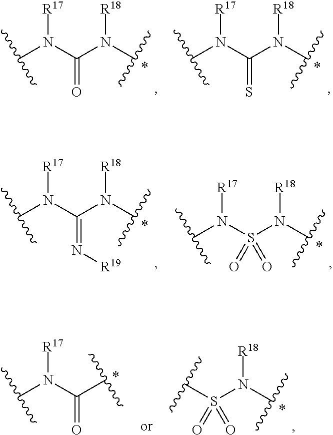

where an asterisk indicates the point of bonding to L⁴ or, in embodiments where L⁴ is absent, R¹⁵.

R¹⁷ and R¹⁸ may independently be H, optionally substituted C₁-C₆ alkyl, optionally substituted C₂-C₆ alkenyl or optionally substituted C₂-C₆ alkynyl. R¹⁷ and R¹⁸ may independently be H, C₁-C₃ alkyl, C₂-C₃ alkenyl or C₂-C₃ alkynyl. Preferably, R¹⁷ and R¹⁸ are H or methyl.

R¹⁹ may be H, C₁-C₃ alkyl, C₂-C₃ alkenyl, C₂-C₃ alkynyl or CN. R¹⁹ may be H, methyl or CN. Preferably, R¹⁹ is H or CN.

In an alternative embodiment, L¹ is absent or is an optionally substituted C₁-C₆ alkylene, an optionally substituted C₂-C₆ alkenylene or an optionally substituted C₂-C₆ alkynylene. Preferably, L¹ is absent or a C₁-C₃ alkylene. L¹ may be absent or CH₂. L² may be absent. L³ may be O. Accordingly, in some embodiments, -L¹-L²-L³- may be —O—* or —CH₂O—*, where an asterisk indicates the point of bonding to L⁴ or, in embodiments where L⁴ is absent, R¹⁵.

In a further alternative embodiment, L¹ may be an optionally substituted C₃-C₆ cycloalkylene, an optionally substituted C₆-C₁₂ arylene, an optionally substituted 5 to 10 membered heteroarylene or an optionally substituted 3 to 8 membered heterocyclylene. L¹ may be an optionally substituted C₃-C₆ cycloalkylene, an optionally substituted C₆ arylene, an optionally substituted 5 or 6 membered heteroarylene or an optionally substituted 3 to 6 membered heterocyclylene. L¹ may be a C₅-C₆ cycloalkylene, a C₆ arylene, a 5 or 6 membered heteroarylene or a 5 to 6 membered heterocyclylene. The cycloalkylene may be cyclopropylene, cyclobutylene, cyclopentylene or cyclohexylene. L¹ may be a 5 membered heteroarylene. The heteroarylene may be pyrrolylene, pyrazolylene, imidazolylene, 1,2,4-triazolylene, 1,2,3-triazolylene, furanylene, thiophenylene, oxazolylene, isoxazolylene, thiazolylene or isothiazolylene. L¹ may be a 6 membered heterocyclylene. The heterocyclylene may be pyrrolidinylene, pyrazolidinylene, imidazolidinylene, tetrahydrofuranylene, a,3-dioxolanylene, tetrahydrothiophenylene, piperidinylene, piperazinylene, tetrahydropyranylene, thianylene, morpholinylene or thiomorpholinylene. L² may be absent. L³ may be absent. Accordingly, in some embodiments, -L¹-L²-L³- may be

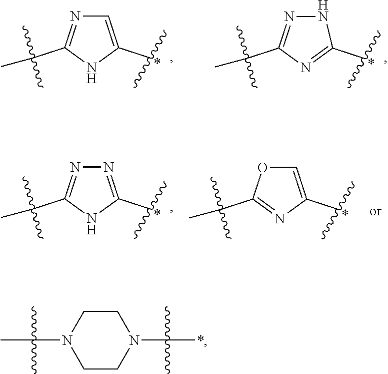

where an asterisk indicates the point of bonding to L⁴ or, in embodiments where L⁴ is absent, R¹⁵.

In some embodiments, L⁴ is absent, an optionally substituted C₁-C₆ alkylene, an optionally substituted C₂-C₆ alkenylene or an optionally substituted C₂-C₆ alkynylene. Preferably, L⁴ is absent or a C₁-C₃ alkenylene. More preferably, L⁴ is absent or is CH₂, CH₂CH₂ or CH₂CH₂CH₂.

In alternative embodiments, L⁴ is an optionally substituted C₃-C₆ cycloalkylene, an optionally substituted C₆-C₁₂ arylene, an optionally substituted 5 to 10 membered heteroarylene or an optionally substituted 3 to 8 membered heterocyclylene. Preferably, L⁴ is an optionally substituted C₃-C₆ cycloalkylene, an optionally substituted C₆ arylene, an optionally substituted 5 to 6 membered heteroarylene or an optionally substituted 3 to 6 membered heterocyclylene. More preferably, L⁴ is a C₅-C₆ cycloalkylene, a C₆ arylene, a 5 to 6 membered heteroarylene or a 5 to 6 membered heterocyclylene. The cycloalkylene may be cyclopropylene, cyclobutylene, cyclopentylene or cyclohexylene. L⁴ may be a 5 membered heteroarylene. The heteroarylene may be pyrrolylene, pyrazolylene, imidazolylene, 1,2,4-triazolylene, 1,2,3-triazolylene, furanylene, thiophenylene, oxazolylene, isoxazolylene, thiazolylene or isothiazolylene. The heterocyclylene may be pyrrolidinylene, pyrazolidinylene, imidazolidinylene, tetrahydrofuranylene, a,3-dioxolanylene, tetrahydrothiophenylene, piperidinylene, piperazinylene, tetrahydropyranylene, thianylene, morpholinylene or thiomorpholinylene. Accordingly, in some embodiments, L⁴ may be

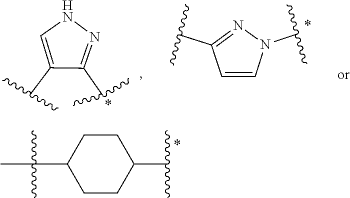

where an asterisk indicates the point of bonding to R¹⁵.

Accordingly, in some embodiments, -L¹-L²-L³-L⁴- may be —OCH₂CH₂—*, —CH₂OCH₂—*,

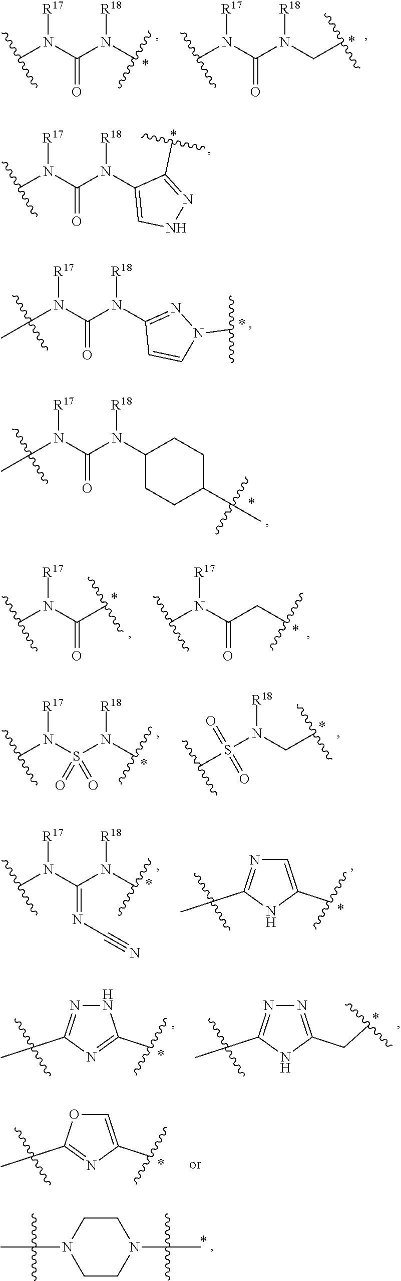

where an asterisk indicates the point of bonding to R¹⁵. Preferably, R¹⁷ and R¹⁸ are independently H or CH₃.

In one embodiment, R¹⁵ is a mono or bicyclic optionally substituted C₆-C₁₂ aryl. The optionally substituted C₆-C₁₂ aryl may be an optionally substituted phenyl, 5,6,7,8-tetrahydronaphthalenyl or 2,3-dihydro-1H-indenyl. The aryl may be unsubstituted or substituted with one or more substituents selected from the group consisting of optionally substituted C₁-C₆ alkyl, halogen, OH, oxo, OP(O)(OR²⁰)(OR²¹), optionally substituted C₁-C₆ alkoxy, NR²⁰R²¹, CONR²OR²¹, CN, C(O)R²⁰, COOR²⁰, NO₂, azido, SO₂R²⁰, C(O)R²⁰ and NR²⁰COR²¹. When the aryl is substituted with an optionally substituted alkyl, the alkyl may be unsubstituted or substituted with one or more substituents selected from the group consisting of halogen, OH, C₁-C₆ alkoxy, NR²⁰R²¹, C(O)R²⁰, CN, oxo, OP(O)(OR²⁰)(OR²¹), OC(O)R²⁰, COOR²⁰, C₁-C₆ alkenyl, C₁-C₆ alkynyl, ═NOR²⁰, NR²⁰C(O)R²¹, SO₂R²⁰ and SO₂NR²OR²¹. Halogen may be F. R²⁰ and R²¹ may independently be H or methyl. Accordingly, the aryl may be substituted with one or more substituents selected from the group consisting of F, CN, NH₂, C(O)CH₃, CONH₂, CH₃ and CH₂COOH.

In an alternative embodiment, R¹⁵ is a mono or bicyclic optionally substituted 5 to 10 membered heteroaryl, an optionally substituted C₃-C₆ cycloalkyl or an optionally substituted 3 to 8 membered heterocycle. The optionally substituted 5 to 10 membered heteroaryl may be optionally substituted pyrrolyl, optionally substituted furanyl, optionally substituted thiophenyl, optionally substituted oxazolyl, optionally substituted thiazolyl, optionally substituted isoxazolyl, optionally substituted isothiazolyl, optionally substituted imidazolyl, optionally substituted pyrazolyl, optionally substituted pyridinyl, optionally substituted pyridazinyl, optionally substituted pyrimidinyl, optionally substituted pyrazinyl, optionally substituted indolinyl, optionally substituted indolinyl, optionally substituted 1H-indolyl, optionally substituted 7-azaindolyl, optionally substituted 1H-pyrrolo[3,2-b]pyridinyl, optionally substituted benzofuranyl, optionally substituted azaindolyl, optionally substituted benzisoxazolyl, optionally substituted azabenzimidazolyl, optionally substituted indazolyl, optionally substituted benzo[b]thiophenyl, optionally substituted benzimidazolyl, optionally substituted, benzo[d]oxazolyl, optionally substituted benzo[d]thiazolyl, optionally substituted 1,4-benzodioxanyl, optionally substituted 1,2,3,4-tetrahydroquinolinyl, optionally substituted quinazolinyl, optionally substituted quinolinyl, optionally substituted isoquinolinyl, optionally substituted 1,2,3,4-tetrahydroisoquinolinyl, optionally substituted 3,4-dihydro-2H-1,4-benzoxazyl or optionally substituted 7,8-dihydropyrido[4,3-d]pyrimidinyl. The optionally substituted 3 to 8 membered heterocycle may be optionally substituted tetrahydrofuranyl, optionally substituted tetrahydrothiophenyl, optionally substituted pyrrolidinyl, optionally substituted piperidinyl, optionally substituted piperazinyl, optionally substituted tetrahydropyranyl, optionally substituted thianyl, optionally substituted morpholinyl, optionally substituted thiomorpholinyl, optionally substituted 1,2-oxazinyl, optionally substituted 1,3-oxazinyl, optionally substituted 1,4-oxazinyl, optionally substituted azepanyl, optionally substituted 1,2-diazepinyl, optionally substituted 1,3-diazepinyl, optionally substituted 1,4-diazepinyl or optionally substituted 3,4-dihydro-2H-benzo[b][1,4]oxazine. The heteroaryl, cycloalkyl or heterocycle may be unsubstituted or substituted with one or more substituents selected from the group consisting of optionally substituted C₁-C₆ alkyl, halogen, OH, oxo, OP(O)(OR²⁰)(OR²¹), optionally substituted C₁-C₆ alkoxy, NR²OR²¹, CONR²⁰R²¹, CN, C(O)R²⁰, COOR²⁰, NO₂, azido, SO₂R²⁰, C(O)R²⁰ and NR²⁰COR²¹. When the heteroaryl, cycloalkyl or heterocycle is substituted with an optionally substituted alkyl, the alkyl may be unsubstituted or substituted with one or more substituents selected from the group consisting of halogen, OH, C₁-C₆ alkoxy, NR²⁰R²¹, C(O)R²⁰, CN, oxo, OP(O)(OR²⁰)(OR²¹), OC(O)R²⁰, COOR²⁰, CONR²OR²¹, C₁-C₆ alkenyl, C₁-C₆ alkynyl, ═NOR²⁰, NR²⁰C(O)R²¹, SO₂R²⁰ and SO₂NR²⁰R²¹. Halogen may be F or Cl. Preferably, halogen is F. R²⁰ and R²¹ may independently be H or methyl. Accordingly, the heteroaryl, cycloalkyl or heterocycle may be substituted with one or more substituents selected from the group consisting of F, oxo, CN, NH₂, C(O)CH₃, CONH₂, CH₃ and CH₂COOH. For instance, the optionally substituted 5 to 10 membered heteroaryl may be optionally substituted with a methyl group, and optionally one or more further substituents. Accordingly, the optionally substituted 5 to 10 membered heteroaryl may be an optionally substituted 1-methylindolyl, an optionally substituted 2-methyl-1H-indolyl, an optionally substituted 5-methyl-1H-indolyl, optionally substituted N-methylimidazolyl, optionally substituted N-methylpyrazolyl or optionally substituted N-methylbenzimidazolyl.

Accordingly, R¹⁵ may be phenyl,

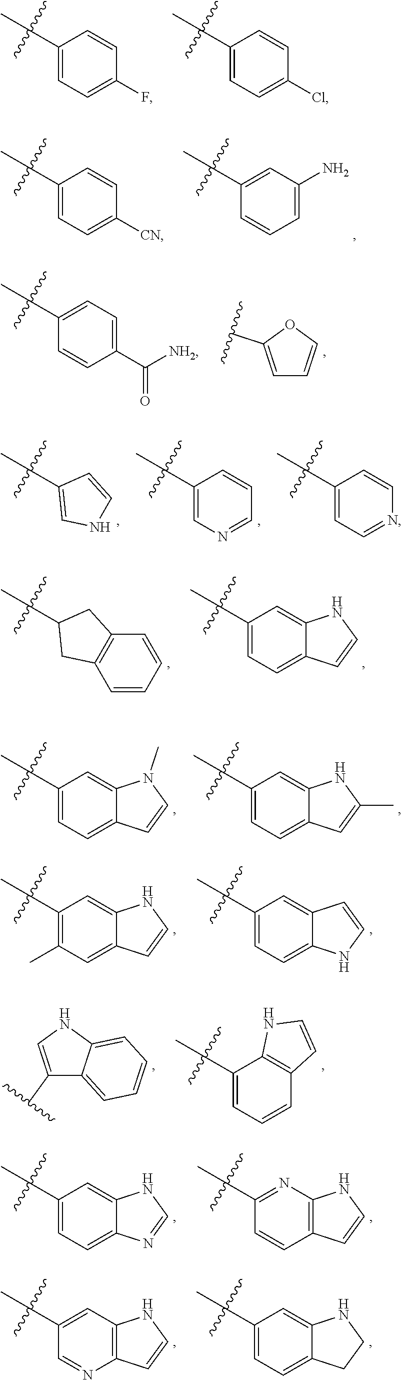

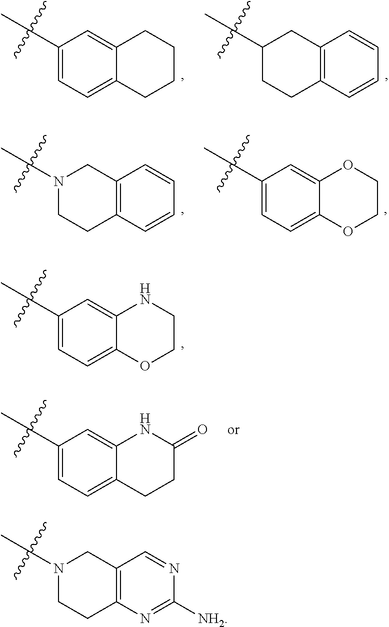

In some embodiments, R¹⁵ is 1H-indolyl or a phenyl substituted with NR²OR²¹. Preferably, R¹⁵ is 1H-indolyl or a phenyl substituted with NH₂.

R⁴ may be H, halogen, OH, CN, optionally substituted C₁-C₆ alkyl, optionally substituted C₂-C₆ alkenyl or optionally substituted C₂-C₆ alkynyl. R⁴ may be H, halogen, OH, CN, C₁-C₃ alkyl, C₂-C₃ alkenyl or C₂-C₃ alkynyl. Preferably, R⁴ is H.

R⁵ may be -L⁵-L⁶-R¹⁶.

Preferably, L⁵ is an optionally substituted C₁-C₃ alkylene, an optionally substituted C₂-C₃ alkenylene or an optionally substituted C₂-C₃ alkynylene. The alkylene, alkenylene or alkynylene may be unsubstituted or substituted with one or more of halogen, OH, CN, C(O)R²⁰, COOR²⁰, OC(O)R²⁰, CONR²OR²¹, NR²OR²¹, NR²⁰C(O)R²¹, ═NOR²⁰, SR²⁰, SO₂R²⁰, OSO₂R²⁰, SO₂NR²⁰R²¹ and oxo. R²⁰ and R²¹ may be independently be H, optionally substituted C₁-C₃ alkyl, optionally substituted C₂-C₃ alkenyl, optionally substituted C₂-C₃ alkynyl, optionally substituted mono or bicyclic C₃-C₆ cycloalkyl or optionally substituted mono or bicyclic 3 to 8 membered heterocycle. Preferably, R²⁰ and R²¹ are independently H, methyl or cyclopropyl. Preferably, L⁵ is CH₂, CH₂CH₂, CO,

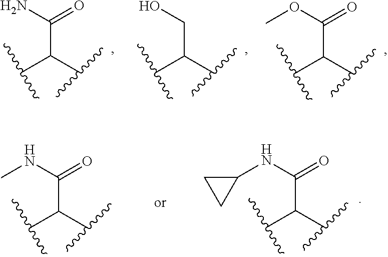

Alternatively, L⁵ may be absent.

In some embodiments, L⁶ is absent.

Alternatively, L⁶ may be O, S, S═O, SO₂ or NR¹⁹. R¹⁹ may be H, an optionally substituted C₁-C₃ alkyl, an optionally substituted C₂-C₃ alkenyl or an optionally substituted C₂-C₃ alkynyl. Preferably, L⁶ is O or S, and most preferably is O.

R¹⁶ may be optionally substituted mono or bicyclic C₃-C₆ cycloalkyl, mono or bicyclic optionally substituted C₆-C₁₂ aryl, mono or bicyclic optionally substituted 5 to 10 membered heteroaryl or optionally substituted mono or bicyclic 3 to 8 membered heterocycle. Preferably, R¹⁶ is a mono or bicyclic optionally substituted C₆-C₁₂ aryl, a mono or bicyclic optionally substituted 5 to 10 membered heteroaryl or optionally substituted mono or bicyclic 3 to 8 membered heterocycle. Mono or bicyclic optionally substituted C₆-C₁₂ aryl may be optionally substituted phenyl. Optionally substituted mono or bicyclic C₃-C₆ cycloalkyl may be cyclopropyl, cyclobutyl, cyclopentyl or cyclohexyl. Mono or bicyclic optionally substituted 5 to 10 membered heteroaryl may be optionally substituted oxazolyl, optionally substituted thiazolyl, optionally substituted isoxazolyl, optionally substituted isothiazolyl, optionally substituted imidazolyl, optionally substituted pyrazolyl, optionally substituted 1,2,3-oxadiazolyl, optionally substituted 1,2,4-oxadiazolyl, optionally substituted 1,2,5-oxadiazolyl, optionally substituted 1,3,4-oxadiazolyl, optionally substituted pyridinyl, optionally substituted pyridazinyl, optionally substituted pyrimidinyl, optionally substituted pyrazinyl, optionally substituted 1H-indolyl, optionally substituted azaindolyl, optionally substituted benzisoxazolyl, optionally substituted 4-azabenzimidazolyl, optionally substituted 5-benzimidazolyl, optionally substituted indazolyl, optionally substituted benzimidazolyl, optionally substituted benzofuranyl, optionally substituted benzo[b]thiophenyl, optionally substituted benzo[d]isoxazolyl, optionally substituted benzo[d]isothiazolyl, optionally substituted imidazo[1,2-a]pyridinyl, optionally substituted quinazolinyl, optionally substituted quinolinyl, optionally substituted isoquinolinyl, optionally substituted benzothiazole, optionally substituted 1,3-benzodioxolyl, optionally substituted benzofuranyl, optionally substituted 2,1,3-benzothiadiazolyl, optionally substituted 3,4-dihydro-2H,1,4-benzoxazinyl, or optionally substituted benzo-1,4-dioxanyl. Mono or bicyclic 3 to 8 membered heterocycle may be an optionally substituted pyrrolidinyl, optionally substituted tetrahydrofuranyl, optionally substituted tetrahydrothiophenyl, optionally substituted piperidinyl, an optionally substituted piperazinyl, an optionally substituted tetrahydropyranyl, an optionally substituted dioxanyl, an optionally substituted thianyl, an optionally substituted dithianyl or an optionally substituted morpholinyl.

When R¹⁶ is an aryl, the aryl may be unsubstituted or substituted with one or more substituents selected from the group consisting of optionally substituted C₁-C₆ alkyl, optionally substituted C₂-C₆ alkenyl, optionally substituted C₂-C₆ alkynyl, optionally substituted C₁-C₆ alkoxy, halogen, OH, CN, oxo, C(O)R²⁰, COOR²⁰, OC(O)R²⁰, CONR²OR²¹, NR²⁰R²¹, NR²⁰C(O)R²¹, ═NOR²⁰, SR²⁰, SO₂R²⁰, OSO₂R²⁰, SO₂NR²OR²¹, OP(O)(OR²⁰)(OR²¹), optionally substituted C₆-C₁₂ aryl, optionally substituted 5 to 10 membered heteroaryl, optionally substituted C₃-C₆ cycloalkyl and optionally substituted 3 to 8 membered heterocycle. Halogen may be F or Cl. When R¹⁶ is a cycloalkyl, heteroaryl or heterocycle, the cycloalkyl, heteroaryl or heterocycle may be unsubstituted or substituted with one or more substituents selected from the group consisting of optionally substituted C₁-C₆ alkyl, optionally substituted C₂-C₆ alkenyl, optionally substituted C₂-C₆ alkynyl, optionally substituted C₁-C₆ alkoxy, halogen, OH, CN, oxo, C(O)R²⁰, COOR²⁰, OC(O)R²⁰, CONR²OR²¹, NR²⁰R²¹, NR²⁰C(O)R²¹, ═NOR²⁰, SR²⁰, SO₂R²⁰, OSO₂R²⁰, SO₂NR²⁰R²¹, OP(O)(OR²⁰)(OR²¹), optionally substituted C₆-C₁₂ aryl, optionally substituted 5 to 10 membered heteroaryl, optionally substituted C₃-C₆ cycloalkyl and optionally substituted 3 to 8 membered heterocycle. Halogen may be F or Cl. When the cycloalkyl, aryl, heteroaryl or heterocycle is substituted, directly or indirectly, with an optionally substituted alkyl, alkenyl, alkynyl or alkoxy, the alkyl, alkenyl, alkynyl or alkoxy may be unsubstituted or substituted with one or more substituents selected from the group consisting of halogen, OH, C₁-C₆ alkoxy, NR²⁰R²¹, CONR²OR²¹, C(O)R²⁰, CN, oxo, OP(O)(OR²⁰)(OR²¹), OC(O)R²⁰, COOR²⁰, C₁-C₆ alkenyl, C₁-C₆ alkynyl, ═NOR²⁰, NR²⁰C(O)R²¹, SO₂R²⁰ and SO₂NR²OR²¹. Preferably, when the cycloalkyl, aryl, heteroaryl or heterocycle is substituted, directly or indirectly, with an optionally substituted alkyl, alkenyl, alkynyl or alkoxy, the alkyl, alkenyl, alkynyl or alkoxy is unsubstituted or substituted with one or more of halogen and OH. When the cycloalkyl, aryl, heteroaryl or heterocycle is substituted with an optionally substituted aryl or optionally substituted heteroaryl it may be substituted with an optionally substituted phenyl or an optionally substituted 5 or 6 membered heteroaryl. R²⁰ and R²¹ may independently be H, optionally substituted C₁-C₃ alkyl, optionally substituted C₂-C₃ alkenyl or optionally substituted C₂-C₃ alkynyl. Preferably, R²⁰ and R²¹ are independently H and optionally substituted methyl, and more preferably are H, CH₃ or CF₃. Accordingly, the cycloalkyl, aryl, heteroaryl or heterocycle may be unsubstituted or substituted with one or more of F, Cl, oxo, OH, CN, NH₂, methyl, t-butyl, CF₃, CH₂OH, OCH₃, OCHF₂, OCF₃, SCF₃, COCH₃, COOH, COOCH₃, CONH₂, SO₂CH₃, 1,2,4-triazolyl and phenyl. For instance, the optionally substituted 5 to 10 membered heteroaryl may be optionally substituted with a methyl group, and optionally one or more further substituents. Accordingly, the optionally substituted 5 to 10 membered heteroaryl may be optionally substituted 1-methylindolyl, optionally substituted N-methylimidazolyl, optionally substituted N-methylpyrazolyl or optionally substituted N-methylbenzimidazolyl. The aryl, heteroaryl or heterocycle is preferably unsubstituted or substituted with 1 or 2 substituents.

Accordingly, R¹⁶ may be cyclopropyl, cyclopentyl, phenyl,

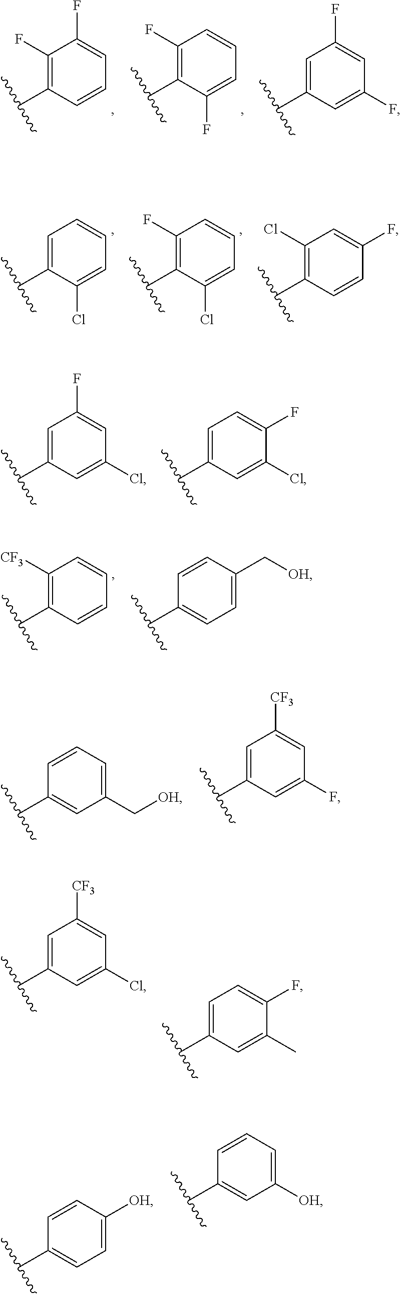

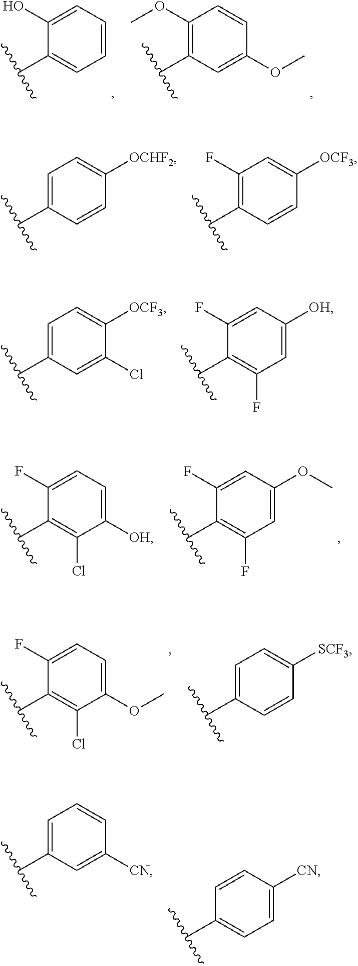

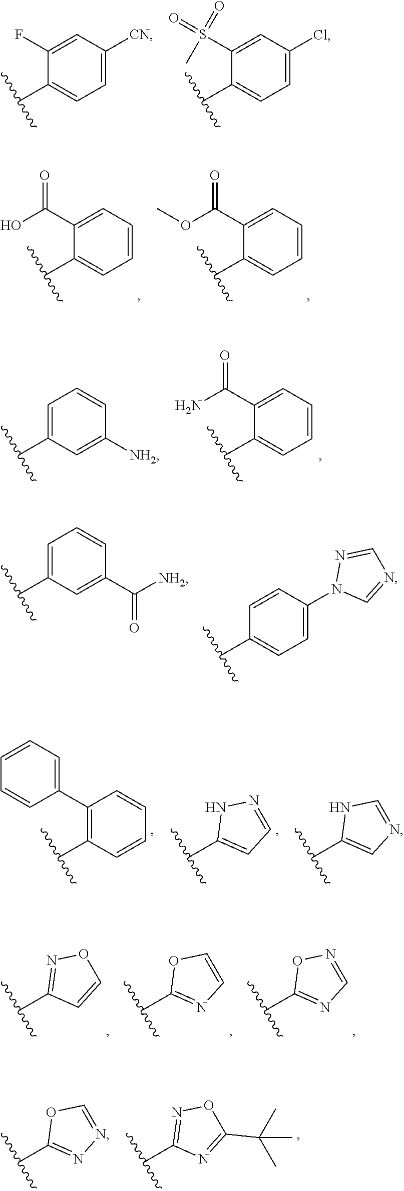

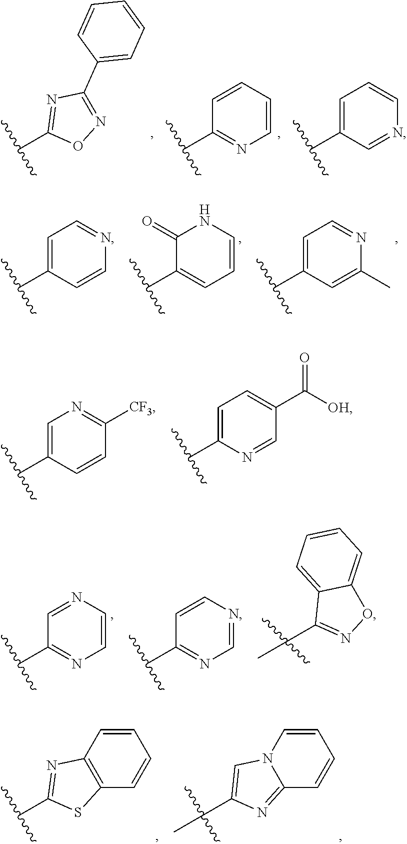

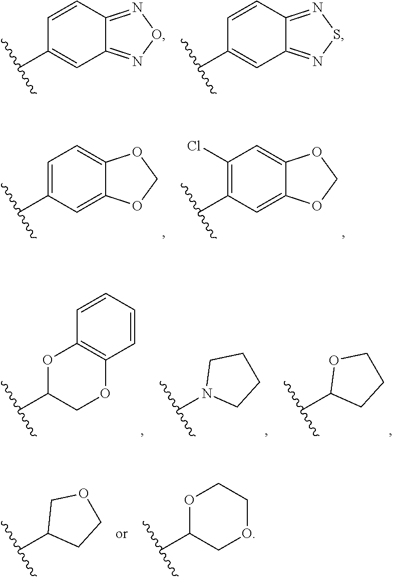

In an alternative embodiment, R⁵ is H, optionally substituted C₁-C₆ alkyl, optionally substituted C₂-C₆ alkenyl or optionally substituted C₂-C₆ alkynyl. R⁵ may be H, optionally substituted C₁-C₃ alkyl, optionally substituted C₂-C₃ alkenyl or optionally substituted C₂-C₃ alkynyl. The alkyl, alkenyl or alkynyl may be unsubstituted or substituted with one or more of halogen, OH, CN and oxo. R⁵ may be H, CH₃ or CH₂CN.

X⁶ may be CO or CR⁷R⁸. R⁷ and R⁸ may independently be H, halogen, OH, CN, COOR¹³, CONR¹³R¹⁴, NR¹³R¹⁴, NR¹³COR¹⁴, optionally substituted C₁-C₆ alkyl, optionally substituted C₂-C₆ alkenyl or optionally substituted C₂-C₆ alkynyl. R⁷ and R⁸ may independently be H, halogen, OH, CN, COOR¹³, CONR¹³R¹⁴, NR¹³R¹⁴, NR¹³COR¹⁴, optionally substituted C₁-C₃ alkyl, optionally substituted C₂-C₃ alkenyl or optionally substituted C₂-C₃ alkynyl. R¹³ and R¹⁴ are preferably H, optionally substituted C₁-C₃ alkyl, optionally substituted C₂-C₃ alkenyl or optionally substituted C₂-C₃ alkynyl, and most preferably H. The alkyl, alkenyl or alkynyl may be unsubstituted or substituted with one or more of halogen, OH, oxo, CN, C(O)R²⁰, COOR²⁰, OC(O)R²⁰, CONR²⁰R²¹, NR²OR²¹, NR²⁰C(O)R²¹, ═NOR²⁰, SR²⁰, SO₂R²⁰, OSO₂R²⁰, SO₂NR²OR²¹ and OP(O)(OR²⁰)(OR²¹). R²⁰ and R²¹ may independently be H or methyl. Preferably, R⁷ and R⁸ are independently H, CN, CONH₂, CH₂NH₂, CH₂CH₂OH, or

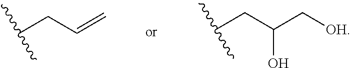

In one embodiment, X⁶ is CO.

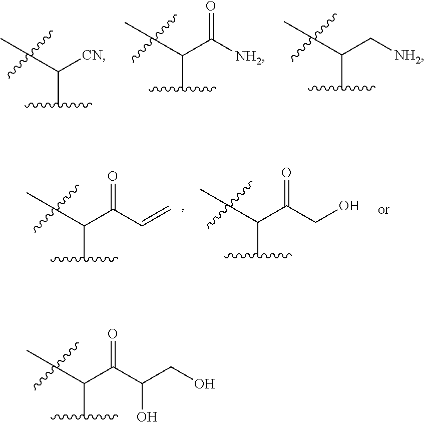

In an alternative embodiment, X⁶ is CH₂,

In one embodiment, n is 0. X⁷ may be CR¹¹R¹². R¹¹ and R¹² may independently be H, halogen, OH, CN, optionally substituted C₁-C₃ alkyl, optionally substituted C₂-C₃ alkenyl or optionally substituted C₂-C₃ alkynyl. Preferably, R¹¹ and R¹² are independently H or methyl. Most preferably, R¹¹ and R¹² are H.

In an alternative embodiment, n is 1.

In one embodiment, Z is CR⁹R¹⁰ and X⁷ is S, SO, SO₂, O or NR¹¹. R⁹ and R¹⁰ may independently be H, halogen, OH, CN, optionally substituted C₁-C₆ alkyl, optionally substituted C₂-C₆ alkenyl or optionally substituted C₂-C₆ alkynyl. R⁹ and R¹⁰ may independently be H, halogen, OH, CN, COOR¹³, CONR¹³R¹⁴, NR¹³R¹⁴, NR¹³COR¹⁴, optionally substituted C₁-C₃ alkyl, optionally substituted C₂-C₃ alkenyl or optionally substituted C₂-C₃ alkynyl. R¹³ and R¹⁴ may independently be H, optionally substituted C₁-C₃ alkyl, optionally substituted C₂-C₃ alkenyl or optionally substituted C₂-C₃ alkynyl. The alkyl, alkenyl or alkynyl may be unsubstituted or substituted with one or more of halogen, OH, oxo, CN, C(O)R²⁰, COOR²⁰, OC(O)R²⁰, CONR²⁰R²¹, NR²⁰R²¹, NR²⁰C(O)R²¹, ═NOR²⁰, SR²⁰, SO₂R²⁰, OSO₂R²⁰, SO₂NR²⁰R²¹ and OP(O)(OR²⁰)(OR²¹). R²⁰ and R²¹ may independently be H or methyl. Preferably, R⁹ and R¹⁰ are independently H, methyl, CH₂CONH₂ or CH₂CN. More preferably, R⁹ and R¹⁰ are H. R¹¹ may be H, optionally substituted C₁-C₆ alkyl, optionally substituted C₂-C₆ alkenyl or optionally substituted C₂-C₆ alkynyl. Ru may be H, C₁-C₃ alkyl, C₂-C₃ alkenyl or C₂-C₃ alkynyl. Preferably, Ru is H or methyl. More preferably, X⁷ is S, O, SO or NR¹¹. Most preferably, X⁷ is S or O.

In an alternative embodiment, Z is NR⁹ and X⁷ is CR¹¹R¹². R⁹ may be H, optionally substituted C₁-C₆ alkyl, optionally substituted C₂-C₆ alkenyl or optionally substituted C₂-C₆ alkynyl. R⁹ may be H, C₁-C₃ alkyl, C₂-C₃ alkenyl or C₂-C₃ alkynyl. Preferably, R⁹ is methyl. R¹¹ and R¹² may independently be H, halogen, OH, CN, optionally substituted C₁-C₆ alkyl, optionally substituted C₂-C₆ alkenyl or optionally substituted C₂-C₆ alkynyl. R¹¹ and R¹² may independently be H, halogen, OH, CN, C₁-C₃ alkyl, C₂-C₃ alkenyl or C₂-C₃ alkynyl. Preferably, R¹¹ and R¹² are H or methyl. In embodiments where X⁷ is CR¹¹R¹² and R¹¹ and R¹² are different, the carbon to which R¹¹ and R¹² are bonded defines a chiral centre. The chiral centre may be an S or R chiral centre. In some embodiments, the chiral centre is an S chiral centre.

In some embodiments, X² is CR², X³ is CR³ and n is 1. Z may be CR⁹R¹⁰ and X⁷ may be S, SO, SO₂, O or NR¹¹. Alternatively, Z may be NR⁹ and X⁷ may be CR¹¹R¹². Accordingly, the compound may be a compound of formula (II) or (III):

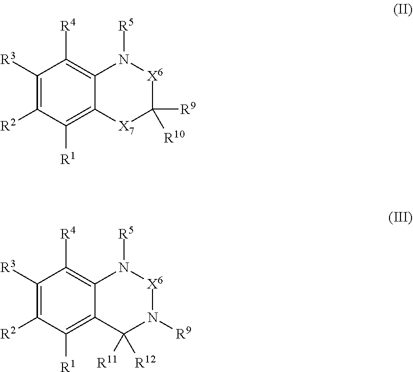

In alternative embodiments, X² is CR², X³ is CR³ and n is o. X⁷ may be CR¹¹R¹². Accordingly, the compound may be a compound of formula (IV):

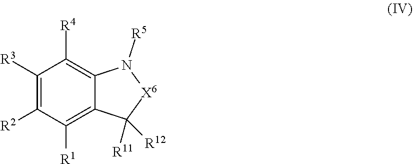

In some embodiments, R² is -L¹-L²-L³-L⁴-R¹⁵. In alternative embodiments, R³ is -L¹-L²-L³-L⁴-R¹⁵. Accordingly, the compound of formula (II), (III) or (IV) may be a compound of formula (IIa), (IIb), (IIIa), (IIIb), (IVa) or (IVb):

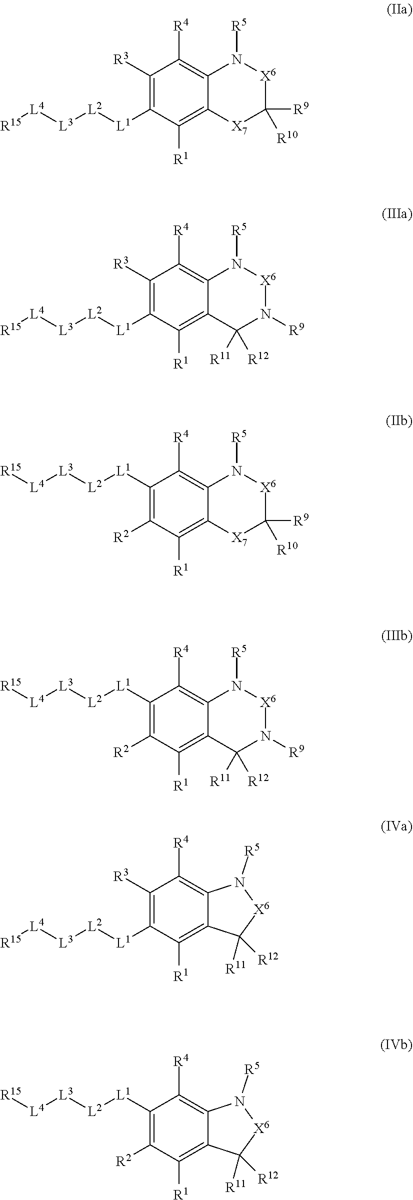

In one embodiment of a compound of formula (II), (III), (IIa), (IIb), (IIIa), (IIIb), (IVa) or (IVb) R⁵ is H, optionally substituted C₁-C₆ alkyl, optionally substituted C₂-C₆ alkenyl or optionally substituted C₂-C₆ alkynyl. R⁵ may be H, optionally substituted C₁-C₃ alkyl, optionally substituted C₂-C₃ alkenyl or optionally substituted C₂-C₃ alkynyl. The alkyl, alkenyl or alkynyl may be unsubstituted or substituted with one or more of halogen, OH, CN and oxo. Preferably, R⁵ is H or CH₃.

In an alternative embodiment of a compound of formula (II), (III), (IIa), (IIb), (IIIa), (IIIb), (IVa) or (IVb), R⁵ is -L⁵-L⁶-R¹⁶. Accordingly, the compound may be a compound of formula (IIc), (IId), (IIIc), (IIId), (IVc) or (IVd):

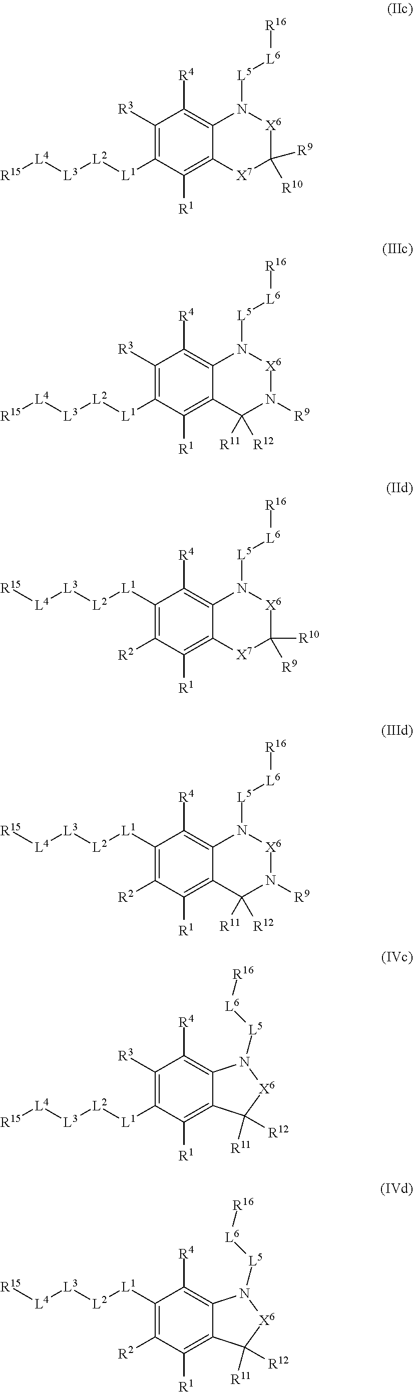

In some embodiments, L⁶ may be absent and R⁵ may be -L⁵-R¹⁶. Accordingly, the compound may be a compound of formula (IIci), (IIdi), (IIIci), (IIIdi), (IVci) or (IVdi):

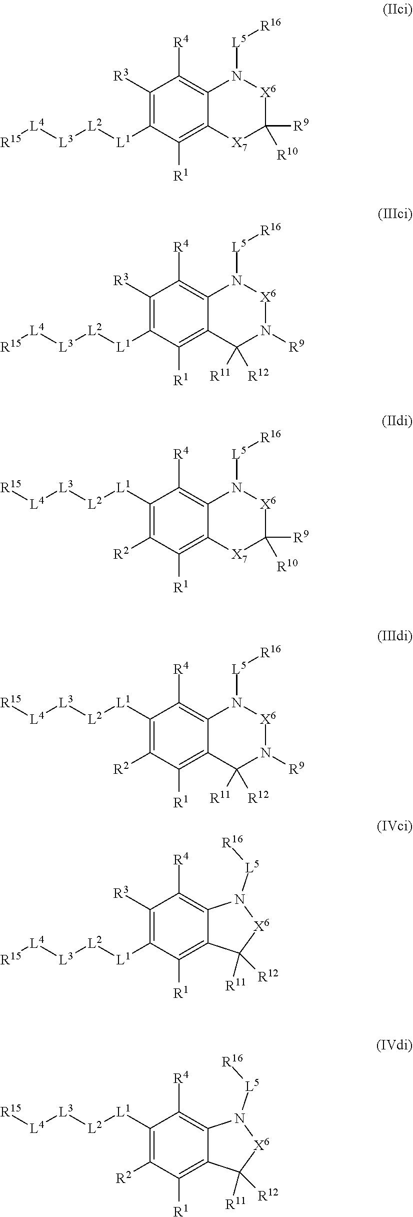

In a compound of formula (II), (III), (IIa) to (IIdi), (IIIa) to (IIIdi), (IV) or (IVa) to (IVdi), X⁶ may be C═O or CR⁷R⁸. In some embodiments, X⁶ is C═O.

In a compound of formula (II) or (IIa) to (IId), X⁷ may be S or O. Preferably, X⁷ is S.

The term ‘STING’ refers to STimulator of INterferon Genes, an adaptor protein that is functionally activated by cyclic dinucleotides which leads to the production of interferons and inflammatory cytokines.

It will be appreciated that an ‘antagonist’, or ‘inhibitor’ as it relates to a ligand and STING, comprises a molecule, combination of molecules, or a complex, that inhibits, counteracts, downregulates, and/or desensitizes STING activity. ‘Antagonist’ encompasses any reagent that inhibits a constitutive activity of STING. A constitutive activity is one that is manifest in the absence of a ligand/STING interaction. ‘Antagonist’ also encompasses any reagent that inhibits or prevents a stimulated (or regulated) activity of STING.

Preferably, the compound of formula (I) is an inhibitor of the STING protein.

It will be appreciated that the compounds described herein or a pharmaceutically acceptable salt, solvate, tautomeric form or polymorphic form thereof may be used in a medicament which may be used in a monotherapy (i.e. use of the compound alone), for modulating the STING protein and/or treating, ameliorating or preventing a disease.

Alternatively, the compounds or a pharmaceutically acceptable salt, solvate, tautomeric form or polymorphic form thereof may be used as an adjunct to, or in combination with, known therapies for modulating the STING protein and/or treating, ameliorating or preventing a disease.

The compound of Formula (I) may be combined in compositions having a number of different forms depending, in particular, on the manner in which the composition is to be used. Thus, for example, the composition may be in the form of a powder, tablet, capsule, liquid, ointment, cream, gel, hydrogel, aerosol, spray, micellar solution, transdermal patch, liposome suspension or any other suitable form that may be administered to a person or animal in need of treatment. It will be appreciated that the vehicle of medicaments according to the invention should be one which is well-tolerated by the subject to whom it is given.

Medicaments comprising the compounds described herein may be used in a number of ways. Suitable modes of administration include oral, intra-tumoral, parenteral, topical, inhaled/intranasal, rectal/intravaginal, and ocular/aural administration. Formulations suitable for the aforementioned modes of administration may be formulated to be immediate and/or modified release. Modified release formulations include delayed-, sustained-, pulsed-, controlled-, targeted and programmed release.

The compounds of the invention may be administered orally. Oral administration may involve swallowing, so that the compound enters the gastrointestinal tract, or buccal or sublingual administration may be employed by which the compound enters the blood stream directly from the mouth. Formulations suitable for oral administration include solid formulations such as tablets, capsules containing particulates, liquids, or powders, lozenges (including liquid-filled), chews, multi- and nano-particulates, gels, solid solution, liposome, films, ovules, sprays, liquid formulations and buccal/mucoadhesive patches.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986, by Liang and Chen (2001).

For tablet dosage forms, depending on dose, the drug may make up from 1 weight % to 80 weight % of the dosage form, more typically from 5 weight % to 60 weight % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet. Other possible ingredients include anti-oxidants, colourants, flavouring agents, preservatives and taste-masking agents.

Exemplary tablets contain up to about 80% drug, from about 10 weight % to about 90 weight % binder, from about o weight % to about 85 weight % diluent, from about 2 weight % to about 10 weight % disintegrant, and from about 0.25 weight % to about 10 weight % lubricant. Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated. The formulation of tablets is discussed in “Pharmaceutical Dosage Forms: Tablets”, Vol. 1, by H. Lieberman and L. Lachman (Marcel Dekker, New York, 1980).

Suitable modified release formulations for the purposes of the invention are described in U.S. Pat. No. 6,106,864. Details of other suitable release technologies such as high energy dispersions and osmotic and coated particles are to be found in “Pharmaceutical Technology On-line”, 25(2), 1-14, by Verma et al (2001). The use of chewing gum to achieve controlled release is described in WO 00/35298.

The compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of compounds of formula (I) used in the preparation of parenteral solutions may be increased by the use of appropriate formulation techniques, such as the incorporation of solubility-enhancing agents. Formulations for parenteral administration may be formulated to be immediate and/or modified release. Modified release formulations include delayed-, sustained-, pulsed-, controlled-, targeted and programmed release. Thus compounds of the invention may be formulated as a solid, semi-solid, or thixotropic liquid for administration as an implanted depot providing modified release of the active compound. Examples of such formulations include drug-coated stents and poly(dl-lactic-coglycolic)acid (PGLA) microspheres.

The compounds of the invention may also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88 (10), 955-958, by Finnin and Morgan (October 1999).

Other means of topical administration include delivery by electroporation, iontophoresis, phonophoresis, sonophoresis and microneedle or needle-free (e.g. Powderject™, Bioject™, etc.) injection.

The compounds of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

The pressurised container, pump, spray, atomizer, or nebuliser contains a solution or suspension of the compound(s) of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, and supercritical fluid processing to form nanoparticles, high pressure homogenisation, or spray drying.

Capsules (made, for example, from gelatin or hydroxypropylmethylcellulose), blisters and cartridges for use in an inhaler or insufflator may be formulated to contain a powder mix of the compound of the invention, a suitable powder base such as lactose or starch and a performance modifier such as L-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the compound of the invention per actuation and the actuation volume may vary from 1 μl to 100 μl. A typical formulation may comprise a compound of formula (I), propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for inhaled/intranasal administration.

In the case of dry powder inhalers and aerosols, the dosage unit is determined by means of a valve which delivers a metered amount. Units in accordance with the invention are typically arranged to administer a metered dose or “puff” containing from 1 μg to 100 mg of the compound of formula (I). The overall daily dose will typically be in the range 1 μg to 200 mg which may be administered in a single dose or, more usually, as divided doses throughout the day.

The compounds of the invention may be administered rectally or vaginally, for example, in the form of a suppository, pessary, microbicide, vaginal ring or enema. Cocoa butter is a traditional suppository base, but various alternatives may be used as appropriate.

The compounds of the invention may also be administered directly to the eye or ear, typically in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

The compounds of the invention may also be administered directly to a site of interest by injection of a solution or suspension containing the active drug substance. The site of interest may be a tumour and the compound may by administer via intratumoral injection. Typical injection solutions are comprised of propylene glycol, sterile water, ethanol and sodium chloride. Alternative solvents which may be used instead of propylene glycol include glycerol and polyethylene glycol.

The compounds of the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

It will be appreciated that the amount of the compound that is required is determined by its biological activity and bioavailability, which in turn depends on the mode of administration, the physiochemical properties of the compound, and whether it is being used as a monotherapy, or in a combined therapy. The frequency of administration will also be influenced by the half-life of the compound within the subject being treated. Optimal dosages to be administered may be determined by those skilled in the art, and will vary with the particular compound in use, the strength of the pharmaceutical composition, the mode of administration, and the advancement of the disease. Additional factors depending on the particular subject being treated will result in a need to adjust dosages, including subject age, weight, sex, diet, and time of administration.

Generally, for administration to a human, the total daily dose of the compounds of the invention is typically in the range 100 μg to 10 g, such as 1 mg to 1 g, for example 10 mg to 500 mg. For example, oral administration may require a total daily dose of from 25 mg to 250 mg. The total daily dose may be administered in single or divided doses and may, at the physician's discretion, fall outside of the typical range given herein. These dosages are based on an average human subject having a weight of about 60 kg to 70 kg. The physician will readily be able to determine doses for subjects whose weight falls outside this range, such as infants and the elderly.

The compound may be administered before, during or after onset of the disease to be treated.

Known procedures, such as those conventionally employed by the pharmaceutical industry (e.g. in vivo experimentation, clinical trials, etc.), may be used to form specific formulations comprising the compounds according to the invention and precise therapeutic regimes (such as daily doses of the compounds and the frequency of administration). The inventors believe that they are the first to describe a pharmaceutical composition for treating a disease, based on the use of the compounds of the invention.

Hence, in an seventh aspect of the invention, there is provided a pharmaceutical composition comprising a compound according to the first aspect, or a pharmaceutically acceptable salt, solvate, tautomeric form or polymorphic form thereof, and a pharmaceutically acceptable vehicle.

The invention also provides, in an eighth aspect, a process for making the composition according to the seventh aspect, the process comprising contacting a therapeutically effective amount of a compound of the first aspect, or a pharmaceutically acceptable salt, solvate, tautomeric form or polymorphic form thereof, and a pharmaceutically acceptable vehicle.

A “subject” may be a vertebrate, mammal, or domestic animal. Hence, compounds, compositions and medicaments according to the invention may be used to treat any mammal, for example livestock (e.g. a horse), pets, or may be used in other veterinary applications. Most preferably, however, the subject is a human being.

A “therapeutically effective amount” of compound is any amount which, when administered to a subject, is the amount of drug that is needed to treat the target disease, or produce the desired effect, i.e. inhibit the STING protein.

For example, the therapeutically effective amount of compound used may be from about 0.01 mg to about 800 mg, and preferably from about 0.01 mg to about 500 mg. It is preferred that the amount of compound is an amount from about 0.1 mg to about 250 mg, and most preferably from about 0.1 mg to about 20 mg.

A “pharmaceutically acceptable vehicle” as referred to herein, is any known compound or combination of known compounds that are known to those skilled in the art to be useful in formulating pharmaceutical compositions.

In one embodiment, the pharmaceutically acceptable vehicle may be a solid, and the composition may be in the form of a powder or tablet. A solid pharmaceutically acceptable vehicle may include one or more substances which may also act as flavouring agents, lubricants, solubilisers, suspending agents, dyes, fillers, glidants, compression aids, inert binders, sweeteners, preservatives, dyes, coatings, or tablet-disintegrating agents. The vehicle may also be an encapsulating material. In powders, the vehicle is a finely divided solid that is in admixture with the finely divided active agents (i.e. the compound according to the first aspect) according to the invention. In tablets, the active compound may be mixed with a vehicle having the necessary compression properties in suitable proportions and compacted in the shape and size desired. The powders and tablets preferably contain up to 99% of the active compound. Suitable solid vehicles include, for example calcium phosphate, magnesium stearate, talc, sugars, lactose, dextrin, starch, gelatin, cellulose, polyvinylpyrrolidine, low melting waxes and ion exchange resins. In another embodiment, the pharmaceutical vehicle may be a gel and the composition may be in the form of a cream or the like.

However, the pharmaceutical vehicle may be a liquid, and the pharmaceutical composition is in the form of a solution. Liquid vehicles are used in preparing solutions, suspensions, emulsions, syrups, elixirs and pressurized compositions. The compound according to the invention may be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilisers, emulsifiers, buffers, preservatives, sweeteners, flavouring agents, suspending agents, thickening agents, colours, viscosity regulators, stabilizers or osmo-regulators. Suitable examples of liquid vehicles for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the vehicle can also be an oily ester such as ethyl oleate and isopropyl myristate. Sterile liquid vehicles are useful in sterile liquid form compositions for parenteral administration. The liquid vehicle for pressurized compositions can be a halogenated hydrocarbon or other pharmaceutically acceptable propellant.

Liquid pharmaceutical compositions, which are sterile solutions or suspensions, can be utilized by, for example, intramuscular, intrathecal, epidural, intraperitoneal, intravenous and particularly subcutaneous injection. The compound may be prepared as a sterile solid composition that may be dissolved or suspended at the time of administration using sterile water, saline, or other appropriate sterile injectable medium.

The compound and compositions of the invention may be administered in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like. The compounds used according to the invention can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

It will be known to those skilled in the art that active drug ingredients may be converted into a prodrug, which is a metabolically labile derivative that is converted within the body into the active drug substance. Also included within the scope of the invention are prodrugs which are compounds of formula (I) which contain metabolically or hydrolytically labile moieties which in vivo are converted into the active drug of formula (I). The processes by which the prodrug is converted into the active drug substance include, but are not limited to, ester or carbonate or carbamate hydrolysis, phosphate ester hydrolysis, S-oxidation, N-oxidation, dealkylation and metabolic oxidation as described in Beaumont et. al., Curr. Drug Metab., 2003, 4, 461-485 and Huttenen et. al., Pharmacol. Revs., 2011, 63, 750-771. Such prodrug derivatives may offer improved solubility, stability or permeability compared to the parent drug substance, or may better allow the drug substance to be administered by an alternative route of administration, for example as an intravenous solution.

Also included within the scope of the invention are soft drugs or antedrugs which are compounds of formula (I) which contain metabolically or hydrolytically labile moieties which in vivo are converted into inactive derivatives. The processes by which the active drug substance is converted into an inactive derivative include, but are not limited to, ester hydrolysis, S-oxidation, N-oxidation, dealkylation and metabolic oxidation as described for example in Pearce et al., Drug Metab. Dispos., 2006, 34, 1035-1040 and B. Testa, Prodrug and Soft Drug Design, in Comprehensive Medicinal Chemistry II, Volume 5, Elsevier, Oxford, 2007, pp. 1009-1041 and Bodor, N. Chem. Tech. 1984, 14, 28-38.

The scope of the invention includes all pharmaceutically acceptable isotopically-labelled compounds of the invention wherein one or more atoms are replaced by atoms having the same atomic number, but an atomic mass or mass number different from the atomic mass or mass number which predominates in nature.

Examples of isotopes suitable for inclusion in the compounds of the invention include isotopes of hydrogen, such as ²H and 3H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of the invention, for example those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection. Substitution with isotopes such as deuterium, i.e. ²H, may afford certain therapeutic advantages resulting from greater metabolic stability, for example, increased in vivo half-life or reduced dosage requirements, and hence may be preferred in some circumstances. Substitution with positron emitting isotopes, such as ¹¹C, ¹⁸F, ¹⁵O and ¹³N, can be useful in Positron Emission Topography (PET) studies for examining substrate receptor occupancy.

Isotopically-labelled compounds of formula (I) can generally be prepared by conventional techniques known to those skilled in the art or by processes analogous to those described in the accompanying Examples and Preparations using an appropriate isotopically-labelled reagent in place of the non-labelled reagent previously employed.

In a further aspect of the invention, there is provided a compound of formula (V):

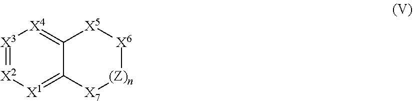

wherein X¹ is CR¹ or N;
X² is CR² or N;
X³ is CR³ or N;
X⁴ is CR⁴ or N;
X⁵ is NR⁵ or CR⁵R⁶;
X⁶ is NR⁷, C═O, C═S or CR⁷R⁸;
the or each Z is independently CR⁹R¹⁰ or NR⁹;
X⁷ is S, SO, SO₂, O, NR¹¹ or CR¹¹R¹²;
n is 0, 1 or 2;
R¹, R⁴, R⁶, R⁸, R⁹, R¹⁰, R¹¹ and R¹² are each independently selected from the group consisting of H, halogen, OH, CN, COOR¹³, CONR¹³R¹⁴, NR¹³R¹⁴, NR¹³COR¹⁴, optionally substituted C₁-C₆ alkyl, optionally substituted C₁-C₆ alkylsulfonyl, optionally substituted mono or bicyclic C₃-C₆ cycloalkyl, optionally substituted C₂-C₆ alkenyl, optionally substituted C₂-C₆ alkynyl, optionally substituted C₁-C₆ alkoxy, optionally substituted C₁-C₆ alkoxycarbonyl group, mono or bicyclic optionally substituted C₆-C₁₂ aryl, mono or bicyclic optionally substituted 5 to 10 membered heteroaryl, optionally substituted mono or bicyclic 3 to 8 membered heterocycle, optionally substituted aryloxy, optionally substituted heteroaryloxy and optionally substituted heterocyclyloxy; one of R² and R³ is -L¹-L²-L³-L⁴-R¹⁵ and, when X² is CR² and X³ is CR³, the other of R² and R³ is selected from the group consisting of H, halogen, OH, CN, COOR¹³, CONR¹³R¹⁴, NR¹³R¹⁴, NR¹³COR¹⁴, optionally substituted C₁-C₆ alkyl, optionally substituted C₁-C₆ alkylsulfonyl, optionally substituted mono or bicyclic C₃-C₆ cycloalkyl, optionally substituted C₂-C₆ alkenyl, optionally substituted C₂-C₆ alkynyl, optionally substituted C₁-C₆ alkoxy, optionally substituted C₁-C₆ alkoxycarbonyl group, mono or bicyclic optionally substituted C₆-C₁₂ aryl, mono or bicyclic optionally substituted 5 to membered heteroaryl, optionally substituted mono or bicyclic 3 to 8 membered heterocycle, optionally substituted aryloxy, optionally substituted heteroaryloxy and optionally substituted heterocyclyloxy;

R⁵ and R⁷ are each independently selected from the group consisting of H, halogen, OH, CN, COOR¹³, CONR¹³R¹⁴, NR¹³R¹⁴, NR¹³COR¹⁴, optionally substituted C₁-C₆ alkyl, optionally substituted C₁-C₆ alkylsulfonyl, optionally substituted mono or bicyclic C₃-C₆ cycloalkyl, optionally substituted C₂-C₆ alkenyl, optionally substituted C₂-C₆ alkynyl, optionally substituted C₁-C₆ alkoxy, optionally substituted C₁-C₆ alkoxycarbonyl group, mono or bicyclic optionally substituted C₆-C₁₂ aryl, mono or bicyclic optionally substituted 5 to 10 membered heteroaryl, optionally substituted mono or bicyclic 3 to 8 membered heterocycle, optionally substituted aryloxy, optionally substituted heteroaryloxy, optionally substituted heterocyclyloxy and L⁵-R¹⁶; wherein a maximum of one of R⁵ and R⁷ is -L⁵-R¹⁶;

R¹³ and R¹⁴ are each independently selected from the group consisting of H, halogen, OH, CN, COOH, CONH₂, NH₂, NHCOH, optionally substituted C₁-C₆ alkyl, optionally substituted C₁-C₆ alkylsulfonyl, optionally substituted mono or bicyclic C₃-C₆ cycloalkyl, optionally substituted C₂-C₆ alkenyl, optionally substituted C₂-C₆ alkynyl, optionally substituted C₁-C₆ alkoxy, optionally substituted C₁-C₆ alkoxycarbonyl group, mono or bicyclic optionally substituted C₆-C₁₂ aryl, mono or bicyclic optionally substituted 5 to membered heteroaryl, optionally substituted mono or bicyclic 3 to 8 membered heterocycle, optionally substituted aryloxy, optionally substituted heteroaryloxy and optionally substituted heterocyclyloxy;

L¹ is absent or is NR¹⁷, O, an optionally substituted C₁-C₆ alkylene, an optionally substituted C₂-C₆ alkenylene, an optionally substituted C₂-C₆ alkynylene, an optionally substituted C₃-C₆ cycloalkylene, an optionally substituted C₆-C₁₂ arylene, an optionally substituted 5 to 10 membered heteroarylene or an optionally substituted 3 to 8 membered heterocyclylene;

L² is absent or is C═O, C═S, C═NR¹⁹ or SO₂;

L³ is absent or is NR¹⁸, O, an optionally substituted C₁-C₆ alkylene, an optionally substituted C₂-C₆ alkenylene, an optionally substituted C₂-C₆ alkynylene, an optionally substituted C₃-C₆ cycloalkylene, an optionally substituted C₆-C₁₂ arylene, an optionally substituted 5 to 10 membered heteroarylene or an optionally substituted 3 to 8 membered heterocyclylene;

L⁴ is absent or is an optionally substituted C₁-C₆ alkylene, an optionally substituted C₂-C₆ alkenylene, an optionally substituted C₂-C₆ alkynylene, an optionally substituted C₃-C₆ cycloalkylene, an optionally substituted C₆-C₁₂ arylene, an optionally substituted 5 to membered heteroarylene or an optionally substituted 3 to 8 membered heterocyclylene;

L⁵ is absent or an optionally substituted C₁-C₆ alkylene, an optionally substituted C₂-C₆ alkenylene, an optionally substituted C₂-C₆ alkynylene, S═O, SO₂ or NR¹⁹;

R¹⁵ is H, optionally substituted C₁-C₆ alkyl, optionally substituted C₂-C₆ alkenyl, optionally substituted C₂-C₆ alkynyl, optionally substituted mono or bicyclic C₃-C₆ cycloalkyl, mono or bicyclic optionally substituted C₆-C₁₂ aryl, mono or bicyclic optionally substituted 5 to 10 membered heteroaryl or optionally substituted mono or bicyclic 3 to 8 membered heterocycle;

R¹⁶ is H, optionally substituted C₂-C₆ alkenyl, optionally substituted C₂-C₆ alkynyl, optionally substituted mono or bicyclic C₃-C₆ cycloalkyl, mono or bicyclic optionally substituted C₆-C₁₂ aryl, mono or bicyclic optionally substituted 5 to 10 membered heteroaryl or optionally substituted mono or bicyclic 3 to 8 membered heterocycle; and

R¹⁷ to R¹⁹ are independently H, an optionally substituted C₁-C₆ alkyl, an optionally substituted C₂-C₆ alkenyl or an optionally substituted C₂-C₆ alkynyl;

wherein, when X² is N, X³ is CR³; and

when L¹ is absent and L² is C═O, L³ is not NR¹;

or a pharmaceutically acceptable complex, salt, solvate, tautomeric form or polymorphic form thereof.

X¹ may be CR¹. X⁴ may be CR⁴.

In one embodiment, X⁵ is NR⁵ or CR⁵R⁶ and R⁵ is -L⁵-R¹⁶. X⁵ may be NR⁵ and R⁵ may be -L⁵-R¹⁶.

In an alternative embodiment, X⁵ is NR⁵ or CR⁵R⁶ and R⁵ and R⁶ are independently H, optionally substituted C₁-C₆ alkyl, optionally substituted C₂-C₆ alkenyl or optionally substituted C₂-C₆ alkynyl. R⁵ and R⁶ may independently be H, optionally substituted C₁-C₃ alkyl, optionally substituted C₂-C₃ alkenyl or optionally substituted C₂-C₃ alkynyl.

The alkyl, alkenyl or alkynyl may be unsubstituted or substituted with one or more of halogen, OH, CN and oxo. Preferably, X⁵ is NR⁵. R⁵ may be H or CH₃.

In some embodiments, X¹ is CR¹, X² is CR², X³ is CR³, X⁴ is CR⁴, X⁵ is NR⁵ and n is 1. Z may be CR⁹R¹⁰ and X⁷ may be S, SO, SO₂, O or NR¹¹. Alternatively, Z may be NR⁹ and X⁷ may be CR¹¹R¹².

All features described herein (including any accompanying claims and abstract), and/or all of the steps of any method or process so disclosed, may be combined with any of the above aspects in any combination, except combinations where at least some of such features and/or steps are mutually exclusive.

General Schemes

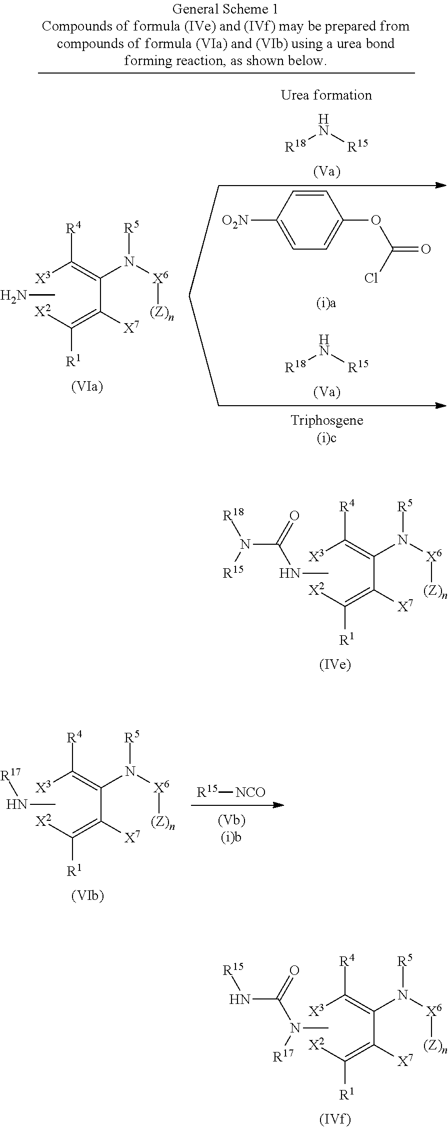

Typical reaction conditions for the activation of the aromatic amine of the compounds of formula (VIa) or (VIb) employ 4-nitrophenyl chloroformate or triphosgene to generate an activated intermediate which can be attacked by a suitable nucleophile such as amine (Va) to give a urea compound of formula (IVe) or (IVf). Preferred organic bases include DIPEA or TEA in a suitable organic solvent such as DCM, DMF, DMA or MeCN. The reaction may be shaken or stirred at room temperature.

Alternatively, the compounds of formula (IVe) or (IVf) can also be prepared with an isocyanate R¹⁵NCO (Vb) in a suitable solvent such as THF, DMF or MeCN and a preferred organic base such as TEA or DIPEA. The reaction may be shaken or stirred at room temperature.

Compounds of formula (V) and (VI) are commercially available or may be synthesized by those skilled in the art. In particular, methods of synthesizing compounds of formula (VI) are described in General Schemes 2 to 4.

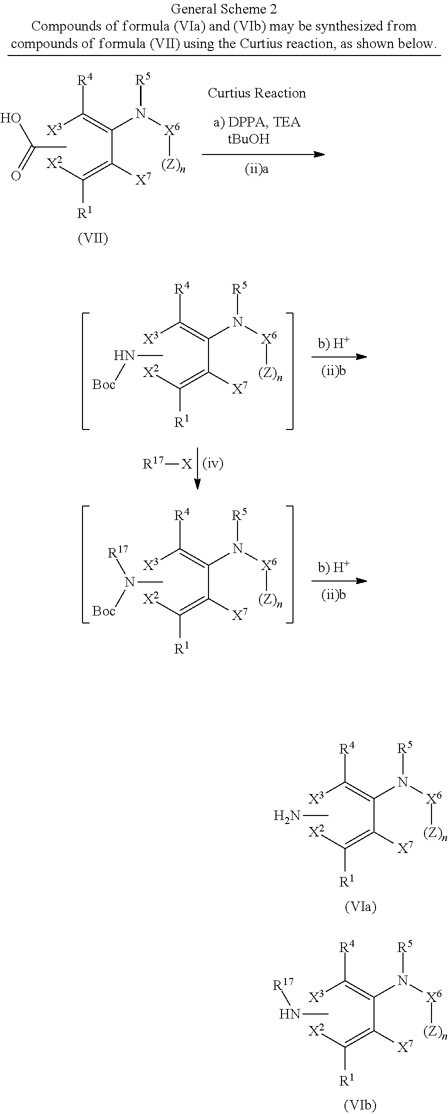

Typical reaction conditions included treating a compound of formula (VII) with the reagent diphenylphosphoryl azide (DPPA) and a base such as TEA to produce the corresponding acyl azide which was further refluxed in t-butanol to furnish the BOC protected amines as intermediates. The corresponding intermediates either can be de-protected in an acidic environment to give the free amines of formula (VIa) or can be first substituted with suitable agents such as R¹⁷—X using methods described in General Procedure (iv) then de-protected in an acidic environment to give the N-substituted amines of formula (VIb).

Compounds of formula (VII) are commercially available or may be synthesized by those skilled in the art. In particular, methods of synthesizing compounds of formula (VII) are described in General Schemes 3-4.

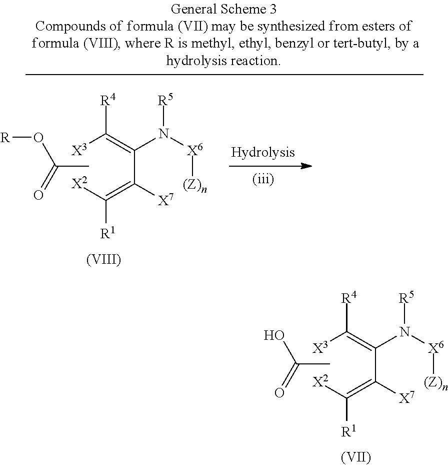

The compound of formula (VIII) may be reacted with a suitable alkali or base to cause it to undergo hydrolysis and provide a compound of formula (VII). The suitable alkali or base may be LiOH, KOH, NaOH or K₂CO₃, and the reaction may be conducted in an aqueous solution.

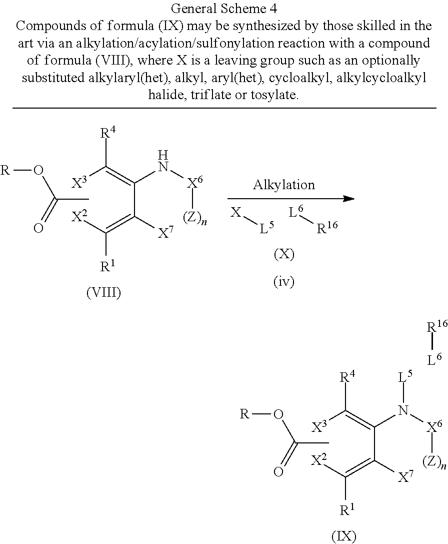

Compounds of formula (VIII) may be reacted with compounds of formula (X) in the presence of a suitable base such as NaH, NaHCO₃ or TEA to furnish compounds of formula (IX). Suitable reaction solvents include THF, DMA and DMF.

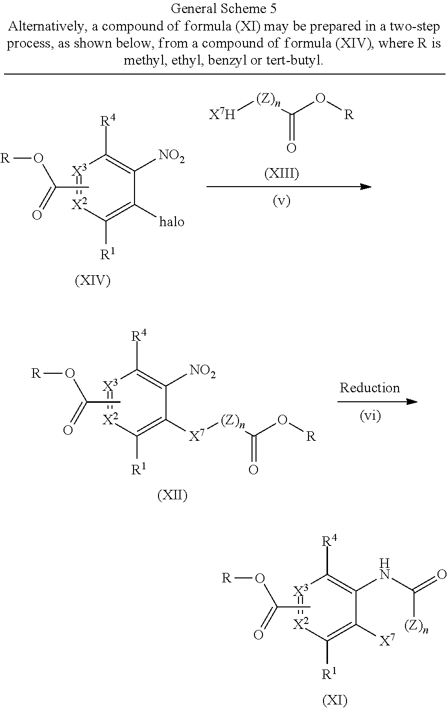

Firstly, compounds of formula (XIV) undergo a nucleophilic substitution reaction with a compound of formula (XIII), where R is methyl, ethyl, benzyl or tert-butyl, to produce a compound of formula (XII). The nucleophilic substitution reaction may be conducted in the presence of a mild base, such as DBU, NaH, TEA, DIPEA, K₂CO₃, Cs₂CO₃ or KHCO₃. The solvent used may be 1,4-dioxane, acetone, MeCN, THF or DMF.

The nitro group of compounds of formula (XII) may then be reduced to an amino group using a suitable reducing agent, such as Fe/AcOH, Zn/HCl, Zn/NH₄Cl, Zn/HCOONH₄, SnCl₂/HCl or Pd/C/H₂, in a suitable solvent such as EtOH, MeOH or THF. The ensuing amino compounds typically undergo in-situ cyclization resulting in the formation of compounds of formula (XI).

It will be appreciated that the compound of formula (XI) is a compound of formula (VIII) where R⁵ is H and X⁶ is C═O.

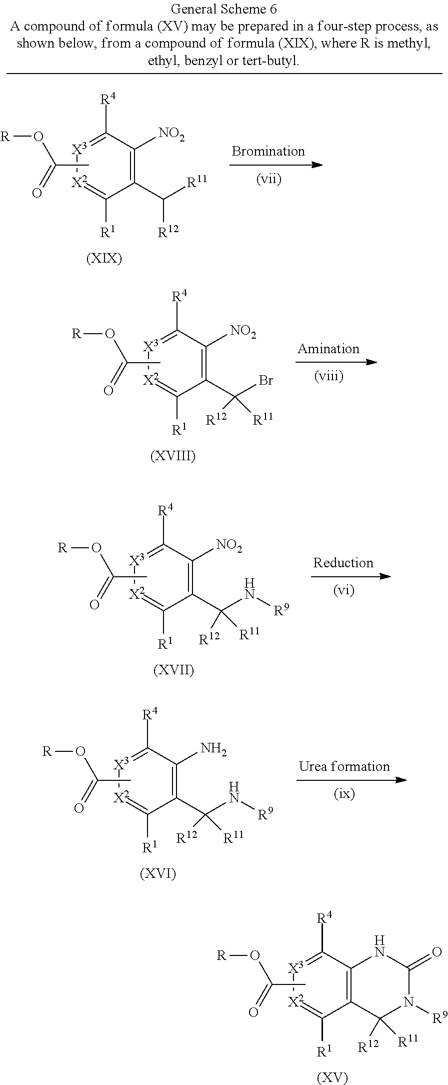

Firstly, the compound of formula (XIX) may be brominated, using either Br₂ or a bromine source, such as NBS, to give a compound of formula (XVIII). This compound can then be aminated, using R⁹NH₂, to provide a compound of formula (XVII). The nitro group on the compound of formula (XVII) can then be reduced using suitable reducing agents, for example those described in General Scheme 5, to provide a compound of formula (XVI). The compound of formula (XVI) may then be reacted with a suitable carbonyl source to provide a compound of formula (XV). The carbonyl source may be 1,1-carbonyl-diimidazole, phosgene or triphosgene.

It will be appreciated that the compound of formula (XV) is a compound of formula (VIII) where R⁵ is H, X⁶ is C═O, Z is NR⁹, X⁷ is CR¹¹R¹² and n is 1.

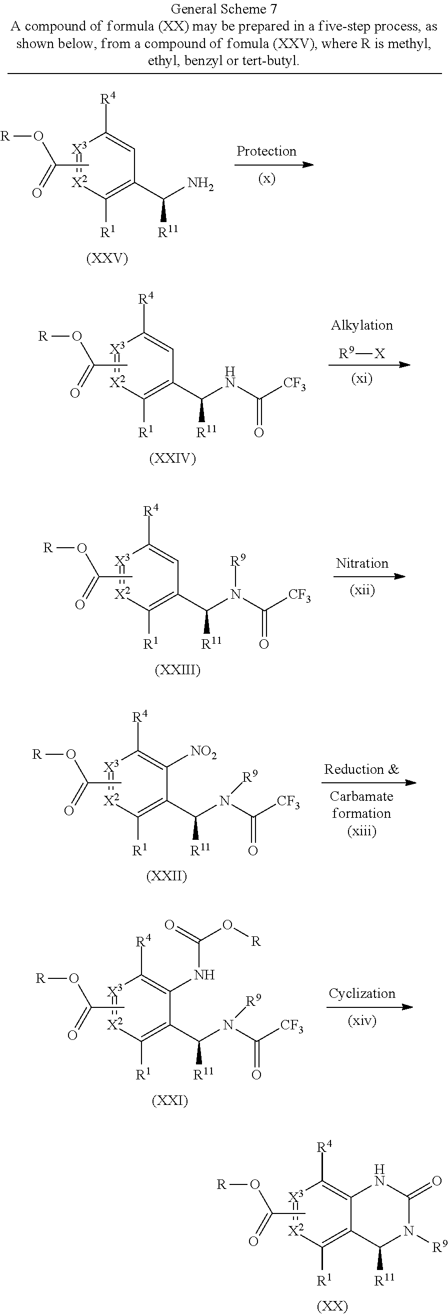

Firstly, the compound of formula (XXV) may be protected with a suitable acetyl group using reagents such as TFAA, BOC-anhydride or acetic anhydride to give a compound of formula (XXIV). This compound may be alkylated using a suitable alkyl halide (R⁹—X) in the presence of a suitable base such as NaH, K₂CO₃, KHCO₃, Cs₂CO₃ or ^{t}BuCOOK/Na to give a compound of formula (XXIII). A subsequent nitration reaction may be performed on compounds of formula (XXIII) with a nitrating mixture, such as nitric acid and sulfuric acid mixtures, to give a compound of formula (XXII). The nitro group on compounds of formula (XXII) can then be reduced either by Pd-catalyzed hydrogenation methods or by using the sodium dithionite and TBASH method as described in General Procedure 6b to give the corresponding amino derivative. Further reaction of this amine with an alkyl chloroformate RO(CO)Cl in the presence of a suitable organic or inorganic base such as pyridine or K₂CO₃ provides a compound of formula (XXI). This compound may then undergo a cyclization process to give a compound of formula (XX) by using a suitable base and solvent combination such as K₂CO₃ and methanol.

It will be appreciated that the compound of formula (XX) is a compound of formula (VIII) where R⁵ is H, X⁶ is C═O, Z is NR⁹, X⁷ is CH(S)R¹¹ and n is 1.

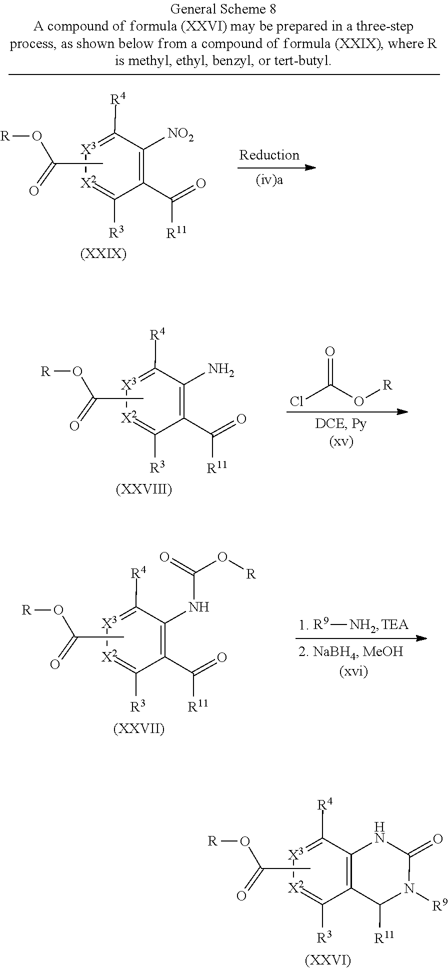

Firstly, the compound of formula (XXIX) can be reduced using any of the methods described in General Scheme 5, for example Fe/Zn-AcOH/HCl to convert the nitro group into an amino group and furnish a compound of formula (XXVIII). This compound may then form a corresponding carbamate using a suitable chloroformate, in the presence of a suitable organic or inorganic base such as pyridine or K₂CO₃ to provide a compound of formula (XXVII). The compound of formula (XXVII) can be converted into a cyclized compound of formula (XXVI) in a series of reactions such as Schiff base formation with a suitable amine R⁹—NH₂ in the presence of an organic base such as TEA or DIPEA followed by reduction of the resulting imine with a mild reducing agent, for example Na(AcO)₃BH, NaCNBH₃ or NaBH₄ in methanol. The resulting amine typically undergoes spontaneous cyclization in-situ to afford the compound of formula (XXVI).

It will be appreciated that the compound of formula (XXVI) is a compound of formula (VIII) where R⁵ is H, X⁶ is C═O, Z is NR⁹, X⁷ is CHR¹¹ and n is 1.

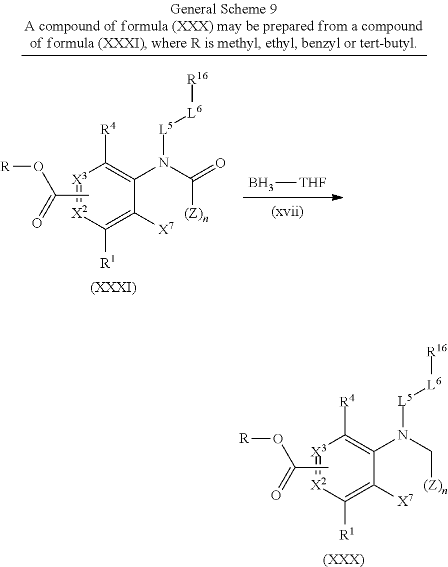

The lactam carbonyl group of a compound of formula (XXXI) can be reduced to the corresponding methylene group of a compound of formula (XXX) using borane-THF solution in a suitable solvent such as THF, typically at low temperatures.

It will be appreciated that the compound of formula (XXX) is a compound of formula (VIII) where X⁶ is CH₂.

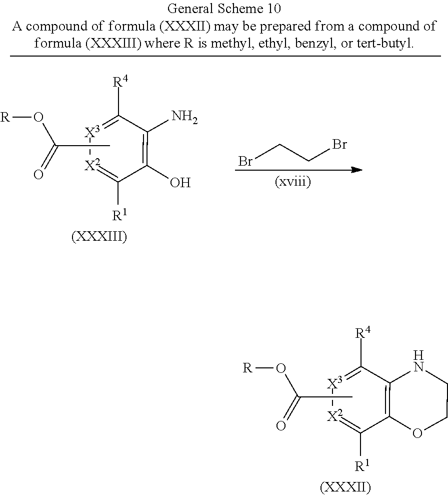

Compounds of formula (XXXIII) may undergo cyclization with 1,2-dibromoethane in a basic reaction medium to give a fused-morpholine derivative compound of formula (XXXII).

It will be appreciated that the compound of formula (XXXII) is a compound of formula (VIII) where X⁶ and Z are CH₂, and X⁷ is O.

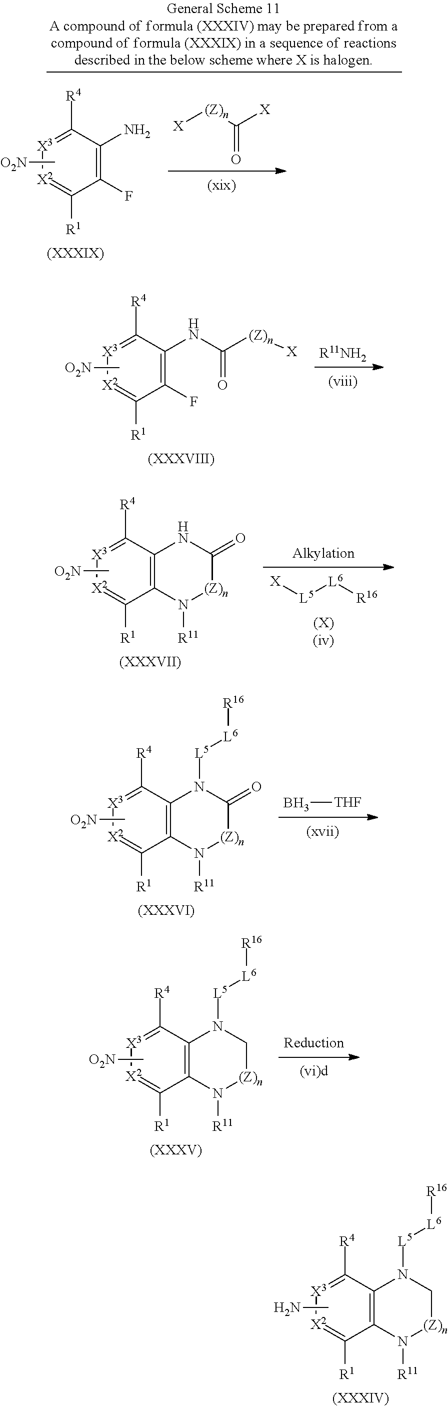

A compound of formula (XXXIX) may undergo acylation with a suitable acylating agent in acetone or alcoholic solvents to produce a compound of formula (XXXVIII) which can be cyclized in situ after introducing an amine R¹¹NH₂ to give a compound of formula (XXXVII). The compound of formula (XXXVII) may be reacted with compounds of formula (X) where X is a suitable leaving group such as halide, tosylate or triflate in the presence of a suitable base such as NaH, NaHCO₃ or TEA to furnish compounds of formula (XXXVI). Suitable reaction solvents include THF, DMA and DMF. The lactam carbonyl group of a compound of formula (XXXVI) can be reduced to the corresponding methylene group of a compound of formula (XXXV) using borane-THF solution in a suitable solvent such as THF, typically at low temperatures. The nitro group of compound of formula (XXXV) can be reduced to its corresponding amino group of a compound of formula (XXXIV) using NiCl₂.6H₂O and sodium borohydride in a polar solvent such as methanol.

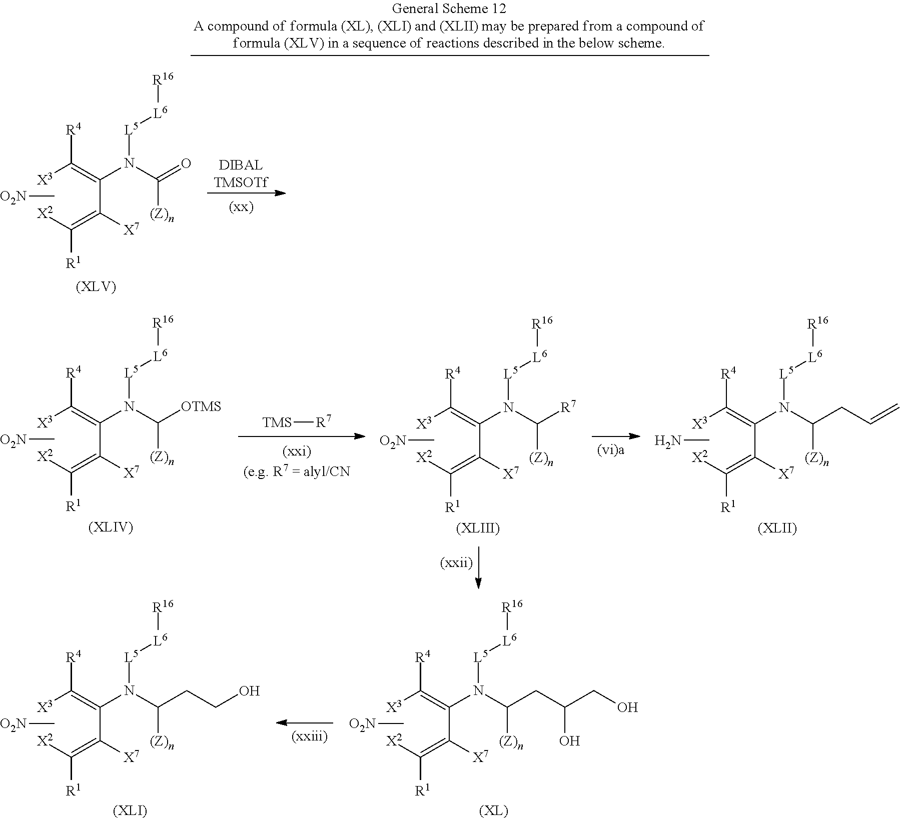

A compound of formula (XLV) may be reduced to the corresponding alcohol with reducing agents such as DIBAL and then subsequently converted into a leaving group, for example a silyl ether (OTMS) with TMSOTf to give a compound of formula (XLIV). The leaving group can be replaced by a suitable nucleophile to generate a compound of formula (XLIII). The suitable nucleophile could be CN or allyl. An allyl containing compound of formula (XLIII) can then undergo hydroxylation with OsO₄ to give a compound of formula (XL). The compound of formula (XL) can be oxidized to the corresponding aldehyde with NaIO₄ and then subsequently reduced to the corresponding primary alcohol (XLI) with suitable reducing reagents such as NaBH₄. The nitro group of a compound of formula (XLIII) can also be reduced to the corresponding amine (XLII) with a suitable reducing reagent such as Fe/AcOH or Zn/AcOH or Fe/NH₄Cl.

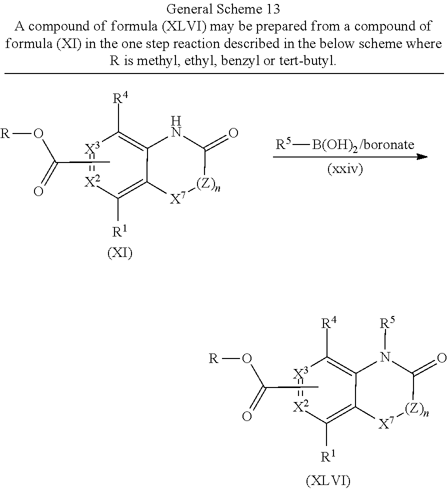

A compound of formula (XI) may undergo a Chan-Lam coupling reaction with a suitable boronic acid/boronate ester in the presence of a suitable catalyst and base to give a compound of formula (XLVI).

It will be appreciated that the compound of formula (XLVI) is a compound of formula (VIII) where X⁶ is C═O.

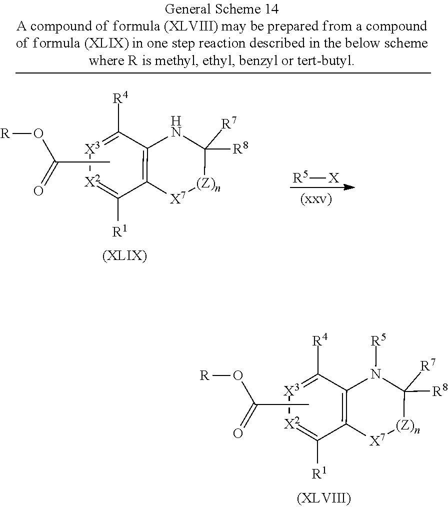

A compound of formula (XLIX) may undergo a Buchwald coupling reaction with a suitable aromatic halide (R⁵—X) to give a compound of formula (XLVIII).

It will be appreciated that the compound of formula (XLVIII) is a compound of formula (VIII) where X⁶ is CR⁷R⁸.

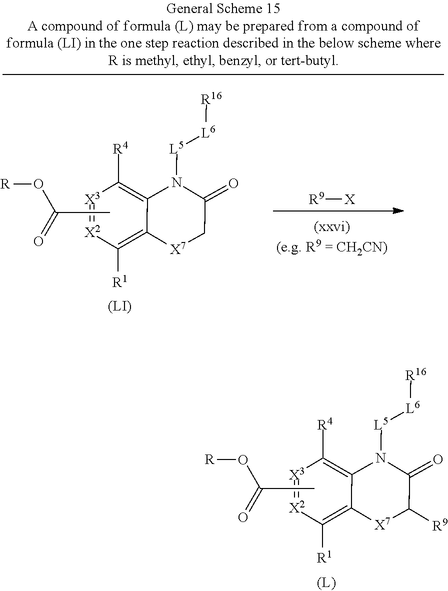

A compound of formula (LI) may be treated with a suitable base such as LiHMDS to generate an anion at the most acidic methylene position which can then be alkylated with a suitable electrophile such as XCH₂CN to generate a compound of formula (L).

It will be appreciated that the compound of formula (L) is a compound of formula (VIII) where X⁶ is C═O, Z is CHR⁹ and n is 1.

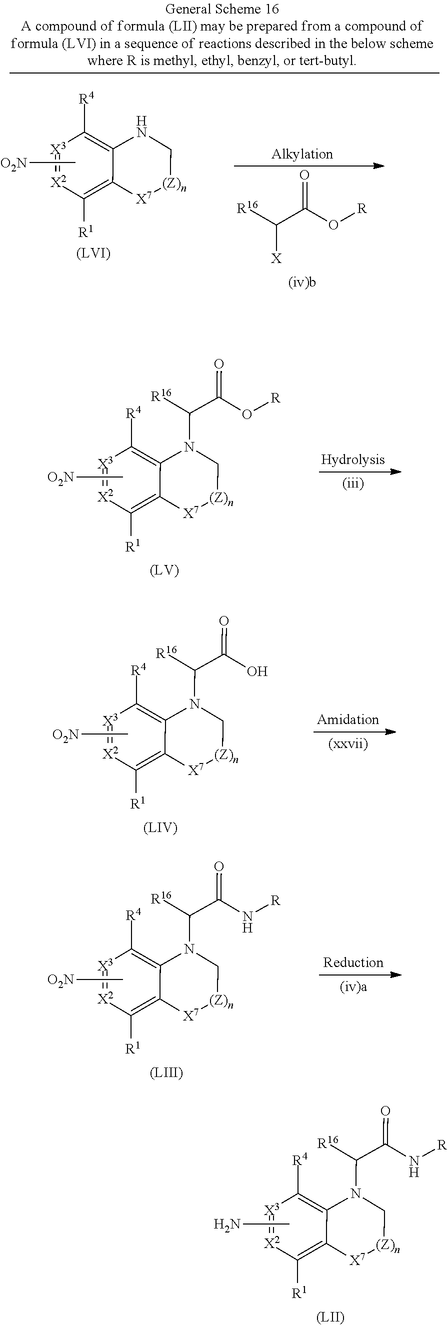

Firstly, a compound of formula (LVI) may be alkylated with suitable alkylating agents in the presence of a suitable base in a suitable solvent such as ACN, THF or DMF to give a compound of formula (LV) which can undergo ester hydrolysis to produce a compound of formula (LIV). The acid functional group can then be converted into the corresponding amide under typical amide coupling reaction conditions with a suitable amine to afford the compound of formula (LIII). Finally, the nitro group of a compound of formula (LIII) may be reduced to the corresponding amine in a compound of formula (LII) with suitable reducing reagents.

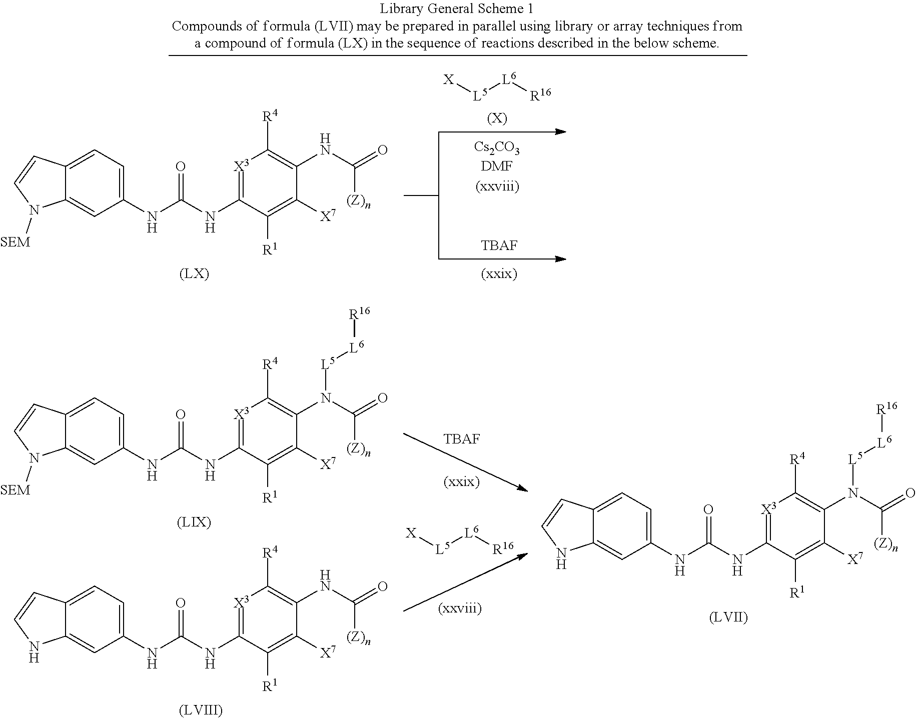

A compound of formula (LX) may be alkylated with suitable compounds of formula (X) in which X is a leaving group in the presence of a suitable base such as NaH, Cs₂CO₃, NaHCO₃ or TEA to furnish compounds of formula (LIX) as described in General Scheme 4, but carried out typically in 0.1-0.2 mmol scale. Suitable reaction solvents include THF, DMA and DMF. The alkylated compounds of formula (LIX) may then have their SEM protecting group removed by treating with a fluoride source such as TBAF or HF, or with a suitable acid such as TFA to provide the final products of formula (LVII). The progress of the reactions were monitored by LCMS and after completion, the reaction mixture was purified by prep-HPLC. Alternatively, the sequence of reactions may be reversed in that the SEM group may first be removed from compounds of formula (LX) to give the indole derivatives of formula (LVIII) and then the alkylation reaction carried out to give the products (LVII).

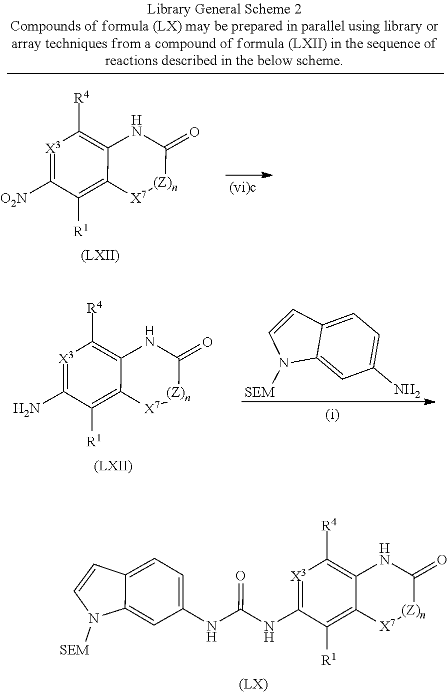

Compounds of formula (LXII) may be reduced using a suitable reducing agent such as Fe/AcOH, Zn/AcOH, Zn/HCl, Zn/NH₄C₁, Zn/HCOONH₄, SnCl₂/HCl or by hydrogenation in the presence of a suitable catalyst such as Pd/C, PtO₂, or any Rh or Ru based catalyst systems in a suitable solvent such as EtOH, MeOH or THF to give the amines of formula (LXI). Compounds of formula (LXI) may then be reacted with any suitable amine (Va) as described in General Scheme 1 to give a urea compound of formula (LX). Preferred organic bases for this reaction include DIPEA or TEA in a suitable organic solvent such as DCM, DMF, DMA or MeCN with amine activation typically carried out using 4-nitrophenyl chloroformate or triphosgene in a 0.1-0.2 mmol scale. The reaction may be shaken or stirred at room temperature. The progress of the reactions were monitored by LCMS and after completion, the reaction mixture was purified by prep-HPLC.

General Synthetic Procedures

General Purification and Analytical Methods

All final compounds were purified by either Combi-flash or prep-HPLC purification, and analysed for purity and product identity by UPLC or LCMS according to one of the below conditions.

Prep-HPLC

Preparative HPLC was carried out on a Waters auto purification instrument using a Gemini C18 column (250×21.2 mm, 10 μm) operating at ambient temperature with a flow rate of 16.0-25.0 mL/min.

Mobile phase 1: A=0.1% formic acid in water, B=Acetonitrile; Gradient Profile: Mobile phase initial composition of 80% A and 20% B, then to 60% A and 40% B after 3 min., then to 30% A and 70% B after 20 min., then to 5% A and 95% B after 21 min., held at this composition for 1 min. for column washing, then returned to the initial composition for 3 min.

Mobile phase 2: A=10 mM Ammonium Acetate in water, B=Acetonitrile; Gradient Profile: Mobile phase initial composition of 90% A and 10% B, then to 70% A and 30% B after 2 min., then to 20% A and 80% B after 20 min., then to 5% A and 95% B after 21 min., held at this composition for 1 min. for column washing, then returned to the initial composition for 3 min.

LCMS Method

General 5 min method: Gemini C18 column (50×4.6 mm, 5 μm) operating at ambient temperature and a flow rate of 1.2 mL/min. Mobile phase: A=10 mM Ammonium Acetate in water, B=Acetonitrile; Gradient profile: from 90% A and 10% B to 70% A and 30 B in 1.5 min, and then to 10% A and 90% B in 3.0 min, held at this composition for 1.0 min, and finally back to the initial composition for 2.0 min.

UPLC Method

UPLC was carried out on a Waters UPLC using Kinetex EVo C18 column (100×2.1 mm, 1.7 μm) at ambient temperature and a flow rate of 1.5 ml/min.

Mobile phase 1: A=5 mM Ammonium Acetate in water, B=5 mM Ammonium Acetate in 90:10 Acetonitrile/water; Gradient profile from 95% A and 5% B to 65% A and 35% B in 2 min., then to 10% A and 90% B in 3.0 min., held at this composition for 2.0 min. and finally back to the initial composition for 6.0 min.

Mobile phase 2: A=0.05% formic acid in water, B=Acetonitrile; Gradient profile from 95% A and 5% B over 1 min., then 90% A and 10% B for 1 min., then 2% A and 98% B for 4 min. and then back to the initial composition for 6 min.

General Procedure 1 (Method a)

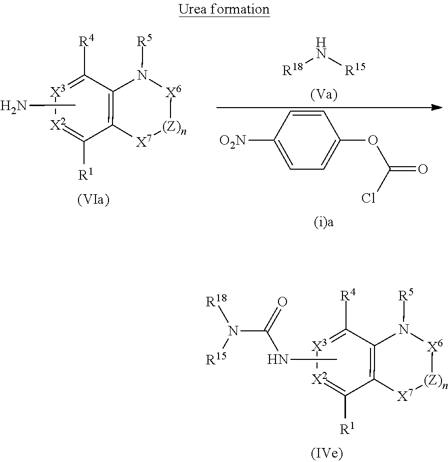

To a stirred solution of an aromatic amine of formula (VIa) (1.0 eq.) in a suitable solvent, such as THF, DMF, MeCN or DCM (8 mL/mmol) was added p-nitrophenyl chloroformate (1.2 eq.) at 0-5° C. and the whole stirred for 1-3 h at RT. Then amine R¹⁵—NH—R¹⁸ (Va) (1.1 eq.) and TEA or DIPEA (6 eq.) were added dropwise successively at 0-5° C. and the whole further stirred for 1-5 h at RT. Progress of the reaction was monitored by TLC/LCMS and after completion the reaction mass was diluted with water and extracted with EtOAc. The combined organic layers were washed with a dilute solution of a suitable inorganic base such as NaHCO₃ or 1N NaOH followed by 1N HCl and finally with brine. The organic layer was dried over anhydrous Na₂SO₄ and evaporated in vacuo to give a residue which was purified by column chromatography or combi-flash or prep-HPLC to afford a compound of formula (IVe) (yield 6-70%) as solids. A similar procedure can be followed to synthesize all urea of formula (IVe).

General Procedure 1 (Method b)

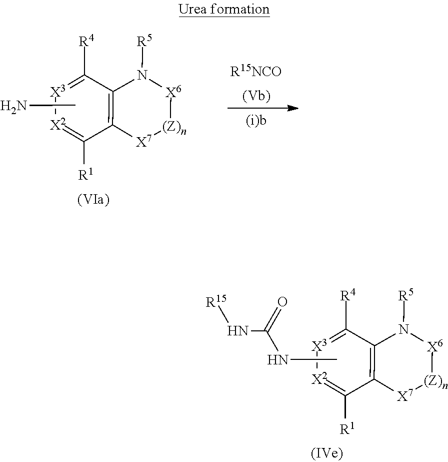

To a stirred solution of an aromatic amine of formula (VIa) (1.0 eq.) in a suitable solvent such as THF, DMF, MeCN or DCM (5.5 mL/mmol) was added R¹⁵NCO (Vb) (1.08 eq.) followed by TEA (1.08 eq.) at 0-5° C. and the whole stirred for 5-10 min. at the same temperature. The reaction mixture was brought slowly to RT and stirred for 1-2 h. Progress of the reaction was monitored by TLC and LC-MS. After completion, the reaction mixture was diluted with water and extracted with EtOAc. The combined organic layers were washed with brine, dried over anhydrous Na₂SO₄, filtered and evaporated under reduced pressure to afford a crude solid which was purified by column chromatography or combi-flash or prep-HPLC to afford a compound of formula (IVe) (yield 10-70%) as solids. A similar procedure can be followed to synthesize all ureas of formula (IVe).

General Procedure 1 (Method c)

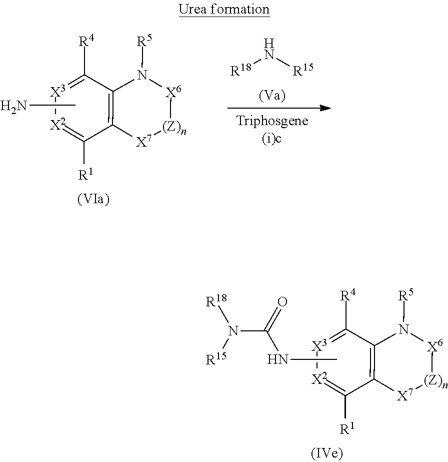

To a stirred solution of a compound of formula (Va) (68 mg, 0.519 mmol) in THF (10 mL/mmol) was added triphosgene (0.5 eq.) at 0-5° C. The combined mixture was stirred at RT for 1 h. Completion of the first stage of the reaction was confirmed by TLC or UPLC-MS before an aromatic amine compound of formula (VIa) (0.9 mmol) and TEA (2.5 eq.) were added into the reaction mixture and stirring continued at RT for 1-2 h. Progress of the reaction was monitored by TLC and or UPLC-MS. After completion of the reaction, the solvent was evaporated in vacuo to afford the crude material which was purified by column chromatography or prep-HPLC to give a compound of formula (IVe) (12-50% yield) as a solid.

General Procedure 2

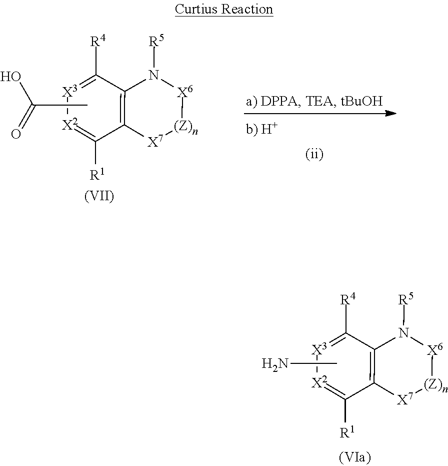

To a stirred solution of a compound of formula (VII) (1.0 eq.) in a suitable solvent such as MeCN, THF or DCM (3.5 mL/mmol) under an inert atmosphere was added TEA (1.5 eq.) followed by DPPA (2.0 eq.) at 0-5° C. and the whole stirred for 5-10 min. at the same temperature. The reaction mixture was then brought to RT and stirred for 4-6 h. Formation of the corresponding acyl azide was confirmed by TLC and UPLC-MS by quenching an aliquot of the reaction mixture with methanol. The solvents were evaporated in vacuo and tert-butanol (3.5 mL/mmol) added to the resulting residue. This mixture was then refluxed overnight. Completion of the reaction was monitored by TLC and LC-MS, which showed the formation of a BOC-protected amine compound of formula (VIa) with complete consumption of the compound of the starting material of formula (VII). After completion of the reaction, the solvent was evaporated in vacuo to obtain a crude oil which was adsorbed on silica gel and purified by Combi flash to afford the intermediate BOC-protected amine compounds of formula (VIa) (40-80% yield) as off white solids.

The resulting compound was dissolved in 1,4-dioxane (5.5 mL/mmol) and a solution of 4M HCl in 1,4-dioxane (5.5 mL/mmol) added at 0-5° C. and the whole stirred for 5-10 min. Then the reaction mixture was allowed to warm slowly to room temperature overnight. Completion of the reaction was confirmed by UPLC-MS and after completion the solvent was evaporated in vacuo. The resulting crude was then washed with NaHCO₃ solution and extracted with EtOAc. The organics were washed with brine, dried over anhydrous Na₂SO₄ and concentrated in vacuo to give compounds of formula (VIa) (yield 50-90%) as deep yellow solids.

General Procedure 3

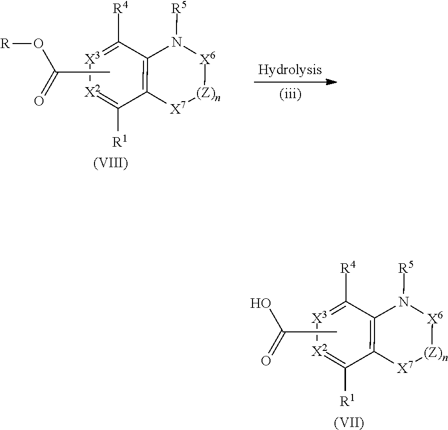

To a stirred solution of ester (VIII) (1.0 eq.) in a mixture of MeOH or THF (6.5 mL/mmol) and water (0.8 mL/mmol) was added LiOH, NaOH or KOH (2.0 eq.) at RT and the resulting reaction mixture was stirred at RT for 2-16 h. TLC showed complete consumption of the ester (VIII). The solvents were evaporated in vacuo and the resulting residue was washed with ether. The residue was then acidified with 1N HCl to pH 5-6, which resulted in the formation of a precipitate, which was filtered and washed with water and then dried by azeotropic distillation or under reduced pressure at 50-60° C. to afford the desired carboxylic acids of formula (VII) (70-85% yield) as solids.

General Procedure 4

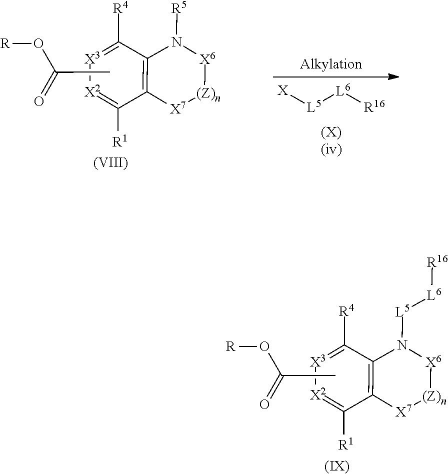

Option A

To a stirred solution of a compound of formula (VIII) (1.0 eq.) in DMF or THF (4 mL/mmol) was added K₂CO₃, Cs₂CO₃, Na₂CO₃, NaOH or NaH (1.1 eq.). In the case where NaOH was used, TBAB (0.1 eq.) was also added as a phase transfer catalyst, followed by addition of a compound of formula (X) (1.05 eq.) and the mixture allowed to stir at RT for 0.5-1 h. The reaction was monitored by TLC. After completion of the reaction the reaction mixture was quenched with a saturated solution of NH₄Cl, diluted with ice-cold water and extracted with EtOAc or MTBE. The organic layers were washed with brine, dried over anhydrous Na₂SO₄ and evaporated in vacuo to afford the crude product which was purified by Combi-flash using mixtures of EtOAc in hexanes as eluent to give compounds of formula (IX) (60-80% yield) as colourless oils.

Option B

Alternatively, to a stirred solution of a compound of formula (VIII) (1.0 eq.) in DCM or MeCN or THF (4 mL/mmol) was added TEA or DIPEA (2.0 eq.) or without the base followed by addition of a compound of formula (X) (1.5 eq.) and the whole allowed to stir at RT for 0.5 to 1 h. The progress of the reaction was monitored by TLC. After completion of the reaction, the mixture was diluted with water, extracted with EtOAc, and the combined organic layers were washed with brine and dried over anhydrous Na₂SO₄. The organic layers were evaporated in vacuo to obtain the crude product which was purified by Combi-flash using mixtures of EtOAc in hexanes as eluent to afford compounds of formula (IX) (60-80% yield) as colourless oils.

General Procedure 5

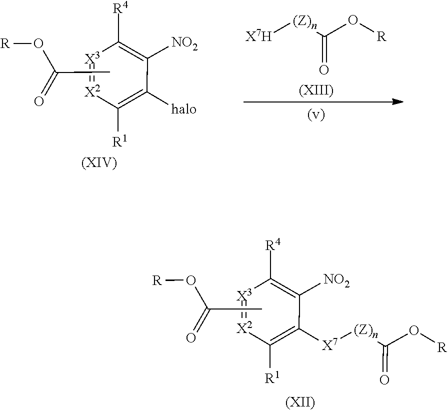

To a stirred solution of a compound of formula (XIV) (1.0 eq.) and a suitable nucleophile (XIII) (1.25 eq.) in a suitable solvent, such as 1,4-dioxane, MeCN, DMF or THF (3 mL/mmol), was added dropwise or portionwise a suitable base such as TEA, DBU, NaH or K₂CO₃ (1.5 eq.) with ice bath cooling and the combined mixture allowed to stir at 0-25° C. for 1-16 h. Progress of the reaction was monitored by TLC or LCMS and on completion of the reaction the mixture was quenched with a saturated aqueous solution of NH₄Cl and extracted with EtOAc. The combined organic layers were washed with brine, dried over anhydrous Na₂SO₄ and evaporated in vacuo to dryness. The crude compounds of formula (XII) (60-95% yield) obtained as solids were pure enough to be used directly in the next step without any further purification.

General Procedure 6

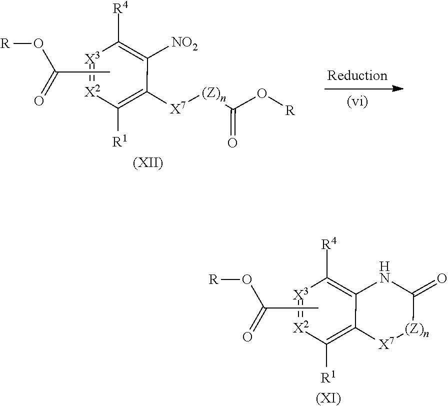

Option a (Reduction by Fe/Zn-AcOH/HCl/NH4Cl)

To a stirred solution of a compound of formula (XII) (1.0 eq.) in EtOH or MeOH (2 mL/mmol) was added a suitable acid, such as AcOH or aq. HCl (3 mL/mmol) followed by iron powder or zinc powder (4.0 eq.) at RT. In some cases NH4Cl was also used as source of hydrogen. The reaction mixture was stirred at 75-85° C. for 1-5 h. The reaction was monitored by TLC or LCMS and after completion the reaction mixture was poured into ice-cold water and filtered through a short celite bed. The filtrate was extracted with EtOAc and then washed with aqueous NaHCO₃ and then brine. The collected organic layers were dried over anhydrous Na₂SO₄ and concentrated in vacuo to afford compounds of formula (XI) (60-80% yield) as crude solid, which were used in the next step without any further purification.

Option B: (Reduction by Sodium Dithionate)

To a stirred solution of a compound of formula (XII) (1.0 eq.) in a mixture of either MeCN/H₂O or THF/H₂O (12 mL/mmol, 2:1) was added sodium hydrosulphite (8.0 eq.), tetra-butyl ammonium hydrosulphate (0.5 eq.) and K₂CO₃ (6.0 eq.) at RT and the mixture then stirred for 1 h. Progress of the reaction was monitored by TLC and or LCMS. After completion of the reaction the solvents were evaporated in vacuo to give an oily liquid which was dissolved in 1N HCl and extracted with EtOAc. The combined organic layers were washed with brine and dried over anhydrous Na₂SO₄. The organics were filtered and evaporated in vacuo to give a compound of formula (XI) (80-90% yield) as solids.

Option C: (Reduction by Pd/C/H₂)

To a stirred solution of a compound of formula (XII) (1.0 eq.) in EtOAc, MeOH or EtOH (9.4 mL/mmol, 120 mL) was added 10% Pd—C(50% w/w in water) (77.8 mg/mmol) under an inert atmosphere at room temperature. The reaction mixture was purged with H₂ gas using balloon pressure and then allowed to further stir for 3-5 h at room temperature. The course of the reaction was monitored by TLC and/or LCMS. After completion of the reaction the mixture was diluted with EtOAc, filtered carefully through a bed of celite and washed with EtOAc 4-5 times until the mother liquor showed no compound remaining by TLC. Then the collected organic layers were dried over anhydrous Na₂SO₄, filtered and concentrated under reduced pressure to give a compound of formula (XI) (80-85% yield) as semi-solids. The products were pure enough to use in the next step without any further purification.

Option D: (Reduction by NiCl₂.6H₂O/NaBH₄)

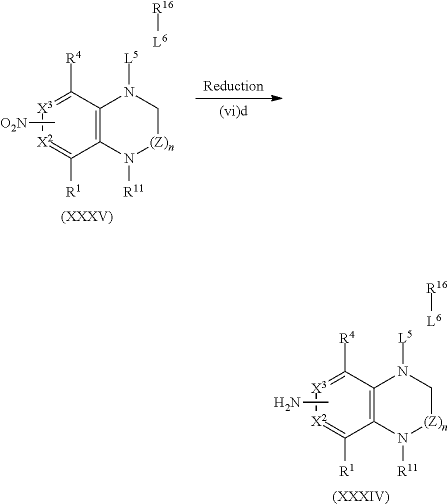

To a stirred solution of a compound of formula (XXXV) (1.0 eq. 0.53 mmol) in MeOH (9 mL/mmol) was added Boc₂O (1.5 eq.) followed by NiCl₂.6H₂O (0.5 eq.) and NaBH₄ (2.5 eq.) at 5-10° C. The combined mixture was then allowed to warm to RT over 3-5 h. Progress of the reaction was monitored by TLC and UPLC-MS which showed formation of the intermediate product. After completion, the reaction mixture was diluted with chilled water and extracted with EtOAc. The combined organic layers were washed with brine, dried over anhydrous Na₂SO₄, filtered and evaporated under reduced pressure to afford the crude product which was purified by Combi-flash to provide the Boc-protected amine compound (90-96% yield, 0.51 mmol). This material was dissolved in DCM (9 mL/mmol) and TFA (4 mL/mmol) and the whole was stirred at RT for 4-6 h. UPLC-MS showed formation of the desired product. The solvent was evaporated in vacuo to give the crude product which was neutralized with aqueous sodium carbonate solution and extracted with EtOAc. The combined extracts were washed with brine, dried over anhydrous Na₂SO₄, filtered and evaporated under reduced pressure to afford the compound of formula (XXXIV) (80-85% yield) as a semi-solid.

General Procedure 7

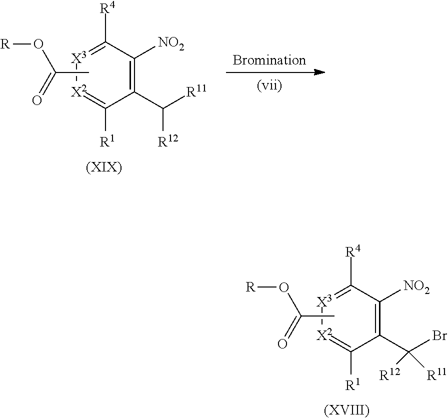

To a stirred solution of a compound of formula (XIX) (1.0 eq.) in a suitable solvent such as carbon tetrachloride or trifluoromethylbenzene (100 mL) was added NBS (1.2 eq.) and AIBN or benzoyl peroxide (0.1 eq.). The reaction mixture was heated at 70-100° C. for 12-16 h. After complete consumption of the starting material, the reaction mixture was quenched with a saturated solution of Na₂S₂O₃ and extracted with EtOAc. The combined organic layers were washed with brine and dried over anhydrous Na₂SO₄. The crude product obtained after concentration of the organic layer in vacuo was purified by column chromatography to afford a compound of formula (XVIII) in 30-40% yield.

General Procedure 8

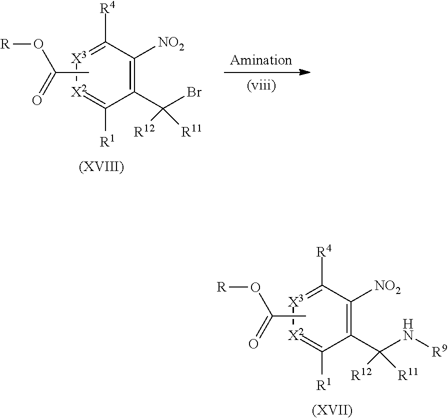

To a stirred solution of a compound of formula (XVIII) (1.0 eq.) in a suitable solvent such as THF (5 mL/mmol) was added a suitable amine such as MeNH₂, (3 mL/mmol, 2M solution in THF) at RT and the combined mixture was stirred at the same temperature or elevated temperature (60-90° C.) for 10-16 h. After completion of the reaction, the reaction mixture was diluted with water and extracted with EtOAc. The combined organic layers were washed with a saturated brine solution, dried over anhydrous Na₂SO₄ and concentrated in vacuo to afford a compound of formula (XVII) (60-70% yield) as gummy solids.

General Procedure A

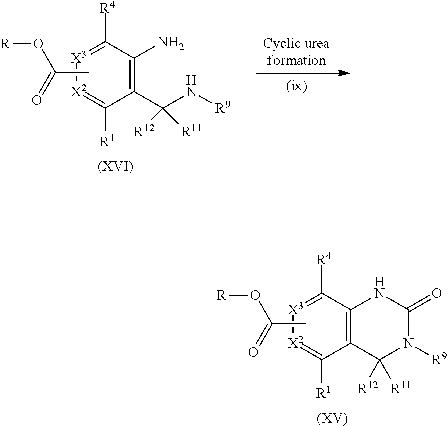

To a stirred solution of a compound of formula (XVI) (1.0 eq.) in a suitable solvent, such as DCM or THF (5 mL/mmol) was added a suitable carbonyl source equipped with suitable leaving groups, such as 1,1-carbonyl-diimidazole, phosgene or triphosgene (1.1 eq.) followed by a suitable base, such as TEA or DIPEA (3.0 eq.) at 0-5° C. and the reaction mixture was stirred at room temperature under an inert atmosphere for 2-4 h. The reaction mixture was quenched by the addition of a saturated aqueous NaHCO₃ solution and extracted with DCM. The combined organic layers were dried over anhydrous Na₂SO₄, filtered and concentrated in vacuo to provide a crude residue which was purified by silica gel column chromatography and eluted with 1% MeOH in DCM to afford a compound of formula (XV) (20-30% yield) as solids.

General Procedure 10

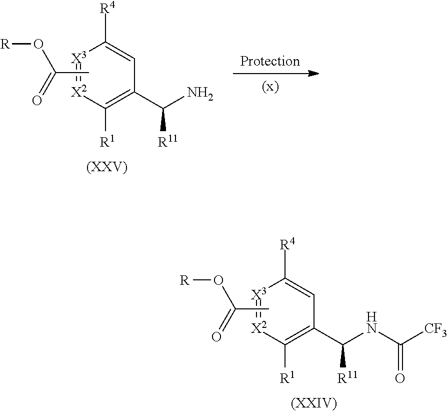

To a stirred solution of a compound of formula (XXV) (1.0 eq.) in toluene (1.8 mL/mmol) was added TFAA (2.0 eq.) at 10-15° C. dropwise over 20-30 min. and the resulting reaction mixture was stirred at 25-30° C. for 1-5 h. Progress of the reaction was monitored by UPLC-MS. After completion, the reaction mixture was poured into crushed ice and extracted with EtOAc. The combined organic layers were washed successively with a saturated aqueous solution of NaHCO₃, brine and then dried over anhydrous Na₂SO₄. The filtered organics were evaporated under reduced pressure to afford compounds of formula (XXIV) (85-90% yield) as solids. The products were pure enough to use in the next step without any further purification.

General Procedure 11

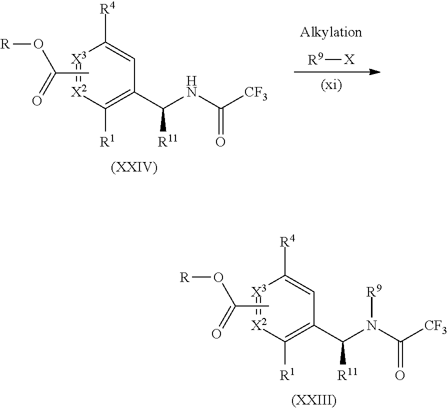

To a stirred solution of NaH (1.2 eq., 60% suspension in oil) in DMF (1.65 mL/mmol) was added a mixture of a compound of formula (XXIV) (1.0 eq.) and an alkyl or aryl halide (R⁹—X) (2.0 eq.) in DMF (1.1 mL/mmol) dropwise using a dropping funnel over 20-30 min. at 10-15° C. and the resulting reaction mixture then stirred for 2 h at 20-25° C. Completion of the reaction was confirmed by UPLC-MS. The reaction mixture was poured into an ice-water mixture and extracted with EtOAc. The combined organics were washed with 1N HCl, a saturated solution of NaHCO₃ and then brine. The organic layer was dried over anhydrous Na₂SO₄ and evaporated under reduced pressure to afford a compound of formula (XXIII) (90-95% yield) as solids. The product was pure enough to use in the next step without any further purification.

General Procedure 12

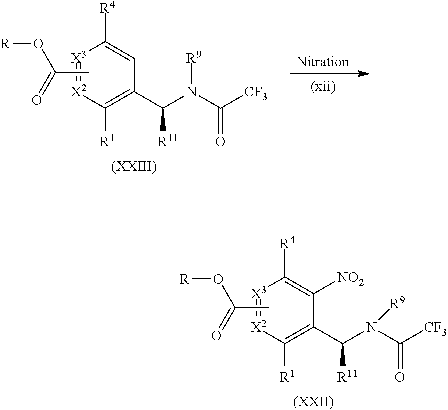

A compound of formula (XXIII) (1.0 eq.) was added into a pre-prepared nitrating mixture of concentrated sulfuric acid (2.17 mL/mmol) and fuming nitric acid (0.73 mL/mmol) portionwise whilst maintaining the internal temperature between 0-5° C. over a period of 30 min. The resulting mixture was stirred at 20-25° C. for 1-2 h. Completion of the reaction was confirmed by UPLC-MS and after consumption of the starting material the reaction mixture was poured into an ice-water mixture and extracted with EtOAc. The combined organics were washed with a saturated solution of NaHCO₃ followed by a saturated brine solution, dried over anhydrous Na₂SO₄ and evaporated under reduced pressure to afford a compound of formula (XXII) (yield 85-95%) as thick oil. The product was pure enough to use in the next step without any further purification.

General Procedure 13

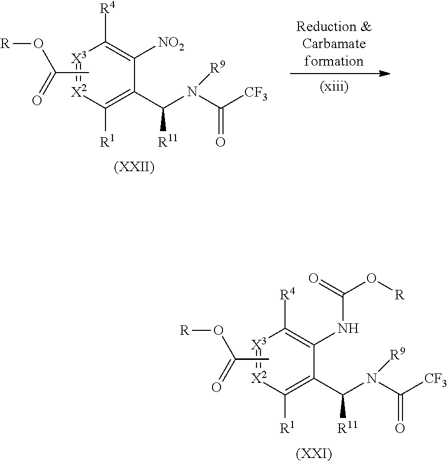

Option A

To a stirred solution of a compound of formula (XXII) (1.0 eq.) in 1,4-dioxane (3.34 mL/mmol, degassed with nitrogen) was added 10% Pd—C(0.167 g/mmol, 50% w/w in water) under an inert atmosphere and the resulting reaction mixture was stirred under H₂ gas balloon pressure at RT for overnight. Progress of the reaction was monitored by TLC and UPLC-MS which showed complete conversion of the nitro group into its corresponding amino group. The balloon was removed and solid K₂CO₃ (1.66 eq.) was added into the reaction vessel followed by the dropwise addition of ethyl chloroformate (1.34 eq.) at RT. The resulting reaction mixture was further stirred overnight. UPLC-MS showed completion of the reaction; the reaction mixture was filtered through a celite bed and the bed was washed with DCM. The filtrate was evaporated in vacuo to give a crude product which was dissolved in EtOAc, washed with water followed by brine, dried over anhydrous Na₂SO₄ and evaporated in vacuo to afford a crude product as a thick oil which was purified by trituration with n-hexane and dried to afford a compound of formula (XXI) (80-85% yield) as solids.

Option B

To a stirred solution of a compound of formula (XXII) (1.0 eq.) in THF (6.68 mL/mmol) was added a solution of K₂CO₃ (6.0 eq.) in water (3 mL/mmol) at 10-15° C. followed by portionwise addition of sodium dithionite (8.0 eq.), TBASH (0.5 eq.) and water (0.4 mL/mmol). The resulting reaction mixture was stirred at RT (20-25° C.) for a further 2-3 h. The reaction was monitored by UPLC-MS and after completion the reaction mixture was left to settle to allow separation of the organic and aqueous layers. The aqueous layer was then extracted with THF. The combined organic layers were dried over anhydrous Na₂SO₄ and then pyridine (0.8 mL/mmol) was added. The mixture was then evaporated at −40° C. under reduced pressure to afford the crude product which was dissolved in DCM (6.7 mL/mmol) and another portion of pyridine (0.8 mL/mmol) added followed by dropwise addition of ethyl chloroformate (5.0 eq.) at 10-15° C. The resulting reaction mixture was further stirred at RT for 2-3 h. UPLC-MS showed completion of the reaction. The reaction mixture was diluted with water and allowed to settle to allow separation of the layers. The aqueous layer was washed with DCM and the combined organics were washed with 0.5N HCl, a saturated solution of NaHCO₃ and finally with brine. The obtained organic layer was dried over anhydrous Na₂SO₄ and evaporated in vacuo to afford the crude product as a yellowish thick oil. The oil was purified by trituration with hexane to give a compound of formula (XXI) (85-90% yield) as solids.

General Procedure 14

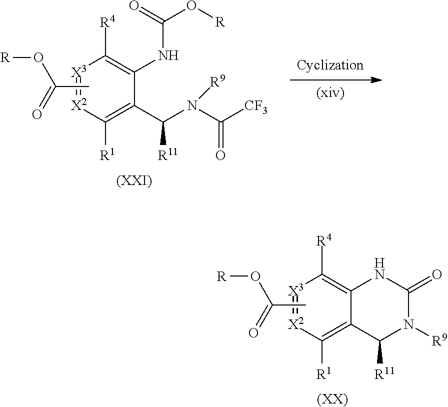

To a stirred solution of a compound of formula (XXI) (1.0 eq.) in methanol (3.8 mL/mmol) was added K₂CO₃ (2.0 eq.) at RT and the resulting reaction mixture was heated to 60-65° C. for 2-3 h. Progress of the reaction was monitored by UPLC-MS and after completion, the reaction mass was cooled to 5-10° C. and acidified with 2N HCl to pH ˜3-4. The solvents were evaporated under reduced pressure at 40-45° C. to give the crude product which was dissolved in EtOAc, washed successively with a saturated brine solution, 2N HCl, NaHCO₃ solution and finally again with brine, dried over anhydrous Na₂SO₄ and evaporated under reduced pressure to afford the crude compound as a brownish solid. This was purified by trituration with n-hexane to afford a compound of formula (XX) (80-85% yield) as solids.

General Procedure 15

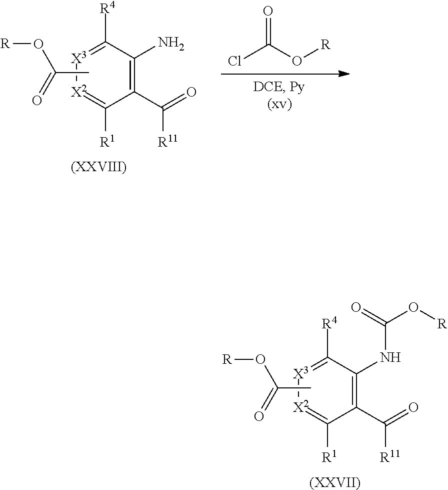

To a stirred solution of a compound of formula (XXVIII) (1.0 eq.) in DCE (1.8 mL/mmol) was added pyridine (2.2 eq.) and alkyl(aryl)chloroformate (1.2 eq.) at 0-5° C. and the mixture stirred at RT for 1-2 h. Progress of the reaction was monitored by TLC and LC-MS. Upon completion, the reaction mixture was quenched with 1N HCl solution and extracted with DCM followed by a brine wash. The organic layer was dried over anhydrous Na₂SO₄ and concentrated in vacuo to afford a compound of formula (XXVII) (70-75% yield) as solids which were used in the next step without any further purification.

General Procedure 16

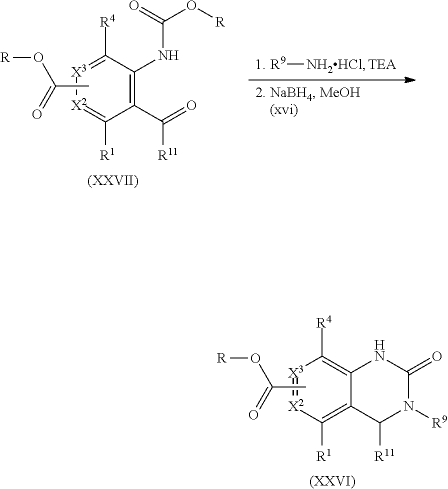

To a stirred solution of an amine R⁹—NH₂.HCl (1.0 eq.) in MeOH (5 mL/mmol) was added TEA (1.2 eq.) under an inert atmosphere at RT and the whole was stirred for 30 min. Then, a compound of formula (XXVII) (1.0 eq.) was added and stirring was continued for 20-24 h. During this period, the solution became a suspension. NaBH₄ (1.5 eq.) was added and the reaction mixture was further stirred for another 20-24 h. Completion of the reaction was monitored by TLC and LC-MS and after completion the reaction mixture was diluted with water and extracted with EtOAc followed by a brine wash. The organic layer was dried over anhydrous Na₂SO₄ and concentrated in vacuo to afford a compound of formula (XXVI) as solids.

General Procedure 17

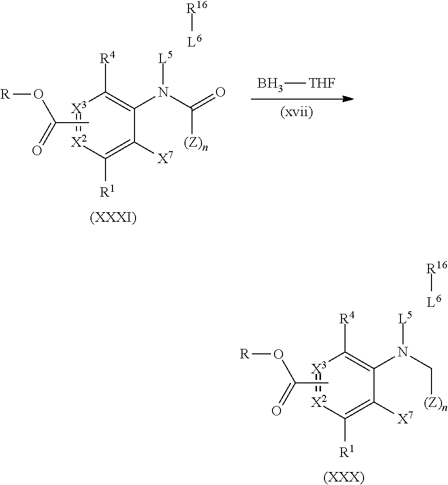

A stirred solution of a compound of formula (XXXI) (1.0 eq. 0.96 mmol) in THF (5 mL/mmol) was cooled to 0-5° C. and borane-THF complex (1M solution in THF) (10 mL/mmol, 10 eq.) added portionwise. After the addition was complete, the mixture was allowed to warm to RT, and then heated to reflux for 1-2 h. Progress of the reaction was monitored by UPLC-MS which showed formation of a compound of formula (XXX). After completion the reaction mixture was diluted with methanol and refluxed for 5-10 min., the solvent was evaporated to give a crude material which was purified by Combi-flash or column chromatography to afford a compound of formula (XXX) as colorless oil.

General Procedure 18

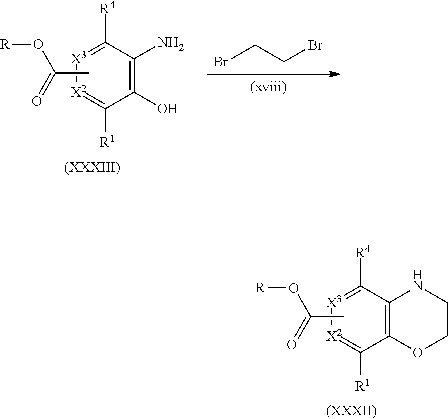

To a stirred solution of a compound of formula (XXXIII) (1.0 eq.) in DMF or THF (1.6 mL/mmol) was added K₂CO₃, Cs₂CO₃, Na₂CO₃, NaOH or NaH (4.0 eq.) at RT and then 1,2-dibromoethane (4.0 eq.) was added and the reaction mass maintained at 80-85° C. for 10-16 h. Progress of the reaction was monitored by TLC and UPLC-MS which showed formation of the desired product. After completion of the reaction, the reaction mixture was diluted with water and extracted with EtOAc. The combined organics were washed with brine, dried over anhydrous Na₂SO₄ and evaporated in vacuo to afford a crude material which was purified by Combi-flash to afford compounds of formula (XXXII) (50-55% yield) as solids.

General Procedure 19

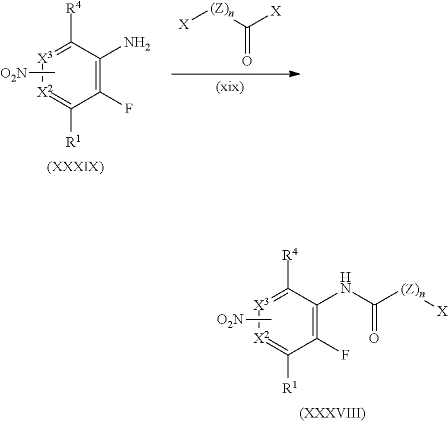

To a stirred solution of a compound of formula (XXXIX) (1.0 eq.) in acetone (3.2 mL/mmol) was added a suitable haloacetyl halide (1.3 eq.) at RT and the combined mixture was stirred at RT for 1-2 h. Progress of the reaction was monitored by TLC and UPLC-MS and after completion the reaction mixture was quenched with ice-cold water to give a solid precipitate which was filtered, washed with water and then dried in a vacuum oven to afford a compound of formula (XXXVIII) (85-90% yield) as a brownish solid.

General Procedure 20

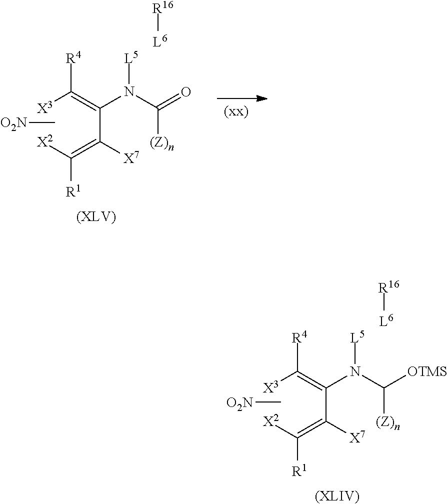

To a stirred solution of a compound of formula (XLV) (1.0 eq.) in DCM (10 mL/mmol) was added DIBAL-H (1.5 eq.) at −78° C. under a nitrogen atmosphere. The whole was stirred for 1-2 h at the same temperature and then pyridine (3.5 eq.) and TMSOTf (3.0 eq.) were added to the reaction mixture. The temperature of the reaction was then slowly allowed to rise to 0-5° C. Progress of the reaction was monitored by TLC and after completion of the reaction, Et₂O (285 mL/mmol) was added and the mixture was filtered. The collected organic layer was then concentrated in vacuo to afford compound of formula (XLIV) as crude solids.

General Procedure 21

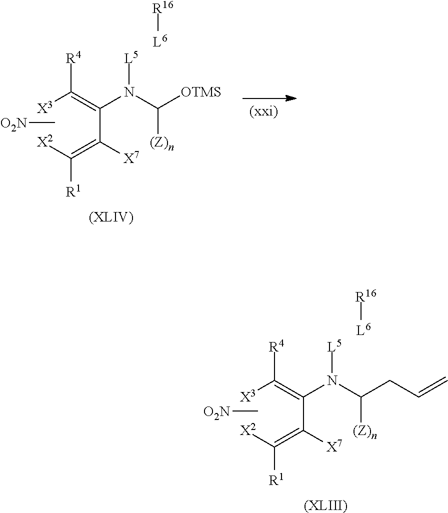

To a stirred solution of a compound of formula (XLIV) (1.0 eq.) in DCM (10 mL/mmol) was added allyl-TMS (4.0 eq.) and BF3.Et2O (4.0 eq.) at −78° C. under nitrogen. The temperature was then slowly raised to 0-5° C. Progress of the reaction was checked by UPLC-MS and after completion of the reaction it was quenched with water and extracted with EtOAc. The combined organic layer was collected, dried over anhydrous Na₂SO₄, filtered and evaporated to dryness. The crude product was purified by column chromatography to afford the title compounds of formula (XLIII) (70-75% yield) as pure solids.

General Procedure 22

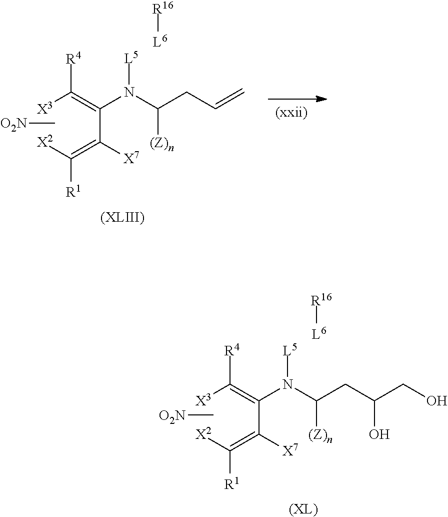

To a stirred solution of a compound of formula (XLIII) (1.0 eq.) in ^{t}BuOH/H₂O solution (12 mL/mmol, 1:1) was added OsO₄ (0.09 eq.) and NMO (1.4 eq.). The resulting reaction mixture was stirred at RT for 10-12 h. Progress of the reaction was checked by LCMS and after completion of the reaction it was further diluted with EtOAc. The organic layer was separated and washed with 10% HCl, water and finally with brine. It was then dried and concentrated in vacuo to afford a compound of formula (XL) as a crude solid.

General Procedure 22

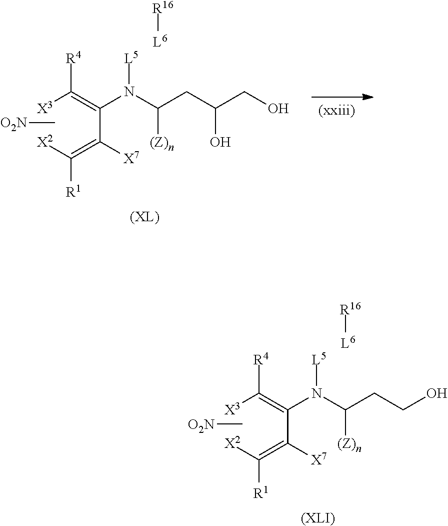

To a stirred solution of a compound of formula (XL) (1.0 eq.) in ^{t}BuOH/H₂O solution (12 mL/mmol, 1:1) was added NaIO₄ (4.0 eq.) at RT. The resulting reaction mixture was stirred at RT for 10-12 h. Progress of the reaction was checked by LCMS and after completion of the reaction it was diluted with water and extracted with EtOAc. The separated organic layer was dried and concentrated in vacuo to afford the crude corresponding aldehyde which was dissolved in methanol (12 mL/mmol) and NaBH₄ (2.0 eq.) added at 0-5° C. The reaction mixture was further stirred at RT for 1-2 h. After completion of the reaction it was quenched with NH₄Cl solution and extracted with EtOAc. The separated organic layers were dried and concentrated in vacuo to afford compound of formula (XLI) as crude solids.

General Procedure 24

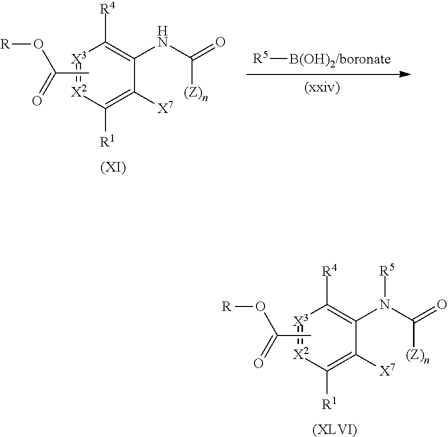

To a stirred solution of a compound of formula (XI) (1.0 eq.) in EDC (1.1 mL/mmol) was added R⁵—B(OH)₂/boronate (1.5 eq.) in EDC or toluene (1.1 mL/mmol), DBU (2.0 eq.) and a solution of Cu(OAc) (2.0 eq.) at RT. The resulting reaction mixture was stirred at RT for 20-24 h. Progress of the reaction was monitored by LCMS and after completion the reaction mixture was diluted with water and extracted with EtOAc. The organic layer was washed with brine, dried over anhydrous Na₂SO₄ and evaporated in vacuo to afford the crude material which was purified by Combi-flash to afford a compound of formula (XLVI) (34-40% yield) as a solid.

General Procedure 25

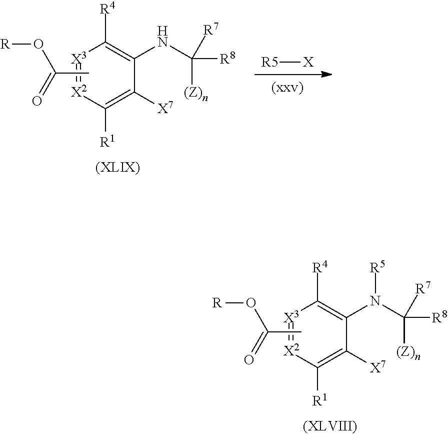

To a stirred solution of a compound of formula (XLIX) (1.0 eq.) in toluene or dioxane or EDC (6 mL/mmol) was added R⁵—X (where X is a suitable leaving group) (1.5 eq.), cesium carbonate (2.0 eq.) and BINAP (0.2 eq.) at RT. The whole was degassed with nitrogen for 20 min., then palladium acetate (0.1 eq.) was added into the reaction mixture and stirring continued at 100-110° C. for 20-24 h. Progress of the reaction was monitored by UPLC-MS and after completion the reaction mixture was concentrated in vacuo to give a crude material which was purified by column chromatography to afford a compound of formula (XLVIII) (30-35% yield) as a solid.

General Procedure 26

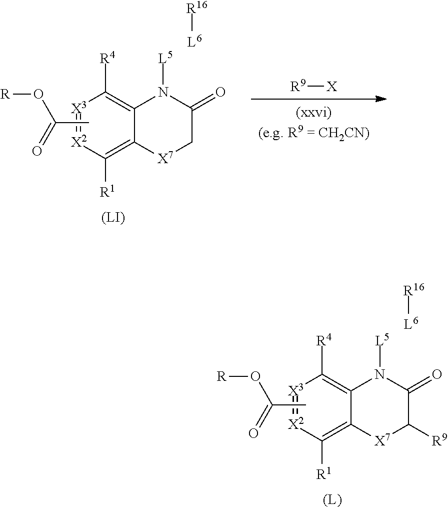

To a stirred solution of a compound of formula (LI) (1.0 eq.) in dry Et₂O or THF (6 mL/mmol) was added LiHMDS (1.5 eq.) at −78° C. under an inert atmosphere and stirred for 5-10 min. R⁹—X e.g. bromoacetonitrile (1.2 eq.) was then added to the reaction mixture and stirring continued for 30 min. at the same temperature. After this time, the reaction mixture was brought slowly to room temperature and stirred for 1-2 h. Progress of the reaction was monitored by UPLC-MS and after completion of the reaction it was quenched with a saturated solution of NH₄Cl and extracted with EtOAc. The combined organic layers were washed with brine, dried over anhydrous Na₂SO₄, filtered and concentrated under reduced pressure to afford the crude product which was purified by combi-flash to afford a compound of formula (L) (45-50% yield) as a solid.

General Procedure 27

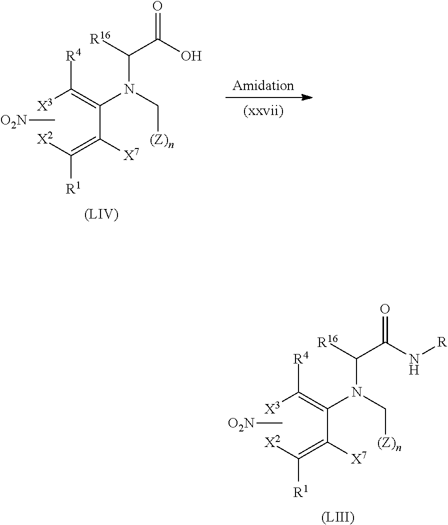

To a stirred solution of a compound of formula (LIV) (1.0 eq.) in DMF (5.5 mL/mmol) was added an amide coupling reagent such as EDC-HCl (1.5 eq.) and DIPEA (3.0 eq.) at 0-5° C. and the reaction mixture was stirred for 5-10 min. at this temperature. R—NH2 (5.0 eq.) was then added and the reaction mixture was stirred at RT for 10-16 h. After completion of the reaction (monitored by TLC), the solvent was evaporated under reduced pressure to give a residue which was extracted with EtOAc and the combined organic layers were dried over anhydrous Na₂SO₄, filtered and evaporated under reduced pressure to afford the crude product. This crude material was purified by column chromatography to give compounds of formula (LIII) (70-75% yield) as solids.

Library General Procedure 28

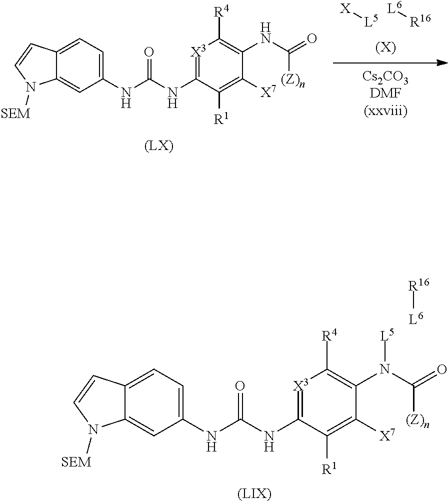

To a stirred solution of a compound of formula (LX) (1.0 eq.) in an appropriate amount of DMF was added cesium carbonate (2.0 eq) at room temperature under a nitrogen atmosphere followed by addition of X-L⁵-L⁶-R¹⁶ (X) (1.5 eq.). The reaction mixture was then stirred at RT for 15-20 h. Progress of the reaction was monitored by LC-MS. After completion of the reaction, the reaction mass was diluted with diethylether and washed with water. The organic layer was dried over anhydrous Na₂SO₄, and concentrated in vacuo to afford a compound of formula (LIX) as a solid which was used in the next step without any further purification.

Library General Procedure 29

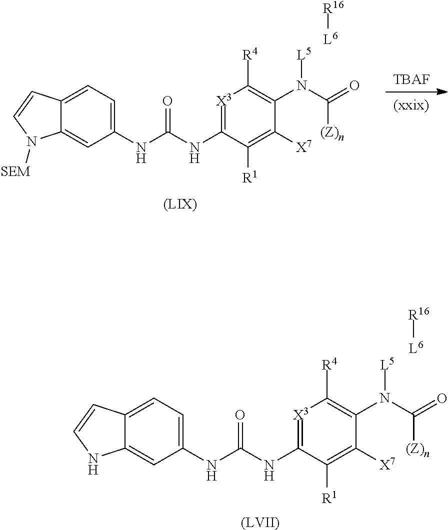

To a stirred solution of a compound of formula (LIX) (1.0 eq.) in a suitable amount of THF was added ethylene diamine (6.0 eq.) at 0-5° C. Thereafter, TBAF (12.0 eq.) was added dropwise at the same temperature. The resulting reaction mixture was stirred at 70-75° C. for 48-72 h. Progress of the reaction was monitored by LC-MS. After completion of the reaction, the reaction mass was diluted with water and extracted with EtOAc. The organic layer was dried over anhydrous Na₂SO₄, and concentrated in vacuo to give the crude product which was purified by column chromatography or preparative-HPLC to afford compounds of formula (LVII) as solids.

### EXAMPLES

Nuclear magnetic resonance (NMR) spectra were in all cases consistent with the proposed structures. Characteristic chemical shifts (S) are given in parts-per-million downfield from tetramethylsilane (for ¹H-NMR) and upfield from trichloro-fluoro-methane (for ¹⁹F NMR) using conventional abbreviations for designation of major peaks: e.g. s, singlet; d, doublet; t, triplet; q, quartet; m, multiplet; br, broad. The following abbreviations have been used for common solvents: CDCl₃, deuterochloroform; d₆-DMSO, deuterodimethylsulphoxide; and CD₃OD, deuteromethanol.

Mass spectra, MS (m/z), were recorded using electrospray ionisation (ESI). Where relevant and unless otherwise stated the m/z data provided are for isotopes ¹⁹F, ³⁵Cl, ⁷⁹Br and ¹²⁷I.

All chemicals, reagents and solvents were purchased from commercial sources and used without further purification. All reactions were performed under an atmosphere of nitrogen unless otherwise noted.

Flash column chromatography was carried out using pre-packed silica gel cartridges in a Combi-Flash platform. Prep-HPLC purification was carried out according to the General purification and analytical methods described above. Thin layer chromatography (TLC) was carried out on Merck silica gel 60 plates (5729). All final compounds were >95% pure as judged by the LCMS or UPLC analysis methods described in the General Purification and Analytical methods above unless otherwise stated.

### Example 1: 1-(4-Benzyl-3-oxo-3,4-dihydro-2H-benzo[b][1,4]thiazin-6-yl)-3-(1H-indol-6-yl)urea

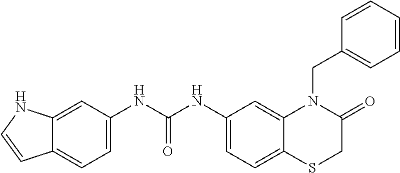 [SMILES: O=C(Nc1ccc2c(c1)N(Cc1ccccc1)C(=O)CS2)Nc1ccc2cc[nH]c2c1]

Example 1 was prepared according to the methods described in General Procedures 1-6, and the methods described below.

### Preparation 1: Methyl 3-oxo-3,4-dihydro-2H-1,4-benzothiazine-6-carboxylate

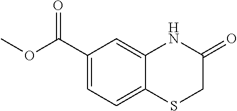

### Step 1: Methyl 4-((2-ethoxy-2-oxoethyl)thio)-3-nitrobenzoate

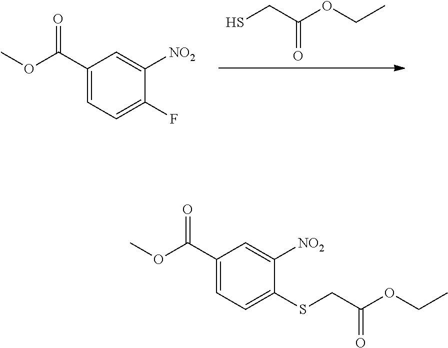

Methyl 4-fluoro-3-nitrobenzoate (10.0 g, 50.2 mmol) was taken up in MeCN (2.0 L) and TEA (7.61 g, 75.38 mmol) was added to the solution. The reaction mixture was cooled to 0-5° C. and ethyl thioglycolate (7.25 g, 62.7 mmol) was added dropwise. The reaction mixture was stirred for 30 min. at ice-cold temperature. It was then diluted with EtOAc and washed with a saturated solution of NH₄Cl and brine. The organic layer was dried over anhydrous Na₂SO₄ and evaporated in vacuo to dryness to give the title compound (14.0 g, 46.82 mmol, 93% yield) as a yellow solid, which was pure enough to be used in the next step without any further purification. LCMS m/z: 300.06 [M+H].

### Step 2: Methyl 3-oxo-3,4-dihydro-2H-benzo[b-1,4]thiazine-6-carboxylate

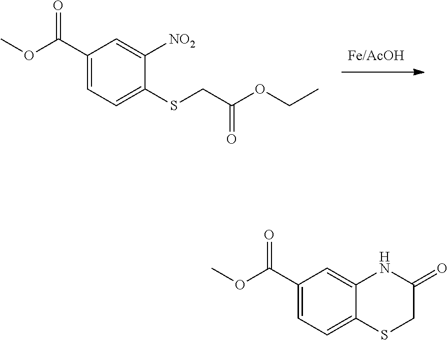

To a stirred solution of methyl 4-((2-ethoxy-2-oxoethyl)thio)-3-nitrobenzoate (Step 1) (5.0 g, 16.7 mmol) in acetic acid (50 mL) was added iron powder (3.73 g, 66.8 mmol). The resulting reaction mixture was stirred at 80° C. for 3 h. On completion (monitored by TLC), the reaction was cooled to room temperature and poured onto 1N HCl (250 mL) and then stirred for 1 h. The resulting white precipitate was filtered off and washed with water. The residue obtained was re-dissolved in 5% MeOH in DCM (50 mL) and filtered through a bed of celite. The filtrate was evaporated to dryness in vacuo to afford the title compound (3.5 g, 15.6 mmol, 91% yield) as a pale yellow solid. LCMS m/z: 222.05 [M−H].

### Preparation 2: Methyl 4-benzyl-3-oxo-3,4-dihydro-2H-benzo[b][1,4]thiazine-6-carboxylate

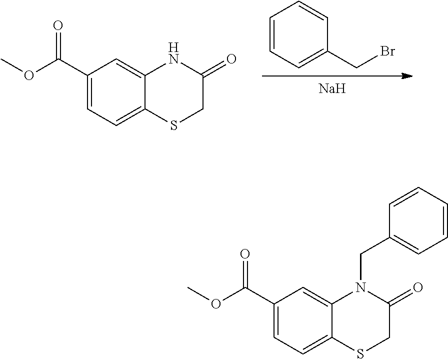

To a stirred solution of methyl 3-oxo-3,4-dihydro-2H-benzo[b-1,4]thiazine-6-carboxylate (Preparation 1, Step 2) (5.0 g, 22.2 mmol) in DMF (50 mL) at 0-5° C. was added NaH (0.98 g, 24.4 mmol) portionwise and the whole stirred for another 5-10 min. at the same temperature. Then, benzyl bromide (2.8 mL, 23.3 mmol) was added and the reaction mixture was stirred for 1 h. Completion of the reaction was monitored by TLC and LC-MS. After completion, the reaction mixture was quenched with a saturated solution of NH₄Cl and diluted with ice-cold water. The aqueous reaction mixture was extracted with MTBE and washed with brine. The separated organic layer was then dried over anhydrous Na₂SO₄ and concentrated under reduced pressure to afford the title compound (9.0 g) as a crude pale yellow solid which was used in the next step without any further purification. LCMS m/z: 314.16 [M+H].

### Preparation 3: 4-Benzyl-3-oxo-3,4-dihydro-2H-benzo[b][1,4]thiazine-6-carboxylic acid

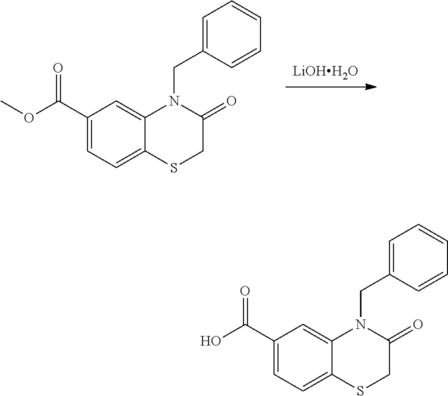 [SMILES: COC(=O)c1ccc2c(c1)N(Cc1ccccc1)C(=O)CS2 ; "LiOH·H2O" ; O=C(O)c1ccc2c(c1)N(Cc1ccccc1)C(=O)CS2]

To a stirred solution of methyl 4-benzyl-3-oxo-3,4-dihydro-2H-benzo[b][1,4]thiazine-6-carboxylate (Preparation 2) (9.0 g, 28.8 mmol) in a mixture of solvents THF/MeOH/H₂O (160 mL, 2:1:1) was added LiOH.H₂O (4.8 g, 115.2 mmol) at RT and the combined mixture stirred for 2 h at the same temperature. Progress of the reaction was monitored by TLC and LC-MS, showing complete consumption of the starting material. The solvents were evaporated in vacuo and the resulting residue was diluted with water and washed with EtOAc. The aqueous layer was collected and acidified with 1N HCl to pH 5-6 to obtain a precipitate which was filtered, collected and dried by azeotropic distillation with MeCN to afford the title compound (5.0 g) as a crude white solid. LCMS m/z: 300.13 [M+H].

### Preparation 4: tert-Butyl (4-benzyl-3-oxo-3,4-dihydro-2H-benzo[b][1,4]thiazin-6-yl)carbamate

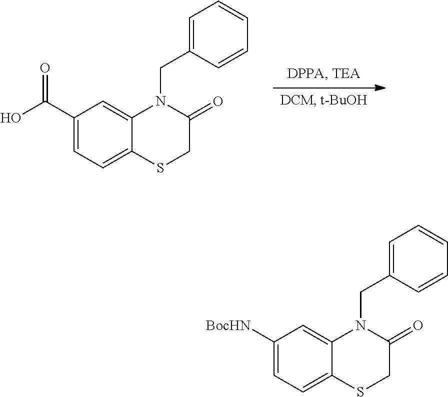

To a stirred solution of 4-benzyl-3-oxo-3,4-dihydro-2H-benzo[b][1,4]thiazine-6-carboxylic acid (Preparation 3) (4.5 g, 14.4 mmol) in DCM (50 mL) was added TEA (3 mL, 21.6 mmol) under an inert atmosphere at 0-5° C. followed by DPPA (6.3 mL, 28.8 mmol) and stirring then continued for 5 min. at the same temperature. The reaction mixture was brought slowly to room temperature and stirred for 4 h. Formation of the corresponding acyl azide was confirmed by TLC and UPLC-MS by quenching an aliquot of the reaction mixture into methanol. The solvents were evaporated, tert-butanol (50 mL) was added to the reaction mixture and the whole was refluxed overnight. Completion of the reaction was monitored by TLC and LC-MS, which showed formation of the desired product with complete consumption of the starting material. The solvents were evaporated in vacuo to obtain a crude oil which was adsorbed onto silica gel and purified by combi flash to afford the title compound (4.2 g, 80% yield) as an off white solid. LCMS m/z: 317.15 [M+H].

Preparation 5: 6-Amino-4-benzyl-2H-benzo[b][1,4]thiazin-(4H)-one

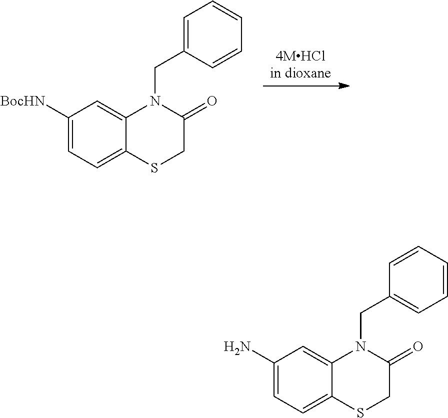 [SMILES: CC(C)(C)OC(=O)Nc1ccc2c(c1)N(Cc1ccccc1)C(=O)CS2 ; "4M·HClin dioxane" ; Nc1ccc2c(c1)N(Cc1ccccc1)C(=O)CS2]

To a stirred solution of tert-butyl (4-benzyl-3-oxo-3,4-dihydro-2H-benzo[b][1,4]thiazin-6-yl)carbamate (Preparation 4) (1.0 g, 2.7 mmol) in 1,4-dioxane (15 mL) was added HCl (15 mL, 4M HCl solution in 1,4-dioxane) at 0-5° C. and the combined mixture stirred for 5 min. The reaction mixture was then stirred overnight at room temperature. UPLC showed consumption of the starting material. The solvent was evaporated in vacuo. The resulting crude residue was then washed with NaHCO₃ solution and extracted with EtOAc. It was then evaporated in vacuo to give the title compound (750 mg, 90.5% yield) as a deep yellow solid. LCMS m/z: 271.23 [M+H].

### Preparation 6: 1-(4-Benzyl-3-oxo-3,4-dihydro-2H-benzo[b][1,4]thiazin-6-yl)-3-(1H-indol-6-yl)urea (Example 1)

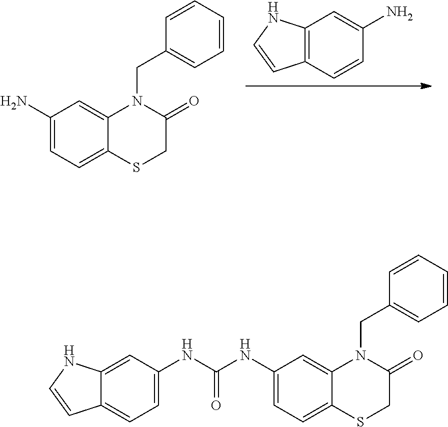

To a stirred solution of 6-amino-4-benzyl-2H-benzo[b][1,4]thiazin-3(4H)-one (Preparation 5) (0.650 g, 2.39 mmol) in THF (15 mL) was added p-nitrophenyl-chloroformate (0.580 g, 2.87 mmol) at 0-5° C. and the combined mixture was stirred for 5 min. and then allowed to warm slowly to room temperature over 1 h at which point carbamate formation was confirmed by TLC. 6-aminoindole (0.349 g, 2.64 mmol) was added followed by TEA (1 mL, 7 mmol) at 0-5° C. and the reaction mixture was stirred at room temperature for a further 1 h. Urea formation was detected by UPLC-MS and TLC and after completion the reaction mixture was diluted with water and extracted with EtOAc. The combined organic layers were washed with 1 N NaOH solution followed by brine, dried over anhydrous Na₂SO₄, filtered and evaporated under reduced pressure to give the crude product which was purified by prep-HPLC to afford the title compound (270 mg, 27% yield) as a white solid. Purity by UPLC: 99.32%; ¹H NMR (400 MHz; DMSO-d₆): δ 3.64 (s, 2H), 5.18 (s, 2H), 6.32 (s, 1H), 6.80 (d, J=8.32 Hz, 1H), 7.18-7.40 (m, 10H), 7.75 (s, 1H), 8.57 (s, 1H), 8.67 (s, 1H), 10.92 (s, 1H); LCMS m/z: 429.35 [M+H].

### Example 69: 1-(4-Benzyl-3-oxo-3,4-dihydro-2H-benzo[b][1,4]oxazin-7-yl)-3-(1H-indol-6-yl)urea

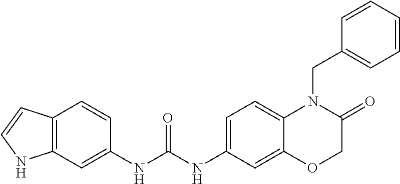 [SMILES: O=C(Nc1ccc2c(c1)OCC(=O)N2Cc1ccccc1)Nc1ccc2cc[nH]c2c1]

Example 69 was prepared according to General Procedure 1-6 and the methods described below.

### Preparation 44: Methyl 3-oxo-3,4-dihydro-2H-benzo[b][1,4]oxazine-7-carboxylate

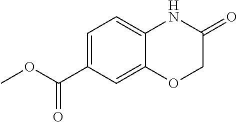

### Step 1: Methyl 3-(2-methoxy-2-oxoethoxy)-4-nitrobenzoate

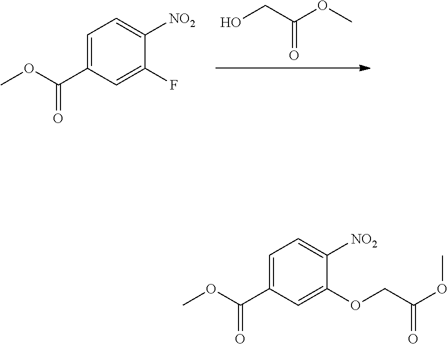

To a stirred solution of NaH (1.5 g, 37.6 mmol, 60% suspension in mineral oil) in 1,4-dioxane (50 mL) was added commercially available methyl 2-hydroxyacetate (3.39 g, 37.6 mmol) at 5-10° C. and the combined mixture stirred for 30 min. Methyl 3-fluoro-4-nitrobenzoate (5.0 g, 25.11 mmol) in 1,4-dioxane (25 mL) was added and the whole stirred at RT for 16 h. Progress of the reaction was monitored by TLC and UPLC-MS and after completion the reaction mixture was diluted with ice-cold water and stirred for 15 min. The precipitated solid was filtered, washed with water and dried in a vacuum oven at 60° C. for 2.5 h to afford the title compound (5.0 g) as a pale yellow crude solid. UPLC-MS m/z: 269.98 [M+H].

### Step 2: Methyl 3-oxo-1,4-dihydro-2H-benzo[b][1,4]oxazine-7-carboxylate

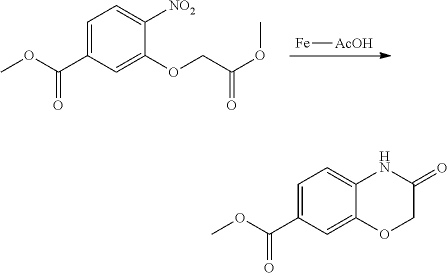

To a stirred solution of methyl 3-(2-methoxy-2-oxoethoxy)-4-nitrobenzoate (Preparation 44, Step 1) (5.0 g, 18.57 mmol) in AcOH (25 mL) was added iron powder (4.15 g, 74.304 mmol) at RT. The resulting reaction mixture was stirred at 90° C. for 2 h. TLC and UPLC-MS showed formation of the desired compound and after completion of the reaction, the reaction mixture was quenched by pouring into ice-cold water (500 mL) and stirring for 30 min. The precipitated solid was filtered and washed several times with water. The washed solid material was then dried in a vacuum oven at 60° C. for 6 h to afford the title compound (3.8 g) as an ash colored solid. UPLC-MS m/z: 207.98 [M+H].

### Preparation 45: 7-Amino-4-benzyl-2H-benzo[b][1,4]oxazin-(4H)-one

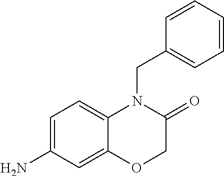 [SMILES: Nc1ccc2c(c1)OCC(=O)N2Cc1ccccc1]

### Step 1: Methyl 4-benzyl-3-oxo-3,4-dihydro-2H-benzo[b][1,4]oxazine-7-carboxylate

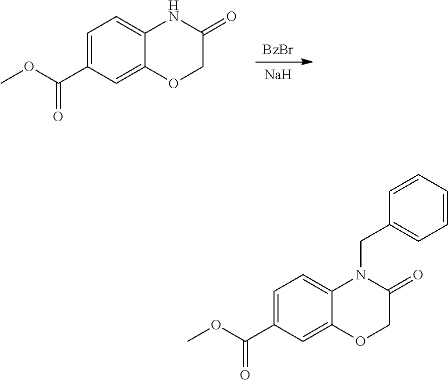

To a stirred solution of methyl 3-oxo-3,4-dihydro-2H-benzo[b][1,4]oxazine-7-carboxylate (Preparation 44, Step 2) (2.0 g, 9.65 mmol) in DMF (20 mL) was added NaH (425 mg, 10.62 mmol) followed by benzyl bromide (1.27 mL, 10.62 mmol) at 0-10° C. The whole was slowly allowed to warm to RT over 1 h. TLC and UPLC-MS showed formation of the desired product and after completion of the reaction the mixture was diluted with chilled water to give a solid precipitate which was filtered and dried in a vacuum oven to afford the title compound (2.6 g, 90% yield) as an ash coloured solid. UPLC-MS m/z: 298.88 [M+H].

### Step 2: 4-Benzyl-3-oxo-3,4-dihydro-2H-benzo[b][1,4]oxazine-7-carboxylic acid

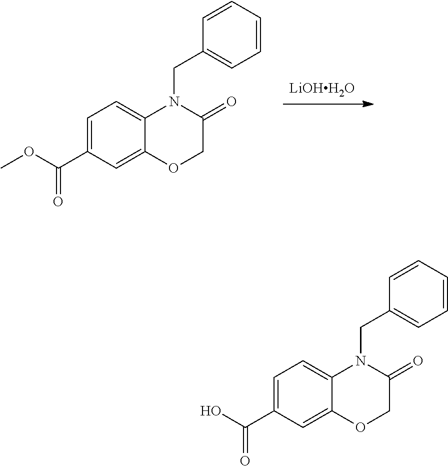 [SMILES: COC(=O)c1ccc2c(c1)OCC(=O)N2Cc1ccccc1 ; "LiOH·H2O" ; O=C(O)c1ccc2c(c1)OCC(=O)N2Cc1ccccc1]

To a stirred solution of methyl 4-benzyl-3-oxo-3,4-dihydro-2H-benzo[b][1,4]oxazine-7-carboxylate (Preparation 45, Step 1) (2.6 g, 8.75 mmol) in a mixture of THF (30 mL) and MeOH (15 mL) was added a solution of LiOH.H₂O (1.83 g, 43.73 mmol) in water (15 mL) at RT. The mixture was stirred at RT for 124 h. Progress of the reaction was monitored by UPLC-MS and after completion the solvents were evaporated in vacuo to give a residue which was diluted with water and washed with diethyl ether. The aqueous layer was acidified with 6N HCl to give a precipitate which was filtered and dried in a rotary evaporator with acetonitrile as co-solvent to afford the title compound (2.2 g, 890% yield) as an off white crude solid. UPLC-MS m/z: 284.02 [M+H].

### Step 3: tert-Butyl (4-benzyl-3-oxo-3,4-dihydro-2H-benzo[b][1,4]oxazin-7-yl)carbamate

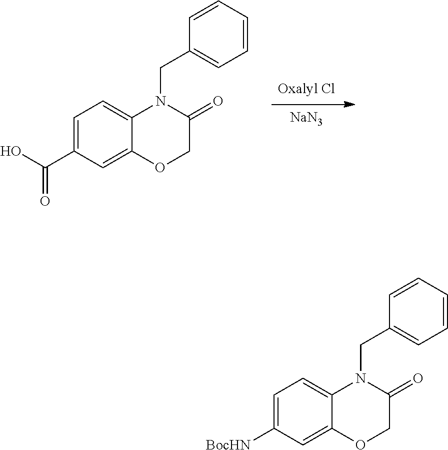 [SMILES: O=C(O)c1ccc2c(c1)OCC(=O)N2Cc1ccccc1 ; "Oxalyl Cl" ; CC(C)(C)OC(=O)Nc1ccc2c(c1)OCC(=O)N2Cc1ccccc1 ; "NaN3"]

To a stirred solution of 4-benzyl-3-oxo-3,4-dihydro-2H-benzo[b][1,4]oxazine-7-carboxylic acid (Preparation 45, Step 2) (200 mg, 0.7 mmol) in DCM (5 mL) was added DMF (0.05 mL) and oxalyl chloride (0.092 mL, 1.06 mmol) at 0-5° C. The combined mixture was stirred at RT for 1 h. TLC showed formation of the corresponding acid chloride. The solvent was evaporated in vacuo to afford an orange crude mass which was treated with a saturated solution of NaN₃ (91.78 mg, 1.41 mmol) in water (5 mL) and further stirred at RT for 1 h. TLC showed completion of the reaction. The reaction mixture was diluted with water and extracted with MTBE. The organic layer was washed with aqueous sodium bicarbonate solution and brine, dried over anhydrous Na₂SO₄, filtered and evaporated in vacuo to afford the corresponding crude acyl azide (250 g) intermediate which was dissolved in t-BuOH (10 mL) and stirred at 90° C. for 1 h. After complete consumption of the azide intermediate (monitored by UPLC-MS), the solvent was evaporated in vacuo to afford the crude product which was purified by Combi-flash (20 g column) using 20% EtOAc in hexane as eluent to give the title compound (120 g, 42% yield) as a white solid. UPLC-MS m/z: 355.13 [M+H].

### Step 4: 7-Amino-4-benzyl-2H-benzo[b][1,4]oxazin-(4H)-one

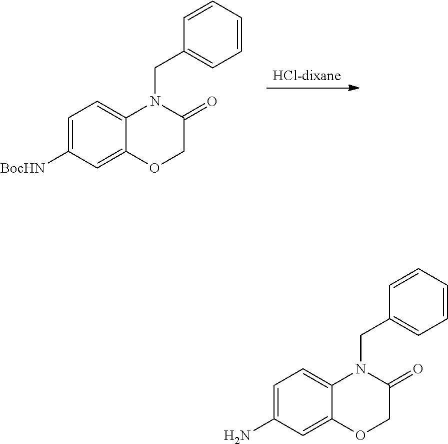 [SMILES: CC(C)(C)OC(=O)Nc1ccc2c(c1)OCC(=O)N2Cc1ccccc1 ; "HCl-dixane" ; Nc1ccc2c(c1)OCC(=O)N2Cc1ccccc1]

A solution of tert-butyl (4-benzyl-3-oxo-3,4-dihydro-2H-benzo[b][1,4]oxazin-7-yl)carbamate (Preparation 45, Step 3) (120 mg, 0.34 mmol) in a 4M solution of HCl in dioxane (3.5 mL) was stirred at RT for 2 h under an inert atmosphere. UPLC-MS showed formation of the desired product. The solvent was evaporated to give the title compound (120 mg) as a crude yellow sticky mass which was used in the next step without any further purification. UPLC-MS m/z: 254.98 [M+H].

### Preparation 46: 1-(4-Benzyl-3-oxo-3,4-dihydro-2H-benzo[b][1,4]oxazin-7-yl)-3-(1H-indol-6-yl)urea (Example 69)

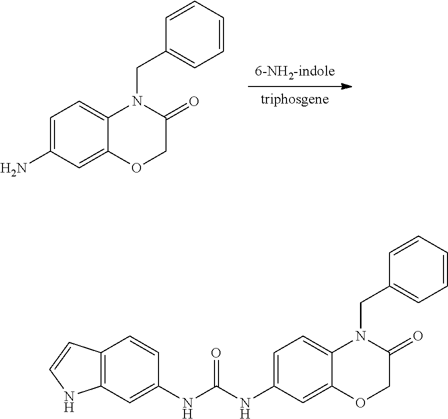 [SMILES: Nc1ccc2c(c1)OCC(=O)N2Cc1ccccc1 ; "6-NH2-indole" ; "triphosgene" ; O=C(Nc1ccc2c(c1)OCC(=O)N2Cc1ccccc1)Nc1ccc2cc[nH]c2c1]

To a stirred solution of 6-amino indole (68 mg, 0.519 mmol) in THF (5 mL) was added triphosgene (70 mg, 0.235 mmol) at 0-5° C. The combined mixture was stirred at RT for 1 h. Completion of the first stage of the reaction was confirmed by TLC, after which 7-amino-4-benzyl-2H-benzo[b][1,4]oxazin-3(4H)-one (Preparation 45, Step 4) (120 mg, 0.472 mmol) and TEA (0.225 mL, 1.557 mmol) were added into the reaction mixture and stirring continued at RT for 1 h. Progress of the reaction was monitored by TLC and after completion the solvent was evaporated in vacuo to afford the crude material which was purified by prep-HPLC to give the title compound (23 mg, 12% yield) as a pale brownish solid. Purity by UPLC: 96.28%; ¹H NMR (400 MHz; DMSO-d₆): δ 4.76 (s, 2H), 5.13 (s, 2H), 6.30 (t, J=1.04 Hz, 1H), 6.90-6.94 (m, 2H), 6.97-6.99 (m, 1H), 7.17-7.19 (m, 1H), 7.25-7.40 (m, 7H), 7.79-7.83 (m, 1H), 9.78 (s, 1H), 9.95 (s, 1H), 10.88 (s, 1H); UPLC-MS m/z: 413.11 [M+H].

### Examples 2-4, 6, 9, 12, 16-19, 22-33, 51-53, 59-60, 87-96, 99, 116, 124-142, 144-162, 164-166 and 193-195

The examples in the table below were prepared according to the above methods used to make Example 1 and 69 as described in General Procedures 1-6 using the appropriate amines. Purification was as stated in the aforementioned methods

| | | IUPAC | | LCMS | Purity |
|---|---|---|---|---|---|
| Ex | Structure | Name | 1H-NMR | [M + H] | (%) |
| 2 | 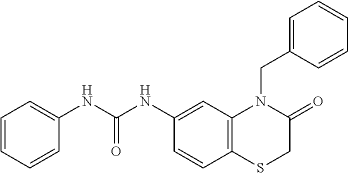 [SMILES: O=C(Nc1ccccc1)Nc1ccc2c(c1)N(Cc1ccccc1)C(=O)CS2] | 1-(4-benzyl- 3-oxo-3,4- dihydro- 2H- benzo[b][1, 4]thiazin-6- yl)-3- phenylurea | (500 MHz; DMSO-d₆): δ 3.64 (s, 2H), 5.17 (s, 2H), 6.96 (t, J = 7.25 Hz, 1H), 7.20-7.30 (m, 7H), 7.33- 7.35 (m, 3H), 7.39-7.41 (m, 2H), 8.65 (s, 1H),8.70 (s, 1H) | 390.2 | 98.94 |
| 3 | 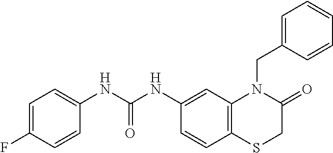 [SMILES: O=C(Nc1ccc(F)cc1)Nc1ccc2c(c1)N(Cc1ccccc1)C(=O)CS2] | 1-(4-benzyl- 3-oxo-3,4- dihydro- 2H- benzo[b][i, 4]thiazin-6- yl)-3-(4- fluorophenyl) urea | (500 MHz; DMSO-d₆): δ 3.64 (s, 2H), 5.17 (s, 2H), 7.11-7.19 (m, 2H), 7.24- 7.32 (m, 8H), 7.40-7.41 (m, 2H), 8.75 (d, J = 8.45 Hz, 2H) | 408.15 | 99.33 |
| 4 | 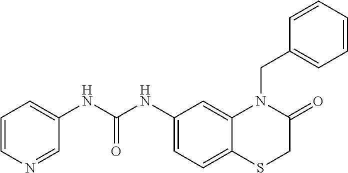 [SMILES: O=C(Nc1cccnc1)Nc1ccc2c(c1)N(Cc1ccccc1)C(=O)CS2] | 1-(4-benzyl- 3-oxo-3,4- dihydro- 2H- benzo[b][1, 4]thiazin-6- yl)-3- (pyridin-3- yl)urea | (500 MHz; DMSO-d₆): δ 3.65 (s, 2H), 5.17 (s, 2H), 7.20-7.30 (m, 5H), 7.31- 7.41 (m, 4H), 7.89 (d, J = 8.2 Hz, 1H), 8.18 (d, J = 3.65 Hz, 1H), 8.55 (d, J = 1.84 Hz, 1H), 8.83 (d, J = 7.85 Hz, 2H) | 391.18 | 91.21 |
| 6 | 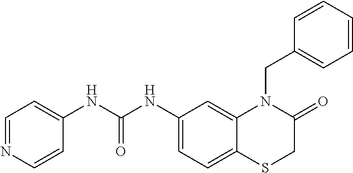 [SMILES: O=C(Nc1ccncc1)Nc1ccc2c(c1)N(Cc1ccccc1)C(=O)CS2] | 1-(4-benzyl- 3-oxo-3,4- dihydro- 2H- benzo[b][1, 4]thiazin-6- yl)-3- (pyridin-4- yl)urea | (500 MHz; DMSO-d₆): δ 3.65 (s, 2H), 5.17 (s, 2H), 7.20-7.25 (m, 5H), 7.32- 7.37 (m, 3H), 7.38-7.39 (m, 2H), 8.34 (d, J = 6.25 Hz, 2H), 8.90 (s, 1H), 9.09 (s, 1H) | 391.-17 | 98.66 |
| 9 | 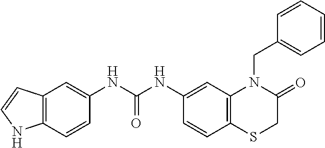 [SMILES: O=C(Nc1ccc2c(c1)N(Cc1ccccc1)C(=O)CS2)Nc1ccc2[nH]ccc2c1] | 1-(4-benzyl- 3-oxo-3,4- dihydro- 2H- benzo[b][1, 4]thiazin-6- yl)-3-(1H- indol-5- yl)urea | (500 MHz; DMSO-d₆): δ 3.63 (s, 2H), 5.18 (s, 2H), 6.32 (s, 1H), 7.03 (d, J = = 8.45 Hz, 1H), 7.19-7.35 (m, 10H), 7.62 (s, 1H), 8.38 (s, 1H), 8.60 (s, 1H), 10.93 (s, 1H) | 429.29 | 98.95 |
| 12 | 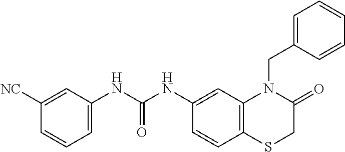 [SMILES: N#Cc1cccc(NC(=O)Nc2ccc3c(c2)N(Cc2ccccc2)C(=O)CS3)c1] | 1-(4-benzyl- 3-oxo-3,4- dihydro- 2H- benzo[b][1, 4]thiazin-6- yl)-3-(4- cyanobenzyl) urea | (400 MHz; DMSO-d₆): δ 3.60 (s, 2H), 4.33 (d, J = 6.04 Hz, 2H), 5.13 (s, 2H), 6.79 (t, J = 5.8 Hz, 1H), 7.12-7.29 (m, 8H), 7.44 (d, J = 8.32 Hz, 2H), 7.79 (d, J = 8.36 Hz, 2H), 8.78 (s, 1H) | 429-33 | 98.26 |
| 16 | 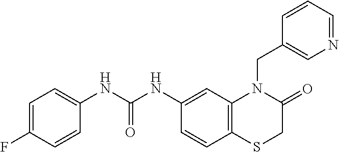 [SMILES: O=C(Nc1ccc(F)cc1)Nc1ccc2c(c1)N(Cc1cccnc1)C(=O)CS2] | l-(4- fluorophenyl)- 3-(3-oxo- 4-(pyridin- 3-ylmethyl)- 3,4-dihydro- 2H-benzo[b] [1,4]thiazin-6- yl)urea | (400 MHz; DMSO-d₆): δ 3.65 (s, 2H), 5.19 (s, 2H), 7.11-7.64 (m, 9H), 8.46- 8.50 (m, 2H), 8.82-8.84 (m, 2H) | 409.32 | 97.57 |
| 17 | 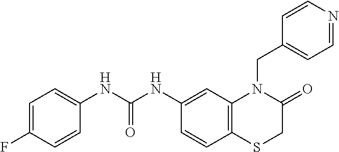 [SMILES: O=C(Nc1ccc(F)cc1)Nc1ccc2c(c1)N(Cc1ccncc1)C(=O)CS2] | l-(4- fluorophenyl)- 3-(3-oxo-4- (pyridin-4- ylmethyl)- 3,4- dihydro-2H- benzo[b][1, 4]thiazin-6- yl)urea | (400 MHz; DMSO-d₆): δ 3.68 (s, 2H), 5.19 (s, 2H), 7.09-7.18 (m, 4H), 7.32-7.38 (m, 5H), 8.46- 8.56 (bs, 2H), 8.88-8.88 (m, 2H) | 409.38 | 97.83 |
| 18 | 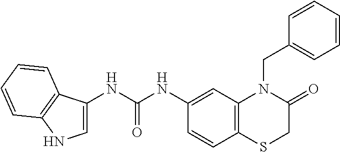 [SMILES: O=C(Nc1ccc2c(c1)N(Cc1ccccc1)C(=O)CS2)Nc1c[nH]c2ccccc12] | 1-(4-benzyl- 3-oxo-3,4- dihydro- 2H- benzo[b][1, 4]thiazin-6- yl)-3-(1H- indol-3- yl)urea | (500 MHz; DMSO-d₆): δ 3.64 (s, 2H), 5.18 (s, 2H), 6.99 (t, J = 7.45 Hz, 1H), 7.09 (t, J = 7.4 Hz, 1H), 7.24-7.45 (m, 9H), 7.46-7.48 (m, 2H), 8.46 (s, 1H), 8.64 (s, 1H), 10.72 (s, 1H) | 429.4 | 96.73 |
| 19 | 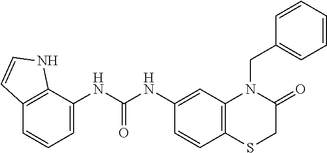 [SMILES: O=C(Nc1ccc2c(c1)N(Cc1ccccc1)C(=O)CS2)Nc1cccc2cc[nH]c12] | 1-(4-benzyl- 3-oxo-3,4- dihydro- 2H- benzo[b][1, 4]thiazin-6- yl)-3-(1H- indol-7- yl)urea | (500 MHz; DMSO-d₆): δ 3.64 (s, 2H), 5.18 (s, 2H), 6.42 (bs, 1H), 6.92- 6.93 (m, 1H), 7.10-7.12 (m, 1H), 7.23-7.28 (m, 5H), 7-31-7-36 (m, 5H), 8.65 (bs, 1H), 9.05 (s, 1H), 10.76 (s, 1H) | 429.27 | 99.0 |
| 22 | 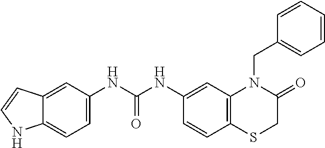 [SMILES: O=C(Nc1ccc2c(c1)N(Cc1ccccc1)C(=O)CS2)Nc1ccc2[nH]ccc2c1] | 1-(4-benzyl- 3-oxo-3,4- dihydro- 2H- benzo[b][1, 4]thiazin-7- yl)-3-(1H- indol-6- yl)urea | (400 MHz; DMSO-d₆): δ 3.66 (s, 2H), 5.21 (s, 2H), 6.32-6.33 (m, 1H), 6.82- 6.85 (dd, J1 = 1.84 Hz, J2 = 8.44 Hz 1H), 7.08 (d, J = 8.96 Hz, 1H), 7.13 7.24 (m, 1H), 7-30-7-33 (m, 4H), 7-39-7-40 (m, 2H), 7.41 (m, 1H), 7.64- | 429.31 | 97.98 |
| | | | 7.65 (m, 1H), 7.76 (s, | | |
| | | | 1H), 8.60 (s, 1H), 8.66 | | |
| | | | (s, 1H), 10.92 (s, 1H) | | |
| 23 | 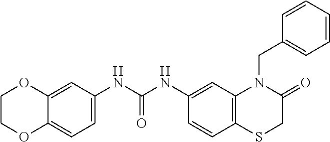 [SMILES: O=C(Nc1ccc2c(c1)OCCO2)Nc1ccc2c(c1)N(Cc1ccccc1)C(=O)CS2] | 1-(4-benzyl- 3-oxo-3,4- dihydro- 2H-benzo[b] [1,4]thiazin- 6-yl)-3-(2,3- dihydrobenzo [b][1,4] dioxin-6- yl)urea | (500 MHz; DMS0-d₆): δ 3.63 (s, 2H), 4.18-4.21 (m, 4H), 5.16 (s, 2H), 6.74 (s, 2H), 7.03 (s, 1H), 7.17-7.34 (m, 8H), 8.50 (s, 1H), 8.66 (s, 1H) | 448.34 | 98.96 |
| 24 | 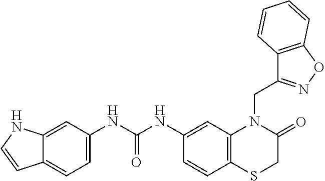 [SMILES: O=C(Nc1ccc2c(c1)N(Cc1noc3ccccc13)C(=O)CS2)Nc1ccc2cc[nH]c2c1] | 1-(4- (benzo[d] isoxazol-3- ylmethyl)- 3-oxo-3,4- dihydro- 2H-benzo [b][1,4] thiazin-6- yl)-3-(1H- indol-6- yl)urea | (400 MHz; DMSO-d₆): δ 3.64 (s, 2H), 5.60 (s, 2H), 6.32 (s, 1H), 6.972- 6.81 (m, 1H), 7.21-7.22 (m, 1H), 7.229-7.32 (m, 2H), 7.38-7.46 (m, 3H), 7.65-7.69 (m, 1H), 7.74- 7.46 (m, 2H), 7.86-7.88 (m, 1H), 8.58 (s, 1H), 8.69 (s, 1H), 10.91 (s, 1H) | 470.37 | 99.01 |
| 25 | 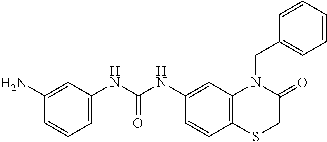 [SMILES: Nc1cccc(NC(=O)Nc2ccc3c(c2)N(Cc2ccccc2)C(=O)CS3)c1] | 1-(3- aminophenyl)- 3-(4-benzyl- 3-oxo-3,4- dihydro- 2H-benzo [b][1,4] thiazin-6- yl)urea | (400 MHz; DMSO-d₆): δ 3.33 (s, 2H), 5.02 (bs, 2H), 5.17 (s, 2H), 6.16- 6.18 (m, 1H), 6.48-6.50 (m, 1H), 6.70-6.71 (m, 1H), 6.87 (t, J = 7.64 Hz 1H), 7.16-7.33 (m, 8H), 8.35 (s, 1H), 8.59 (s, 1H) | 405.38 | 99.26 |
| 26 | 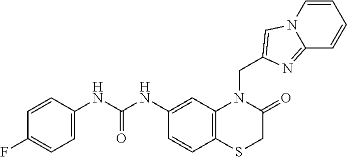 [SMILES: O=C(Nc1ccc(F)cc1)Nc1ccc2c(c1)N(Cc1cn3ccccc3n1)C(=O)CS2] | 1-(4- fluorophenyl)- 3-(4- (imidazo [1,2-a]pyridin- 2-ylmethyl)- 3-oxo-3,4- dihydro-2H- benzo[b][1, 4]thiazin-6- yl)urea | (500 MHz; DMSO-d₆): δ 3.58 (s, 2H), 5.20 (s, 2H), 6.86 (t, J = 6.3 Hz, 1H), 7.07-7.11 (m, 2H), 7.20-7.24 (m, 1H), 7.30- 7.35 (m, 2H), 7.40-7.42 (m, 3H), 7.50-7.52 (m, 1H), 7.75 (s, 1H), 8.47 (d, J = 6.5 Hz, 1H), 8.83 (s, 1H), 8.91 (s, 1H) | 448.30 | 95.04 |
| 27 | 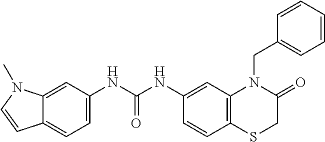 [SMILES: Cn1ccc2ccc(NC(=O)Nc3ccc4c(c3)N(Cc3ccccc3)C(=O)CS4)cc21] | 1-(4-benzyl- 3-oxo-3,4- dihydro- 2H-benzo[b] [1,4]thiazin- 6-yl)-3-(1- methyl-1H- indol-6- yl)urea | (500 MHz; DMSO-d₆): δ 3.64 (s, 2H), 3.72 (s, 3H), 5.19 (s, 2H), 6.33 (s, 1H), 6.87 (d, J = 8.05 Hz, 1H), 7.21-7.42 (m, 10H), 7.68 (s, 1H), 8.58 (s, 1H), 8.66 (s, 1H) | 443.37 | 97.20 |
| 28 | 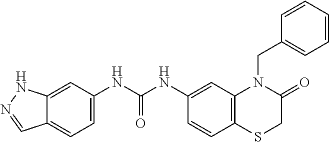 [SMILES: O=C(Nc1ccc2c(c1)N(Cc1ccccc1)C(=O)CS2)Nc1ccc2cn[nH]c2c1] | 1-(4-benzyl- 3-oxo-3,4- dihydro- 2H- benzo[b][1, 4]thiazin-6- yl)-3-(1H- indazol-6- yl)urea | (400 MHz; DMSO-d₆): δ 3.64 (s, 2H), 5.19 (s, 2H), 6.88 (s, 1H), 7.25-7-33 (m, 8H), 7.65 (s, 1H), 7.92 (s, 2H), 8.86-8.94 (m, 2H), 12.80 (s, 1H) | 430.34 | 99.51 |
| 29 | 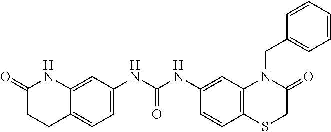 [SMILES: O=C1CCc2ccc(NC(=O)Nc3ccc4c(c3)N(Cc3ccccc3)C(=O)CS4)cc2N1] | 1-(4-benzyl- 3-oxo-3,4- dihydro-2H- benzo[b][1, 4]thiazin-6- yl)-3-(2- oxo-1,2,3,4- tetrahydro- quinolin-7- yl)urea | (400 MHz; DMSO-d₆): δ 2.39-2.43 (m 2H), 2.76- 2.80 (m, 2H), 3.63 (s, 2H), 5.17 (s, 2H), 6.93- 6.94 (m, 1H), 7.0-7.04 (m, 2H), 7-17-7-35 (m, 8H), 8.65 (d, J = 4.4 Hz, 2H), 10.05 (s, 1H) | 459.36 | 96.71 |
| 30 | 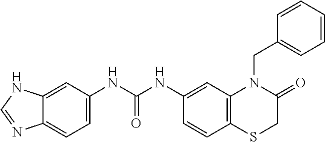 [SMILES: O=C(Nc1ccc2c(c1)N(Cc1ccccc1)C(=O)CS2)Nc1ccc2nc[nH]c2c1] | 1-(1H-benzo[d] imidazol-6-yl)- 3-(4-benzyl-3- oxo-3,4- dihydro-2H- benzo[b][1, 4]thiazin-6- yl)urea | (400 MHz; DMSO-d₆): δ 3.64 (s, 2H), 5.18 (s, 2H), 6.95-7.52 (s, 10H), 7.87 (s, 1H), 8.09 (s, 1H), 8.75 (s, 2H), 12.25 (s, 1H) | 430.38 | 98.85 |
| 31 | 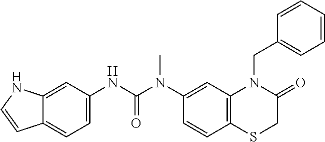 [SMILES: CN(C(=O)Nc1ccc2cc[nH]c2c1)c1ccc2c(c1)N(Cc1ccccc1)C(=O)CS2] | 1-(4-benzyl- 3-oxo-3,4- dihydro-2H- benzo[b][1, 4]thiazin-6- yl)-3-(1H- indol-6-yl)- 1-methylurea | (400 MHz; DMSO-d₆): δ 3.14 (s, 3H), 3.68 (s, 2H), 5.21 (s, 2H), 6.32 (s, 1H), 6.88 (d, J = 8 Hz, 1H), 7.01 (d, J = 7.72 Hz, 1H), 7.14-7.22 (m, 7H), 7.34-7-42 (m, 2H), 7.44- 7.56 (m, 1H), 7.98 (s, 1H), 10.92 (s, 1H) | 443.41 | 99.02 |
| 32 | 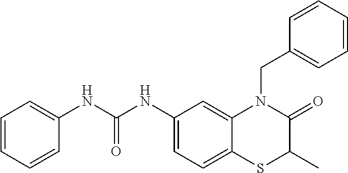 [SMILES: CC1Sc2ccc(NC(=O)Nc3ccccc3)cc2N(Cc2ccccc2)C1=O] | 1-(4-benzyl- 2-methyl-3- oxo-3,4- dihydro-2H- benzo[b][1, 4]thiazin-6- yl)-3- phenylurea | (500 MHz; DMSO-d₆): δ 1.39 (d, J = 6.8 Hz, 3H), 3.79-3.83 (q, J = 6.6 Hz, 1H), 5.17 (s, 2H), 6.96 (t, J = 7.2 Hz, 1H), 7.20- 7.41 (m, 12H), 8.73 (s, 1H), 8.79 (s, 1H) | 404.14 | 99.12 |
| 33 | 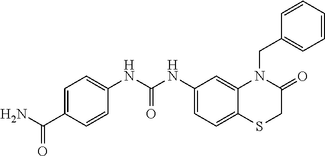 [SMILES: NC(=O)c1ccc(NC(=O)Nc2ccc3c(c2)N(Cc2ccccc2)C(=O)CS3)cc1] | 4-(3-(4- benzyl-3- oxo-3,4- dihydro-2H- benzo[b][1, 4]thiazin-6- yl)ureido) benzamide | (500 MHz; DMSO-d₆): δ 3.64 (s, 2H), 5.18 (s, 2H), 7.20-7.35 (m, 9H), 7.46 (d, J = 8.4 Hz, 2H), 7.79-7-83 (m, 3H), 8.99 (s, 1H), 9.10 (s, 1H) | 433.18 | 98.49 |
| 51 | 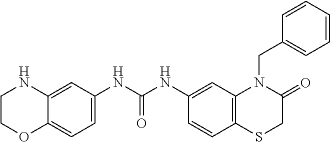 [SMILES: O=C(Nc1ccc2c(c1)NCCO2)Nc1ccc2c(c1)N(Cc1ccccc1)C(=O)CS2] | 1-(4-benzyl- 3-oxo-3,4- dihydro-2H- benzo[b][1, 4]thiazin-6- yl)-3-(3,4- dihydro-2H- benzo[b][1, 4]oxazin-6- yl)urea | (500 MHz; DMSO-d₆): δ 3.24 (s, 2H), 3.63 (s, 2H), 4.05 (s, 2H), 5.16 (s, 2H), 5.80 (s, 1H), 6.38-6.40 (m, 1H), 6.50- 6.52 (m, 1H), 6.75 (s, 1H), 7.15 (d, J = 6.64 Hz, 1H), 7.22-7.34 (m, 7H), 8.32 (s, 1H), 8.60 (s, 1H) | 447.37 | 98.75 |
| 52 | 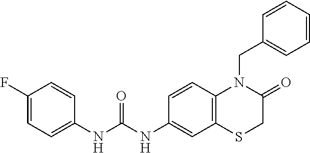 [SMILES: O=C(Nc1ccc(F)cc1)Nc1ccc2c(c1)SCC(=O)N2Cc1ccccc1] | 1-(4-benzyl- 3-oxo-3,4- dihydro- 2H- benzo[b][1, 4]thiazin-7- yl)-3-(4- fluorophenyl) urea | (400 MHz; DMSO-d₆): δ 3.66 (s, 2H), 5.20 (s, 2H), 7.08-7.13 (m, 4H), 7.19-7.23 (m, 3H), 7.29- 7.31 (m, 2H), 7.42-7.46 (m, 2H), 7.62 (s, 1H), 8.78-8.81 (m, 2H) | 408.30 | 98.2 |
| 53 | 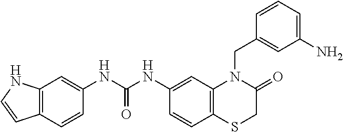 [SMILES: Nc1cccc(CN2C(=O)CSc3ccc(NC(=O)Nc4ccc5cc[nH]c5c4)cc32)c1] | l-(4-(3- aminobenzyl)- 3-oxo-3,4- dihydro-2H- benzo[b][1, 4]thiazin-6- yl)-3-(1H- indol-6- yl)urea | (400 MHz; DMSO-d₆): δ 3.59 (s, 2H), 5.03 (bs, 4H), 6.32-6.42 (m, 4H), 6.79-6.82 (m, 1H), 6.95 (t, J = 8.64 Hz, 1H), 7.20-7.28 (m, 4H), 7.31- 7.40 (m, 1H), 7.77 (s, 1H), 8.67 (s, 1H), 8.79 (s, 1H), 10.91 (s, 1H) | 444.08 | 98.81 |
| 59 | 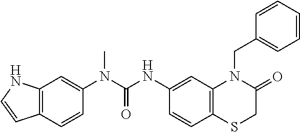 [SMILES: CN(C(=O)Nc1ccc2c(c1)N(Cc1ccccc1)C(=O)CS2)c1ccc2cc[nH]c2c1] | 3-(4-benzyl- 3-oxo-3,4- dihydro-2H- benzo[b][1, 4]thiazin-6- yl)-l-(1H- indol-6-yl)- 1-methylurea | (500 MHz; DMSO-d₆): δ 3.23 (s, 3H), 3-58 (s, 2H), 5.10 (s, 2H), 6.47 (s, 1H), 6.90 (d, J = 7.65 Hz, 1H), 7.18-7.19 (m, 1H), 7.21-7.23 (m, 4H), 7.25- 7.31 (m, 3H), 7.41-7.45 (m, 2H), 7.56 (d, J = 7.95 | 443.07 | 99.51 |
| | | | Hz, 1H), 7.86 (s, 1H), | | |
| | | | 11.19 (s, 1H) | | |
| 60 | 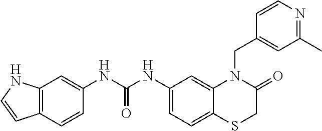 [SMILES: Cc1cc(CN2C(=O)CSc3ccc(NC(=O)Nc4ccc5cc[nH]c5c4)cc32)ccn1] | 1-(1H-indol- 6-yl)-3-(4-(2- methylpyridin- 4-yl)methyl)- 3-oxo-3,4- dihydro-2H- benzo[b][1, 4]thiazin-6- yl)urea | (400 MHz; DMSO-d₆): δ 2.44 (s, 3H), 3.68 (s, 2H), 5.13 (s, 2H), 6.32 (s, 1H), 6.79-6.82 (dd, J1 = 1.88 Hz, J2 = 5.82 Hz, 1H), 7.03-7.05 (m, 1H), 7.13 (s, 1H), 7.180-7.185 (m, 1H), 7.20-7.22 (m, 2H), 7.23 (m, 1H), 7.33 | 444.09 | 97.15 |
| | | | (d, J = 8.44 Hz, 1H), | | |
| | | | 7.72-7.73 (m, 1H), 8.38- | | |
| | | | 8.39 (m, 1H), 8.58 (s, | | |
| | | | 1H), 8.70 (s, 1H), 10.91 | | |
| | | | (s, 1H) | | |
| 87 | 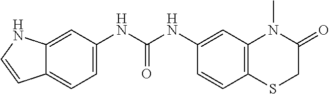 [SMILES: CN1C(=O)CSc2ccc(NC(=O)Nc3ccc4cc[nH]c4c3)cc21] | 1-(1H-indol- 6-yl)-3-(4- methyl-3- oxo-3,4- dihydro-2H- benzo[b][1, | (500 MHz; DMSO-d₆): δ 3.49 (s, 5H), 6.34 (s, 1H), 6.89 (d, 1H, J = 6.75 Hz), 7.10 (s, 1H), 7.22 (s, 1H), 7.28 (d, 1H, J = 6.85 Hz), 7.40 (s, 1H), 7.58 (s, 1H), | 353.26 | 96.08 |
| | | 4]thiazin-6- | 7.82 (s, 1H), 8.89 (s, 1H), | | |
| | | yl)urea | 9.07 (s, 1H), 10.89 (s, | | |
| | | | 1H) | | |
| 88 | 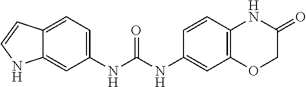 [SMILES: O=C1COc2cc(NC(=O)Nc3ccc4cc[nH]c4c3)ccc2N1] | 1-(1H-indol- 6-yl)-3-(3- oxo-3,4- dihydro-2H- benzo[b][1, 4]oxazin-7- | (400 MHz; DMSO-d₆): δ 4.54 (s, 2H), 6.32 (s, 1H), 6.85-6.78 (m, 2H), 6.95- 6.92 (m, 1 H), 6.24 (d, 2H, J = 11 Hz), 7.40 (d, 1H, J = 8.5 Hz), 7.78 (s, | 323.04 | 97.67 |
| | | yl)urea | 1H), 8.6 (s, 1H), 8.65 (s, | | |
| | | | 1H), 10.56 (s, 1H), 10.91 | | |
| | | | (s, 1H). | | |
| 89 | 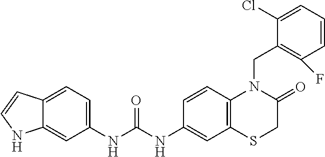 [SMILES: O=C(Nc1ccc2c(c1)SCC(=O)N2Cc1c(F)cccc1Cl)Nc1ccc2cc[nH]c2c1] | 1-(4-(2- chloro-6- fluorobenzyl)- 3-oxo-3,4- dihydro-2H- benzo[b][1, 4]thiazin-7- yl)-3-(1H- indol-6- yl)urea | (400 MHz; DMSO-d₆): δ 3.55 (s, 2H), 5-34 (s, 2H), 6.32 (s, 1H), 6.84 (dd, 1H, J = 8.44 Hz, 1.8 Hz), 7.22-7.13 (m, 4H), 7.34-7.25 (m, 2H), 7.40 (d, 1H, J = 8.3 Hz), 7.57 (d, 1H, J = 2.28 Hz), 7.75 (s, 1H), 8.67 (s, 1H), 8.73 (s, 1H), 10.92 (s, 1H). | 481.02 | 98.52 |
| 90 | 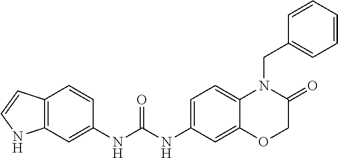 [SMILES: O=C(Nc1ccc2c(c1)OCC(=O)N2Cc1ccccc1)Nc1ccc2cc[nH]c2c1] | 1-(4-benzyl- 2,2-dimethyl- 3-oxo-3,4- dihydro-2H- benzo[b][1, 4]oxazin-7- yl)-3-(1H- indol-6- yl)urea | (400 MHz; DMSO-d₆): δ 1.51 (s, 6H), 5.12 (s, 2H), 6.33 (s, 1H), 6.84 (d, 1H, J = 8.52 Hz), 6.91 (s, 2H), 7.23-7.41 (m, 8H), 7.76 (s, 1H), 8.56 (s, 1H), 8.61 (s, 1H), 10.94 (s, 1H). | 441.16 | 95.07 |
| 91 | 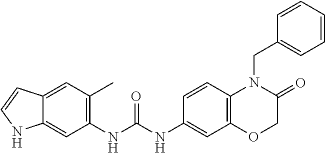 [SMILES: Cc1cc2cc[nH]c2cc1NC(=O)Nc1ccc2c(c1)OCC(=O)N2Cc1ccccc1] | 1-(4-benzyl- 3-oxo-3,4- dihydro-2H- benzo[b][1, 4]oxazin-7- yl)-3-(5- methyl-1H- indol-6- yl)urea | (400 MHz; DMSO-d₆): δ 2.27 (s, 3H), 4.79 (s, 2H), 5.13 (s, 2H), 6.27 (s, 1H), 6.89 (m, 2H), 7.20- 7.37 (m, 8H), 7.80 (s, 1 H), 7.85 (s, 1H), 8.99 (s, 1H), 10.94 (s, 1H). | 427.13 | 99.1 |
| 92 | 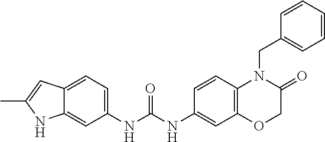 [SMILES: Cc1cc2ccc(NC(=O)Nc3ccc4c(c3)OCC(=O)N4Cc3ccccc3)cc2[nH]1] | 1-(4-benzyl- 3-oxo-3,4- dihydro-2H- benzo[b][1, 4]oxazin-7- yl)-3-(2- methyl-1H- indol-6- yl)urea | (400 MHz; DMSO-d₆): δ 2.34 (s, 3H), 4.78 (s, 2H), 5.13 (s, 2H), 6.01 (s, 1H), 6.76 (m, 1H), 6.87- 6.93 (m, 2H), 7.23-7.36 (m, 7H), 7.66 (s, 1H), 8.59 (s, 1H), 8.60 (s, 1H), 10.74 (s, 1H). | 427.15 | 96.55 |
| 93 | 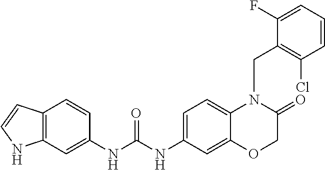 [SMILES: O=C(Nc1ccc2c(c1)OCC(=O)N2Cc1c(F)cccc1Cl)Nc1ccc2cc[nH]c2c1] | l-(4-(2- chloro-6- fluorobenzy 1)-3-oxo- 3,4- dihydro- 2H- benzo[b][i, 4]oxazin-7- yl)-3-(1H- indol-6- | (400 MHz; DMSO-d₆): δ 4.67 (s, 2H), 5.28 (s, 2H), 6.32 (s, 1H), 6.84 (d, 1H, J = 8.4 Hz), 6.94 (s, 2H), 7.21-7.40 (m, 6H), 7.77 (s, 1H), 8.63 (s, 1H), 8.66 (s, 1 H), 10.92 (s, 1H). | 465.05 | 99.16 |
| | | yl)urea | | | |
| 94 | 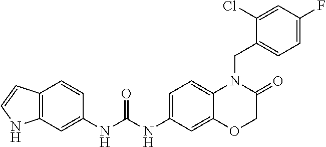 [SMILES: O=C(Nc1ccc2c(c1)OCC(=O)N2Cc1ccc(F)cc1Cl)Nc1ccc2cc[nH]c2c1] | 1-(4-(2- chloro-4- fluorobenzy 1)-3-oxo-3,4- dihydro-2H- benzo[b][1,4] oxazin-7-yl)- 3-(1H-indol-6- yl)urea | (400 MHz; DMSO-d₆): δ 4.81 (s, 2H), 5.08 (s, 2H), 6.31 (d, 1H, J = 1.96 Hz), 6.72 (d, 1H, J = 8.8 Hz), 6.81-6.84 (dd, 1H, Jl = 1.72 Hz, J2 = 8.4 Hz), 6.90-6.93 (dd, 1H, J = 2.2 Hz, J2 =8.68 Hz), 6.14-6.17 (m, 2H), 7.20 | 465.04 | 99.37 |
| | | | (t, 1H, J = 2.64 Hz), 7.36 | | |
| | | | (d, 1H, J = 2.32 Hz), 7.39 | | |
| | | | (d, 1H, J = 8.48 Hz), | | |
| | | | 7.52-7.55 (dd, 1H, J1 = | | |
| | | | 2.32 Hz, J2 = 8.72 Hz), | | |
| | | | 7.77 (s, 1H), 8.58 (bs, | | |
| | | | 1H), 8.69 (bs, 1H), 10.90 | | |
| | | | (s, 1H). | | |
| 95 | 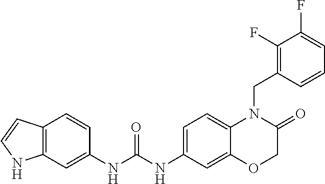 [SMILES: O=C(Nc1ccc2c(c1)OCC(=O)N2Cc1cccc(F)c1F)Nc1ccc2cc[nH]c2c1] | 1-(4-(2,3- difluorobenzyl)- 3-oxo-3,4- dihydro-2H- benzo[b][1, 4]oxazin-7- yl)-3-(1H- indol-6- yl)urea | (400 MHz; DMSO-d₆): δ 4.77 (s, 2H), 5.19 (s, 2H), 6.32 (s, 1H), 6.81-6.83 (dd, J1 = 1.72 Hz, J2 = 8.44 Hz, 1H), 6.89-6.95 (m, 2H), 6.98 (t, J = 7.2 Hz, 1H), 7.12-7.17 (m, 1H), 7.20 (t, J = 5 Hz, 1H), 7.31-7.40 (m, 3H), 7.76 (s, 1H), 8.540 (s, 1H), 8.63 (s, 1H), 10.90 (s, 1H). | 449.1 | 98.57 |
| 96 | 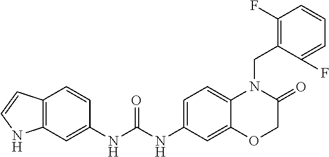 [SMILES: O=C(Nc1ccc2c(c1)OCC(=O)N2Cc1c(F)cccc1F)Nc1ccc2cc[nH]c2c1] | 1-(4-(2,6- difluorobenzyl)- 3-oxo-3,4- dihydro-2H- benzo [b][1, 4]oxazin-7- yl)-3-(1H- indol-6- yl)urea | (400 MHz; DMSO-d₆): δ 4.66 (s, 2H), 5.23 (s, 2H), 6.32 (s, 1H), 6.80- 6.82 (dd, J1 = 1.6 Hz, J2 = 8.36 Hz, 1H), 6.94- 6.97 (m, 2H), 7.01-7.09 (m, 2H), 7.20 (t, J = 3.88 Hz, 1H), 7.27 (d, J = 2.12 Hz, 1H), 7.35-7.40 (m, 2H), 7.75 (s, 1H), 8.50 (s, 1H), 8.54 (s, 1H), 10.90 (s, 1H). | 449.1 | 99.65 |
| 99 | 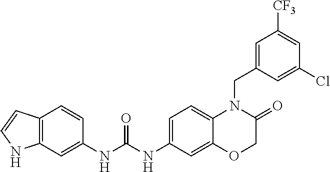 [SMILES: O=C(Nc1ccc2c(c1)OCC(=O)N2Cc1cc(Cl)cc(C(F)(F)F)c1)Nc1ccc2cc[nH]c2c1] | l-(4-(3- chloro-5- (trifluoromethyl) benzyl)-3-oxo- 3,4-dihydro- 2H-benzo[b][1, 4]oxazin-7- yl)-3-(1H- indol-6- yl)urea | (400 MHz; DMSO-d₆): δ 4.82 (s, 2H), 5.22 (s, 2H), 6.32 (s, 1H), 6.81 (d, 1H, J = 8.4 Hz), 6.92 (s, 2H), 7.20 (s, 1H), 7.32 (s, 1H), 7.39 (d, 1H, J = 8.52Hz), 6.66 (d, 2H, J = 3.96 Hz), 7.77 (d, 2H, J = 9.2 Hz), 8.51 (s, 1H), 8.58 (s, 1H), 10.90 (s, 1H). | 515.12 | 99.69 |
| 116 | 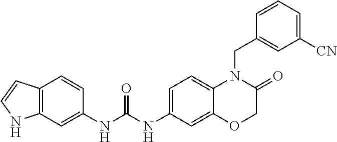 [SMILES: N#Cc1cccc(CN2C(=O)COc3cc(NC(=O)Nc4ccc5cc[nH]c5c4)ccc32)c1] | l-(4-(3- cyanobenzyl)- 3-oxo-3,4- dihydro-2H- benzo[b][1, 4]oxazin-7- yl)-3-(1H- indol-6- yl)urea | (400 MHz; DMSO-d₆): δ 4.81 (s, 2H), 5.17 (s, 2H), 6.31 (s, 1H), 6.80-6.92 (m, 3H), 7.20 (d, J = 2.44 Hz, 1H), 7.32 (d, J = 1.88 Hz, 1H), 7.39 (d, J = 8.48 Hz, 1H), 7.50-7.58 (m, 1H), 7.61-7.63 (m, 1H), 7.63-7.77 (m, 3H), | 438.23 | 100 |
| | | | 8.52 (s, 1H), 8.60 (s, 1H), | | |
| | | | 10.90 (s, 1H). | | |
| 124 | 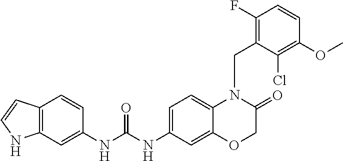 [SMILES: COc1ccc(F)c(CN2C(=O)COc3cc(NC(=O)Nc4ccc5cc[nH]c5c4)ccc32)c1Cl] | l-(4-(2-chloro-6- fluoro-3- methoxybenzyl)- 3-oxo-3,4- dihydro-2H- benzo[b][1, 4]thiazin-7- yl)-3-(1H- indol-6- yl)urea | (400 MHz; DMSO-d₆): δ 3.53 (s, 2H), 3.79 (s, 3H), 5.33 (s, 2H), 6.31 (s, 1H), 6.86 (d, 1H, J = 8.44Hz), 7.01-7.05 (m, 1H), 7.08-7.14 (m, 2H), 7.16-4.20 (m, 2H), 7.38 (d, 1H, J = 8.4 Hz), 7.57 (d, 1H, J = 1.96 Hz), 7.74 (s, 1H), 8.90-8.95 (d, | 511.17 | 98.13 |
| | | | 2H, 19.72 Hz), 10.89 (s, | | |
| | | | 1H). | | |
| 125 | 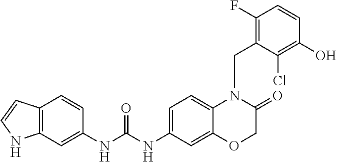 [SMILES: O=C(Nc1ccc2c(c1)OCC(=O)N2Cc1c(F)ccc(O)c1Cl)Nc1ccc2cc[nH]c2c1] | l-(4-(2-chloro-6- fluoro-3- hydroxybenzyl)- 3-oxo-3,4- dihydro-2H- benzo[b][1, 4]thiazin-7- yl)-3-(1H- indol-6- yl)urea | (400 MHz; DMSO-d₆): δ 3.32 (s, 1H), 3.53 (s, 2H), 5.30 (s, 2H), 6.32 (s, 1H), 6.82-6.85 (m, 2H), 6.94 (t, 1H, J = 9.36 Hz), 7.08 (d, 1H, J = 8.92 Hz), 7.15-7.21 (m, 2H), 7.39 (d, 1H, J = 8.44 Hz), 7.56 (d, 1H, J = 2.24 Hz), 7.75 (s, 1H), 8.72-8.75 (d, 2H, J = 12.64 Hz), 10.90 (s, 1H). | 497.15 | 99.07 |
| 126 | 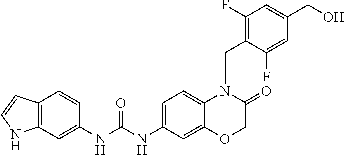 [SMILES: O=C(Nc1ccc2c(c1)OCC(=O)N2Cc1c(F)cc(CO)cc1F)Nc1ccc2cc[nH]c2c1] | 1-(4-(2,6- difluoro-4- methoxybenzyl)- 3-oxo-3,4- dihydro-2H- benzo[b][1, 4]thiazin-7- yl)-3-(1H- indol-6- yl)urea | (400 MHz; DMSO-d₆): δ 3.51 (s, 2H), 3.71 (s, 3H), 5.22 (s, 2H), 6.30 (s, 1H), 6.63-6.67 (m, 2H), 6.90-6.92 (dd, 1H, J1 = 1.4 Hz, J2 = 8.48 Hz), 7.15-7.19 (m, 2H), 7.23- 7.26 (dd, 1H, J1 = 2.16 Hz, J2 = 8.36 Hz), 7.37 (d, 1H, J = 8.44 Hz), | 495.14 | 96.12 |
| | | | 7.59 (d, 1H, J = 2.24 Hz), | | |
| | | | 7.77 (s, 1H), 9.34 (bs, | | |
| | | | 1H), 9.54 (s, 1H), 10.87 | | |
| | | | (s, 1H). | | |
| 127 | 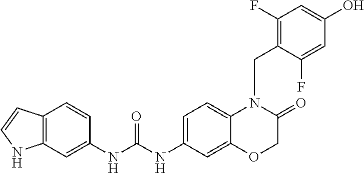 [SMILES: O=C(Nc1ccc2c(c1)OCC(=O)N2Cc1c(F)cc(O)cc1F)Nc1ccc2cc[nH]c2c1] | 1-(4-(2,6- difluoro-4- hydroxybenzyl)- 3-oxo-3,4- dihydro-2H- benzo[b][1, 4]thiazin-7- yl)-3-(1H- indol-6- yl)urea | (400 MHz; DMSO-d₆): δ 3.51 (s, 2H), 5.18 (s, 2H), 6.32-6.3 (m, 3H), 6.81- 6.84 (dd, 1H, J1 = 1.72 Hz, J2 = 8.44 Hz), 7.14- 7.21 (m, 3H), 7.39 (d, 1H, J = 8.4 Hz), 7.55 (d, 1H, J = 2.24 Hz), 7.75 (s, 1H), 8.56 (d, 2H, J = 8.56 Hz), 10.31 (bs, 1H), 10.90 (s, 1H). | 481.15 | 97.18 |
| 128 | 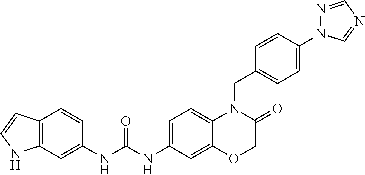 [SMILES: O=C(Nc1ccc2c(c1)OCC(=O)N2Cc1ccc(-n2cncn2)cc1)Nc1ccc2cc[nH]c2c1] | 1-(4-(4-(1H- 1,2,4- triazol-1- yl)benzyl)- 3-oxo-3,4- dihydro-2H- benzo[b][1, 4]oxazin-7- yl)-3-(1H- indol-6- yl)urea | (400 MHz; DMSO-d₆): δ 4.80 (s, 2H), 5.19 (s, 2H), 6.31 (s, 1H), 6.81 (d, 1H, J = 4.28 Hz), 6.92 (m, 2H), 7.20 (s, 1H), 7.31 (s, 1H) 7.38 (d, 1H, J = 6.28 Hz), 7.47 (d, 2H, J = 8.12 Hz), 7.75 (s, 1H), 7.81 (d, 2H, J =8.28 Hz), 8.21 (s, 1H), 8.53 (s, 1H), 8.61 (s, 1H), 9.24 (s, 1H), 10.89 (s, 1H). | 480.22 | 100 |
| 129 | 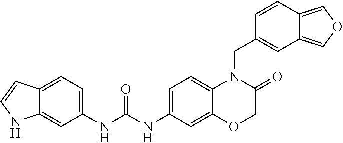 [SMILES: O=C(Nc1ccc2c(c1)OCC(=O)N2Cc1ccc2cocc2c1)Nc1ccc2cc[nH]c2c1] | l-(4-(benzo[c][1, 2,5]oxadiazol-5- ylmethyl)- 3-oxo-3,4- dihydro-2H- benzo[b][1,4] oxazin-7-yl)-3- (1H-indol-6- yl)urea | (400 MHz; DMSO-d₆): δ 4.810 (s, 2H), 5.22 (s, 2H), 6.31 (s, 1H), 6.81 (d, 1H, J = 8.4 Hz), 6.92 (s, 2H), 7.19 (d, 1H, J = 3 Hz), 7.29 (s, 1H), 7.38 (d, 1H, J = 8.4 Hz), 7.54 (d, 1H, J = 9.36 Hz), 7.69 (s, 1H), 7.81 (s, 1H), 8.04 | 455.14 | 99.44 |
| | | | (d, 1H, J = 9.36 Hz), 8.53 | | |
| | | | (s, 1H), 8.64 (s, 1H), | | |
| | | | 10.89 (s, 1H). | | |
| 130 | 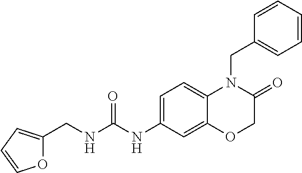 [SMILES: O=C(NCc1ccco1)Nc1ccc2c(c1)OCC(=O)N2Cc1ccccc1] | 1-(4-benzyl- 3-oxo-3,4- dihydro-2H- benzo[b][1, 4]oxazin-7- yl)-3-(furan-2- ylmethyl)urea | (400 MHz; DMSO-d₆): δ 4.24 (d, J = 5.44 Hz, 2H), 4.74 (s, 2H), 5.10 (s, 2H), 6.22 (d, 1H, J = 2.84 Hz), 6.37-6.38 (m. 1H), 6.58 (t, 1H, J = 5.68 Hz), 6.81-6.83 (dd, 1H, J1 = 2.12 Hz, J2 = 8.76 Hz), 6.87 (d, 1H, J = 8.76 Hz), 7.22-7.26 (m, 4H), | 378.15 | 99.29 |
| | | | 7.30-7.34 (m, 2H), 7.56 | | |
| | | | (d, 1H, J = 1.0 Hz), 8.58 | | |
| | | | (s, 1H). | | |
| 131 | 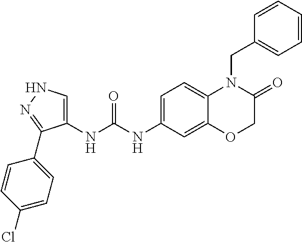 [SMILES: O=C(Nc1ccc2c(c1)OCC(=O)N2Cc1ccccc1)Nc1c[nH]nc1-c1ccc(Cl)cc1] | 1-(4-benzyl- 3-oxo-3,4- dihydro- 2H- benzo[b][1, 4]oxazin-7- yl)-3-(3-(4- chlorophen yl)-1H- pyrazol-4- yl)urea | (400 MHz; DMSO-d₆): δ 4.75 (s, 2H), 5.11 (s, 2H), 6.85-6.91 (m, 2H), 7.22- 7.27 (m, 4H), 7.31-7.34 (m, 2H), 7.50 (bs, 2H), 7.68-7.70 (m, 2H), 7.89- 7.95 (m, 2H), 8.85 (s, 1H), 12.83 (s, 1H). | 474.16 | 95.39 |
| 132 | 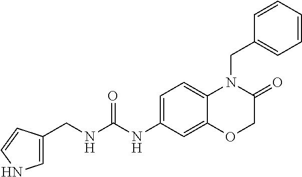 [SMILES: O=C(NCc1cc[nH]c1)Nc1ccc2c(c1)OCC(=O)N2Cc1ccccc1] | 1-((1H- pyrrol-3- yl)methyl)- 3-(4-benzyl-3- oxo-3,4- dihydro-2H- benzo[b][1, 4]oxazin-7- yl)urea | (400 MHz; DMSO-d₆): δ 4.06 (d, J = 5.04 Hz, 2H), 4.73 (s, 2H), 5.10 (s, 2H), 5.97 (d, 1H, J = 1.88 Hz), 619 (t, 1H, J = 5.16 Hz), 6.65 (bs, 2H), 6.77- 6.80 (dd, 1H, J1 = 2.12 Hz, J2 = 8.72 Hz), 6.86 (d, 1H, J= 8.8 Hz), 7.22- 7.26 (m, 4H), 7.30-7.34 (m, 2H), 8.45 (s, 1H), 10.57 (bs, 1H). | 377.17 | 98.66 |
| 133 | 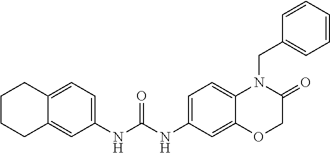 [SMILES: O=C(Nc1ccc2c(c1)CCCC2)Nc1ccc2c(c1)OCC(=O)N2Cc1ccccc1] | 1-(4-benzyl- 3-oxo-3,4- dihydro-2H- benzo[b][1, 4]oxazin-7- yl)-3-(5,6,7,8- tetrahydro- naphthalen- 2-yl)urea | (400 MHz; DMSO-d₆): δ 1.70-1.706 (m, 4H), 2.62.65 (m, 4H), 4.76 (s, 2H), 5.12 (s, 2H), 6.86- 6-93 (m, 3H), 7.07-7.09 (dd, 1H, J1 = 2.04 Hz, J2 = 8.16 Hz), 7.13 (bs, 1H), 7.24-7.28 (m, 4H), 7.31-7.35 (m, 2H), 8.48 (s, 1H), 8.63 (s, 1H). | 428.23 | 95.78 |
| 134 | 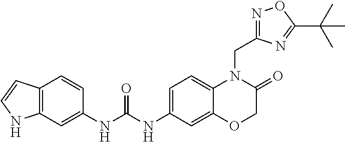 [SMILES: CC(C)(C)c1nc(CN2C(=O)COc3cc(NC(=O)Nc4ccc5cc[nH]c5c4)ccc32)no1] | l-(4-((5- (tert-butyl)- 1,2,4-oxadiazol- 3-yl)methyl)- 3-oxo-3,4- dihydro-2H- benzo[b][, 4]oxazin-7- yl)-3-(1H- | (400 MHz; DMSO-d₆): δ 1.35 (s, 9H), 4.70 (s, 2H), 5.22 (s, 2H), 6.33 (s, 1H), 6.84 (dd, 1H, J = 1.56 Hz, 8.4 Hz), 7.07-06.98 (m, 2H), 7.20 (t, 1H, J = 2.65 Hz), 7.30 (d, 1H, J = 2.12 Hz), 7.40 (d, 1H, J= 8.4 Hz), 7.75 (s, 1H), | 461.5 | 98.99 |
| | | indol-6- | 8.51 (s, 1H), 8.61 (s, 1H), | | |
| | | yl)urea | 10.89 (s, 1H). | | |
| 135 | 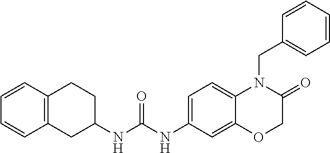 [SMILES: O=C(Nc1ccc2c(c1)OCC(=O)N2Cc1ccccc1)NC1CCc2ccccc2C1] | 1-(4-benzyl- 3-oxo-3,4- dihydro-2H- benzo[b][1, 4]oxazin-7- yl)-3-(1,2,3,4- tetrahydro- naphthalen- 2-yl)urea | (400 MHz; DMSO-d₆): δ 1.65-1.71 (m, 1H), 1.91- 1.95 (m, 1H), 2.57-2.63 (m, 1H), 2.80 (t, 2H, J = 6.4 Hz), 2.97-3.02 (dd, 1H, J1 = 4.96 Hz, J2 = 16.32 Hz), 3-89-3-93 (m, 1H), 4.73 (s, 2H), 5.10 (s, 2H), 6.22 (d, 1H, J = 7.6 | 428.24 | 98.75 |
| | | | Hz), 6.77-6.79 (dd, 1H, | | |
| | | | J1 = 2.2 Hz, J2 = 8.72 | | |
| | | | Hz), 6.86 (d, 1H, J = 8.8 | | |
| | | | Hz), 7.05-7.09 (m, 4H), | | |
| | | | 7.22-7.26 (m, 4H), 7.30- | | |
| | | | 7.34 (m, 2H), 8.37 (s, | | |
| | | | 1H). | | |
| 136 | 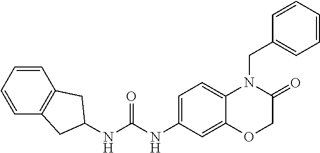 [SMILES: O=C(Nc1ccc2c(c1)OCC(=O)N2Cc1ccccc1)NC1Cc2ccccc2C1] | 1-(4-benzyl- 3-oxo-3,4- dihydro-2H- benzo[b][1, 4]oxazin-7- yl)-3-(2,3- dihydro-1H- inden-2- yl)urea | (400 MHz; DMSO-d₆): δ 2.71-2.76 (dd 2H, J1 = 5.24 Hz, J2 = 15.88 Hz), 3.13-3.18 (dd, 2H, J1 = 7.08 Hz, J2 = 15.84 Hz), 4.35-4.40 (m, 1H), 4.73 (s, 2H), 5.10 (s, 2H), 6.47 (d, 1H, J = 7.2 Hz), 6.77- 6.80 (dd, 1H, J1 = 2.12 | 414.19 | 97.29 |
| | | | Hz, J2 = 8.72 Hz), 6.86 | | |
| | | | (d, 1H, J = 8.8 Hz), 7.11- | | |
| | | | 7.16 (m, 2H), 7.22-7.28 | | |
| | | | (m, 6H), 7.30-7.34 (m, | | |
| | | | 2H), 8.35 (s, 1H). | | |
| 137 | 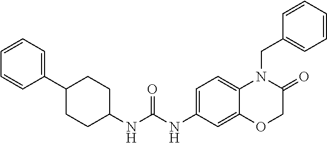 [SMILES: O=C(Nc1ccc2c(c1)OCC(=O)N2Cc1ccccc1)NC1CCC(c2ccccc2)CC1] | 1-(4-benzyl- 3-oxo-3,4- dihydro-2H- benzo[b][1, 4]oxazin-7- yl)-3-(4- phenyl- cyclohexyl)urea | (400 MHz; DMSO-d₆): δ 1.25-1.29 (m, 1H), 1.51- 1.55 (m, 1H), 1.62-1.72 (m, 4H), 1.78-1.81 (m, 1H), 1.93-1.96 (m, 1H), 3.46-3.48 (m, 1H), 3.49 (bs, 1H), 4.74(s, 2H), 5.10 (s, 2H), 6.20 (d, 1H, J = 7.88 Hz), 6.59 (d, 1H, | 456.24 | 55.3 + 43-86 (Diastereo- meric mixture) |
| | | | J = 7.68 Hz), 6.79-6.82 | | |
| | | | (m, 1H), 6.85-6.88 (m, | | |
| | | | 1H), 7.14-7.19 (m, 1H), | | |
| | | | 7.22-7.34 (m, 9H), 8.47 | | |
| | | | (d, 1H, J = 6.84 Hz). | | |
| 138 | 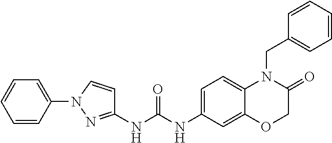 [SMILES: O=C(Nc1ccc2c(c1)OCC(=O)N2Cc1ccccc1)Nc1ccn(-c2ccccc2)n1] | 1-(4-benzyl- 3-oxo-3,4- dihydro-2H- benzo[b][1, 4]oxazin-7- yl)-3-(1- phenyl-1H- pyrazol-3- yl)urea | (400 MHz; DMSO-d₆): δ 4.78 (s, 2H), 5.13 (s, 2H), 6.57 (s, 1H, J = 2.48 Hz), 6.89-6.95 (m, 2H), 7.22- 7-35 (m, 7H), 7.46 (t, 2H, J = 7.76Hz), 7.74 (d, 2H, J = 7.88 Hz), 8.38 (d, 1H, J = 2.52 Hz), 8.91 (s, 1H), 9.24 (s, 1H). | 440.22 | 97.07 |
| 139 | 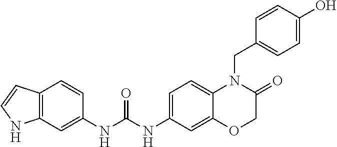 [SMILES: O=C(Nc1ccc2c(c1)OCC(=O)N2Cc1ccc(O)cc1)Nc1ccc2cc[nH]c2c1] | 1-(4-(4- hydroxybenzyl)- 3-oxo-3,4- dihydro-2H- benzo[b][1, 4]oxazin-7- yl)-3-(1H- indol-6- yl)urea | (400 MHz; DMSO-d₆): δ 4.73 (s, 2H), 4.99 (s, 2H), 6.31 (s, 1H), 6.70 (d, 2H, J = 8.4 Hz), 6.82-6.84 (dd, 1H, J1 = 1.32 Hz, J2 = 8.4 Hz), 6.90-6.97 (m, 2H), 7.09 (d, 2H, J = 8.44 Hz), 7.20 (t, 1H, J = 2.48 Hz), | 429.19 | 96.19 |
| | | | 7.29 (d, 1H, J = 1.96 Hz), | | |
| | | | 7.40 (d, 1 H, J = 8.44 | | |
| | | | Hz), 7.76 (s, 1H), 8.67 | | |
| | | | (s, 1H), 8.75 (s, 1H), 9.25 | | |
| | | | (bs, 1H), 10.89 (s, 1H). | | |
| 140 | 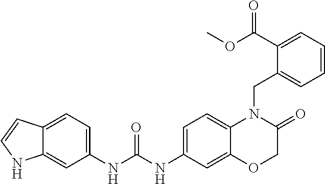 [SMILES: COC(=O)c1ccccc1CN1C(=O)COc2cc(NC(=O)Nc3ccc4cc[nH]c4c3)ccc21] | methyl 2- ((7-(3-(1H- indol-6- yl)ureido)- 3-oxo-2,3- dihydro-4H- benzo[b][1, 4]oxazin-4- yl)methyl) benzoate | (400 MHz; DMSO-d₆): δ 3.90 (s, 3H), 4.82 (s, 2H), 5.41 (s, 2H), 6.31 (s, 1H), 6.68 (d, 1H, J = 8.76 Hz), 6.81-6.87 (m, 2H), 7.12 (d, 1H, J = 7.84 Hz), 7.35-7.43 (m, 3H), 7.51 (t, 1H, J = 7.56H), 7.76 (s, 1H), 7.99 (d, 1H, J = 7.56 Hz), 8.57 (s, 1H), 8.57 (s, 1H), 8.67 (s, 1H), 10.89 (s, 1H). | 469.23 [M − H] | 99.12 |
| 141 | 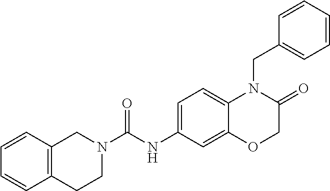 [SMILES: O=C(Nc1ccc2c(c1)OCC(=O)N2Cc1ccccc1)N1CCc2ccccc2C1] | N-(4-benzyl-3- oxo-3,4- dihydro-2H- benzo[b][1, 4]oxazin-7- yl)-3,4- dihydro- isoquinoline- 2(1H)- carboxamide | (400 MHz; DMSO-d₆): δ 2.82 (t, 2H, J = 8.36 Hz), 3.65 (t, 2H, J = 8.08 Hz), 4-59 (s, 2H), 4.74 (s, 2H), 5.11 (s, 2H), 6.89 (d, 1H, J = 8.8 Hz), 7.02- 7.04 (dd 1H, J1 = 2.16 Hz, J2 = 8.8 Hz) , 7.14- 7.17 (bs, 4H), 7.22-7.27 (m, 4H), 7.30-7.34 (m, 2H), 8.52 (s, 1H). | 414.22 | 98.81 |
| 142 | 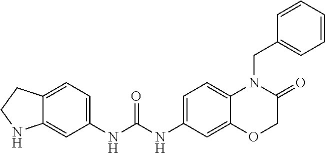 [SMILES: O=C(Nc1ccc2c(c1)NCC2)Nc1ccc2c(c1)OCC(=O)N2Cc1ccccc1] | 1-(4-benzyl- 3-oxo-3,4- dihydro-2H- benzo[b][1, 4]oxazin-7- yl)-3- (indolin-6- yl)urea | (400 MHz; DMSO-d₆): δ 2.80 (t, 2H, J = 8.36 Hz), 3.37 (t, 2H, J = 8.08 Hz), 4.76 (s, 2H), 5.11 (s, 2H), 5.45 (s, 1H), 6.46 (d, 1H, J = 7.64 Hz), 6.76 (s, 1H), 6.85-6.91 (m, 3H), 7.24-7.27 (m, 4H), 7.31-7.34 (m, 2H), 8.46 (s, 1H), 8.65 (s, 1H). | 415.21 | 97.17 |
| 144 | 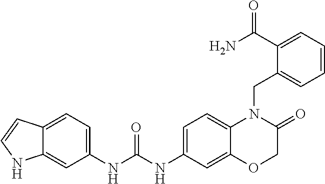 [SMILES: NC(=O)c1ccccc1CN1C(=O)COc2cc(NC(=O)Nc3ccc4cc[nH]c4c3)ccc21] | 2-((7-(3- (1H-indol-6- yl)ureido)- 3-oxo-2,3- dihydro-4H- benzo[b][1, 4]oxazin-4- yl)methyl) benzamide | (400 MHz; DMSO-d₆): δ 4.81 (s, 2H), 5.27 (s, 2H), 6.31 (s, 1H), 6.74 (d, 1H, J = 8.76 Hz), 6.80-6.87 (m, 2H), 7.04 (d, 1H, J = 7.24 Hz), 7.20 (t, 1H, J = 2.64 Hz), 7.32-7.40 (m, 4H), 7.53-7.5 (m, 1H), 7.58 (s, 1 H), 7.76 (s, 1H), 8.02 (s, 1H), 8.57 (s, 1H), 8.64 (s, 1H), 10.89 (s, 1H). | 456.2 | 98.74 |
| 145 | 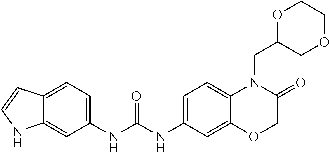 [SMILES: O=C(Nc1ccc2c(c1)OCC(=O)N2CC1COCCO1)Nc1ccc2cc[nH]c2c1] | l-(4-((1,4- dioxan-2- yl)methyl)- 3-oxo-3,4- dihydro-2H- benzo[b][1, 4]oxazin-7- yl)-3-(1H- indol-6- yl)urea | (400 MHz; DMSO-d₆): δ 3.28-3.33 (m, 1H), 3.46- 3.50 (m, 2H), 3.60-3.62 (m, 1H), 3-72-3.75 (m, 2H), 3.87-3.88 (m, 1H), 3-93-3-94 (m, 1H), 4.62 (s, 2H), 6.32 (s, 2H), 6.83-6.86 (dd, 1H, J1 = 1.28 Hz, J2 = 8.36 Hz), 7.02-7.04 (dd, 1H, J1 = 12.08 Hz, J2 = 8.72 Hz), | 423.18 | 99.31 |
| | | | 7.18-721 (m, 2H), 7.29 | | |
| | | | (d, 1H, J = 2.16 Hz), | | |
| | | | 7.39 (d, 1H, J = 8.4 Hz ), | | |
| | | | 7.78 (s, 1H), 8.66 (s, 1H), | | |
| | | | 8.76 (s, 1H), 10.90 (s, | | |
| | | | 1H). | | |
| 146 | 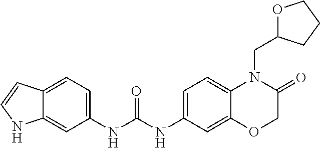 [SMILES: O=C(Nc1ccc2c(c1)OCC(=O)N2CC1CCCO1)Nc1ccc2cc[nH]c2c1] | 1-(1H-indol- 6-yl)-3-(3- oxo-4- ((tetrahydro- furan-2- yl)methyl)- 3,4-dihydro- 2H-benzo[b][1, 4]oxazin-7- yl)urea | (400 MHz; DMSO-d₆): δ 1.57-1.61 (m, 1H), 1.78- 1.88 (m, 1H), 1.89-1.94 (m, 2H), 3.59-3.63 (m, 1H), 3.72-3.77 (m, 1H), 3.93 (d, 2H, J = 5.92 Hz), 4.05-4.08 (m, 1H), 4.61 (s, 2H), 6.32 (s, 1H), 6.83-6.85 (dd, 1H, J1 = 1.32 Hz, J2 = 8.44 Hz), | 407.23 | 97.98 |
| | | | 7.00-7.03 (dd, 1H, J1 = | | |
| | | | 2.16 Hz, J2 = 8.76 Hz ), | | |
| | | | 7.20 (s, 1H), 7.22 (d, 1H, | | |
| | | | J = 8.96 Hz), 7.28 (d, | | |
| | | | 1H, J = 2.16 Hz), 7.40 (d, | | |
| | | | 1H, J = 8.4 Hz), 7.78 (s, | | |
| | | | 1H), 8.62 (s, 1H), 8.70 (s, | | |
| | | | 1H), 10.90 (s, 1H) | | |
| 147 | 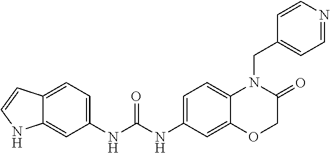 [SMILES: O=C(Nc1ccc2c(c1)OCC(=O)N2Cc1ccncc1)Nc1ccc2cc[nH]c2c1] | 1-(iH-indol- 6-yl)-3-(3- oxo-4- (pyridin-4- ylmethyl)- 3,4-dihydro- 2H-benzo[b][1, 4]oxazin-7- yl)urea | (400 MHz; DMSO-d₆): δ 4.81 (s, 2H), 5.15 (s, 2H), 6.31 (s, 1H), 6.80- 6.90 (m, 3H), 7.20-7.40 (m, 5H), 8.49-8.52 (t, 3H, J = 5 Hz), 8.58 (s, 1H), 10.90 (s, 1H). | 414.35 | 99.2 |
| 148 | 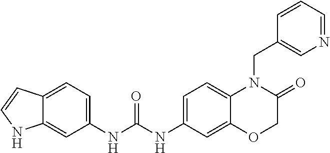 [SMILES: O=C(Nc1ccc2c(c1)OCC(=O)N2Cc1cccnc1)Nc1ccc2cc[nH]c2c1] | 1-(1H-indol- 6-yl)-3-(3- oxo-4- (pyridin-3- ylmethyl)-3,4- dihydro-2H- benzo [b][1, 4]oxazin-7- yl)urea | (400 MHz; DMSO-d₆): δ 4.78 (s, 2H), 5.17 (s, 2H), 6.32 (s, 1H), 6.80- 6.83 (m, 1H), 6.90-6.98 (m, 2H), 7.20 (t, 1H, J = 2.72 Hz), 7.30-7.40 (m, 3H), 7.67 (d, 1H, J = 7.92 Hz), 7.76 (s, 1H), 8.46- 8.50 (m, 2H), 8.56-8.58 (m, 2H), 10.90 (s, 1H). | 414.35 | 98.54 |
| 149 | 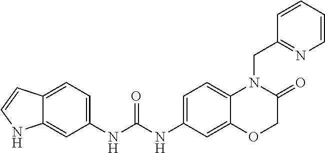 [SMILES: O=C(Nc1ccc2c(c1)OCC(=O)N2Cc1ccccn1)Nc1ccc2cc[nH]c2c1] | 1-(iH-indol- 6-yl)-3-(3- oxo-4- (pyridin-2- ylmethyl)-3,4- dihydro-2H- benzo[b][1, 4]oxazin-7- yl)urea | (400 MHz; DMSO-d₆): δ 4.76 (s, 2H), 5.17 (s, 2H), 6.32 (s, 1H), 6.80 (d, 1H, J = 7.08 Hz)), 6.88 (s, 2H), 7.20 (s, 1H), 7.27-7.40 (m, 4H), 7.76 (t, 2H, J = 7.12 Hz), 8.48-8.55 (m, 3H), 10.90 (s, 1H). | 414.38 | 99.86 |
| 150 | 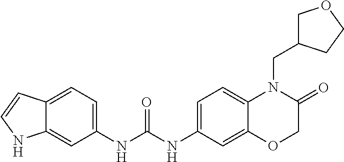 [SMILES: O=C(Nc1ccc2c(c1)OCC(=O)N2CC1CCOC1)Nc1ccc2cc[nH]c2c1] | 1-(1H-indol- 6-yl)-3-(3- oxo-4- ((tetrahydro furan-3- yl)methyl)-3,4- dihydro-2H- benzo [b][1, 4]oxazin-7- yl)urea | (400 MHz; DMSO-d₆): δ 1.55-1-65 (m, 1H), 1.82- 1.95 (m, 2H), 2.55-2.65 (m, 1H), 3.44-3.47 (m, 1H), 3.60-3.63 (m, 2H), 3.77-3.79 (m, 1H), 3.89- 3.94 (m, 2H), 4.62 (s, 2H), 6.32 (s, 1H), 6.82- 6.85 (dd, 1H, J1 = 1.52 Hz, J2 = 8.4 Hz), 7.03- | 407.2 | 98.58 |
| | | | 7.06 (dd, 1H, J1 = 2.2 | | |
| | | | Hz, J2 = 8.72 Hz), 7.17 | | |
| | | | (s, 1H), 7.19-7.22 (m, | | |
| | | | 2H), 7.28 (d, 1H, J = | | |
| | | | 2.24 Hz), 7.40 (d, 1 H, | | |
| | | | J = 8.4 Hz), 7.77 (s, 1H), | | |
| | | | 8.51 (s, 1H), 8.62 (s, 1H), | | |
| | | | 10.90 (s, 1H). | | |
| 151 | 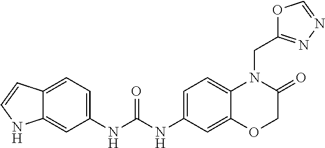 [SMILES: O=C(Nc1ccc2c(c1)OCC(=O)N2Cc1nnco1)Nc1ccc2cc[nH]c2c1] | 1-(4-((1,3,4′- oxadiazol-2- yl)methyl)- 3-oxo-3,4- dihydro-2H- benzo[b][1, 4]oxazin-7- yl)-3-(1H- indol-6- | (400 MHz; DMSO-d₆): δ 4.72 (s, 2H), 5.42 (s, 2H), 6.31 (s, 1H), 6.87 (d, 1H, J = 8.44 Hz), 7.05 (d, 1H, J = 2Hz), 7.13 (d, 1H, J = 8.8 Hz), 7.20 (t, 1H, J = 2.48 Hz), 7.34 (d, 1H, J = 2.16 Hz), 7.38 (d, 1H, J = 8.44 | 405.18 | 99.04 |
| | | yl)urea | Hz), 7.78 (s, 1H), 8.80 (s, | | |
| | | | 1H), 9.1 (s, 1H), 9.22 (s, | | |
| | | | 1H), 10.89 (s, 1H). | | |
| 152 | 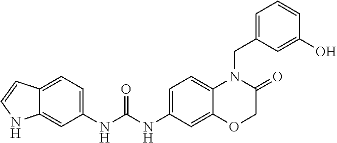 [SMILES: O=C(Nc1ccc2c(c1)OCC(=O)N2Cc1cccc(O)c1)Nc1ccc2cc[nH]c2c1] | 1-(4-(3- hydroxybenzyl)- 3-oxo-3,4- dihydro-2H- benzo[b][1, 4]oxazin-7- yl)-3-(1H- indol-6- yl)urea | (400 MHz; DMSO-d₆): δ 4.75 (s, 2H), 5.04 (s, 2H), 6.32 (s, 1H), 6.62 (s, 1H), 6.64 (s, 1H), 6.69 (d, 1H, J = 7.64 Hz), 6.82 (d, 1H, J = 7.12 Hz), 6.90 (s, 2H), 7.11 (t, 1H, J = 7.64 Hz), 7.20 (s, 1H), 7.31 (s, 1H), 7.38 (d, | 427.0 | 98.75 |
| | | | 1H, J = 8.4 Hz), 7.76 (s, | | |
| | | | 1H), 8.50 (s, 1H), 8.57 (s, | | |
| | | | 1H), 9.38 (s, 1H), 10.89 | | |
| | | | (s, 1H) | | |
| 153 | 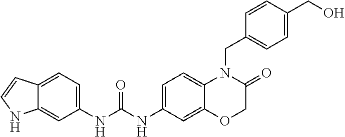 [SMILES: O=C(Nc1ccc2c(c1)OCC(=O)N2Cc1ccc(CO)cc1)Nc1ccc2cc[nH]c2c1] | 1-(4-(4- (hydroxymethyl) benzyl)-3-oxo- 3,4-dihydro-2H- benzo[b][1, 4]oxazin-7- yl)-3-(1H- indol-6- yl)urea | (400 MHz; DMS0-d₆): δ 4.44 (s, 2H), 4.76 (s, 2H), 5.12 (d, 3H, J = 7.6 Hz), 6.32 (s, 1H), 6.82 (d, 1H, J = 8.4 Hz), 6.87- 6.93 (m, 2H), 7.20-7.40 (m, 7H), 7.75 (s, 1H), 8.50 (s, 1H), 8.56 (s, 1H), 10.90 (s, 1H) | 443.1 | 98.72 |
| 154 | 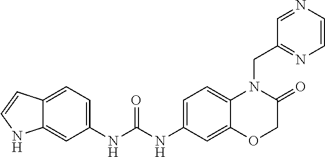 [SMILES: O=C(Nc1ccc2c(c1)OCC(=O)N2Cc1cnccn1)Nc1ccc2cc[nH]c2c1] | 1-(1H-indol- 6-yl)-3-(3- oxo-4- (pyrazin-2- ylmethyl)-3,4- dihydro-2H- benzo[b][i, 4]oxazin-7- yl)urea | (400 MHz; DMSO-d₆): δ 4.76 (s, 2H), 5.27 (s, 2H, J = 7.6 Hz), 6.32 (s, 1H), 6.82 (d, 1H, J = 8.4 Hz), 6.91-6.98 (m, 2H), 7.20 (s, 1H), 7.31 (s, 1H), 7.39 (d, 1H, J = 8.4 Hz), 8.56- 8.68 (m, 5H), 10.90 (s, 1H) | 415.0 | 94.46 |
| 155 | 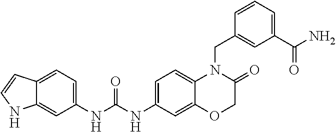 [SMILES: NC(=O)c1cccc(CN2C(=O)COc3cc(NC(=O)Nc4ccc5cc[nH]c5c4)ccc32)c1] | 3-((7-(3- (1H-indol-6- yl)ureido)- 3-oxo-2,3- dihydro-4H- benzo[b][1, 4]oxazin-4- yl)methyl) benzamide | (400 MHz; DMSO-d₆): δ 4.79 (s, 2H), 5.16 (s, 2H, J = 7.6 Hz), 6.32 (s, 1H), 6.82 (d, 1H, J = 8.4 Hz), 6.89 (s, 2H), 7.20-7.41 (m, 6H), 7.76-7.97 (m, 4H), 8.51 (s, 1H), 8.58 (s, 1H), 10.90 (s, 1H) | 415.0 | 99.39 |
| 156 | 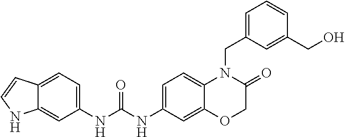 [SMILES: O=C(Nc1ccc2c(c1)OCC(=O)N2Cc1cccc(CO)c1)Nc1ccc2cc[nH]c2c1] | 1-(4-(3- (hydroxymethyl) benzyl)-3-oxo- 3,4-dihydro- 2H-benzo[b][1, 4]oxazin-7- yl)-3-(1H- indol-6- yl)urea | (400 MHz; DMSO-d₆): δ 4.46 (d, 2H, J = 5.7 Hz), 4-77 (s, 2H), 5.11-5.17 (m, 3H), 6.32 (s, 1H), 6.82 (d, 1H, J = 8.4 Hz), 6.90 (s, 2H), 7.15-7.40 (m, 6H), 7.75 (s, 1H), 8.50 (s, 1H), 8.56 (s, 1H), 10.90 (s, 1H) | 443.4 | 99.27 |
| 157 | 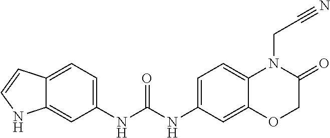 [SMILES: N#CCN1C(=O)COc2cc(NC(=O)Nc3ccc4cc[nH]c4c3)ccc21] | 1-(4- (cyanomethyl)- 3-oxo-3,4- dihydro-2H- benzo[b][1, 4]oxazin-7- yl)-3-(1H- indol-6- yl)urea | (400 MHz; DMS0-d₆): δ 4.74 (s, 2H), 5.07 (s, 2H), 6.30 (s, 1H), 6.87 (d, 1H, J = 8.36 Hz), 7.13 (d, 1H, J = 8.68 Hz), 7.19 (d, 2H, J = 8.28 Hz), 7.35 (s, 1H), 7.40 (d, 1H, J = 8.32 Hz), 7.78 (s, 1H), 8.74 (s, 1H), 8.89 (s, | 362.14 | 96.91 |
| | | | 1H), 10.88 (s, 1H). | | |
| 158 | 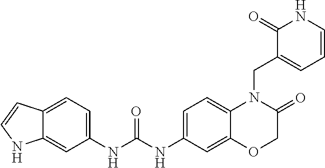 [SMILES: O=C(Nc1ccc2c(c1)OCC(=O)N2Cc1ccc[nH]c1=O)Nc1ccc2cc[nH]c2c1] | 1-(1H-indol- 6-yl)-3-(3- oxo-4-((2- oxo-1,2- dihydropyridin- 3-yl)methyl)- 3,4-dihydro- 2H-benzo[b][1, 4]oxazin-7- yl)urea | (400 MHz; DMS0-d₆): δ 4.76 (s, 2H), 4.80 (s, 2H), 6.13 (t, 1H, J = 6.64 Hz), 6.32 (s, 1H), 6.78- 6.92 (m, 3H), 7.09-7.40 (m, 5H), 7.77 (s, 1H), 8.51 (s, 1H), 8.60 (s, 1H), 10.89 (s, 1H), 11.78 (s, 1H) | 430.39 | 99.76 |
| 159 | 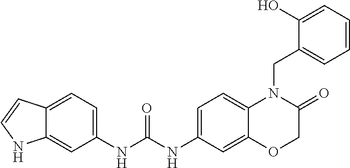 [SMILES: O=C(Nc1ccc2c(c1)OCC(=O)N2Cc1ccccc1O)Nc1ccc2cc[nH]c2c1] | 1-(4-(2- hydroxybenzyl)- 3-oxo-3,4- dihydro-2H- benzo [b][1, 4]oxazin-7- yl)-3-(1H- indol-6- yl)urea | (400 MHz; DMSO-d₆): δ 4.77 (s, 2H), 4.99 (s, 2H), 6.32 (s, 1H), 6.69- 6.87 (m, 6H), 7.05-7.40 (m, 4H), 7.76 (s, 1H), 8.51 (s, 1H), 8.58 (s, 1H), 9.82 (s, 1H), 10.90 (s, 1H) | 429.3 | 98.17 |
| 160 | 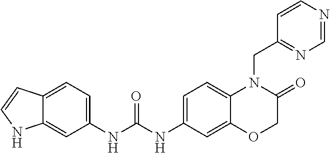 [SMILES: O=C(Nc1ccc2c(c1)OCC(=O)N2Cc1ccncn1)Nc1ccc2cc[nH]c2c1] | 1-(1H-indol- 6-yl)-3-(3- oxo-4- (pyrimidin-4- ylmethyl)-3,4- dihydro-2H- benzo[b][1, 4]oxazin-7- yl)urea | (400 MHz; DMSO-d₆): δ 4.78 (s, 2H), 5.19 (s, 2H), 6.32 (s, 1H), 6.80-6.91 (m, 3H), 7.20-7.46 (m, 4H), 7.76 (s, 1H), 8.50 (s, 1H), 8.58 (s, 1H), 8.75 (d, 1H, J = 5.16 Hz), 9.13 (s, 1H) | 415.2 | 98.71 |
| 161 | 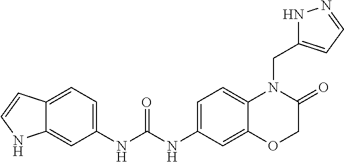 [SMILES: O=C(Nc1ccc2c(c1)OCC(=O)N2Cc1ccn[nH]1)Nc1ccc2cc[nH]c2c1] | 1-(4-((1H- pyrazol-5- yl)methyl)- 3-oxo-3,4- dihydro-2H- benzo[b][1, 4]oxazin-7- yl)-3-(1H- indol-6- yl)urea | (400 MHz; DMSO-d₆): δ 4.68 (s, 2H), 5.03 (s, 2H), 6.10 (s, 1H), 6.32 (s, 1H) 6.80-6.94 (m, 2H), 7.13-7.40 (m, 4H), 7.64 (s, 1H), 7.77 (s, 1H), 8.51 (s, 1H), 8.57 (s, 1H), 10.90 (s, 1H), 12.67 (s, 1H) | 403.2 | 99.42 |
| 162 | 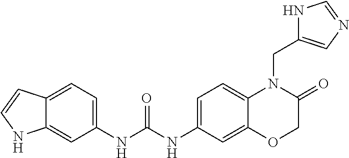 [SMILES: O=C(Nc1ccc2c(c1)OCC(=O)N2Cc1cnc[nH]1)Nc1ccc2cc[nH]c2c1] | 1-(4-((1H- imidazol-5- yl)methyl)- 3-oxo-3,4- dihydro-2H- benzo[b][1, 4]oxazin-7- yl)-3-(1H- indol-6- yl)urea | (400 MHz; DMSO-d₆): δ 4.65 (s, 2H), 4.93 (s, 2H), 6.32 (s, 1H) 6.80- 6.97 (m, 3H), 7.20-7.40 (m, 4H), 7.55 (s, 1H), 7.77 (s, 1H), 8.49 (s, 1H), 8.55 (s, 1H), 10.90 (s, 1H), 11.94 (s, 1H) | 403.39 | 99.62 |
| 164 | 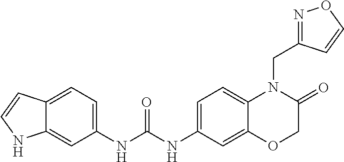 [SMILES: O=C(Nc1ccc2c(c1)OCC(=O)N2Cc1ccon1)Nc1ccc2cc[nH]c2c1] | 1-(1H-indol- 6-yl)-3-(4- (isoxazol-3- ylmethyl)- 3-oxo-3,4- dihydro-2H- benzo[b][1, 4]oxazin-7- yl)urea | (400 MHz; DMSO-d₆): δ 4.73 (s, 2H), 5.19 (s, 2H), 6.32 (s, 1H), 6.48 (d, 1H, J = 1.48 Hz), 6.82 (dd, 1H, J1 = 1.68 Hz, J2 = 8.4 Hz), 6.94-7.02 (m, 2H), 7.21 (t, 1H, J = 2.68 Hz), 7.32 (d, 1H, 2.16 Hz), 7.38 (d, 1H, J = 8.44 Hz), 7.77 (s, 1H), | 404.2 | 99.92 |
| | | | 8.50 (s, 1H), 8.59 (s, 1H), | | |
| | | | 8.88 (s, 1H), 10.90 (s, | | |
| | | | 1H) | | |
| 165 | 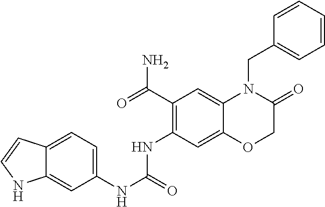 [SMILES: NC(=O)c1cc2c(cc1NC(=O)Nc1ccc3cc[nH]c3c1)OCC(=O)N2Cc1ccccc1] | 7-(3-(H- indol-6- yl)ureido)- 4-benzyl-3- oxo-3,4- dihydro-2H- benzo[b][1, 4]oxazine-6- carboxamide | (400 MHz; DMSO-d₆): δ 4.84 (s, 2H), 5.24 (s, 2 H), 6.31 (s, 1H), 6.96 (d, 1H, J = 9.6 Hz), 7.20- 7.49 (m, 9H), 7.75 (s, 1H), 8.00 (s, 1H), 8.09 (s, 1H), 9.50 (s, 1H), 10.70 (s, 1H), 10.90 (s, 1H) | 456.31 | 99.45 |
| 166 | 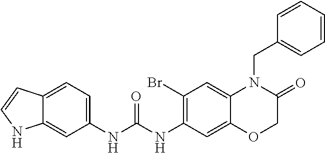 [SMILES: O=C(Nc1ccc2cc[nH]c2c1)Nc1cc2c(cc1Br)N(Cc1ccccc1)C(=O)CO2] | 1-(4-benzyl- 6-bromo-3- oxo-3,4- dihydro-2H- benzo[b][1, 4]oxazin-7- yl)-3-(1H- indol-6- yl)urea | (400 MHz; DMSO-d₆): δ 4.82 (s, 2H), 5.16 (s, 2 H), 6.33 (s, 1H), 6.82- 6.85 (m, 1H), 7.21-7.42 (m, 8H), 7.78 (s, 1H), 7.82 (s, 1H), 7.97 (s, 1H), 9.31 (s, 1H), 10.94 (s, 1H) | 491.18 | 99.23 |
| 193 | 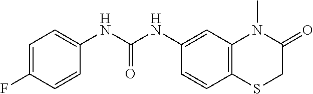 [SMILES: CN1C(=O)CSc2ccc(NC(=O)Nc3ccc(F)cc3)cc21] | l-(4- fluorophenyl)- 3-(4- methyl-3- oxo-3,4- dihydro-2H- | (500 MHz; DMSO-d₆): δ 3.48 (s, 3 H), 7.08-7.14 (m, 4 H), 7.29 (d, J = 8.4 Hz, 1H), 7.46-7.48 (m, 4H), 8.74 (s, 1 H), 8.82 (s, 1 H) | 332.24 | 90.49 |
| | | benzo[b][1, | | | |
| | | 4]thiazin-6- | | | |
| | | yl)urea | | | |
| 194 | 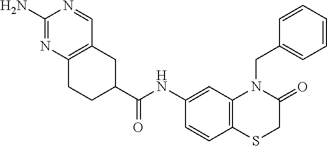 [SMILES: Nc1ncc2c(n1)CCC(C(=O)Nc1ccc3c(c1)N(Cc1ccccc1)C(=O)CS3)C2] | 2-amino-N- (4-benzyl- 3-oxo-3,4- dihydro-2H- benzo[b][1, 4]thiazin-6- yl)-7,8- dihydropyrido [4,3-d] | (400 MHz; DMSO-d₆): δ 2.64 (t, J = 5.96 Hz , 2H), 3.60 (s, 2H), 3.67 (s, 2H), 4.40 (s, 2H), 5.13 (s, 2H), 6.44 (s, 2H), 7.20-7.30 (m, 6H), 7.45 (d, J = 2 Hz, 1H), 8.04 (s, 1H), 8.10 (s, 1H), 8.72 (s, 1H). | 447.33 | 91.22 |
| | | pyrimidine-6(5H)- | | | |
| | | carboxamide | | | |
| 195 | 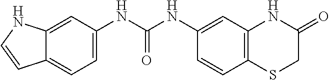 [SMILES: O=C1CSc2ccc(NC(=O)Nc3ccc4cc[nH]c4c3)cc2N1] | 1-(1H-indol- 6-yl)-3-(3- oxo-3,4- dihydro-2H- benzo[b][1, | (500 MHz; DMSO-d4): δ 3.42 (s, 2H), 6.33 (s, 1H), 6.85 (d, J = 8.4 Hz, 1H), 7.08 (d, J = 808 Hz, 1H), 7.18-7.24 (m, 2H), | 339.3 | 95.25 |
| | | 4]thiazin-6- | 7.40 (d, J = 8.16 Hz, | | |
| | | yl)urea | 2H), 7.79 (s, 1H), 8.64 (s, | | |
| | | | 1H), 8.82 (s, 1H), 10.53 | | |
| | | | (s, 1H), 10.93 (s, 1H) | | |

### Examples 97-98, 100-115, 117-123, 143 and 163

Examples 97-98, 100-115, 117-123, 143 and 163 were prepared using Library General Procedure 28 and 29 using the appropriate aryl halide. Purification was as stated in the aforementioned methods.

| | | | LCMS | Purity |
|---|---|---|---|---|
| Ex | Structure | IUPAC Name | [M + H] | (%) |
| 97 | 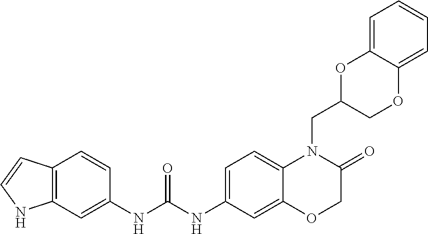 [SMILES: O=C(Nc1ccc2c(c1)OCC(=O)N2CC1COc2ccccc2O1)Nc1ccc2cc[nH]c2c1] | 1-(4-((2,3- dihydrobenzo[b][1,4] dioxin-2-yl)methyl)-3- oxo-3,4-dihydro-2H- benzo[b][1,4]oxazin-7- yl)-3-(1H-indol-6- yl)urea | 471.13 | 99.9 |
| 98 | 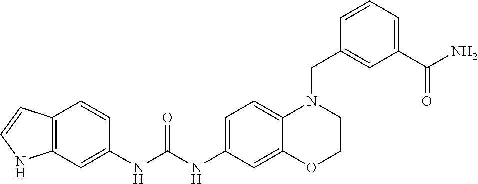 [SMILES: NC(=O)c1cccc(CN2CCOc3cc(NC(=O)Nc4ccc5cc[nH]c5c4)ccc32)c1] | 3-((7-(3-(1H-indol-6- yl)ureido)-3-oxo-2,3- dihydro-4H- benzo[b][1,4]oxazin-4- yl)methyl)benzamide | 456.12 | 97.07 |
| 100 | 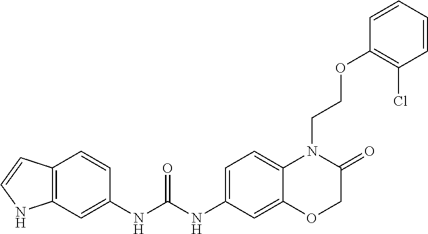 [SMILES: O=C(Nc1ccc2c(c1)OCC(=O)N2CCOc1ccccc1Cl)Nc1ccc2cc[nH]c2c1] | 1-(4-(2-(2- chlorophenoxy)ethyl)- 3-oxo-3,4-dihydro-2H- benzo[b][1,4]oxazin-7- yl)-3-(1H-indol-6- yl)urea | 477.26 | 99.2 |
| 101 | 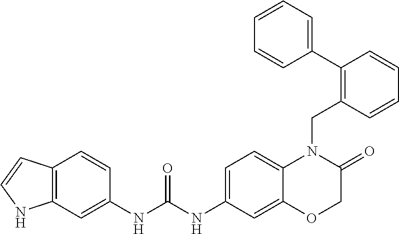 [SMILES: O=C(Nc1ccc2c(c1)OCC(=O)N2Cc1ccccc1-c1ccccc1)Nc1ccc2cc[nH]c2c1] | 1-(4-([1,1′-biphenyl]-2- ylmethyl)-3-oxo-3,4- dihydro-2H- benzo[b][1,4]oxazin-7- yl)-3-(1H-indol-6- yl)urea | 489.39 | 98.73 |
| 102 | 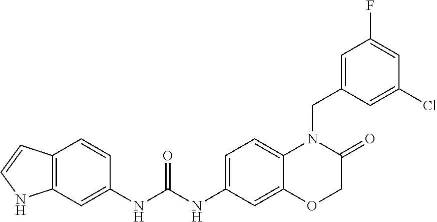 [SMILES: O=C(Nc1ccc2c(c1)OCC(=O)N2Cc1cc(F)cc(Cl)c1)Nc1ccc2cc[nH]c2c1] | 1-(4-(3-chloro-5- fluorobenzyl)-3-oxo- 3,4-dihydro-2H- benzo[b][1,4]oxazin-7- yl)-3-(1H-indol-6- yl)urea | 465.16 | 99.12 |
| 103 | 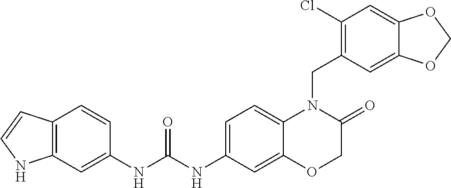 [SMILES: O=C(Nc1ccc2c(c1)OCC(=O)N2Cc1cc2c(cc1Cl)OCO2)Nc1ccc2cc[nH]c2c1] | 1-(4-((6- chlorobenzo[d][1,3] dioxol-5-yl)methyl)-3-oxo- 3,4-dihydro-2H- benzo[b][1,4]oxazin-7- yl)-3-(1H-indol-6- yl)urea | 491.15 | 96.12 |
| 104 | 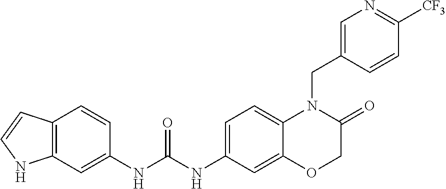 [SMILES: O=C(Nc1ccc2c(c1)OCC(=O)N2Cc1ccc(C(F)(F)F)nc1)Nc1ccc2cc[nH]c2c1] | 1-(1H-indol-6-yl)-3-(3- oxo-4-((6- (trifluoromethyl)pyridin- 3-yl)methyl)-3,4- dihydro-2H- benzo[b][1,4]oxazin-7- yl)urea | 482.2 | 98.05 |
| 105 | 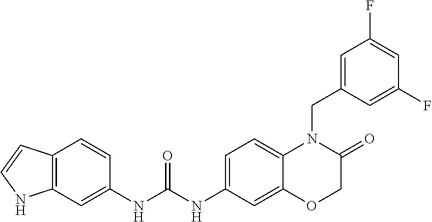 [SMILES: O=C(Nc1ccc2c(c1)OCC(=O)N2Cc1cc(F)cc(F)c1)Nc1ccc2cc[nH]c2c1] | 1-(4-(3,5- difluorobenzyl)-3-oxo- 3,4-dihydro-2H- benzo[b][1,4]oxazin-7- yl)-3-(1H-indol-6- yl)urea | 449.34 | 100 |
| 106 | 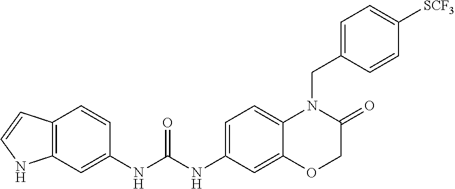 | 1-(1H-indol-6-yl)-3-(3- oxo-4-(4- ((trifluoromethyl)thio) benzyl)-3,4-dihydro- 2H- benzo[b][1,4]oxazin-7- yl)urea | 511.39 [M − H] | 98.66 |
| 107 | 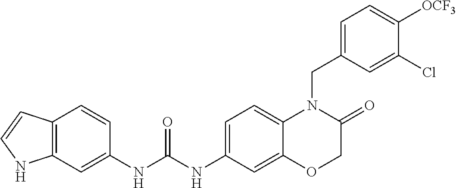 [SMILES: O=C(Nc1ccc2c(c1)OCC(=O)N2Cc1ccc(OC(F)(F)F)c(Cl)c1)Nc1ccc2cc[nH]c2c1] | 1-(4-(3-chloro-4- (trifluoromethoxy)benzyl)- 3-oxo-3,4-dihydro- 2H- benzo[b][1,4]oxazin-7- yl)-3-(1H-indol-6- yl)urea | 531.39 | 99.48 |
| 108 | 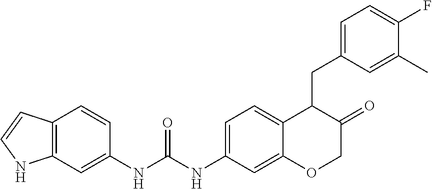 [SMILES: Cc1cc(CC2C(=O)COc3cc(NC(=O)Nc4ccc5cc[nH]c5c4)ccc32)ccc1F] | 1-(4-(4-fluoro-3- methylbenzyl)-3-oxo- 3,4-dihydro-2H- benzo[b][1,4]oxazin-7- yl)-3-(1H-indol-6- yl)urea | 445.32 | 98.58 |
| 109 | 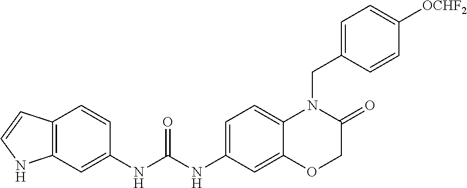 [SMILES: O=C(Nc1ccc2c(c1)OCC(=O)N2Cc1ccc(OC(F)F)cc1)Nc1ccc2cc[nH]c2c1] | 1-(4-(4- (difluoromethoxy)benzyl)- 3-oxo-3,4-dihydro- 2H- benzo[b][1,4]oxazin-7- yl)-3-(1H-indol-6- yl)urea | 479.34 | 100 |
| 110 | 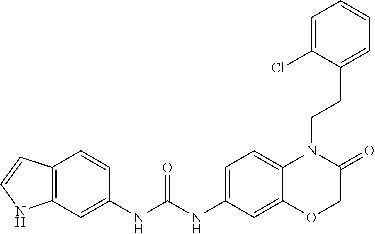 [SMILES: O=C(Nc1ccc2c(c1)OCC(=O)N2CCc1ccccc1Cl)Nc1ccc2cc[nH]c2c1] | 1-(4-(2- chlorophenethyl)-3- oxo-3,4-dihydro-2H- benzo[b][1,4]oxazin-7- yl)-3-(1H-indol-6- yl)urea | 461.3 | 100 |
| 111 | 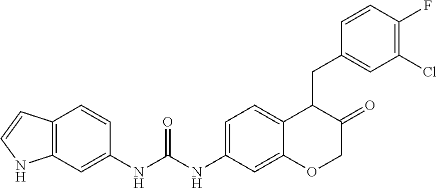 [SMILES: O=C(Nc1ccc2c(c1)OCC(=O)C2Cc1ccc(F)c(Cl)c1)Nc1ccc2cc[nH]c2c1] | 1-(4-(3-chloro-4- fluorobenzyl)-3-oxo- 3,4-dihydro-2H- benzo[b][1,4]oxazin-7- yl)-3-(1H-indol-6- yl)urea | 465.3 | 98.1 |
| 112 | 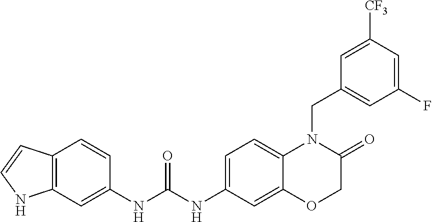 [SMILES: O=C(Nc1ccc2c(c1)OCC(=O)N2Cc1cc(F)cc(C(F)(F)F)c1)Nc1ccc2cc[nH]c2c1] | 1-(4-(3-fluoro-5- (trifluoromethyl)benzyl)- 3-oxo-3,4-dihydro- 2H- benzo[b][1,4]oxazin-7- yl)-3-(1H-indol-6- yl)urea | 499.3 | 98.19 |
| 113 | 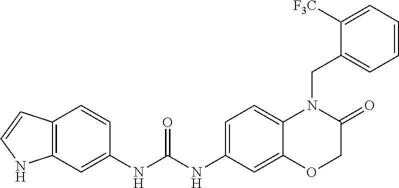 [SMILES: O=C(Nc1ccc2c(c1)OCC(=O)N2Cc1ccccc1C(F)(F)F)Nc1ccc2cc[nH]c2c1] | 1-(1H-indol-6-yl)-3-(3- oxo-4-(2- (trifluoromethyl)benzyl)- 3,4-dihydro-2H- benzo[b][1,4]oxazin-7- yl)urea | 481.36 | 96.78 |
| 114 | 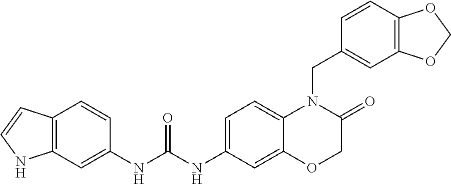 [SMILES: O=C(Nc1ccc2c(c1)OCC(=O)N2Cc1ccc2c(c1)OCO2)Nc1ccc2cc[nH]c2c1] | 1-(4- (benzo[d][1,3]dioxol-5- ylmethyl)-3-oxo-3,4- dihydro-2H- benzo[b][1,4]oxazin-7- yl)-3-(1H-indol-6- yl)urea | 457.13 | 99.08 |
| 115 | 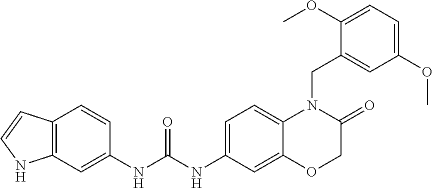 [SMILES: COc1ccc(OC)c(CN2C(=O)COc3cc(NC(=O)Nc4ccc5cc[nH]c5c4)ccc32)c1] | 1-(4-(2,5- dimethoxybenzyl)-3- oxo-3,4-dihydro-2H- benzo[b][1,4]oxazin-7- yl)-3-(1H-indol-6- yl)urea | 473.4 | 91.72 |
| 117 | 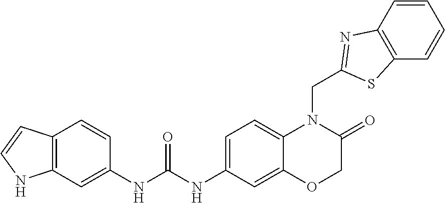 [SMILES: O=C(Nc1ccc2c(c1)OCC(=O)N2Cc1nc2ccccc2s1)Nc1ccc2cc[nH]c2c1] | 1-(4-(benzo[d]thiazol- 2-ylmethyl)-3-oxo-3,4- dihydro-2H- benzo[b][1,4]oxazin-7- yl)-3-(1H-indol-6- yl)urea | 470.33 | 100 |
| 118 | 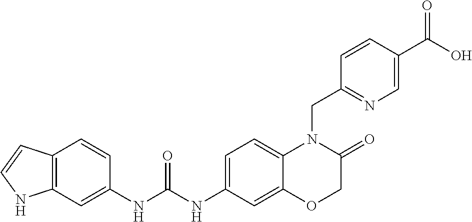 [SMILES: O=C(Nc1ccc2c(c1)OCC(=O)N2Cc1ccc(C(=O)O)cn1)Nc1ccc2cc[nH]c2c1] | 6-((7-(3-(1H-indol-6- yl)ureido)-3-oxo-2,3- dihydro-4H- benzo[b][1,4]oxazin-4- yl)methyl)nicotinic acid | 458.36 | 94.68 |
| 119 | 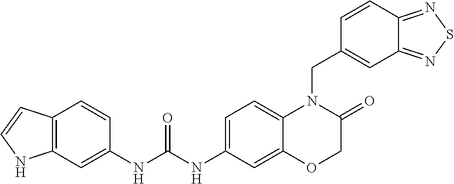 [SMILES: O=C(Nc1ccc2c(c1)OCC(=O)N2Cc1ccc2nsnc2c1)Nc1ccc2cc[nH]c2c1] | 1-(4- (benzo[c][1,2,5]thiadiazol- 5-ylmethyl)-3-oxo- 3,4-dihydro-2H- benzo[b][1,4]oxazin-7- yl)-3-(1H-indol-6- yl)urea | 469.40 [M − H] | 100 |
| 120 | 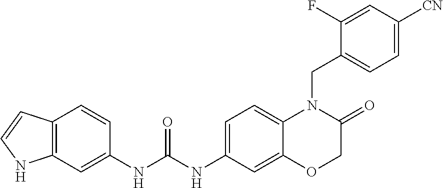 [SMILES: N#Cc1ccc(CN2C(=O)COc3cc(NC(=O)Nc4ccc5cc[nH]c5c4)ccc32)c(F)c1] | 1-(4-(4-cyano-2- fluorobenzyl)-3-oxo- 3,4-dihydro-2H- benzo[b][1,4]oxazin-7- yl)-3-(1H-indol-6- yl)urea | 456.39 | 100 |
| 121 | 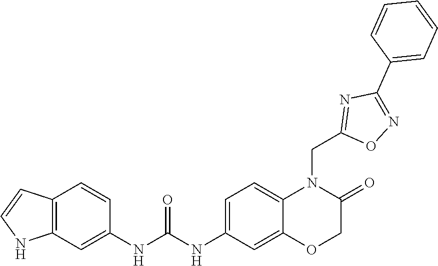 [SMILES: O=C(Nc1ccc2c(c1)OCC(=O)N2Cc1nc(-c2ccccc2)no1)Nc1ccc2cc[nH]c2c1] | 1-(1H-indol-6-yl)-3-(3- oxo-4-((3-phenyl-1,2,4- oxadiazol-5-yl)methyl)- 3,4-dihydro-2H- benzo[b][1,4]oxazin-7- yl)urea | 481.46 | 100 |
| 122 | 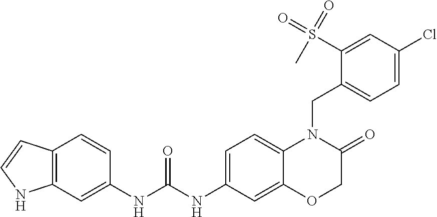 [SMILES: CS(=O)(=O)c1cc(Cl)ccc1CN1C(=O)COc2cc(NC(=O)Nc3ccc4cc[nH]c4c3)ccc21] | 1-(4-(4-chloro-2- (methylsulfonyl)benzyl)- 3-oxo-3,4-dihydro- 2H- benzo[b][1,4]oxazin-7- yl)-3-(1H-indol-6- yl)urea | 525.42 | 100 |
| 123 | 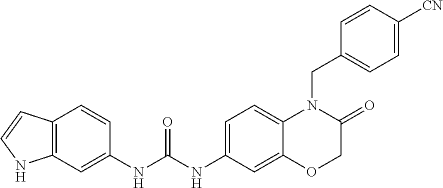 [SMILES: N#Cc1ccc(CN2C(=O)COc3cc(NC(=O)Nc4ccc5cc[nH]c5c4)ccc32)cc1] | 1-(4-(4-cyanobenzyl)- 3-oxo-3,4-dihydro-2H- benzo[b][1,4]oxazin-7- yl)-3-(1H-indol-6- yl)urea | 436.41 [M − H] | 100 |
| 143 | 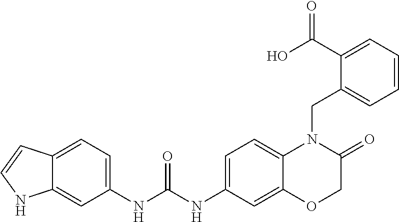 [SMILES: O=C(Nc1ccc2c(c1)OCC(=O)N2Cc1ccccc1C(=O)O)Nc1ccc2cc[nH]c2c1] | 2-((7-(3-(1H-indol-6- yl)ureido)-3-oxo-2,3- dihydro-4H- benzo[b][1,4]oxazin-4- yl)methyl)benzoic acid | 457.20 | 98.52 |
| 163 | 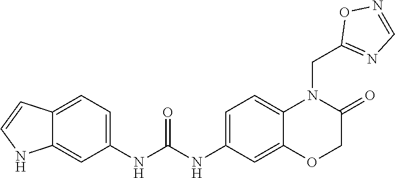 [SMILES: O=C(Nc1ccc2c(c1)OCC(=O)N2Cc1ncno1)Nc1ccc2cc[nH]c2c1] | 1-(4-((1,2,4-oxadiazol- 5-yl)methyl)-3-oxo- 3,4-dihydro-2H- benzo[b][1,4]oxazin-7- yl)-3-(1H-indol-6- yl)urea | 405.18 | 98.69 |

### Example 38: (S)-1-(1-benzyl-3,4-dimethyl-2-oxo-1,2,3,4-tetrahydro-quinazolin-7-yl)-3-(4-fluorophenyl)urea

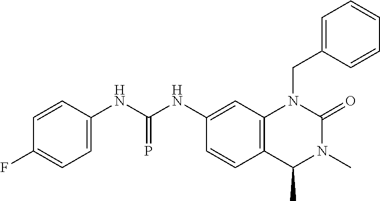

Example 38 was prepared according to the methods described in General Procedures 1-4, 10-14 and the methods described below

### Preparation 7: (S)-Methyl-3,4-dimethyl-2-oxo-1,2,3,4-tetrahydroquinazoline-7-carboxylate

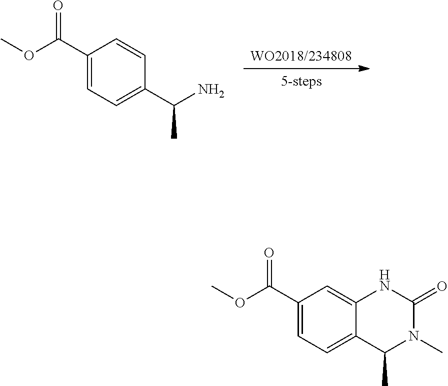 [SMILES: COC(=O)c1ccc([C@H](C)N)cc1 ; "5-steps" ; "WO2018/234808" ; COC(=O)c1ccc2c(c1)NC(=O)N(C)[C@H]2C]

(S)-methyl-3,4-dimethyl-2-oxo-1,2,3,4-tetrahydroquinazoline-7-carboxylate was prepared in five steps according to the methods described in patent WO2018/234808.

### Preparation 8: (S)-Methyl 1-benzyl-3,4-dimethyl-2-oxo-1,2,3,4-tetrahydroquinazoline-7-carboxylate

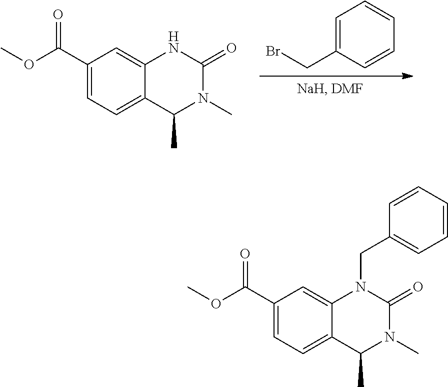

To a stirred solution of (S)-methyl-3,4-dimethyl-2-oxo-1,2,3,4-tetrahydroquinazoline-7-carboxylate (Preparation 7) (1.0 g, 4.26 mmol) in DMF (12 mL) was added NaH (187 mg, 4.69 mmol) followed by benzyl bromide (0.53 mL, 4.48 mmol) at 0-5° C. The combined mixture was stirred at RT for 30 min. TLC showed complete consumption of the starting cyclic urea. Then the reaction mixture was quenched with ice-water to give a precipitate which was filtered, washed with hexane and dried under high vacuum to afford the title compound (1.1 g, 80% yield) as a white solid. LCMS m/z: 325 [M+H].

### Preparation 9: (S)-1-Benzyl-3,4-dimethyl-2-oxo-1,2,3,4-tetrahydroquinazoline-7-carboxylic acid

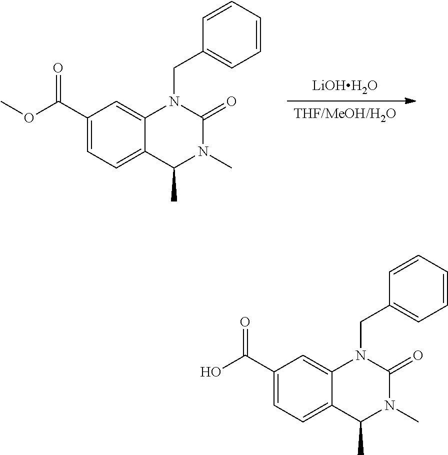 [SMILES: COC(=O)c1ccc2c(c1)N(Cc1ccccc1)C(=O)N(C)[C@H]2C ; "LiOH·H2O" ; "THF/MeOH/H2O" ; C[C@H]1c2ccc(C(=O)O)cc2N(Cc2ccccc2)C(=O)N1C]

To a stirred solution of (S)-methyl 1-benzyl-3,4-dimethyl-2-oxo-1,2,3,4-tetrahydroquinazoline-7-carboxylate (Preparation 8) (0.5 g, 1.54 mmol) in THF (5 mL) and MeOH (2.5 mL) was added a solution of LiOH.H₂O (258 mg, 6.16 mmol) in water (2.5 mL) and the combined mixture stirred at room temperature for 2 h. TLC showed completion of the reaction. The solvents were evaporated and the residue was diluted with water, washed with MTBE and the aqueous layer acidified with 1N HCl to pH 4-5. The aqueous part was extracted with EtOAc, washed with brine, dried over anhydrous MgSO₄, filtered and concentrated in vacuo to afford the title compound (450 mg, crude) as a white solid. LCMS m/z: 311 [M+H].

### Preparation 10: (S)-1-Benzyl-3,4-dimethyl-2-oxo-1,2,3,4-tetrahydroquinazoline-7-carbonyl azide

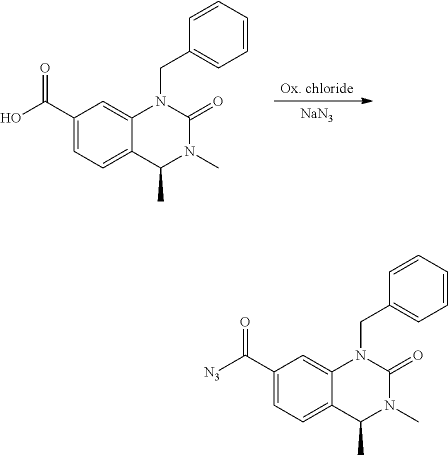 [SMILES: C[C@H]1c2ccc(C(=O)O)cc2N(Cc2ccccc2)C(=O)N1C ; "Ox. chloride" ; C[C@H]1c2ccc(C(=O)N=[N+]=[N-])cc2N(Cc2ccccc2)C(=O)N1C ; "NaN3"]

To a stirred solution of (S)-1-benzyl-3,4-dimethyl-2-oxo-1,2,3,4-tetrahydroquinazoline-7-carboxylic acid (Preparation 9) (200 mg, 0.65 mmol) in dry solvents DMF (50 uL) and DCM (10 mL) was added oxalyl chloride (164 mg, 1.29 mmol) at 0-5° C. The whole was stirred at RT for 1 h. Progress of the reaction was monitored by TLC and LCMS and after completion, the reaction mass was poured into an aqueous solution of NaN₃ (209 mg, 3.22 mmol in 10 mL water) under stirring. After the formation of the acid azide was complete (confirmed by LCMS), the product was extracted with DCM, washed with brine, dried over anhydrous Na₂SO₄, filtered and concentrated in vacuo to afford the title compound (210 mg, crude) as a white solid. LCMS m/z: 336.35 [M+H].

### Preparation 11: (S)-1-(1-Benzyl-3,4-dimethyl-2-oxo-1,2,3,4-tetrahydroquinazolin-7-yl)-3-(4-fluorophenylurea (Example 38)

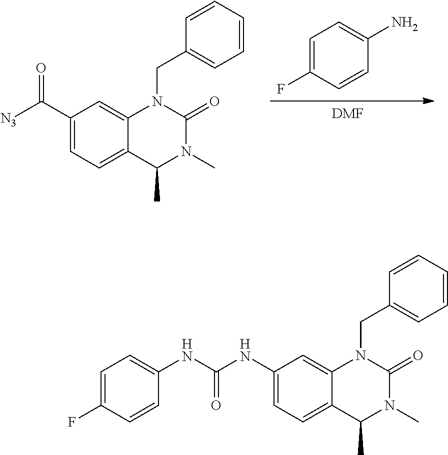

To a stirred solution of (S)-1-benzyl-3,4-dimethyl-2-oxo-1,2,3,4-tetrahydroquinazoline-7-carbonyl azide (Preparation 10) (192 mg, 0.57 mmol) in DMF (6 mL) was added 4-fluoro aniline (254 mg, 2.29 mmol) at RT. The whole was stirred at 83° C. overnight. Progress of the reaction was monitored by TLS/LCMS and after completion, the reaction mixture was diluted with EtOAc and washed with cold water. The organic layer was separated, dried over anhydrous Na₂SO₄, filtered and concentrated in vacuo to afford the title compound (196 mg, 82% yield) as an off white solid. Purity by UPLC: 99.05%; ¹H NMR (400 MHz; DMSO-d₆): δ 1.27 (d, J=6.35 Hz, 3H), 2.98 (s, 3H), 4.52 (q, J=6.4 Hz, 1H), 4.96-5.10 (m, 2H), 6.87 (s, 1H), 7.04-7.12 (m, 4H), 7.21-7.23 (m, 3H), 7.31-7.34 (m, 2H), 7.39-7.42 (m, 2H), 8.55 (s, 1H), 8.61 (s, 1H); LCMS m/z: 419.12 [M+H].

### Example 39: 1-(1-Benzyl-3-methyl-2-oxo-1,2,3,4-tetrahydroquinazolin-7-yl)-3-(4-fluorophenyl)urea

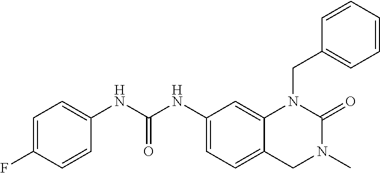 [SMILES: CN1Cc2ccc(NC(=O)Nc3ccc(F)cc3)cc2N(Cc2ccccc2)C1=O]

Example 39 was prepared according to the methods described in General Procedures 1b-4, 15, 16 and the methods described below.

### Preparation 12: Methyl -methyl-2-oxo-1,2,3,4-tetrahydroquinazoline-7-carboxylate

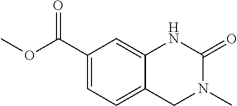

### Step 1: Methyl -amino-4-formylbenzoate

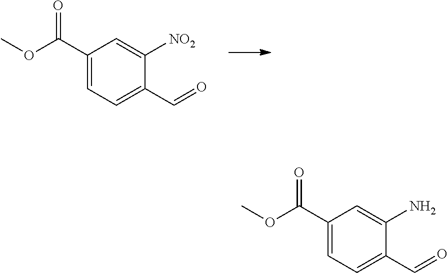

To a stirred solution of commercially available methyl 4-formyl-3-nitrobenzoate (2.0 g, 9.56 mmol) in EtOH (20 mL) was added iron powder (2.14 g, 38.24 mmol) followed by 0.12 N HCl. The reaction mixture was refluxed for 30 min. Progress of the reaction was monitored by TLC and LC-MS and after completion the reaction mixture was quenched with a saturated NaHCO₃ solution and extracted with DCM followed by a brine wash. The organic layer was dried over anhydrous Na₂SO₄, filtered and concentrated in vacuo to afford the title compound (2.0 g, crude) as a yellow solid which was used in the next step without any further purification. LCMS m/z: 180.01 [M+H].

### Step 2: Methyl 3-((ethoxycarbonyl)amino)-4-formylbenzoate

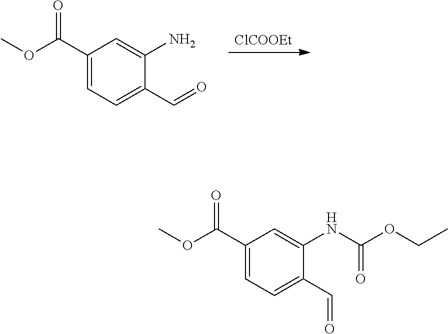 [SMILES: COC(=O)c1ccc(C=O)c(N)c1 ; CCOC(=O)Nc1cc(C(=O)OC)ccc1C=O ; "ClCOOEt"]

To a stirred solution of methyl 3-amino-4-formylbenzoate (Preparation 12, step 1) (2.0 g, 11.16 mmol) in DCE (20 mL) was added pyridine (1.98 mL, 24.56) and ethyl chloroformate (1.27 mL, 13.39 mmol) at 0-5° C. The whole was stirred at 0-5° C. for 1 h. Completion of the reaction was confirmed by TLC and LC-MS. The reaction mixture was quenched by a 1N HCl solution and extracted with DCM followed by a brine wash. The organic layer was dried over anhydrous Na₂SO₄, filtered and concentrated in vacuo to afford the title compound (2.0 g, crude) as a yellow solid which was used in the next step without any further purification. LCMS m/z: 252.03 [M+H].

### Step 3: Methyl 3-methyl-2-oxo-1,2,3,4-tetrahydroquinazoline-7-carboxylate

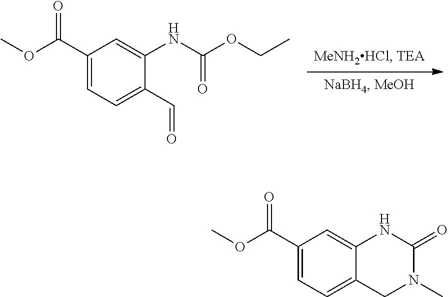

To a stirred solution of methylamine hydrochloride (0.27 g, 3.98 mmol) in MeOH (20 mL) was added TEA (0.67 mL, 4.78 mmol) under an inert atmosphere at RT and the resulting clear solution then stirred for 30 min. Methyl 3-((ethoxycarbonyl)amino)-4-formylbenzoate (Preparation 12, step 2) (1.0 g, 3.987 mmol) was added portionwise and the combined mixture stirred at room temperature for 24 h. During this period, the reaction mixture became a suspension. NaBH₄ (227 mg, 5.97 mmol) was added and the mixture was further stirred for another 24 h. Progress of the reaction was monitored by TLC and LC-MS and after completion the reaction mixture was diluted with water and extracted with EtOAc followed by a brine wash. The organic layer was dried over anhydrous Na₂SO₄, filtered and concentrated in vacuo to afford the title compound (750 mg, crude) as a yellow solid which was used in the next step without any further purification. LCMS m/z: 221.04 [M+H].

### Preparation 13: Methyl 1-benzyl-3-methyl-2-oxo-1,2,3,4-tetrahydroquinazoline-7-carboxylate

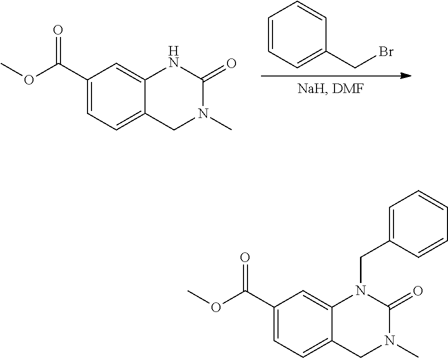

To a stirred solution of methyl 3-methyl-2-oxo-1,2,3,4-tetrahydroquinazoline-7-carboxylate (Preparation 12, step 3) (300 mg, 1.36 mmol) in DMF (3 mL) was added NaH (65 mg, 1.63 mmol) and benzyl bromide (183 μL, 1.50 mmol) at 10° C. under a nitrogen atmosphere. The whole was stirred at room temperature for 1 h. TLC showed complete consumption of the cyclic urea intermediate and the reaction mixture was then quenched over ice-water and extracted with EtOAc. The organic layer was washed with brine, dried over Na₂SO₄ and evaporated under reduced pressure to afford the title compound (390 mg, crude) as a faint brown solid which was used in the next step without any further purification. LCMS m/z: 311.28 [M+H].

### Preparation 14: 1-Benzyl-3-methyl-2-oxo-1,2,3,4-tetrahydroquinazoline-7-carboxylic acid

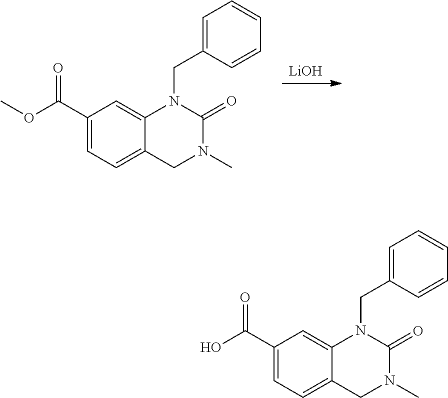

To a stirred solution of methyl 1-benzyl-3-methyl-2-oxo-1,2,3,4-tetrahydroquinazoline-7-carboxylate (Preparation 13) (390 mg, 1.26 mmol) in THF (10 mL) and MeOH (5 mL) was added a solution of LiOH (264 mg, 6.28 mmol) in water (1.5 mL) and the combined mixture stirred at room temperature for 3 h. TLC showed completion of the reaction. The solvents were evaporated in vacuo to give a residue which was diluted with water, washed with MTBE and the aqueous layer was acidified with 6N HCl resulting in a precipitate. The precipitate was filtered off and dried in vacuo to afford the title compound (330 mg, crude) as an off white solid which was used in the next step without any further purification. LCMS m/z: 297.46 [M+H].

### Preparation 15: tert-Butyl (1-benzyl-3-methyl-2-oxo-1,2,3,4-tetrahydroquinazolin-7-yl)carbamate

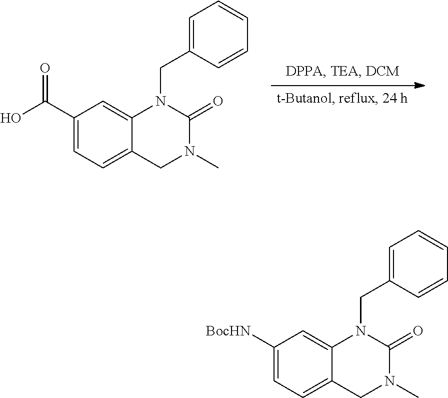 [SMILES: CN1Cc2ccc(C(=O)O)cc2N(Cc2ccccc2)C1=O ; "DPPA, TEA, DCM" ; "t-Butanol, reflux, 24 h" ; CN1Cc2ccc(NC(=O)OC(C)(C)C)cc2N(Cc2ccccc2)C1=O]

To a stirred solution of 1-benzyl-3-methyl-2-oxo-1,2,3,4-tetrahydroquinazoline-7-carboxylic acid (Preparation 14) (0.33 g, 1.11 mmol) in DCM (10 mL) was added TEA (0.241 mL, 1.67 mmol) followed by DPPA (0.483 mL, 2.23 mmol) at 0-5° C. The whole was stirred at room temperature for 3 h. Progress of the reaction was monitored by UPLC-MS and after completion the solvent was evaporated to afford the desired carbamate as a faint brownish solid (350 mg) which was then dissolved in tert-butanol (10 mL) and refluxed for 24 h. UPLC-MS showed completion of the reaction. The solvent was evaporated under reduced pressure and the residue was purified by Combi-flash to afford the title compound (225 mg, yield 55%) as an off white solid. LCMS m/z: 368.6 [M+H].

### Preparation 16: 7-Amino-1-benzyl-3-methyl-3,4-dihydroquinazolin-2(1H)-one

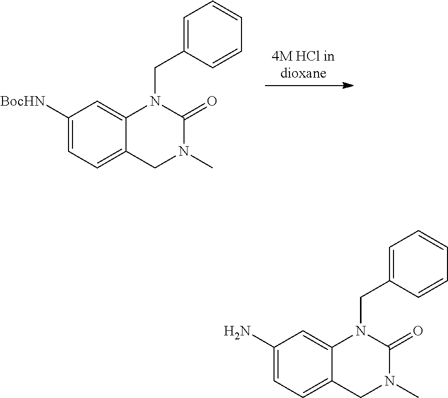 [SMILES: CN1Cc2ccc(NC(=O)OC(C)(C)C)cc2N(Cc2ccccc2)C1=O ; "4M HCl indioxane" ; CN1Cc2ccc(N)cc2N(Cc2ccccc2)C1=O]

To a stirred solution of tert-butyl (1-benzyl-3-methyl-2-oxo-1,2,3,4-tetrahydroquinazolin-7-yl)carbamate (Preparation 15) (225 mg, 0.61 mmol) in THF (5 mL) was added 4M HCl in dioxane (5 mL) dropwise at 0-5° C. under an inert atmosphere. The whole was stirred at room temperature for 24 h. TLC showed formation of the desired compound. The solvent was evaporated in vacuo and the resulting residue was dissolved in water and neutralized with NaHCO₃ solution and extracted with EtOAc. The combined organic layers were dried over anhydrous Na₂SO₄, filtered and then evaporated in vacuo to afford the title compound (190 mg, crude) as a brownish oil which was used in the next step without any further purification. LCMS m/z: 268.4 [M+H].

### Preparation 17: 1-(1-Benzyl-3-methyl-2-oxo-1,2,3,4-tetrahydroquinazolin-7-yl)-3-(4-fluorophenyl)urea (Example 39)

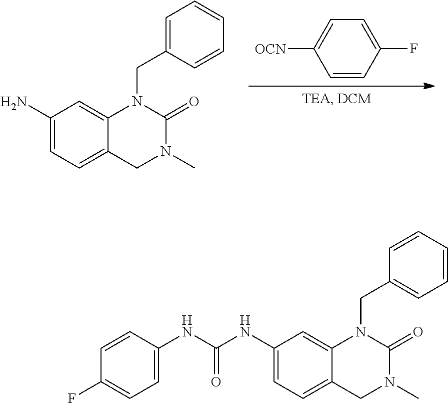

To a stirred solution of 7-amino-1-benzyl-3-methyl-3,4-dihydroquinazolin-2(1H)-one (Preparation 16) (85 mg, 0.317 mmol) in DCM (10 mL) was added 4-fluorophenyl isocyanate (44 μL, 0.308 mmol) and TEA (55 μL, 0.308 mmol) at 0-5° C. The whole was stirred at RT for 1 h. TLC showed complete consumption of the starting material. The solvent was evaporated in vacuo to afford the crude product which was purified by prep-HPLC to give the title compound (14 mg, yield 11%) as a faint yellow solid. Purity by UPLC: 96.6%; ¹H NMR (500 MHz; DMSO-d₆): δ 2.96 (s, 3H), 4.41 (s, 2H), 5.03 (bs, 2H), 6.86 (d, J=1.2 H, 1H), 7.05 (d, J=8.2 Hz, 1H), 7.11 (t, J=8.8 Hz, 3H), 7.21-7.25 (m, 3H), 7.31-7.34 (m, 2H), 7.39-7.42 (m, 2H), 8.68 (s, 1H), 8.73 (s, 1H); LCMS m/z: 405.36 [M+H].

### Example 40

Example 40 was prepared according to the above methods used to make Example 39 as described in General Procedures 1b-4, 15, 16 using the appropriate amines or isocyanate. Purification was as stated in the aforementioned methods.

| | | IUPAC | | LCMS | Purity |
|---|---|---|---|---|---|
| Ex | Structure | Name | 1H-NMR | [M + H] | (%) |
| 40 | 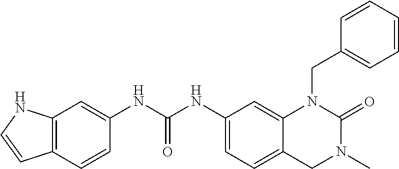 [SMILES: CN1Cc2ccc(NC(=O)Nc3ccc4cc[nH]c4c3)cc2N(Cc2ccccc2)C1=O] | 1-(1- benzyl-3- methyl-2- oxo- 1,2,3.4- tetrahydro quinazolin- 7-yl)-3- (1H-indol- 6-yl)urea | (500 MHz; DMSO-d₆): δ 2.99 (s, 3H), 4-42 (s, 2H), 5.04 (s, 2H), 6.32 (s, 1H), 6.79 (d, J = 1.55 Hz, 1H), 6.80 (d, J = 1.6 Hz, 1H), 6.92 (m, 1H), 7.05 (m, 1H), 7.10-7.12 (m 4H), 7.21-7.26 (m, 2H), 7.33 (d, J = 7.5 Hz, 1H), 7.60 (s, 1H), 8.61- 8.65 (m, 2H), 10.92 (s, 1H) | 426.42 | 95.4 |

### Example 41: 4-(2-Chloro-6-fluorobenzyl)-6-(4-fluorophenethoxy)-2H-benzo[b][1,4]thiazin-3(4H)-one

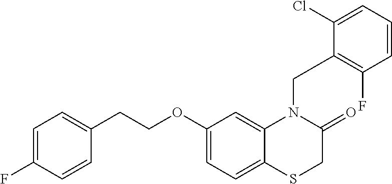 [SMILES: O=C1CSc2ccc(OCCc3ccc(F)cc3)cc2N1Cc1c(F)cccc1Cl]

Example 41 was prepared according to the methods described in General Procedures 4-6 and the methods described below.

### Preparation 18: 6-Methoxy-2H-benzo[b][1,4]thiazin-3(4H)-one

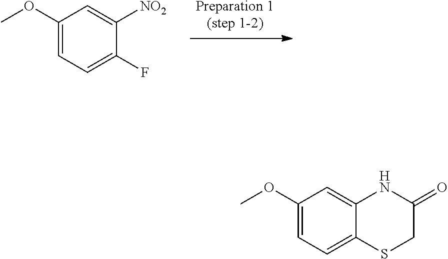

The title compound 6-methoxy-2H-benzo[b][1,4]thiazin-3(4H)-one was prepared in two steps following an identical procedure to that described in Preparation 1, Steps 1-2.

### Preparation 19: 4-(2-Chloro-6-fluorobenzyl)-6-methoxy-2H-benzo[b][1,4]thiazin-3(4H)-one

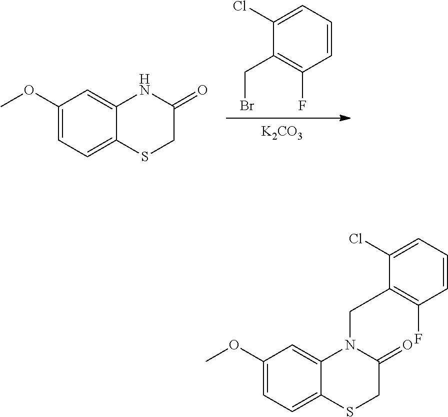

To a stirred solution of 6-methoxy-2H-benzo[b][1,4]thiazin-3(4H)-one (Preparation 18) (1.0 g, 5.102 mmol) in dry DMF (10 mL) was added K₂CO₃ (1.408 g, 10.204 mmol) followed by 2-chloro-6-fluorobenzyl bromide (1.053 mL, 7.653 mmol) at room temperature. The whole was then stirred at 90-120° C. for 12 h. Progress of the reaction was monitored by TLC and LC-MS and after completion the reaction mixture was diluted with water and extracted with EtOAc. The combined organic layer was washed with brine, dried over anhydrous Na₂SO₄, filtered and concentrated in vacuo to give the crude product which was purified by column chromatography to afford the title compound (0.6 g, yield 34.81%) as an off white solid. LCMS m/z: 338 [M+H].

### Preparation 20: 4-(2-Chloro-6-fluorobenzyl)-6-hydroxy-2H-benzo[b][1,4]thiazin-3(4H)-one

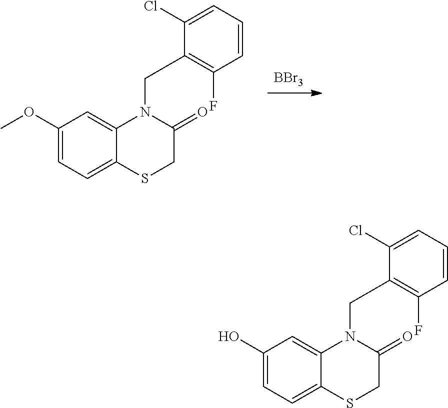

To a stirred solution of 4-(2-chloro-6-fluorobenzyl)-6-methoxy-2H-benzo[b][1,4]thiazin-3(4H)-one (Preparation 19) (400 mg, 1.187 mmol) in dry DCM (5 mL) was added BBr₃ (0.5 mL, 1M solution in DCM) dropwise at 0-5° C. The whole was then stirred at room temperature for 4 h. Progress of the reaction was monitored by TLC and LC-MS and after completion the reaction mixture was evaporated to dryness, then diluted with EtOAc, washed with NaHCO₃ solution followed by water and brine. The organic layer was dried over anhydrous Na₂SO₄, filtered and concentrated in vacuo to give the title compound (200 mg, yield 52%) as an off white solid. LCMS m/z: 324 [M+H].

### Preparation 21: 4-(2-Chloro-6-fluorobenzyl)-6-(4-fluorophenethoxy)-2H-benzo[b][1,4]thiazin-2(4H)-one (Example 41)

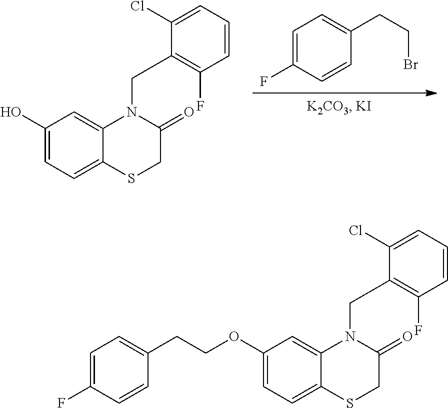

To a stirred solution of 4-(2-chloro-6-fluorobenzyl)-6-hydroxy-2H-benzo[b][1,4]thiazin-3(4H)-one (Preparation 20) (110 mg, 0.341 mmol) in dry acetone (3 mL) was added K₂CO₃ (94 mg, 0.681 mmol) followed by KI (2.002 mg, 0.014 mmol) at room temperature. After 5 min. 1-(2-bromoethyl)-4-fluorobenzene (103.7 mg, 0.511 mmol) was added and the combined mixture was stirred at reflux for 16 h. Progress of the reaction was monitored by TLC and LC-MS which confirmed the desired product formation. The reaction mixture was evaporated to dryness, then diluted with water and extracted with EtOAc. The combined organic layers were washed with water and brine, dried over anhydrous Na₂SO₄, filtered and concentrated in vacuo to give the crude product which was purified by prep-TLC to afford the title compound (25 mg, yield 16.46%) as an off white solid. Purity by HPLC: 96.99%; ¹H NMR (400 MHz; DMSO-d₆): δ 2.97 (s, 2H), 3.48 (s, 2H), 4.11 (d, J=6.36 Hz, 2H), 5.35 (s, 2H), 6.57 (d, J=8.76 Hz, 1H), 6.79 (s, 1H), 7.10-7.15 (m, 3H), 7.22-7.25 (m, 2H), 7.28-7.32 (m, 3H); LCMS m/z: 446.2 [M+H].

### Example 42: 4-(2-Chloro-6-fluorobenzyl)-6-(((4-fluorobenzyl)oxy)methyl)-2H-benzo[b][1,4]thiazin-1(4H)-one

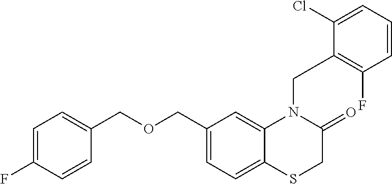 [SMILES: O=C1CSc2ccc(COCc3ccc(F)cc3)cc2N1Cc1c(F)cccc1Cl]

Example 42 was prepared according to the methods described in General Procedures 3-6 and the methods described below.

### Preparation 22: 4-(2-Chloro-6-fluorobenzyl-3-oxo-3,4-dihydro-2H-benzo[b][1,4]thiazine-6-carboxylic acid

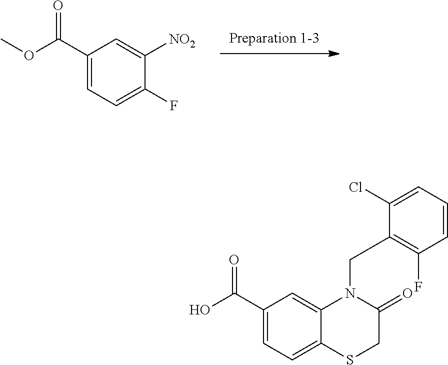 [SMILES: COC(=O)c1ccc(F)c([N+](=O)[O-])c1 ; "Preparation 1-3" ; O=C(O)c1ccc2c(c1)N(Cc1c(F)cccc1Cl)C(=O)CS2]

The title compound 4-(2-chloro-6-fluorobenzyl)-3-oxo-3,4-dihydro-2H-benzo[b][1,4]thiazine-6-carboxylic acid was prepared in four steps using an identical procedure to that described in Preparations 1-3.

### Preparation 22: 4-(2-Chloro-6-fluorobenzyl)-6-(hydroxymethyl)-2H-benzo[b][1,4]thiazin-3(4H)-one

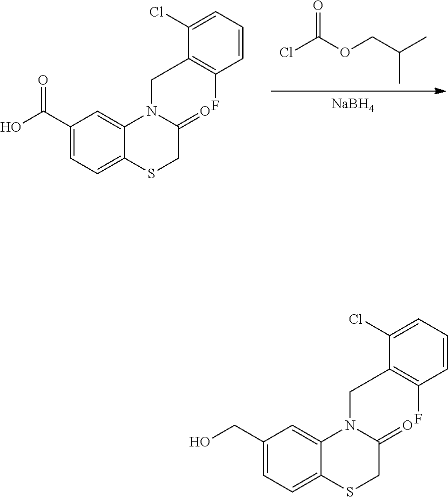

To a stirred solution of 4-(2-chloro-6-fluorobenzyl)-3-oxo-3,4-dihydro-2H-benzo[b][1,4]thiazine-6-carboxylic acid (Preparation 22) (250 mg, 0.712 mmol) in dry THF (10 mL) was added TEA (0.985 mL, 0.712 mmol) at 0-5° C. and the resulting reaction mixture was treated with isobutylchloroformate (96.866 mg, 0.712 mmol). The whole was then stirred at 0-5° C. for a further 2 h. The reaction mixture was filtered and washed with THF. The filtrate was then stirred at 0-5° C. as firstly NaBH₄ (53.889 mg, 1.425 mmol) and then water (3 mL) were added portionwise. The resulting suspension was warmed to room temperature and stirred for 2 h. Progress of the reaction was monitored by TLC and LC-MS which confirmed the desired product formation. The reaction mixture was neutralized with 1N HCl and diluted with water. The aqueous mixture was extracted with EtOAc, washed with water and brine, dried over anhydrous Na₂SO₄, filtered and concentrated in vacuo to give the crude compound which was purified by column chromatography to afford the title compound (200 mg, yield 83.13%) as an off white solid. LCMS m/z: 338 [M+H].

### Preparation 24: 4-(2-Chloro-6-fluorobenzyl)-6-(((4-fluorobenzyloxy)methyl)-2H-benzo[b][1,4]thiazin-3(4H)-one (Example 42)

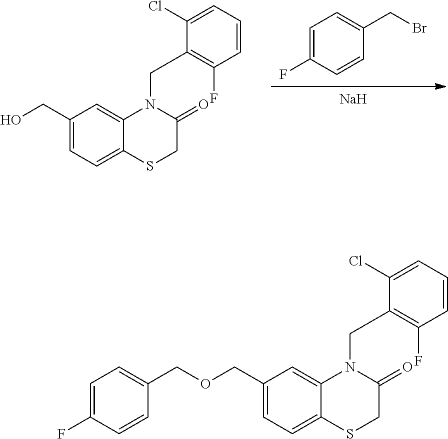

To a stirred suspension of 60% NaH (28.48 mg, 0.712 mmol) in THF (5 mL) was added a solution of 4-(2-chloro-6-fluorobenzyl)-6-(hydroxymethyl)-2H-benzo[b][1,4]thiazin-3(4H)-one (Preparation 23) (200 mg, 0.593 mmol) in THF (1.5 mL) at 0-5° C. The mixture was stirred at room temperature for 10 min. before a solution of 4-fluorobenzyl bromide (224.3 mg, 1.18 mmol) in THF (1 mL) was added to the reaction mixture and stirring continued at room temperature for 2 h. TLC and LCMS showed formation of the desired product, then the reaction mixture was quenched with water, extracted with EtOAc, washed with water and brine, dried over anhydrous Na₂SO₄, filtered and concentrated in vacuo to give the crude product which was purified by column chromatography to afford the title compound (50 mg, yield 19%) as a white solid. Purity by HPLC: 98.89%; ¹H NMR (400 MHz; DMSO-d₆): δ 3.55 (s, 2H), 4.40 (s, 4H), 5.37 (s, 2H), 6.95 (d, J=7.92 Hz, 1H), 7.05-7.10 (m, 1H), 7.16-7.27 (m, 5H), 7.33-7.35 (m, 3H); LCMS m/z: 446.0 [M+H].

### Example 43: 4-(2-Chloro-6-fluorobenzyl)-6-(t-phenyl-1H-imidazol-2-yl)-2H-benzo[b][1,4]thiazin-3(4H)-one

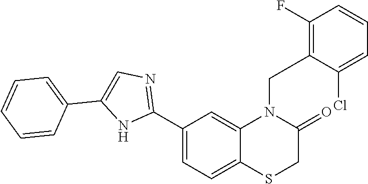 [SMILES: O=C1CSc2ccc(-c3ncc(-c4ccccc4)[nH]3)cc2N1Cc1c(F)cccc1Cl]

Example 43 was prepared according to the methods described in General Procedures 3-6 and the methods described below.

### Preparation 25: 2-Oxo-2-phenylethyl 4-(2-chloro-6-fluorobenzyl-3-oxo-3,4-dihydro-2H-benzo[b][1,4]thiazine-6-carboxylate

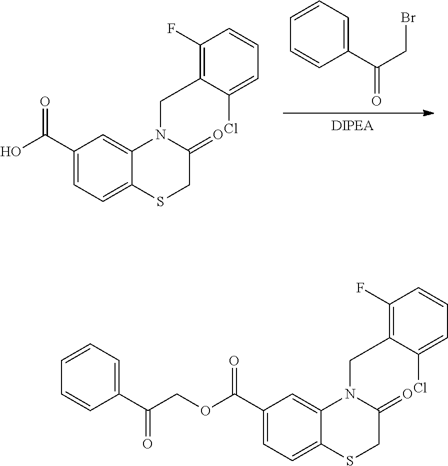

To a stirred solution of 4-(2-chloro-6-fluorobenzyl)-3-oxo-3,4-dihydro-2H-benzo[b][1,4]thiazine-6-carboxylic acid (Preparation 22) (300 mg, 0.822 mmol) in DMF (5 mL) was added DIPEA (429.93 mg, 2.466 mmol) followed by 2-bromo-1-phenylethanone (327.189 mg, 1.644 mmol) and the whole stirred overnight at room temperature. Progress of the reaction was monitored by TLC and LC-MS and after completion the reaction mixture was diluted with cold water and extracted with MTBE. The combined organics were washed with water and brine, dried over anhydrous Na₂SO₄, filtered and concentrated in vacuo to give the crude product which was purified by trituration with pentane and diethyl ether to afford the title compound (250 mg, yield 64.73%) as an off white solid. LCMS m/z: 470 [M+H].

### Preparation 26: 4-(2-Chloro-6-fluorobenzyl)-6-(5-phenyl-1H-imidazol-2-yl-2H-benzo[b][1,4]thiazin-(4H)-one (Example 43)

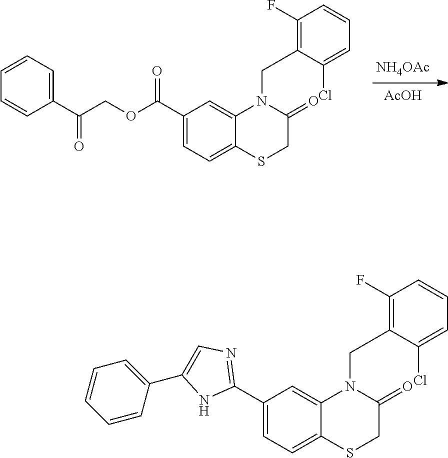 [SMILES: "AcOH" ; O=C(COC(=O)c1ccc2c(c1)N(Cc1c(F)cccc1Cl)C(=O)CS2)c1ccccc1 ; O=C1CSc2ccc(-c3ncc(-c4ccccc4)[nH]3)cc2N1Cc1c(F)cccc1Cl ; "NH4OAc"]

To a stirred solution of 2-oxo-2-phenylethyl 4-(2-chloro-6-fluorobenzyl)-3-oxo-3,4-dihydro-2H-benzo[b][1,4]thiazine-6-carboxylate (Preparation 25) (70 mg, 0.149 mmol) in AcOH (3 mL) was added NH₄OAc (287.3 mg, 3.73 mmol) at room temperature. After the addition was complete, the reaction mixture was heated at 120° C. for 48 h. Progress of the reaction was monitored by TLC and LC-MS and after completion the reaction mixture was concentrated in vacuo to dryness. The residue was diluted with water, neutralized with NaHCO₃ solution then extracted with EtOAc, washed with water and brine, dried over anhydrous Na₂SO₄, filtered and concentrated in vacuo to give the crude product which was purified by column chromatography to afford the title compound (10 mg, yield 14.89%) as a pale yellow solid. Purity by HPLC: 95.41%; ¹H NMR (400 MHz; DMSO-d₆, at 100° C.): δ 3.55 (s, 2H), 5.45 (s, 2H), 7.07 (t, J=8.96 Hz, 1H), 7.19-7.30 (m, 3H), 7.36-7.39 (m, 2H), 7.45 (d, J=8.0 Hz, 1H), 7.53-7.71 (m, 2H), 7.86 (d, J=7.56 Hz, 2H), 7.95 (s, 1H), 12.33 (s, 1H); LCMS m/z: 450.2 [M+H].

### Example 44: 4-(2-Chloro-6-fluorobenzyl)-6-(S-phenyl-1H-1,2,4-triazol-3-yl)-2H-benzo[b][1,4]thiazin-3(4H)-one

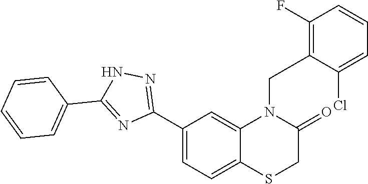 [SMILES: O=C1CSc2ccc(-c3n[nH]c(-c4ccccc4)n3)cc2N1Cc1c(F)cccc1Cl]

Example 44 was prepared according to the methods described in General Procedures 3-6 and the methods described below.

### Preparation 27: tert-Butyl 2-(4-(2-chloro-6-fluorobenzyl)-3-oxo-3,4-dihydro-2H-benzo[b][1,4]thiazine-6-carbonyl)hydrazinecarboxylate

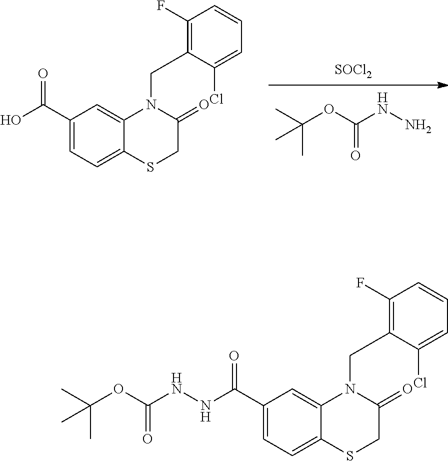

To a stirred solution of 4-(2-chloro-6-fluorobenzyl)-3-oxo-3,4-dihydro-2H-benzo[b][1,4]thiazine-6-carboxylic acid (Preparation 22) (400 mg, 1.14 mmol) was added SOCl₂ (10 mL) slowly at 0-5° C., then the combined reaction mixture was refluxed for 2 h. The mixture was evaporated under reduced pressure to give a crude residue which was diluted with THF. TEA was added dropwise, followed by tert-butyl hydrazinecarboxylate (300.85 mg, 2.279 mmol) at 0-5° C. The combined mixture was stirred at room temperature overnight. Progress of the reaction was monitored by TLC and LC-MS and after completion the reaction mixture was diluted with EtOAc, washed with water, NaHCO₃ solution and brine, dried over anhydrous Na₂SO₄, filtered and concentrated in vacuo to give the title compound (400 mg, yield 75.33%, crude) as an off white solid. LCMS m/z: 466 [M+H].

### Preparation 28: 4-(2-Chloro-6-fluorobenzyl-3-oxo-3,4-dihydro-2H-benzo[b][1,4]thiazine-6-carbohydrazide hydrochloride

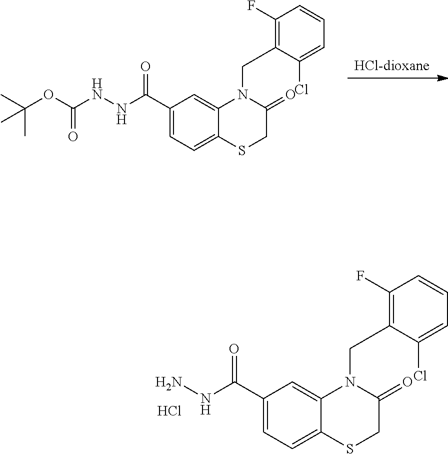 [SMILES: CC(C)(C)OC(=O)NNC(=O)c1ccc2c(c1)N(Cc1c(F)cccc1Cl)C(=O)CS2 ; "HCl-dioxane" ; Cl ; NNC(=O)c1ccc2c(c1)N(Cc1c(F)cccc1Cl)C(=O)CS2]

To tert-butyl 2-(4-(2-chloro-6-fluorobenzyl)-3-oxo-3,4-dihydro-2H-benzo[b][1,4]thiazine-6-carbonyl)hydrazinecarboxylate (Preparation 27) (400 mg, 0.86 mmol) was added 4M HCl (10 mL, 4M solution in dioxane) slowly at 0-5° C. with stirring, and the mixture then allowed to warm slowly to room temperature over 4 h. Progress of the reaction was monitored by TLC and LC-MS and after completion the reaction mixture was concentrated in vacuo to dryness, then diethyl ether added to give a precipitate which was filtered off and dried under an inert atmosphere to afford the title compound (280 mg, yield 89.22%) as an off white solid. LCMS m/z: 366 [M+H].

### Preparation 29: 4-(2-Chloro-6-fluorobenzyl)-6-(5-phenyl-1H-1,2,4-triazol-3-yl)-2H-benzo[b][1,4]thiazin-3(4H)-one (Example 44)

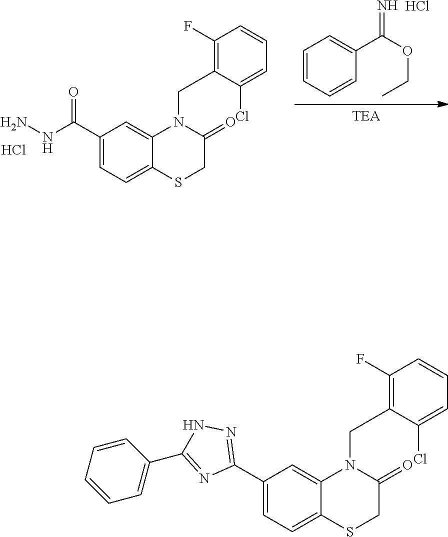

To a stirred solution of 4-(2-chloro-6-fluorobenzyl)-3-oxo-3,4-dihydro-2H-benzo[b][1,4]thiazine-6-carbohydrazide hydrochloride (Preparation 28) (100 mg, 0.215 mmol) and ethyl benzimidate hydrochloride (44.237 mg, 0.237 mmol) in MeCN (6 mL) was added TEA (15.639 mg, 0.155 mmol) dropwise at RT. The whole was stirred at 100° C. for 6 h. Progress of the reaction was monitored by TLC and LC-MS and after completion the reaction mixture was quenched with cold water, extracted with EtOAc, washed with water and brine, dried over anhydrous Na₂SO₄, filtered and concentrated in vacuo to give the crude product which was purified by column chromatography to afford the title compound (15 mg, yield 15.47%) as a white solid. Purity by HPLC: 99.77%; ¹H NMR (400 MHz; DMSO-d₆, at 100° C.): S 3.58 (s, 2H), 5.47 (s, 2H), 7.06 (t, J=8.24 Hz, 1H), 7.21-7.29 (m, 2H), 7.48-7.55 (m, 4H), 7.68 (d, J=8.12 Hz, 1H), 7.96 (s, 1H), 8.07 (d, J=7.32 Hz, 2H), 14.21 (s, 1H); LCMS m/z: 451 [M+H].

### Example 41: 6-(5-Benzyl-4H-1,2,4-triazol-3-yl)-4-(2-chloro-6-fluorobenzyl)-2H-benzo[b][1,4]thiazin-1(4H)-one

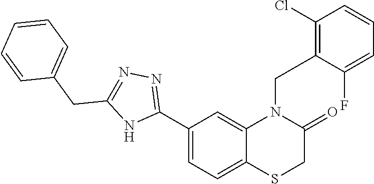 [SMILES: O=C1CSc2ccc(-c3nnc(Cc4ccccc4)[nH]3)cc2N1Cc1c(F)cccc1Cl]

Example 45 was prepared according to the methods described in General Procedures 4-6 and the methods described below.

### Preparation 30: 4-(2-Chloro-6-fluorobenzyl-3-oxo-3,4-dihydro-2H-benzo[b][1,4]thiazine-6-carbonitrile

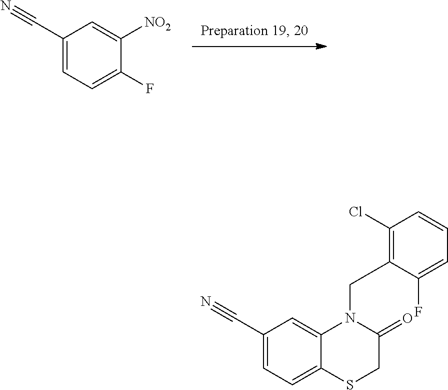 [SMILES: N#Cc1ccc(F)c([N+](=O)[O-])c1 ; "Preparation 19, 20" ; N#Cc1ccc2c(c1)N(Cc1c(F)cccc1Cl)C(=O)CS2]

The title compound 4-(2-chloro-6-fluorobenzyl)-3-oxo-3,4-dihydro-2H-benzo[b][1,4]thiazine-6-carbonitrile was prepared in three steps following an identical procedure to that described in Preparations 18 and 19.

### Preparation 31: 6-(5-Benzyl-4H-1,2,4-triazol-3-yl)-4-(2-chloro-6-fluorobenzyl-2H-benzo[b][1,4]thiazin-(4H)-one (Example 45)

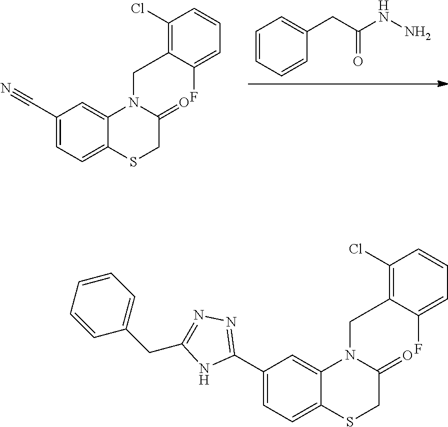

To a stirred solution of 4-(2-chloro-6-fluorobenzyl)-3-oxo-3,4-dihydro-2H-benzo[b][1,4]thiazine-6-carbonitrile (Preparation 30) (50 mg, 0.151 mmol) in n-BuOH (2 mL) was added K₂CO₃ (41.566 mg, 0.301 mmol) followed by 2-phenylacetohydrazide (22.617 mg, 0.151 mmol). The whole was heated at 150° C. for 15 h. Progress of the reaction was monitored by TLC and LC-MS and after completion the reaction mixture was quenched with cold water, extracted with EtOAc, washed with water and brine, dried over anhydrous Na₂SO₄, filtered and concentrated in vacuo to give the crude product which was purified by column chromatography to afford the title compound (20 mg, yield 29%) as an off white solid. Purity by HPLC: 90.01%; ¹H NMR (400 MHz; DMSO-d₆): δ 3.58 (s, 2H), 4.08 (s, 2H), 5.40 (s, 2H), 7.05-7.07 (m, 1H), 7.21-7.28 (m, 4H), 7.32-7.37 (m, 3H), 7.41-7.45 (m, 1H), 7.55-7.57 (m, 1H), 7.86 (s, 1H), 13.95 (s, 1H); LCMS m/z: 465.2 [M+H].

### Example 46: 4-(2-Chloro-6-fluorobenzyl)-6-(4-phenyloxazol-2-yl)-2H-benzo[b][1,4]thiazin-3(4H)-one

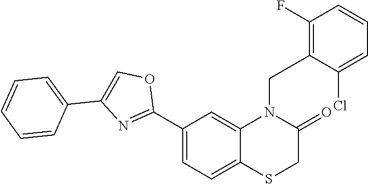 [SMILES: O=C1CSc2ccc(-c3nc(-c4ccccc4)co3)cc2N1Cc1c(F)cccc1Cl]

Example 46 was prepared according to the methods described in General Procedures 3-6 and the methods described below.

### Preparation 32: 2-Oxo-2-phenylethyl 4-(2-chloro-6-fluorobenzyl)-3-oxo-3,4-dihydro-2H-benzo[b][1,4]thiazine-6-carboxylate

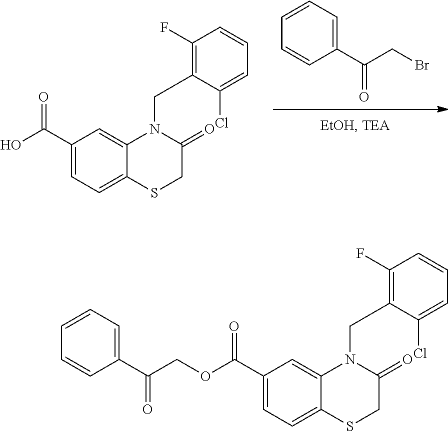

To a stirred solution of 4-(2-chloro-6-fluorobenzyl)-3-oxo-3,4-dihydro-2H-benzo[b][1,4]thiazine-6-carboxylic acid (Preparation 22) (100 mg, 0.285 mmol) in EtOH (2 mL) was added TEA (0.079 mL, 0.57 mmol) followed by 2-bromo-1-phenylethanone (68 mg, 0.342 mmol) at RT under an inert atmosphere. The resulting reaction mixture was stirred at 60° C. for 2 h. Completion of the reaction was confirmed by TLC and LCMS. The reaction mixture was diluted with water and extracted with EtOAc. The combined organic layers were washed with water followed by brine, dried over anhydrous Na₂SO₄, filtered and concentrated in vacuo to give a crude product which was purified by column chromatography to afford the title compound (85 mg, 63% yield) as a light yellow white solid. LCMS m/z: 470 [M+H].

### Preparation 3: 4-(2-Chloro-6-fluorobenzyl)-6-(4-phenyloxazol-2-yl)-2H-benzo[b][1,4]thiazin-3(4H)-one (Example 46)

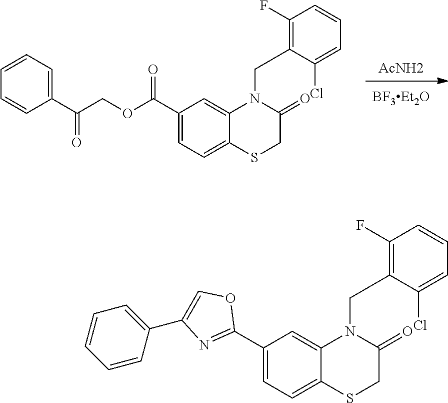 [SMILES: "AcNH2" ; O=C(COC(=O)c1ccc2c(c1)N(Cc1c(F)cccc1Cl)C(=O)CS2)c1ccccc1 ; O=C1CSc2ccc(-c3nc(-c4ccccc4)co3)cc2N1Cc1c(F)cccc1Cl]

To a stirred solution of 2-oxo-2-phenylethyl 4-(2-chloro-6-fluorobenzyl)-3-oxo-3,4-dihydro-2H-benzo[b][1,4]thiazine-6-carboxylate (Preparation 32) (100 mg, 0.213 mmol) in xylene (2 mL) was added BF₃.Et₂O (0.02 mL) followed by AcNH₂ (62.9 mg, 1.066 mmol) at RT. The resulting reaction mixture was heated at 150° C. for 15 h. Progress of the reaction was monitored by LCMS and after completion; the reaction mixture was cooled to RT and quenched with water. The resulting reaction mass was extracted with EtOAc, washed with water followed by brine, dried over anhydrous Na₂SO₄, filtered and concentrated in vacuo to give the crude product which was purified by column chromatography to afford the title compound (70 mg, 73% yield) as an off white solid. Purity by HPLC: 97.23%; ¹H NMR (400 MHz; CDCl₃): δ. 3.50 (s, 2H), 5.56 (s, 2H), 6.85-6.91 (m, 1H), 7.08-7.10 (m, 2H), 7.32-7.36 (m, 1H), 7.39-7.45 (m, 3H), 7.67 (dd, J′=1.24 Hz, J″=7.96 Hz, 1H), 7.80 (d, J=7.44 Hz, 2H), 7.90 (d, J=1.12 Hz, 1H), 7.94 (s, 1H); LCMS m/z: 451.2 [M+H].

### Example 47: N-(4-Benzyl-3-oxo-3,4-dihydro-2H-benzo[b][1,4]thiazin-6-yl)-2-(1H-indol-6-yl)acetamide

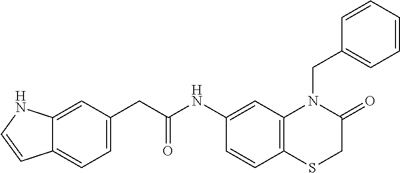 [SMILES: O=C(Cc1ccc2cc[nH]c2c1)Nc1ccc2c(c1)N(Cc1ccccc1)C(=O)CS2]

Example 47 was prepared according to the methods described in General Procedures 2-4, 6 and the methods described below.

### Preparation 34: N-(4-Benzyl-3-oxo-3,4-dihydro-2H-benzo[b][1,4]thiazin-6-yl)-2-(1H-indol-6-yl)acetamide (Example 47)

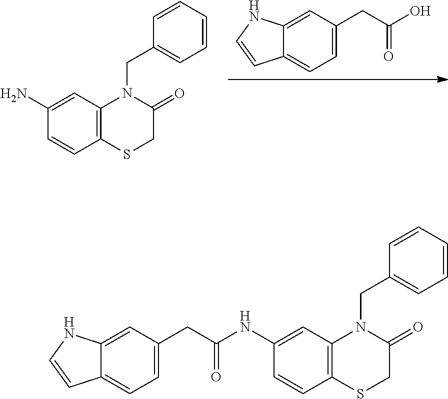

To a stirred solution of 2-(1H-indol-6-yl)acetic acid (30 mg, 0.174 mmol) in THF (1.5 mL) was added HOBT.H₂O (23 mg, 0.174 mmol), DIPEA (146 μl, 0.86 mmol) and EDCI.HCl (49 mg, 0.26 mmol) at 0-5° C. and the combined mixture allowed to stir for 15 min. Then, 6-amino-4-benzyl-2H-benzo[b][1,4]thiazin-3(4H)-one (Preparation 5) (55 mg, 0.204 mmol) was added to the reaction mixture and the whole stirred at RT overnight. Product formation was confirmed by TLC and UPLC. The reaction mixture was evaporated in vacuo to a low volume and then extracted with EtOAc, washed with 1N HCl solution to remove excess amine and further washed with a saturated solution of K₂CO₃ followed by brine. The organic layer was dried with anhydrous Na₂SO₄, filtered and evaporated in vacuo to give the crude product which was purified by prep-HPLC to produce a gummy material. This material was triturated with hexane and diethyl ether to afford the title compound (13 mg, 18% yield) as a white solid. Purity by HPLC: 99.1%; ¹H NMR (500 MHz; DMSO-d₆): S. 3.64 (s, 4H), 5.14 (s, 2H), 6.39 (s, 1H), 6.93 (d, J=6.85 Hz, 1H), 7.22-7.32 (m, 9H), 7.46 (d, J=7.05 Hz, 1H), 7.56 (s, 1H), 10.22 (s, 1H), 11.04 (s, 1H); LCMS m/z: 428.4 [M+H].

### Examples 48 and 55

Examples 48 and 55 were prepared according to the above methods used to make Example 47 as described in General Procedures 2-4, 6 using the appropriate acid. Purification was as stated in the aforementioned methods.

| | | IUPAC | | LCMS | Purity |
|---|---|---|---|---|---|
| Ex | Structure | Name | 1H-NMR | [M + H] | (%) |
| 48 | 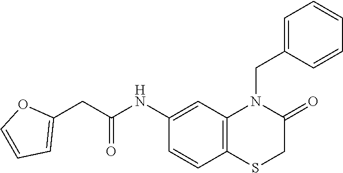 [SMILES: O=C(Cc1ccco1)Nc1ccc2c(c1)N(Cc1ccccc1)C(=O)CS2] | N-(4-benzyl- 3-oxo-3,4- dihydro-2H- benzo[b][1,4] thiazin-6-yl)-2- (furan-2- yl)acetamide | (500 MHz; DMSO-d₆): δ 3-33-3.37 (m, 4H), 5.14 (s, 2H), 6.23 (s, 1H), 6.39 (s, 1H), 7.21- 7.36 (m, 7H), 7.49-7:57 (m, 2H), 10.23 (s, 1H) | 379.2 | 97.64 |
| 55 | 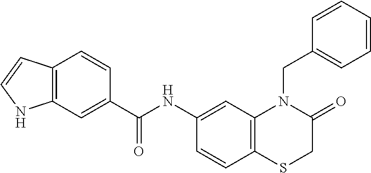 [SMILES: O=C(Nc1ccc2c(c1)N(Cc1ccccc1)C(=O)CS2)c1ccc2cc[nH]c2c1] | N-(4-benzyl- 3-oxo-3,4- dihydro-2H- benzo[b][1,4] thiazin-6-yl)- 1H-indole-6- carboxamide | (500 MHz; DMSO-d₆): δ 3.67 (s, 2H), 5.20 (s, 2H), 6.53 (s, 1H), 7.26- 7.40 (m, 6H), 7-57-7.65 (m, 4H), 7.80 (s, 1H), 8.00 (s, 1H), 10.24 (s, 1H), 11.48 (s, 1H) | 414.29 | 96.33 |

### Example 49: 4-Benzol-N-(furan-2-ylmethyl)-3,4-dihydro-2H-benzo[b][1,4]thiazine-6-sulfonamide

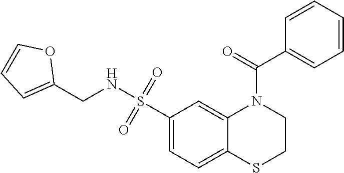 [SMILES: O=C(c1ccccc1)N1CCSc2ccc(S(=O)(=O)NCc3ccco3)cc21]

Example 49 was prepared according to General Procedure 17 and the methods described below.

### Preparation 35: 3-Oxo-3,4-dihydro-2H-benzo[b][1,4]thiazine-6-sulfonyl chloride

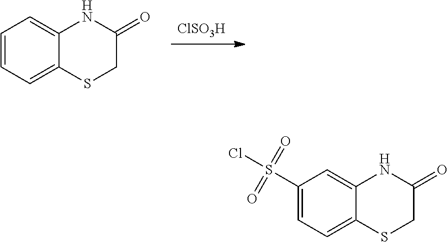

Commercially available 2H-benzo[b][1,4]thiazin-3(4H)-one (1.0 g) was added portionwise to stirring chlorosulfonic acid (3 mL) at 0-5° C. The cooling bath was removed and the combined mixture was stirred at RT for 2 h. During this time the reaction mixture turned a deep blue. Progress of the reaction was monitored by UPLC and TLC. After completion of the reaction, it was poured into cold water and further stirred for 30 min. to give a precipitate which was filtered off and washed with water followed by hexane to afford the title compound (600 mg, 38% yield) as a white solid. LCMS m/z: 262 [M−H].

### Preparation 36: N-(Furan-2-ylmethyl)-3-oxo-3,4-dihydro-2H-benzo[b][1,4]thiazine-6-sulfonamide

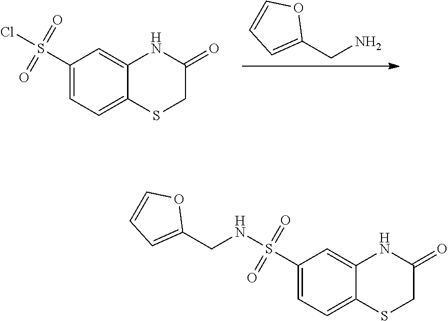

To a stirred solution of furan-2-ylmethanamine (80 mg, 0.823 mmol) in DCM (6 mL) was added TEA (0.287 mL) at 0-5° C. Thereafter 3-oxo-3,4-dihydro-2H-benzo[b][1,4]thiazine-6-sulfonyl chloride (Preparation 35) (216.64 mg, 0.823 mmol) was added into the reaction mixture and the whole stirred for 30 min. at RT. Progress of the reaction was monitored by TLC and LCMS and after completion the reaction mixture was poured into ice-cold water and extracted with DCM. The organic extracts were then washed with brine, dried over anhydrous Na₂SO₄, filtered and evaporated under high vacuum to afford the title compound (220 mg, 84% yield) as a white solid. LCMS m/z: 325 [M+H].

### Preparation 37: N-(Furan-2-ylmethyl)-3,4-dihydro-2H-benzo[b][1,4]thiazine-6-sulfonamide

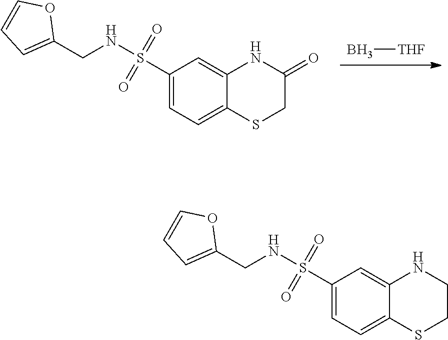

To a stirred solution of N-(furan-2-ylmethyl)-3-oxo-3,4-dihydro-2H-benzo[b][1,4]thiazine-6-sulfonamide (Preparation 36) (300 mg, 0.925 mmol) in THF (4 mL) was added borane-THF solution (318 mg, 3.7 mL, 3.703 mmol, 1M solution in THF) dropwise at 0-5° C. under an inert atmosphere. The resulting reaction mixture was stirred at RT for 12 h. After completion of the reaction (monitored by TLC or LCMS), the reaction mixture was quenched by dropwise addition of MeOH (5 mL) at 0-5° C. The solvent was evaporated under reduced pressure to give a residue which was partitioned between EtOAc and water. The organic layer was separated, washed with brine, dried over anhydrous Na₂SO₄ and evaporated to dryness in vacuo to afford the title compound (120 mg, 42% yield) as a yellow sticky solid which was used in the next step without any further purification.

### Preparation 38: 4-benzoyl-N-(furan-2-ylmethyl)-3,4-dihydro-2H-benzo[b][1,4]thiazine-6-sulfonamide (Example 49)

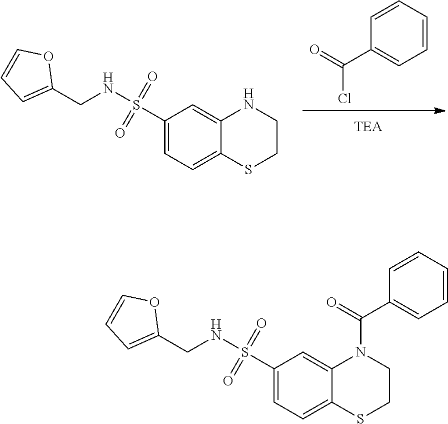 [SMILES: O=S(=O)(NCc1ccco1)c1ccc2c(c1)NCCS2 ; O=C(Cl)c1ccccc1 ; O=C(c1ccccc1)N1CCSc2ccc(S(=O)(=O)NCc3ccco3)cc21]

To a stirred solution of N-(furan-2-ylmethyl)-3,4-dihydro-2H-benzo[b][1,4]thiazine-6-sulfonamide (Preparation 37) (60 mg, 0.193 mmol) in dry DCM (3 mL) was added TEA (58 mg, 0.581 mmol) at 0-5° C. The resulting reaction mixture was stirred for 5 min. then benzoyl chloride (41 mg, 0.290 mmol) was added and stirring was continued for a further 5 h. Progress of the reaction was monitored by TLC and LCMS and after completion the reaction mixture was diluted with water and extracted with EtOAc. The combined organic layers were washed with brine solution, dried over anhydrous Na₂SO₄, filtered and concentrated under reduced pressure to give the crude product which was purified by prep-HPLC to afford the title compound (20 mg, 25% yield) as a white solid. Purity by HPLC: 98.32%; ¹H NMR (500 MHz; DMSO-d₆): δ 3.04 (dd, J′=3.0 Hz, J″=4.85 Hz, 2H), 3.50 (dd, J′=3.8 Hz, J″=6.5 Hz, 2H), 4.93 (s, 2H), 6.18 (d, J=3.1 Hz, 1H), 6.36-6.37 (m, 1H), 6.63 (s, 1H), 6.73-6.76 (m, 1H), 6.99 (s, 1H), 7.03 (d, J=8.2 Hz, 1H), 7.44-7.49 (m, 4H), 7.54-7.59 (m, 2H); LCMS m/z: 415.07 [M+H].

### Example 50: 1-(4-Benzyl-3,4-dihydro-2H-benzo[b][1,4]thiazin-6-yl)-3-(1H-indol-6-yl)urea

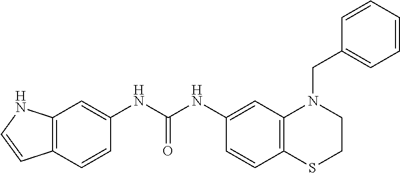 [SMILES: O=C(Nc1ccc2c(c1)N(Cc1ccccc1)CCS2)Nc1ccc2cc[nH]c2c1]

Example 50 was prepared according to General Procedure 1-6, 17 and the methods described below.

### Preparation 39: 4-Benzyl-3,4-dihydro-2H-benzo[b][1,4]thiazin-6-amine

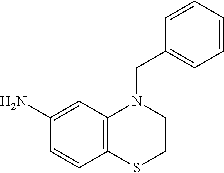 [SMILES: Nc1ccc2c(c1)N(Cc1ccccc1)CCS2]

### Step 1: Methyl 4-benzyl-3,4-dihydro-2H-benzo[b][1,4]thiazine-6-carboxylate

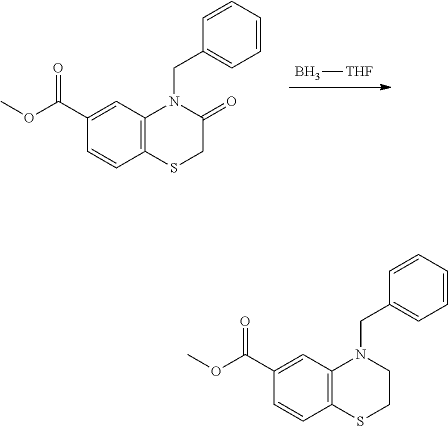

A solution of methyl 4-benzyl-3-oxo-3,4-dihydro-2H-benzo[b][1,4]thiazine-6-carboxylate (Preparation 2) (1.0 g, 3.19 mmol) in borane-THF complex (10.6 mL, 9.5 mmol; 0.9M solution in THF) was stirred at 0-5° C. for 2 h. UPLC-MS showed formation of the desired product. After completion of the reaction, the excess borane was quenched with methanol at the same temperature. The solvent was evaporated in vacuo and the residue was diluted with EtOAc, washed with water and brine, dried over anhydrous Na₂SO₄, filtered and concentrated in vacuo to give a crude material which was purified by column chromatography using 10% EtOAc in hexane as eluent to afford the title compound (900 mg, yield 94%) as a white solid. LCMS m/z: 300.23 [M+H].

### Step 2: 4-Benzyl-3,4-dihydro-2H-benzo[b][1,4]thiazine-6-carboxylic acid

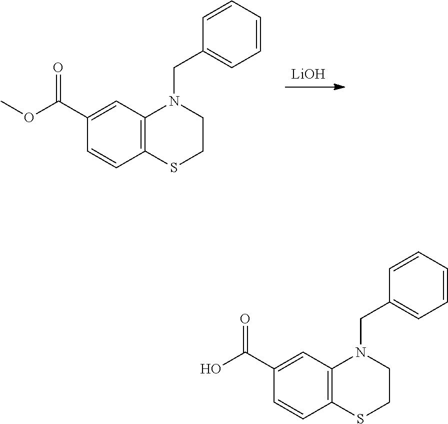

To a stirred solution of methyl 4-benzyl-3,4-dihydro-2H-benzo[b][1,4]thiazine-6-carboxylate (Preparation 39, Step 1) (120 mg, 0.4 mmol) in THF (5 mL) and MeOH (2.5 mL) was added a solution of LiOH (84 mg, 2.0 mmol) in water (2.5 mL) and the mixture was maintained at RT for 16 h. Progress of the reaction was monitored by TLC. After completion of the reaction, the solvent was evaporated in vacuo to give a crude material which was diluted with water and acidified with 6N HCl. The product was extracted with EtOAc and the combined organics were washed with brine, dried over anhydrous Na₂SO₄, filtered and evaporated to afford the title compound (100 mg, crude) as a faint brownish solid which was used in the next step without any further purification. LCMS m/z: 286.22 [M+H].

### Step 3: tert-Butyl (4-benzyl-3,4-dihydro-2H-benzo[b][1,4]thiazin-6-yl)carbamate

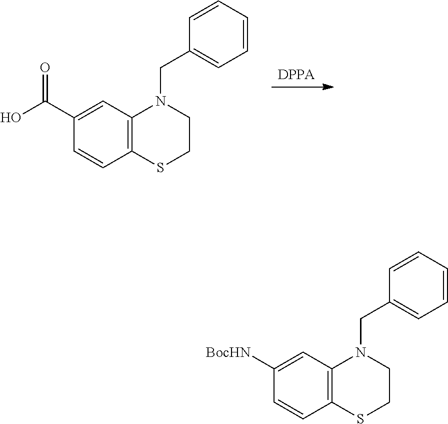

A stirred solution of 4-benzyl-3,4-dihydro-2H-benzo[b][1,4]thiazine-6-carboxylic acid (Preparation 39, Step 2) (100 mg, 0.35 mmol) in DCM (10 mL) was cooled to 0-5° C. and TEA (0.075 mL, 0.53 mmol) added followed by DPPA (0.152 mL, 0.7 mmol) at RT. The combined mixture was stirred at RT for 3 h. UPLC-MS showed consumption of the starting material. The solvent was evaporated in vacuo to give an intermediate product (130 mg) as a faint brownish solid. The solid was dissolved in tert-butanol (10 mL) and refluxed for 24 h. Progress of the reaction was monitored by UPLC-MS and after completion of the reaction, the solvent was evaporated in vacuo to give a residue which was purified by Combi-flash (12 g column) using 55% EtOAc in hexane as eluent to afford the title compound (100 mg, yield 80%) as an off white sticky oil. LCMS m/z: 357.3 [M+H].

### Step 4: 4-Benzyl-3,4-dihydro-2H-benzo[b][1,4]thiazin-6-amine

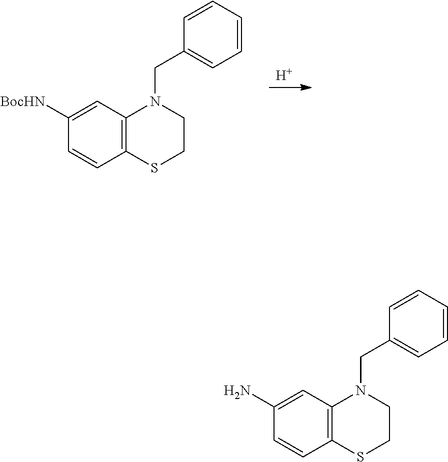

A stirred solution of tert-butyl (4-benzyl-3,4-dihydro-2H-benzo[b][1,4]thiazin-6-yl)carbamate (Preparation 39, Step 3) (100 mg, 0.28 mmol) in THF (2.5 mL) was cooled to 0-5° C. and 4M HCl in dioxane (2.5 mL) added dropwise under an inert atmosphere. The whole was allowed to warm slowly to RT over 24 h. Progress of the reaction was monitored by UPLC-MS. After completion of the reaction, the solvent was evaporated and the obtained crude material was dissolved in water and washed with ether. The aqueous layer was neutralized with a saturated aqueous sodium bicarbonate solution and extracted with EtOAc. The combined organic layers were washed with brine, dried over anhydrous Na₂SO₄ and evaporated in vacuo to afford the title compound (80 mg, crude) as brownish oil which was used as such in the next step. LCMS m/z: 257.22 [M+H].

### Preparation 40: 1-(4-Benzyl-3,4-dihydro-2H-benzo[b][1,4]thiazin-6-yl-3-(1H-indol-6-yl)urea (Example 50)

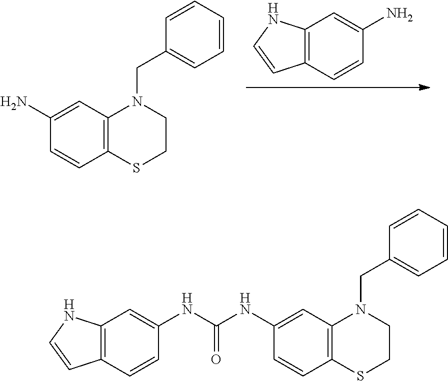

To a stirred solution of 4-benzyl-3,4-dihydro-2H-benzo[b][1,4]thiazin-6-amine (Preparation 39, Step 4) (80 mg, 0.312 mmol) in THF (10 mL) was added p-nitrophenyl chloroformate (63 mg, 0.374 mmol) at 0-5° C. and the mixture was allowed to warm slowly to RT over 1 h. TLC showed completion of the first part of the reaction. 6-amino-indole (45 mg, 0.34 mmol) and TEA (0.067 mL, 0.468 mmol) were added and the combined mixture maintained at RT for a further 1 h. TLC and UPLC-MS showed complete consumption of the intermediate. The solvent was evaporated in vacuo to afford the crude material which was purified by prep-HPLC to afford the title compound (8 mg, yield 6%) as a faint brownish solid. Purity by UPLC: 97.3%; ¹H NMR (400 MHz; DMSO-d₆): δ. 3.04-3.07 (m, 2H), 3.65-3.67 (m, 2H), 4.55 (s, 2H), 6.30-6.31 (m, 1H), 6.75 (t, J=2.16 Hz, 1H), 6.77-6.78 (m, 2H), 6.86 (d, J=8.64 Hz, 1H), 7.19-7.20 (m, 1H), 7.26-7.30 (m, 3H), 7.35-7.38 (m, 3H), 7.74 (t, J=0.86 Hz, 1H), 8.33 (s, 1H), 8.42 (s, 1H), 10.87 (s, 1H); LCMS m/z: 415.28 [M+H].

### Example 62: 1-(4-Benzyl-3,4-dihydro-2H-benzo[b][1,4]oxazin-7-yl)-3-(1H-indol-6-yl)urea

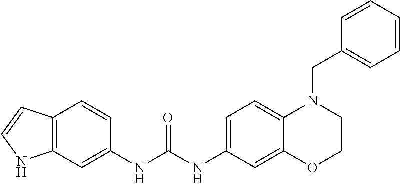 [SMILES: O=C(Nc1ccc2c(c1)OCCN2Cc1ccccc1)Nc1ccc2cc[nH]c2c1]

Example 62 was prepared according to General Procedure 1-6, 18 and the methods described below.

### Preparation 42: 4-Benzyl-3,4-dihydro-2H-benzo[b][1,4]oxazin-7-amine

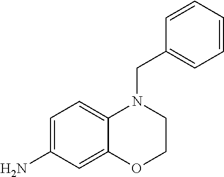 [SMILES: Nc1ccc2c(c1)OCCN2Cc1ccccc1]

### Step 1: Methyl 3,4-dihydro-2H-benzo[b][1,4]oxazine-7-carboxylate

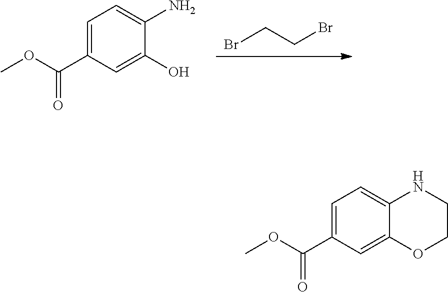

To a stirred solution of commercially available methyl 4-amino-3-hydroxybenzoate (0.5 g, 2.99 mmol) in DMF (5 mL) was added K₂CO₃ (2.75 g, 11.96 mmol) and 1,2-dibromoethane (1.035 mL, 11.96 mmol) at RT. The whole was stirred at 80° C. for 16 h. TLC and UPLC-MS showed formation of the desired product and after completion of the reaction, the mixture was diluted with water and extracted with EtOAc. The combined organics were washed with brine, dried over anhydrous Na₂SO₄, filtered and evaporated in vacuo to give a crude material which was purified by Combi-flash (20 g column) using 20% EtOAc in hexane as eluent to afford the title compound (0.3 g, 52% yield) as a pale yellow solid. UPLC-MS m/z: 193.98 [M+H].

### Step 2: Methyl 4-benzyl-3,4-dihydro-2H-benzo[b][1,4]oxazine-7-carboxylate

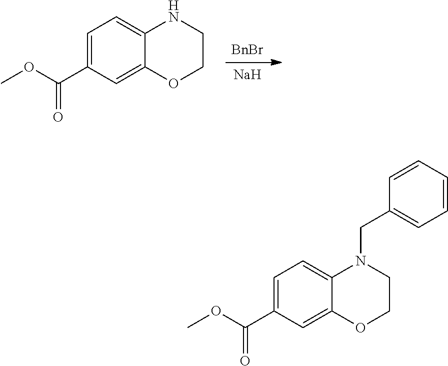

To a stirred solution of methyl 3,4-dihydro-2H-benzo[b][1,4]oxazine-7-carboxylate (Preparation 42, Step 1) (300 mg, 1.55 mmol) in DMF (3 mL) was added NaH (68 mg, 1.71 mmol) portionwise at 0-5° C. After the addition was complete, benzyl bromide (0.204 mL, 1.71 mmol) was added and the whole was allowed to warm slowly to RT over 1.5 h. TLC and UPLC-MS showed formation of the desired product and after complete consumption of the starting material, the reaction mixture was diluted with water and extracted with EtOAc. The combined organic layers were washed with brine, dried over anhydrous Na₂SO₄, filtered and evaporated in vacuo to afford the title compound (420 g, 95% yield) as a pale yellow solid. UPLC-MS m/z: 284.3 [M+H].

### Step 3: 4-Benzyl-3,4-dihydro-2H-benzo[b][1,4]oxazin-7-amine

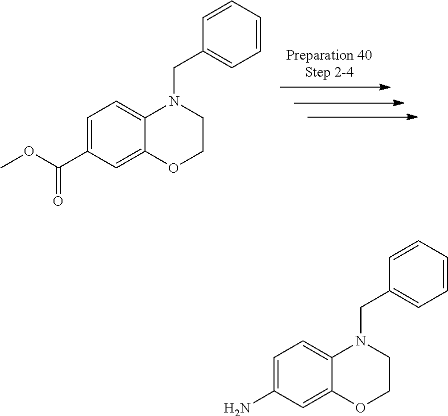 [SMILES: COC(=O)c1ccc2c(c1)OCCN2Cc1ccccc1 ; "Preparation 40Step 2-4" ; Nc1ccc2c(c1)OCCN2Cc1ccccc1]

The title compound was prepared according to the methods described for the preparation of Example 50 (Preparation 40, Steps 2-4), starting from methyl 4-benzyl-3,4-dihydro-2H-benzo[b][1,4]oxazine-7-carboxylate (Preparation 42, Step 2), instead of methyl 4-benzyl-3,4-dihydro-2H-benzo[b][1,4]thiazine-6-carboxylate (Preparation 40, Step 1). UPLC-MS m/z: 241.4 [M+H].

### Preparation 43: 1-(4-Benzyl-3,4-dihydro-2H-benzo[b][1,4]oxazin-7-yl-3-(1H-indol-6-yl)urea (Example 62)

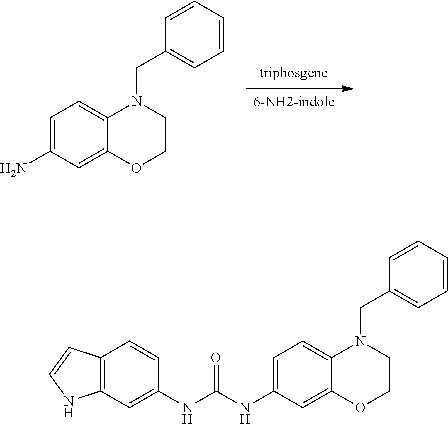 [SMILES: Nc1ccc2c(c1)OCCN2Cc1ccccc1 ; "6-NH2-indole" ; "triphosgene" ; O=C(Nc1ccc2c(c1)OCCN2Cc1ccccc1)Nc1ccc2cc[nH]c2c1]

To a stirred solution of 6-amino indole (60 mg, 0.416 mmol) in THF (5 mL) was added triphosgene (61 mg, 0.208 mmol) at 0-5° C. The resulting reaction mixture was allowed to warm to RT over 1 h. 4-benzyl-3,4-dihydro-2H-benzo[b][1,4]oxazin-7-amine (Preparation 42, Step 3) (100 mg, 0.416 mmol) and TEA (0.198 mL, 1.217 mmol) were then added into the reaction mixture which was further stirred at RT for 1 h. TLC showed complete consumption of the amine and a new polar material was observed. The solvent was evaporated in vacuo to afford the crude which was purified by prep-HPLC to give the title compound (35 mg, 21% yield) as a pale brownish solid. Purity by UPLC: 98.94%; ¹H NMR (400 MHz; DMSO-d₆): δ 3.27-3.34 (m, 2H), 4.20-4.22 (m, 2H), 4.40 (s, 2H), 6.31-6.32 (m, 1H), 6.62 (d, J=8.76 Hz, 1H), 6.71 (dd, J′=8.68 Hz, J″=2.44 Hz, 1H), 6.80 (dd, J′=8.44 Hz, J″=1.84 Hz, 1H), 6.97 (d, J=2.4 Hz, 1H), 7.19-7.20 (m, 1H), 7.22-7.27 (m, 1H), 7.31-7.33 (m, 3H), 7.34-7.35 (m, 1H), 7.37-7.39 (m, 1H), 7.77-7.78 (m, 1H), 8.24 (s, 1H), 8.42 (s, 1H), 10.89 (s, 1H); UPLC-MS m/z: 399.1 [M+H].

### Example 176: 4-Benzyl-N-(1H-indol-6-ylsulfamoyl)-2,3-dihydro-1,4-benzoxazin-6-amine

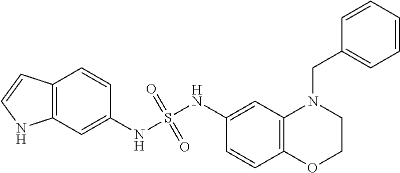 [SMILES: O=S(=O)(Nc1ccc2c(c1)N(Cc1ccccc1)CCO2)Nc1ccc2cc[nH]c2c1]

Example 176 was prepared according to General Procedures 1-6, 17 and the methods described below.

### Preparation 77: N-(4-Benzyl-3,4-dihydro-2H-benzo[b][1,4]oxazin-6-yl-2-oxooxazolidine-3-sulfonamide

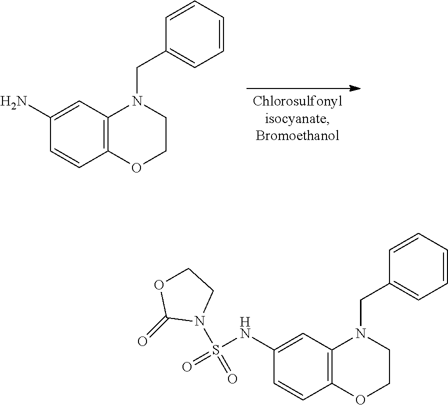 [SMILES: Nc1ccc2c(c1)N(Cc1ccccc1)CCO2 ; "Chlorosulfonylisocyanate,Bromoethanol" ; O=C1OCCN1S(=O)(=O)Nc1ccc2c(c1)N(Cc1ccccc1)CCO2]

Chlorosulfonyl isocyanate (82 mg, 0.58 mmol) was taken in DCM (2 mL) and it was cooled to 0-5° C. Then bromoethanol (46.52 mg, 0.58 mmol) was added to it drop wise and the mixture was stirred at 0-5° C. for 30 min. To this reaction vessel was added a mixture of 4-benzyl-3,4-dihydro-2H-1,4-benzoxazine-6-amine (Preparation 39, Step 4) (140 mg, 0.58 mmol) and Et3N (0.13 ml, 0.96 mmol) in DCM (1 mL) at 0-5° C. The whole reaction mixture was stirred at 0-5° C. for 30 min then warm to RT and stirred for 10 min at RT. Progress of the reaction was checked by LCMS and after completion of the reaction; solvent was evaporated in vacuo to get the title compound (150 mg, crude) as crude solid which was used in the next step without any further purification.

### Preparation 78: 4-Benzyl-N-(1H-indol-6-ylsulfamoyl)-2,3-dihydro-1,4-benzoxazin-6-amine (Example 176)

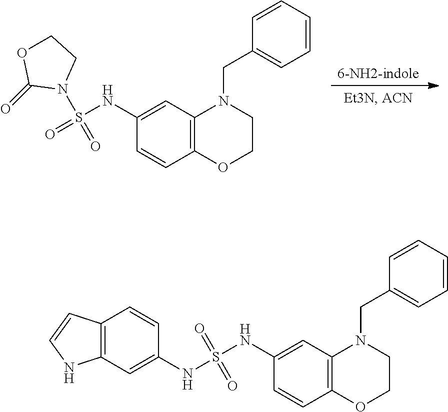

To a solution of N-(4-benzyl-3,4-dihydro-2H-benzo[b][1,4]oxazin-6-yl)-2-oxooxazolidine-3-sulfonamide (Preparation 77) (150.0 mg, 0.38 mmol) in acetonitrile (5 mL) were added 6-amino-indole (61.0 mg, 0.46 mmol) and Et3N (0.16 ml, 0.96 mmol) at RT and the reaction mixture was stirred at RT for 16 h. Progress of the reaction was checked by LCMS and after completion of the reaction; solvents were evaporated under reduced pressure to give crude product which was purified by reverse phase Prep-HPLC to afford the title compound (15 mg, 8.96% yield) as black sticky solid. Purity by UPLC: 98.94%; ¹H NMR (400 MHz; DMSO-d₆): δ 3.27 (m, 2H), 4.12 (s, 2H), 4.30 (s, 2H), 6.34 (s, 1H), 6.36 (s, 1H), 6.45 (s, 1H), 6.56 (d, 1H, J=8.2 Hz), 6.77 (d, 1H, J=8.3 Hz), 7.16-7.36 (m, 8H), 9.37 (s, 1H), 9.61 (s, 1H), 10.96 (s, 1H); UPLC-MS m/z: 435.07 [M+H].

### Examples 56-57, 63-68, 167-174 and 177-180

The examples in the table below were prepared according to the above methods used to make Examples 50, 62 and 176 as described in General Procedures 1-6, 17 and 18 using the appropriate amine. Purification was as stated in the aforementioned methods.

| | | IUPAC | | LCMS | Purity |
|---|---|---|---|---|---|
| Ex | Structure | Name | 1H-NMR | [M + H] | (%) |
| 56 | 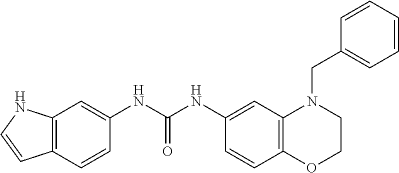 [SMILES: O=C(Nc1ccc2c(c1)N(Cc1ccccc1)CCO2)Nc1ccc2cc[nH]c2c1] | 1-(4-benzyl- 3,4- dihydro- 2H- benzo[b][1, 4]oxazin-6- yl)-3-(1H- indol-6- yl)urea | (500 MHz; DMSO- d₆): δ 3.34 (s, 2H), 4.18 (s, 2H), 4.48 (s, 2H), 6.31 (s, 1H), 6.62-6.63 (m, 2H), 6.77-6.79 (m, 1H), 6.84 (s, 1H), 7.20 (s, 1H), 7-33-7-38 (m, 6H), 7.77 (s, 1H), 8.27 (s, 1H), 8.43 (s, 1H), 10.87 (s, 1H) | 399.14 | 96.73 |
| 57 | 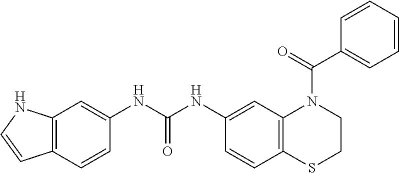 [SMILES: O=C(Nc1ccc2c(c1)N(C(=O)c1ccccc1)CCS2)Nc1ccc2cc[nH]c2c1] | 1-(4- benzoyl- 3,4- dihydro- 2H- benzo[b][1, 4]thiazin-6- yl)-3-(1H- indol-6- yl)urea | (500 MHz; DMSO- d₆): δ 3.2-3.3 (m, 2H), 4.02 (bs, 2H), 6.32 (s, 1H), 6.76- 6.78 (m, 1H), 6.92 (s, 1H), 7.17-7.24 (m, 3H), 7.30-7.42 (m, 6H), 7.70 (s, 1H), 8.46-8.47 (m, 2H), 10.91 (s, 1H) | 429.10 | 96.1 |
| 63 | 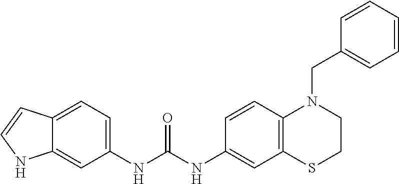 [SMILES: O=C(Nc1ccc2c(c1)SCCN2Cc1ccccc1)Nc1ccc2cc[nH]c2c1] | 1-(4-benzyl- 3,4- dihydro- 2H- benzo[b][1, 4]thiazin-7- yl)-3-(1H- indol-6- yl)urea | (500 MHz; DMSO- d₆): δ 3.11 (t, J = 4.7 Hz, 2H), 3.60 (t, J = 4.65 Hz, 2H), 4.50 (s, 2H), 6.32 (s, 1H), 6.57 (d, J = 8.9 HZ, 1H), 6.82-6.86 (m, 2H), 7.20-7.39 (m, 8H), 7.77 (s, 1H), 8.36 (s, 1H), 8.56 (s, 1H), 10.90 (s, 1H) | 415.07 | 99.1 |
| 64 | 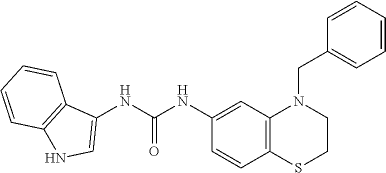 [SMILES: O=C(Nc1ccc2c(c1)N(Cc1ccccc1)CCS2)Nc1c[nH]c2ccccc12] | 1-(4-benzyl- 3,4- dihydro- 2H- benzo[b][1, 4]thiazin-6- yl)-3-(1H- indol-3- yl)urea | (500 MHz; DMSO- d₆): δ 3.06 (t, J = 5 Hz, 2H), 3-67 (t, J = 4.7 Hz, 2H), 4.56 (s, 2H), 6.72 (s, 1H), 6.83-6.88 (m, 2H), 6.99 (t, J = 7-5 Hz, 1H), 7.08 (t, J = 7-35 Hz, 1H), 7.25-7.30 (m, 4H), 7-33-7:39 (m, 2H), 7.44-7.46 | 415.09 | 98.48 |
| | | | (m, 2H), 8.32 (s, 2H), | | |
| | | | 10.69 (s, 1H) | | |
| 65 | 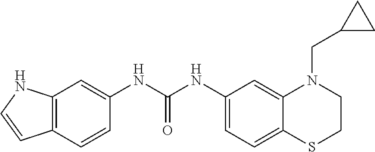 [SMILES: O=C(Nc1ccc2c(c1)N(CC1CC1)CCS2)Nc1ccc2cc[nH]c2c1] | 1-(4- (cyclopropyl- methyl)- 3,4- dihydro- 2H- benzo[b][1, 4]thiazin-6- yl)-3-(1H- indol-6- | (400 MHz; DMSO- d₆): δ 0.28-0.30 (111, 2H), 0.49-0.51 (m 2H), 1.07-1.11 (m, 1H), 2.9-3.02 (m, 2H), 3.17 (d, J = 6.36 Hz, 2H), 3.61-3.64 (m, 2H), 6.31-6.33 (m, 1H), 6.60-6.63 (m, 1H), 6.80-6.83 | 379.1 | 97.73 |
| | | yl)urea | (m, 2H), 7.10 (d, J = | | |
| | | | 2.04 Hz, 1H), 7.21 (d, | | |
| | | | J = 2.44 Hz, 1H), | | |
| | | | 7.38-7.41 (m, 1H), | | |
| | | | 7.80 (d, J = 1.84 Hz, | | |
| | | | 1H), 8.47 (d, J = 9.24 | | |
| | | | Hz, 2H),10.89 (s, | | |
| | | | 1H) | | |
| 66 | 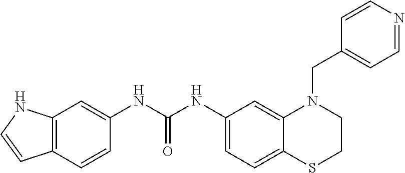 [SMILES: O=C(Nc1ccc2c(c1)N(Cc1ccncc1)CCS2)Nc1ccc2cc[nH]c2c1] | 1-(1H-indol- 6-yl)-3-(4- (pyridin-4- ylmethyl)- 3,4- dihydro- 2H- benzo[b][1, 4]thiazin-6- yl)urea | (400 MHz; DMSO- d₆): δ 3.08-3.11 (m, 2H), 3.68-3.72 (m, 2H), 4.58 (s, 2H), 6.30 (s, 1H), 6.65 (d, J = 1.68 Hz, 1H), 6.74-6.79 (m, 2H), 6.88 (t, J = 8.32 Hz, 1H), 7.19 (t, J = 2.72 Hz, 1H), 7.28-7.30 (m, 2H), 7-35-7-37 | 416.1 | 97.07 |
| | | | (m, 1H), 7.72 (s, 1H), | | |
| | | | 8.34-8.43 (m, 2H), | | |
| | | | 8.52-8.54 (m, 2H), | | |
| | | | 10.87 (s, 1H) | | |
| 67 | 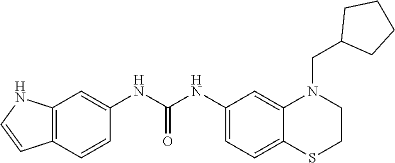 [SMILES: O=C(Nc1ccc2c(c1)N(CC1CCCC1)CCS2)Nc1ccc2cc[nH]c2c1] | 1-(4- (cyclopentyl methyl)- 3,4- dihydro- 2H- benzo[b][1, 4]thiazin-6- yl)-3-(1H- indol-6- | (400 MHz; DMSO- d₆): δ 1.2-1.27 (m, 2H), 1.51-1.53 (m, 2H), 1.54-1.64 (m, 2H), 1.72-1.75 (m, 2H), 2.25-2.33 (m, 1H), 2.97-2.99 (m, 2H), 3.20 (d, J = 7.2 Hz, 2H), 3-59-3.62 (m, 2H), 6.32 (s, 1H), | 407.14 | 99.18 |
| | | yl)urea | 6.63-6.66 (m, 1H), | | |
| | | | 6.78-6.84 (m, 2H), | | |
| | | | 7.95 (s, 1H), 7.20 (s, | | |
| | | | 1H), 7-39 (d, J = 8.44 | | |
| | | | Hz, 1H), 7.78 (s, 1H), | | |
| | | | 8.50 (s, 1H), 8.55 (s, | | |
| | | | 1H), 10.90 (s, 1H) | | |
| 68 | 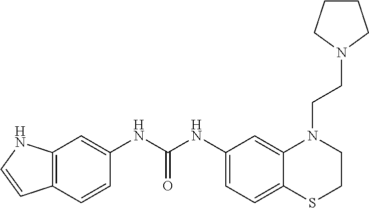 [SMILES: O=C(Nc1ccc2c(c1)N(CCN1CCCC1)CCS2)Nc1ccc2cc[nH]c2c1] | 1-(1H-indol- 6-yl)-3-(4- (2- (pyrrolidin- 1-yl)ethyl)- 3,4- dihydro- 2H- benzo[b][1, 4]thiazin-6- yl)urea | (400 MHz; DMSO- d₆): δ 1.62-1.8 (m, 4H), 2.60-2.65 (m 3H), 2.97-3.08 (m, 2H), 3-33-3-40 (m, 3H), 3.52 (s, 1H), 3.6- 3.62 (m, 3H), 6.32 (s, 1H), 6.66-6.72 (m, 1H), 6.79-6.83 (m, 2H), 6.92 (s, 1H), 7.21 (s, 1H), 7.26-7.27 (m, 1H), 7-39-7-40 (m, 1H), 7.78 (s, 1H), 8.39 | 422.16 | 95.57 |
| | | | (s, 1H), 8.45 (s, 1H), | | |
| | | | 10.89 (s, 1H) | | |
| 167 | 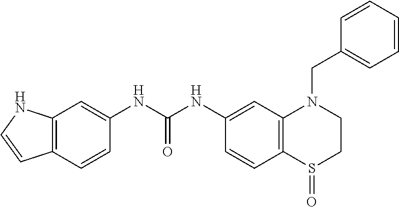 [SMILES: O=C(Nc1ccc2c(c1)N(Cc1ccccc1)CCS2=O)Nc1ccc2cc[nH]c2c1] | 1-(4-benzyl- 1-oxido-3,4- dihydro- 2H- benzo[b][1, 4]thiazin-6- yl)-3-(1H- indol-6- yl)urea | (400 MHz; DMSO- d6): δ 2.85 (t, 1H, J = 13.6 Hz ), 3.07 (d, 1H, J = 13.9 Hz), 3.63 (d, 1H, J = 13.6 Hz), 3.98 (t, 1H, J = 13.2 Hz ), 4.78-4.64 (m, 2H), 6.32 (s, 1 H), 6.85 (d, 2H, 8.16), 7-39-7.13 (m, 9H), 7.77 (s, 1H), 9.16 (s, 1H), 9.32 (s, 1H), 10.89 (s, 1H) | 431.1 | 97.75 |
| 168 | 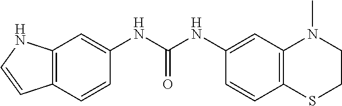 [SMILES: CN1CCSc2ccc(NC(=O)Nc3ccc4cc[nH]c4c3)cc21] | 1-(1H-indol- 6-yl)-3-(4- methyl-3,4- dihydro- 2H- benzo[b][1, 4]thiazin-6- | (500 MHz; DMSO- d₆): δ 2.89 (s, 3H), 3.05 (t, 2H, J = 3.65 Hz), 3.51 (t, 2H, J = 5.2 Hz), 6.33 (s, 1H), 6.68-6.66 (m, 1H), 6.85-6.82 (m, 2H), | 339.06 | 99.14 |
| | | yl)urea | 6.97 (d, 1H, J = 1.52 | | |
| | | | H), 7.22 (t, 1H, J = | | |
| | | | 2.65 Hz), 7.40 (d, 1H, | | |
| | | | J = 8.4 Hz), 7.81 (s, | | |
| | | | 1H), 8.46 (s, 1H), | | |
| | | | 8.50 (s, 1H), 10.89 (s, | | |
| | | | 1H). | | |
| 169 | 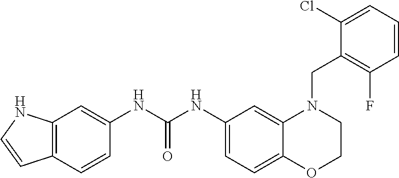 [SMILES: O=C(Nc1ccc2c(c1)N(Cc1c(F)cccc1Cl)CCO2)Nc1ccc2cc[nH]c2c1] | 1-(4-(2- chloro-6- fluorobenzyl)- 3,4- dihydro- 2H- benzo[b][1, 4]oxazin-6- yl)-3-(1H- indol-6- yl)urea | (400 MHz; DMSO- d₆): δ 3.08 (t, 2H, J = 4.08 Hz), 4.065 (m, 2H), 4.47 (s, 2H), 6.32 (s, 1H), 6.62 (d, 1H, J = 8.48 Hz), 6.70 (m, 1H), 6.82 (m, 1H), 7.15 (d, 1H, J = 1.68 Hz), 7.18 (m, 1H), 7.29-7.47 (m, 4H), 7.79 (s, 1H), | 451.06 | 98.57 |
| | | | 8.24 (s, 1H), 8.43 (s, | | |
| | | | 1H), 10.86 (s, 1H). | | |
| 170 | 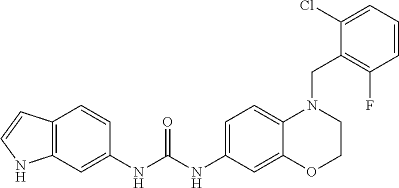 [SMILES: O=C(Nc1ccc2c(c1)OCCN2Cc1c(F)cccc1Cl)Nc1ccc2cc[nH]c2c1] | 1-(4-(2- chloro-6- fluorobenzyl)- 3,4- dihydro- 2H- benzo[b][1, 4]oxazin-7- yl)-3-(1H- indol-6- yl)urea | (400 MHz; DMSO- d₆): δ 3.03 (s, 2H), 4.11 (s, 2H), 4.40 (s, 2H), 6.30 (s, 1H), 6.83 (s, 2H), 6.90 (d, 1H, J = 7.72 Hz), 6.98 (s, 1H), 7.18 (s, 1H), 7.28 (s, 1H), 7.40 (m, 3H), 7.77 (s, 1H), 8.49 (s, 1H), 8.62 (s, 1H), 10.87 (s, 1H). | 451.1 | 98.74 |
| 171 | 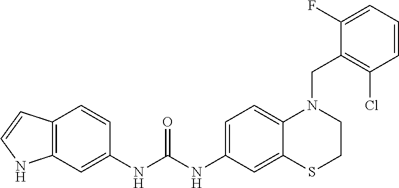 [SMILES: O=C(Nc1ccc2c(c1)SCCN2Cc1c(F)cccc1Cl)Nc1ccc2cc[nH]c2c1] | 1-(4-(2- chloro-6- fluorobenzyl)- 3,4- dihydro- 2H- benzo[b][1, 4]thiazin-7- yl)-3-(1H- indol-6- yl)urea | (400 MHz; DMSO- d₆): δ 3.00 (t, 2H, J = 4.96 Hz), 3.27 (s, 2H), 4.46 (s, 2H), 6.31 (s, 1H), 6.84 (d, 1H, J = 8.48 Hz), 6.91-6.97 (m, 2H), 7.19 (s, 1H), 7.26-7.44 (m, 5H), 7.76 (s, 1H), 8.38 (s, 1H), 8.54 (s, 1H), 10.88 (s, 1H). | 467.08 | 95.27 |
| 172 | 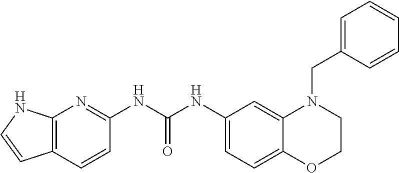 [SMILES: O=C(Nc1ccc2c(c1)N(Cc1ccccc1)CCO2)Nc1ccc2cc[nH]c2n1] | 1-(4-benzyl- 3,4- dihydro- 2H- benzo[b][1, 4]oxazin-6- yl)-3-(1H- pyrrolo[2,3- b]pyridin- 6-yl)urea | (400 MHz; DMSO- d₆): δ 3.35 (t, 2H, J = 4.12 Hz), 4.17 (t, 2H, J = 4.12 Hz), 4.96 (s, 2H), 6.31 (s, 1H), 6.65 (d, 1H, J = 8.68 Hz), 6.73-6.67 (m, 1H), 6.98 (s, 1H), 7.18 (s, 1H), 7.23-7.36 (m, 6H), 7.86 (d, 1H, J = 8.44 Hz), 9.28 (s, | 400.11 | 97.12 |
| | | | 1H), 10.80 (s, 1H), | | |
| | | | 11.5 (s, 1H). | | |
| 173 | 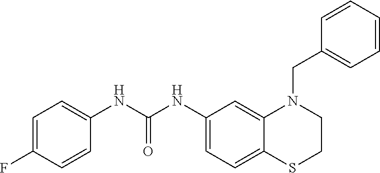 [SMILES: O=C(Nc1ccc(F)cc1)Nc1ccc2c(c1)N(Cc1ccccc1)CCS2] | 1-(4-benzyl- 3,4- dihydro- 2H- benzo[b][1, 4]thiazin-6- yl)-3-(4- fluorophenyl) urea | (400 MHz; DMSO- d₆): δ 3.04 (t, 2H, J = 5.12 Hz), 3.65 (t, 2H, J = 4.84 Hz), 4.53 (s, 2H), 6.72-6.76 (m, 2H), 6.85 (d, 1H, J = 8.28 Hz), 7.05-7.10 (m, 2 H), 7-25-7-39 (m, 7H), 8.41 (s, 1H), 8.58 (s, 1H) | 394.06 | 95.2 |
| 174 | 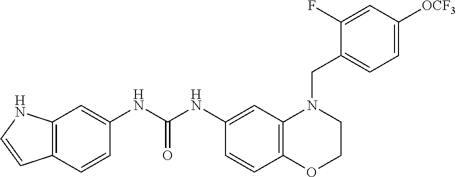 [SMILES: O=C(Nc1ccc2c(c1)N(Cc1ccc(OC(F)(F)F)cc1F)CCO2)Nc1ccc2cc[nH]c2c1] | 1-(4-(2- fluoro-4- (trifluoro- methoxy) benzyl)-3,4- dihydro- 2H- benzo[b][1, 4]oxazin-6- yl)-3-(1H- indol-6- | (400 MHz; DMSO- d₆): δ 3.40 (t, 2H, J = 4.04 Hz), 4.17 (t, 2H, J = 3.92 Hz), 4.52 (s, 2H), 6.30 (s, 1H), 6.62 (d, 1H, J = 8.48 Hz), 6.66-6.67 (d, 1H, J = 2.0 Hz), 6.75-6.78 (m, 2H), 7.18 (t, 1H, J = 2.50 Hz), 7-2-7-25 (m, 1H), 7.36 (d, 1H, | 501.09 | 99.0 |
| | | yl)urea | J = 8.44 Hz), 7.42- | | |
| | | | 7.45 (m, 2H), 7.74 (s, | | |
| | | | 1H), 8.23 (s, 1H), | | |
| | | | 8.36 (s, 1H), 10.85 (s, | | |
| | | | 1H). | | |
| 177 | 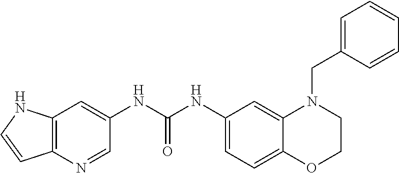 [SMILES: O=C(Nc1ccc2c(c1)N(Cc1ccccc1)CCO2)Nc1cnc2cc[nH]c2c1] | 1-(4-benzyl- 3,4- dihydro- 2H- benzo[b][1, 4]oxazin-6- yl)-3-(1H- pyrrolo[3,2- b]pyridin- 6-yl)urea | (400 MHz; DMSO- d₆): δ 3.39 (s, 2H), 4.15-4.17 (t, J = 4.24 Hz, 2H), 4.46 (s, 2H), 6.43 (s, 1H), 6.59-6.65 (m, 2H), 6.81 (s, 1H), 7.23-7.27 (s, 1H), 7-31-7-37 (m, 4H), 7.46 (t, 1H, J = 2.68 Hz), 8.10 (d, 2H, J = 6.6 Hz), 8.39 (s, | 400.15 | 98.23 |
| | | | 1H), 8.63 (s, 1H), | | |
| | | | 11.05 (s, 1H). | | |
| 178 | 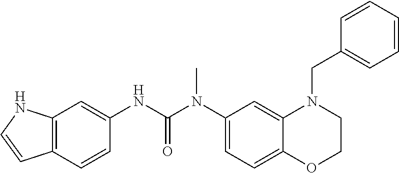 [SMILES: CN(C(=O)Nc1ccc2cc[nH]c2c1)c1ccc2c(c1)N(Cc1ccccc1)CCO2] | 1-(4-benzyl- 3,4- dihydro- 2H- benzo[b][1, 4]oxazin-6- yl)-3-(1H- indol-6-yl)- 1- methylurea | (400 MHz; DMSO- d₆): δ 3.9 (s, 3H), 3.39 (s, 2H), 4.23 (s, 2H), 4.50 (s 2H), 6.29 (bs, 1H), 6.48- 6.51 (dd, 1H, J1 = 2.04 Hz, J2 = 8.32 Hz), 6.7.-6.76 (m, 3H), 7.18-7.21 (m, 2H), 7.20-7.321 (m, 6H), 7.57 (s, 1H), | 413.21 | 99.69 |
| | | | 10.86 (s, 1H). | | |
| 179 | 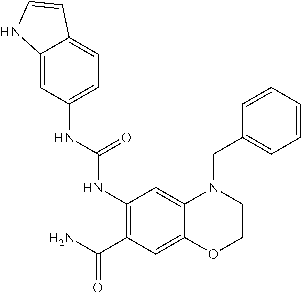 [SMILES: NC(=O)c1cc2c(cc1NC(=O)Nc1ccc3cc[nH]c3c1)N(Cc1ccccc1)CCO2] | 6-(3-(1H- indol-6- yl)ureido)- 4-benzyl- 3,4- dihydro- 2H- benzo[b][1, 4]oxazine- 7- carboxamide | (400 MHz; DMSO- d₆): δ 3.44 (s, 2H), 4.17 (s, 2 H), 4.53 (s, 2H), 6.29 (s, 1H), 6.95 (d, 1H, J = 8.52 Hz), 7.09-7-35 (m, 9H), 7-73 (s, 2H), 7.87 (s, 1H), 9.26 (s, 1H), 10.82 (s, 1H), 10.99 (s, 1H) | 442.31 | 99.82 |
| 180 | 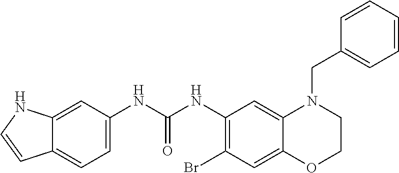 [SMILES: O=C(Nc1ccc2cc[nH]c2c1)Nc1cc2c(cc1Br)OCCN2Cc1ccccc1] | 1-(4-benzyl- 7-bromo- 3,4- dihydro- 2H- benzo[b][1, 4]oxazin-6- yl)-3-(1H- indol-6- yl)urea | (400 MHz; DMSO- d₆): δ 3.37 (t, 2H, J = 4.16 Hz), 4.18 (t, 2 H, J = 42 Hz), 4.46 (s, 2H), 6.31 (s, 1H), 6.78-6.81 (dd, 1H, J = 1.56 Hz, 8.63 Hz), 6.90 (s, 1H), 7.19- 7.49 (m, 8H), 7.71 (s, 1H), 7.77 (s, 1H), 9.07 (d, 1H, J = 4.12 Hz), | 477.18 | 99.22 |
| | | | 10.87 (s, 1H) | | |

### Example 72: 1-(1H-Indol-6-yl)-R-(4-phenyl-1,4-dihydro-2H-benzo[b][1,4]thiazin-6-yl)urea

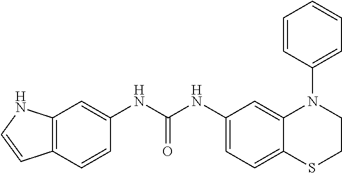 [SMILES: O=C(Nc1ccc2c(c1)N(c1ccccc1)CCS2)Nc1ccc2cc[nH]c2c1]

Example 72 was prepared according to General Procedures 1-6, 17, 25 and the methods described below.

### Preparation 47: 4-Phenyl-3,4-dihydro-2H-benzo[b][1,4]thiazin-6-amine

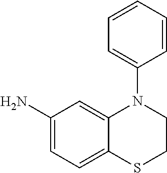 [SMILES: Nc1ccc2c(c1)N(c1ccccc1)CCS2]

### Step 1: Methyl 3,4-dihydro-2H-benzo[b][1,4]thiazine-6-carboxylate

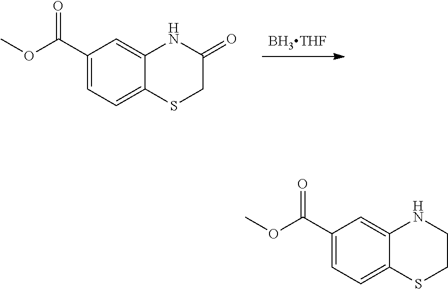

BH₃-THF (30 mL, 27 mmol) was added to methyl 3-oxo-3,4-dihydro-2H-benzo[b-1,4]thiazine-6-carboxylate (Preparation 1, Step 2) (2.0 g, 9.0 mmol) at 0-5° C. with stirring in an inert atmosphere. After the addition was complete, the mixture was brought to RT and stirred for 3 h. Completion of the reaction was confirmed by TLC and UPLC-MS. The reaction mixture was quenched by adding in portions to methanol in a conical flask and stirring until all effervescence had ceased. Then, the reaction mixture was concentrated in vacuo to give a crude material which was mixed with water and extracted with EtOAc. The organic layers were combined, washed with brine, dried over anhydrous Na₂SO₄, filtered and concentrated in vacuo to give the title compound (1.8 g) as a pale yellow crude solid. UPLC-MS m/z: 209.9 [M+H].

### Step 2: Methyl 4-phenyl-3,4-dihydro-2H-benzo[b][1,4]thiazine-6-carboxylate

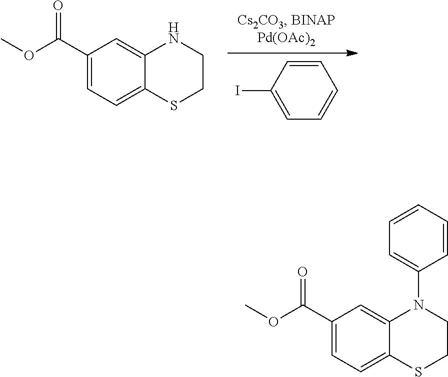

To a stirred solution of methyl 3,4-dihydro-2H-benzo[b][1,4]thiazine-6-carboxylate (Preparation 47, Step 1) (500 mg, 2.39 mmol) in toluene (15 mL) was added phenyl iodide (0.4 mL, 3.6 mmol), cesium carbonate (1.56 g, 4.78 mmol) and BINAP (298 mg, 0.48 mmol) at RT. The whole was degassed with nitrogen for 20 min., then palladium acetate (54 mg, 0.24 mmol) was added into the reaction mixture and stirring continued at 110° C. for 24 h. Progress of the reaction was monitored by UPLC-MS which showed ˜40% formation of the desired product. The reaction mixture was concentrated in vacuo to give a crude material which was purified by column chromatography to afford the title compound (240 mg, 35% yield) as a pale yellow solid along with recovered unreacted starting material. UPLC-MS m/z: 285.98 [M+H].

Step: 4-Phenyl-3,4-dihydro-2H-benzo[b][1,4]thiazin-6-amine hydrochloride

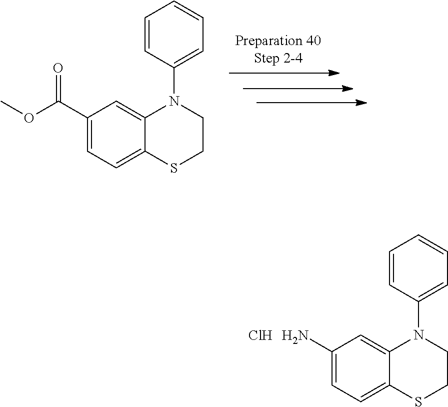 [SMILES: COC(=O)c1ccc2c(c1)N(c1ccccc1)CCS2 ; "Preparation 40Step 2-4" ; Cl ; Nc1ccc2c(c1)N(c1ccccc1)CCS2]

The title compound was prepared according to the methods described for the preparation of Example 50 (Preparation 40, Steps 2-4), starting from methyl 4-phenyl-3,4-dihydro-2H-benzo[b][1,4]thiazine-6-carboxylate (Preparation 50, Step 2), instead of methyl 4-benzyl-3,4-dihydro-2H-benzo[b][1,4]thiazine-6-carboxylate (Preparation 40, Step 1). UPLC-MS m/z: 242.96 [M+H].

### Preparation 48: 3-(1H-Indol-6-yl)-3-(4-phenyl-3,4-dihydro-2H-benzo[b][1,4]thiazin-6-yl)urea (Example 72)

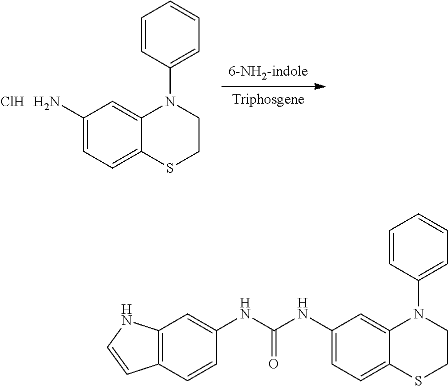 [SMILES: Cl ; Nc1ccc2c(c1)N(c1ccccc1)CCS2 ; "6-NH2-indole" ; "Triphosgene" ; O=C(Nc1ccc2c(c1)N(c1ccccc1)CCS2)Nc1ccc2cc[nH]c2c1]

To a stirred solution of 6-amino-indole (147 mg, 1.11 mmol) in THF (6 mL) was added triphosgene (157 mg, 0.53 mmol) at 0-5° C. The reaction mixture was stirred at RT for 1.5 h. 4-Phenyl-3,4-dihydro-2H-benzo[b][1,4]thiazin-6-amine (Preparation 47, Step 3) (50 mg, 0.18 mmol) was then added followed by TEA (0.77 mL, 5.5 mmol) at 0-5° C. The whole was again stirred at RT for 2 h. Progress of the reaction was monitored by UPLC-MS and after completion the reaction mixture was concentrated in vacuo to give a residue which was diluted with water and extracted with EtOAc. The organic layers were combined, washed with brine, dried over anhydrous Na₂SO₄, filtered and evaporated in vacuo to give the crude product which was purified by prep-HPLC to afford the title compound (12 mg, 17% yield) as a yellow solid. Purity by UPLC: 96.74%; ¹H NMR (400 MHz; DMSO-d₆): δ 3.10 (t, J=3.16 Hz, 2H), 3.85-3.88 (m, 2H), 6.30 (s, 1H), 6.74-6.77 (m, 1H), 6.87 (s, 1H), 6.96-7.00 (m, 2H), 7.09 (t, J=7.36 Hz, 1H), 7.19-7.20 (m, 3H), 7.35-7.39 (m, 3H), 7.71 (s, 1H), 8.43 (s, 1H), 8.50 (s, 1H), 10.86 (s, 1H); UPLC-MS m/z: 401.12 [M+H].

### Example 75: 1-(1H-Indol-6-yl)-3-(3-oxo-4-phenyl-3,4-dihydro-2H-benzo[b][1,4]oxazin-7-yl)urea

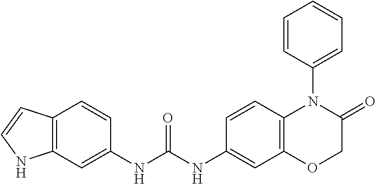 [SMILES: O=C(Nc1ccc2c(c1)OCC(=O)N2c1ccccc1)Nc1ccc2cc[nH]c2c1]

Example 75 was prepared according to General Procedures 1-6, 24 and the methods described below.

### Preparation 53: 7-Amino-4-phenyl-2H-benzo[b][1,4]oxazin-3(4H)-one

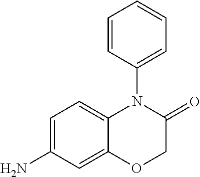 [SMILES: Nc1ccc2c(c1)OCC(=O)N2c1ccccc1]

### Step 1: Methyl 3-oxo-4-phenyl-3,4-dihydro-2H-benzo[b][1,4]oxazine-7-carboxylate

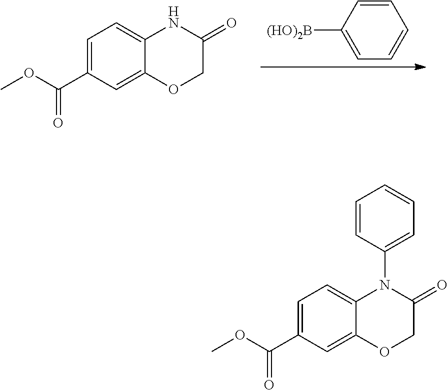 [SMILES: COC(=O)c1ccc2c(c1)OCC(=O)N2 ; OB(O)c1ccccc1 ; COC(=O)c1ccc2c(c1)OCC(=O)N2c1ccccc1]

To a stirred solution of methyl 3-oxo-3,4-dihydro-2H-benzo[b][1,4]oxazine-7-carboxylate (Preparation 44, Step 2) (800 mg, 3.86 mmol) in EDC (4 mL) was added phenylboronic acid (706 mg, 5.79 mmol) in EDC (4 mL), DBU (1.176 mL 7.72 mmol) and solution of Cu(OAc) (1.40 g, 7.72 mmol) at RT. The resulting reaction mixture was stirred at RT for 24 h. UPLC-MS showed ˜50% conversion. The reaction mixture was diluted with water and extracted with EtOAc, the organic layer was washed with brine, dried over anhydrous Na₂SO₄ and evaporated in vacuo to afford the crude material which was purified by Combi-flash (20 g column) using 35% EtOAc in hexane as eluent to afford the title compound (420 mg, 38% yield) as an off white solid.

### Step 2: 3-Oxo-4-phenyl-3,4-dihydro-2H-benzo[b][1,4]oxazine-7-carbonyl azide

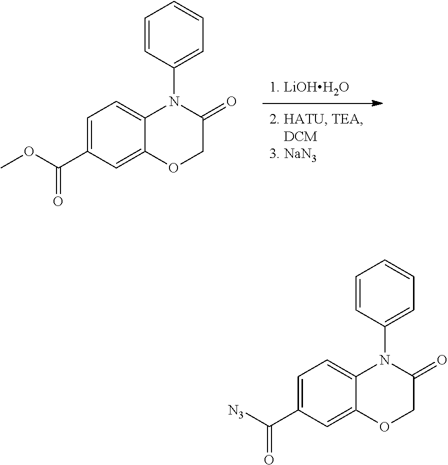 [SMILES: COC(=O)c1ccc2c(c1)OCC(=O)N2c1ccccc1 ; [N-]=[N+]=NC(=O)c1ccc2c(c1)OCC(=O)N2c1ccccc1]

To a stirred solution of methyl 3-oxo-4-phenyl-3,4-dihydro-2H-benzo[b][1,4]oxazine-7-carboxylate (Preparation 53, Step 1) (420 mg, 1.48 mmol) in THF (8 mL) and MeOH (4 mL) was added a solution of LiOH.H₂O (249 mg, 5.93 mmol) in water (4 mL) and the reaction maintained at RT for 2 h. TLC showed completion of the reaction. The solvents were evaporated in vacuo to give a crude material which was dissolved in water, washed with MTBE and the aqueous layer was acidified with 6N HCl. The neutralized aqueous mass was extracted with EtOAc. The combined organic layers were washed with brine, dried over anhydrous Na₂SO₄ and evaporated in vacuo to afford the corresponding intermediate acid (400 mg) which was then dissolved in DCM (45 mL) and HATU (845 mg, 2.22 mmol) and TEA (0.641 mL, 4.45 mmol) added at RT. The whole was stirred at RT for 24 h. UPLC-MS confirmed formation of the intermediate HATU-adduct. Sodium bicarbonate solution (10%) was added and the layers were separated. The organic layer was evaporated to afford a crude mass to which a saturated solution of sodium azide was added and the whole was stirred at RT for 30 min. UPLC-MS showed completion of the reaction, which was then diluted with water and extracted with EtOAc. The organic layer was washed with brine, dried over anhydrous Na₂SO₄ and evaporated in vacuo to afford the title compound (250 mg, crude) as pale yellow solid. UPLC-MS m/z: 194.98 [M+H].

Step 3: 7-Amino-4-phenyl-2H-benzo[b][1,4]oxazin-2(4H)-one

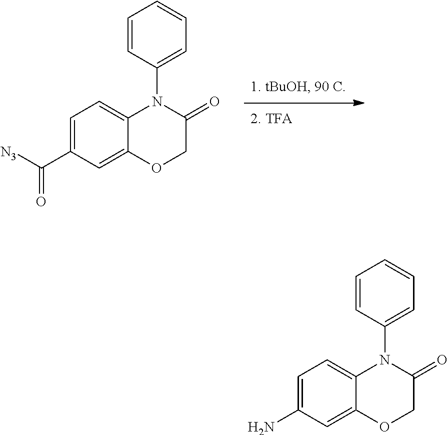 [SMILES: [N-]=[N+]=NC(=O)c1ccc2c(c1)OCC(=O)N2c1ccccc1 ; Nc1ccc2c(c1)OCC(=O)N2c1ccccc1]

To a stirred solution of 3-oxo-4-phenyl-3,4-dihydro-2H-benzo[b][1,4]oxazine-7-carbonyl azide (Preparation 53, Step 2) (0.25 g, 0.85 mmol) in tert-butanol (5 mL) was heated to 90° C. for 1 h. UPLC-MS showed formation of the desired intermediate. The solvent was evaporated to give a crude material which was purified by Combi-flash to afford the corresponding Boc-NH₂ intermediate (130 mg). The Boc-NH₂ intermediate (130 mg, 0.38 mmol) was dissolved in 20% TFA in DCM (10 mL) in an inert atmosphere at RT, and then further stirred at RT for 1 h. UPLC showed formation of the desired compound. The reaction mixture was quenched with a saturated sodium bicarbonate solution (pH ˜8) and extracted with DCM. The organic layer was washed with brine, dried over anhydrous Na₂SO₄ and evaporated in vacuo to afford the title compound (90 mg, crude) as an off white solid. UPLC-MS m/z: 241.3 [M+H].

### Preparation 54: 1-(1H-Indol-6-yl-3-(3-oxo-4-phenyl-3,4-dihydro-2H-benzo[b][1,4]oxazin-7-yl)urea (Example 75)

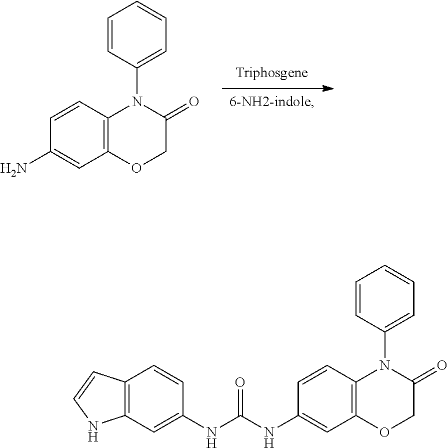 [SMILES: Nc1ccc2c(c1)OCC(=O)N2c1ccccc1 ; "6-NH2-indole," ; "Triphosgene" ; O=C(Nc1ccc2c(c1)OCC(=O)N2c1ccccc1)Nc1ccc2cc[nH]c2c1]

To a stirred solution of 6-amino-indole (54 mg, 0.411 mmol) in THF (5 mL) was cooled to 0-5° C. followed by addition of triphosgene (55 mg, 0.187 mmol) and maintained at RT for 1 h, then added 7-amino-4-phenyl-2H-benzo[b][1,4]oxazin-3(4H)-one 1 (Preparation 53, Step 3) (90 mg, 0.374 mmol) and TEA (0.178 mL, 1.23 mmol) at RT. The resulting reaction mixture was stirred at RT for 1 h. TLC showed completion of reaction, then the reaction mixture was diluted with EtOAc and washed with water followed by 1N HCl and finally with brine. The organic layer was dried over anhydrous Na₂SO₄ and evaporated in vacuo to afford the crude material which was purified by prep-HPLC to give the title compound (38 mg, 25.5% yield) as an off white solid. Purity by UPLC: 99.18%; ¹H NMR (400 MHz; DMSO-d₆): δ 4.80 (s, 2H), 6.22 (d, 1H, J=8.64), 6.33 (s, 1H), 6.85 (t, 2H, J=9.04 Hz) 7.21 (s, 1H), 7.60-7.33 (m, 7H), 7.79 (s, 1H), 8.62 (s, 1H), 8.76 (s, 1H), 10.91 (s, 1H); UPLC-MS m/z: 398.99 [M+H].

### Examples 175, 181-185

The examples in the table below were prepared according to the above methods used to make Examples 72 and 75 as described in General Procedures 1-6, 17, 18 and 24-25 using the appropriate amine. Purification was as stated in the aforementioned methods.

| | | IUPAC | | LCMS | Purity |
|---|---|---|---|---|---|
| Ex | Structure | Name | 1H-NMR | [M + H] | (%) |
| 175 | 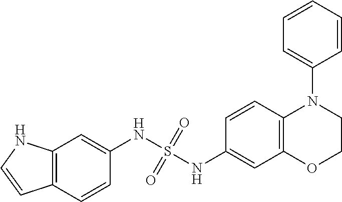 [SMILES: O=S(=O)(Nc1ccc2c(c1)OCCN2c1ccccc1)Nc1ccc2cc[nH]c2c1] | N-1H-indol- 6-yl-N′-(4- phenyl-3,4- dihydro-2H- 1,4- benzoxazin- 7-yl)sulfuric diamide | (400 MHz; DMSO- d₆): δ 3.64 (t, 2H, J = 3.96 Hz), 4.17 (t, 2H, J = 4 Hz ), 6.34 (s, 1H), 6.50-6.53 (dd, 1H, 2.4, 8.68 Hz), 6.72 (d, 1H, 2.44 Hz), 6.74 (s, 1H), 6.83 (dd, 1H, J = 1.76 Hz, 8.48 Hz), 7.017 (s, 1H, 7.4 Hz), 7.14-7.40 (m, 7 H), 9.16 (s, 1H), 9.70 (s, | 421.2 | 98.98 |
| | | | 1H), 10.97 (s, 1H). | | |
| 181 | 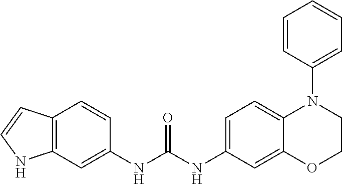 [SMILES: O=C(Nc1ccc2c(c1)OCCN2c1ccccc1)Nc1ccc2cc[nH]c2c1] | 1-(1H-indol- 6-yl)-3-(4- phenyl-3,4- dihydro-2H- benzo[b][1,4] oxazin-7- yl)urea | (400 MHz; DMSO- d₆): δ 3.67 (t, 2H, J = 4.16 Hz), 4.20 (t, 2H, J = 4.2 Hz), 6.33 (s, 1H), 6.84 - 6.74 (m, 3H) 7.03 (t, 1H, J = 14.48 Hz), 7.21-7.14 (m, 4H), 7.41-7.32 (m, 3H) 7.79 (s, 1H), 8.40 (s, 1H), 8.45 (s, 1H), 10.90 (s, 1H). | 385.1 | 99.07 |
| 182 | 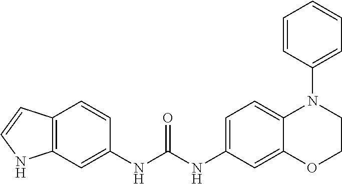 [SMILES: O=C(Nc1ccc2c(c1)OCCN2c1ccccc1)Nc1ccc2cc[nH]c2c1] | 1-(1H-indol- 6-yl)-3-(4- phenyl-3,4- dihydro-2H- benzo[b][1,4] thiazin-7- yl)urea | (400 MHz; DMSO- d₆): δ 3.67 (t, 2H, J = 4.92 Hz), 3.87- 3.85 (m, 2H), 6.78 (d, 1H, J = 8.8 Hz), 6.86-6.83 (m, 1H) 7.31-6.91 (m, 10H), 7.78 (s, 1H), 8.51 (s, 1H), 8.55 (s, 1H), 10.90 (s, 1H). | 401.11 | 99.08 |
| 183 | 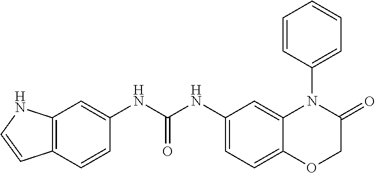 [SMILES: O=C(Nc1ccc2c(c1)N(c1ccccc1)C(=O)CO2)Nc1ccc2cc[nH]c2c1] | 1-(1H-indol- 6-yl)-3-(3- oxo-4- phenyl-3,4- dihydro-2H- benzo[b][1,4] oxazin-6- yl)urea | (500 MHz; DMSO- d₆): δ 4.77 (s, 2H), 6.31 (s, 1H), 6.55 (s, 1 H), 6.86 -6.79 (m, 3 H) 7.48-7.12 (m, 9 H), 7.79 (s, 1H), 8.51 (s, 1H), 10.92 (s, 1H). | 399.1 | 95.78 |
| 184 | 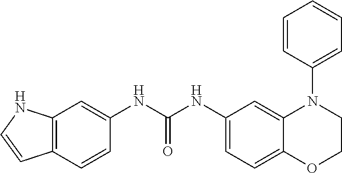 [SMILES: O=C(Nc1ccc2c(c1)N(c1ccccc1)CCO2)Nc1ccc2cc[nH]c2c1] | 1-(1H-indol- 6-yl)-3-(4- phenyl-3,4- dihydro-2H- benzo[b][1,4] oxazin-6- yl)urea | (500 MHz; DMSO- d₆): δ 3.75 (s, 2 H), 4.27 (s, 2H), 6.37 (s, 1H), 6.86-6.79 (m, 3H) 7.48-7.12 (m, 9H), 7.79 (s, 1H), 8.51 (s, 1H), 10.92 (s, 1H). | 385.13 | 98.3 |
| 185 | 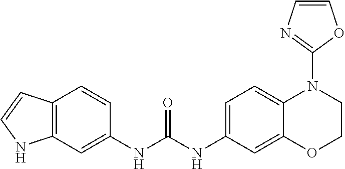 [SMILES: O=C(Nc1ccc2c(c1)OCCN2c1ncco1)Nc1ccc2cc[nH]c2c1] | 1-(1H-indol- 6-yl)-3-(4- (oxazol-2-yl)- 3,4-dihydro- 2H- benzo[b][1,4] oxazin-7- yl)urea | | 376.10 | 91.1 |

### Example 73: 1-(4-Benzyl-1-methyl-1,2,3,4-tetrahydroquinoxalin-6-yl)-3-(1H-indol-6-yl)urea

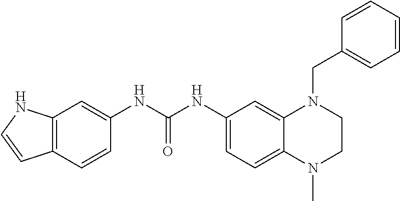 [SMILES: CN1CCN(Cc2ccccc2)c2cc(NC(=O)Nc3ccc4cc[nH]c4c3)ccc21]

Example 73 was prepared according to General Procedures 4-5, 6d, 8, 17 and the methods described below.

### Preparation 49: 4-Benzyl-1-methyl-1,2,3,4-tetrahydroquinoxalin-6-amine

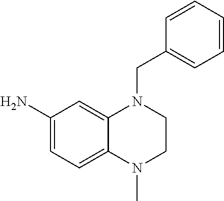 [SMILES: CN1CCN(Cc2ccccc2)c2cc(N)ccc21]

### Step 1: 2-Chloro-N-(2-fluoro-5-nitrophenyl)acetamide

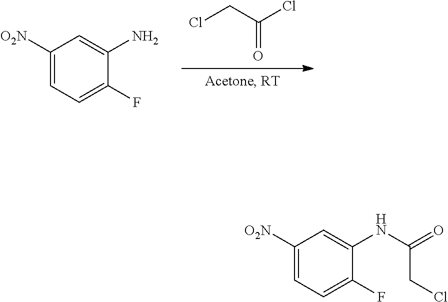

To a stirred solution of commercially available 2-fluoro-5-nitroaniline (1.5 g, 9.61 mmol) in acetone (30 mL) was added chloroacetyl chloride (0.994 mL, 12.49 mmol) at RT and then the reaction mixture was stirred at RT for 1 h. TLC and UPLC-MS showed completion of the reaction. Thereafter ice-cold water was added to the reaction mixture to give a solid precipitate which was filtered, washed with water and then dried in an oven to afford the title compound (2.0 g, crude) as a brownish solid. UPLC-MS m/z: 231.3 [M−H].

### Step 2: 4-Methyl-7-nitro-3,4-dihydroquinoxalin-2(1H)-one

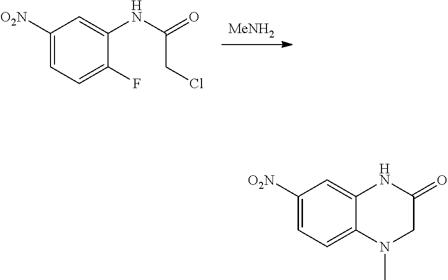

To a stirred solution of 2-chloro-N-(2-fluoro-5-nitrophenyl)acetamide (Preparation 49, Step 1) (2.0 g, 1.07 mmol) in ethanol (5 mL) was added a solution of methyl amine in THF (25.79 mL, 2M solution) at RT and the whole stirred at 90° C. for 16 h. TLC and UPLC-MS showed completion of reaction. Thereafter the solvent was evaporated in vacuo to give a crude product which was purified by Combi-flash (20 g column) using EtOAC as eluent to afford the title compound (1.3 g, 73% yield) as a yellow solid. UPLC-MS m/z: 206 [M−H].

### Step 3: 1-Benzyl-4-methyl-7-nitro-3,4-dihydroquinoxalin-2(1H)-one

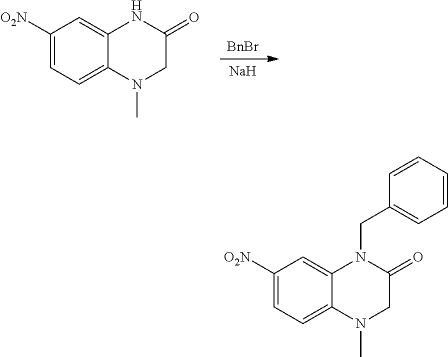

To a stirred solution of 4-methyl-7-nitro-3,4-dihydroquinoxalin-2(1H)-one (Preparation 49, Step 2) (1.0 g, 4.83 mmol) in DMF (15 mL) was added NaH (212 mg, 5.31 mmol) at 0-10° C. followed by benzyl bromide (0.64 mL, 5.31 mmol) and the reaction mixture was allowed to warm slowly to RT over 8 h. TLC and UPLC-MS showed formation of the desired product along with a di-benzylated compound. The reaction mixture was diluted with chilled water and extracted with EtOAc. The combined organic layers were washed with brine, dried over anhydrous Na₂SO₄, filtered and evaporated in vacuo to afford a crude product which was purified by Combi-flash (20 g column). The title compound (400 mg, 28% yield) was eluted with 50% EtOAc in hexane as a pale brown solid and the undesired di-benzylated compound (C and N-benzylated product) was eluted with 35% EtOAc in hexane as a brownish solid. UPLC-MS m/z: 298.88 [M+H].

### Step 4: 4-Benzyl-1-methyl-6-nitro-1,2,3,4-tetrahydroquinoxaline

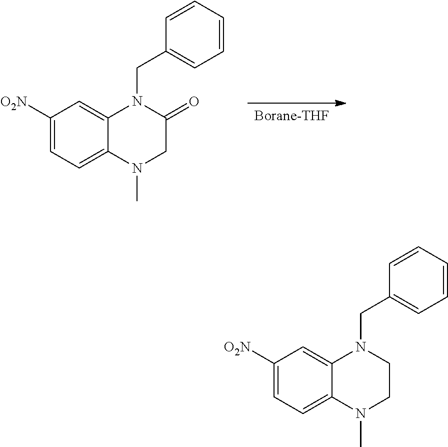 [SMILES: CN1CC(=O)N(Cc2ccccc2)c2cc([N+](=O)[O-])ccc21 ; "Borane-THF" ; CN1CCN(Cc2ccccc2)c2cc([N+](=O)[O-])ccc21]

Borane-THF complex (2.02 mL, 2.024 mmol, 1M solution in THF) was added portionwise to 1-benzyl-4-methyl-7-nitro-3,4-dihydroquinoxalin-2(1H)-one (Preparation 49, Step 3) (200 mg, 0.67 mmol) with stirring at 5-10° C. After the addition was completed, the combined mixture was stirred at RT for 1 h. UPLC-MS showed formation of the desired compound. The reaction mixture was diluted with MeOH (5 mL) and further stirred at RT for 10 min. to quench any excess borane. The solvents were evaporated in vacuo to give a residue which was diluted with water and extracted with EtOAc. The combined organic layers was washed with brine, dried over anhydrous Na₂SO₄, filtered and evaporated under reduced pressure to afford the crude product which was purified by Combi-flash (12 g column) using 30% EtOAc in hexane as eluent to afford the title compound (150 mg, 79% yield) as an orange solid. UPLC-MS m/z: 284.3 [M+H].

### Step 5: 4-Benzyl-1-methyl-1,2,3,4-tetrahydroquinoxalin-6-amine

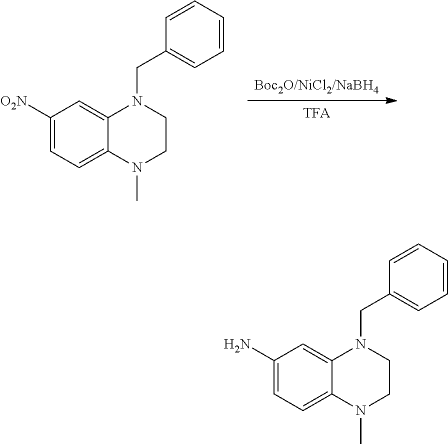 [SMILES: CN1CCN(Cc2ccccc2)c2cc([N+](=O)[O-])ccc21 ; "Boc2O/NiCl2/NaBH4" ; CN1CCN(Cc2ccccc2)c2cc(N)ccc21 ; "TFA"]

To a stirred solution of 4-benzyl-1-methyl-6-nitro-1,2,3,4-tetrahydroquinoxaline (Preparation 49, Step 4) (150 mg, 0.53 mmol) in MeOH (5 mL) was added Boc₂O (0.173 mL, 0.79 mmol) followed by NiCl₂.6H₂O (63 mg, 0.26 mmol) and NaBH₄ (50 mg, 1.32 mmol) at 5-10° C. The combined mixture was then allowed to warm to RT over 5 h. Progress of the reaction was monitored by TLC and UPLC-MS which showed formation of the intermediate product. After completion, the reaction mixture was diluted with chilled water and extracted with EtOAc. The combined organic layers were washed with brine, dried over anhydrous Na₂SO₄, filtered and evaporated under reduced pressure to afford the crude product which was purified by Combi-flash (12 g column) using 35% EtOAc in hexane as eluent to provide the Boc-protected amine compound (180 mg, 96% yield). This material was dissolved in DCM (5 mL) and TFA (2 mL) and the whole was stirred at RT for 4 h. UPLC-MS showed formation of the desired product. The solvent was evaporated in vacuo to give the crude product which was neutralized with aqueous sodium carbonate solution and extracted with EtOAc. The combined extracts were washed with brine, dried over anhydrous Na₂SO₄, filtered and evaporated under reduced pressure to afford the title compound (110 mg, crude) as a brown semi-solid which was used in the next step without any further purification. UPLC-MS m/z: 254.23 [M+H].

### Preparation so: 1-(4-Benzyl-1-methyl-1,2,3,4-tetrahydroquinoxalin-6-yl-3-(1H-indol-6-yl)urea (Example 73)

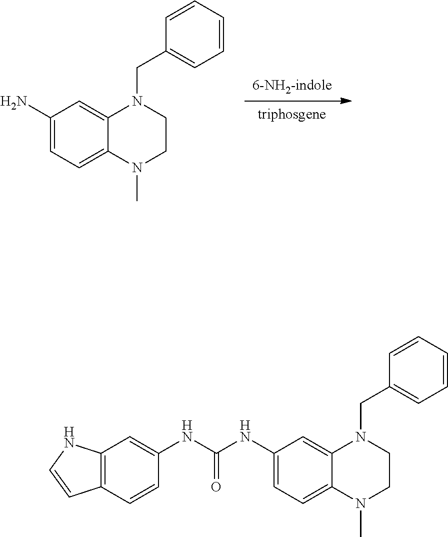 [SMILES: CN1CCN(Cc2ccccc2)c2cc(N)ccc21 ; "6-NH2-indole" ; "triphosgene" ; CN1CCN(Cc2ccccc2)c2cc(NC(=O)Nc3ccc4cc[nH]c4c3)ccc21]

To a stirred solution of 6-amino-indole (63 mg, 0.477 mmol) in THF (5 mL) at RT was added triphosgene (64 mg, 0.217 mmol). The mixture was stirred for 1 h, then 4-benzyl-1-methyl-1,2,3,4-tetrahydroquinoxalin-6-amine (Preparation 49, Step 5) (110 mg, 0.434 mmol) and TEA (0.206 mL, 1.432 mmol) were added to the reaction mixture and the whole stirred at RT for 1 h. TLC showed complete consumption of the amine and a new polar spot was observed. The solvent was evaporated in vacuo to give a residue which was diluted with water and extracted with EtOAc. The combined organic layers were washed with brine, dried over anhydrous Na₂SO₄, filtered and evaporated under reduced pressure to give the crude product which was purified by prep-HPLC to afford the title compound (40 mg, 22% yield) as a greenish solid. Purity by UPLC: 97.7%; ¹H NMR (400 MHz; DMSO-d₆): δ 2.75 (s, 3H), 3.16 (t, J=4.96 Hz, 2H), 3.49 (t, J=4.52 Hz, 2H), 4.45 (s, 2H), 6.30 (s, 1H), 6.42 (d, J=8.48 Hz, 1H), 6.53 (d, J=2.24 Hz, 1H), 6.65-6.67 (m, 1H), 6.73-6.76 (m, 1H), 7.17 (d, J=2.48 Hz, 1H), 7.19-7.37 (m, 6H), 7.75 (s, 1H), 8.05 (s, 1H), 8.30 (s, 1H), 10.84 (s, 1H); UPLC-MS m/z: 410.21 [M−H].

### Example 74: 1-(1-Benzylindolin-6-yl)-3-(1H-indol-6-yl)urea

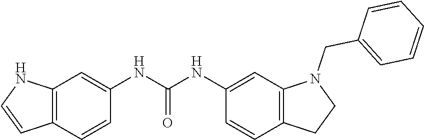 [SMILES: O=C(Nc1ccc2c(c1)N(Cc1ccccc1)CC2)Nc1ccc2cc[nH]c2c1]

Example 74 was prepared according to General Procedures 1-6 and the methods described below.

### Preparation 51: 1-Benzylindolin-6-amine hydrochloride

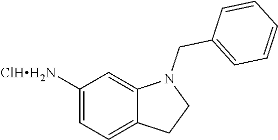

### Step 1: Methyl 1-benzylindoline-6-carboxylate

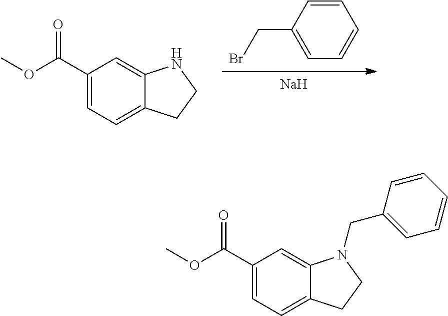

To a stirred solution of commercially available methyl indoline-6-carboxylate (50 mg, 0.28 mmol) in DMF (1 mL) was added NaH (12.4 mg, 0.31 mmol) at 0-5° C. under an inert atmosphere. After 15 min., benzyl bromide (0.035 ml, 0.3 mmol) was added to the reaction mixture and stirring was continued at RT for 2 h. Completion of the reaction was confirmed by UPLC-MS. The reaction mixture was diluted with water (20 mL) and extracted with MTBE. The combined organic layers were washed with brine, dried over anhydrous Na₂SO₄ and concentrated in vacuo to give the title compound (72 mg) as a crude yellow solid which was used in the next step without any further purification. UPLC-MS m/z: 268 [M+H].

### Step 2: 1-Benzylindolin-6-amine hydrochloride

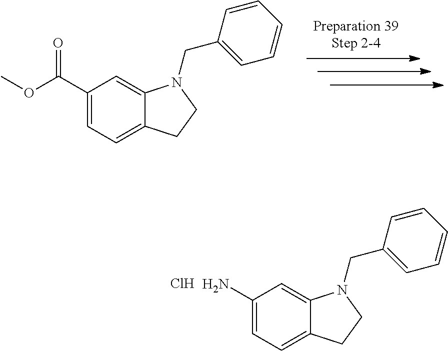 [SMILES: COC(=O)c1ccc2c(c1)N(Cc1ccccc1)CC2 ; "Preparation 39Step 2-4" ; Cl ; Nc1ccc2c(c1)N(Cc1ccccc1)CC2]

The title compound was prepared according to the methods described for the preparation of Example 50 (Preparation 39, Steps 2-4), starting from methyl 1-benzylindoline-6-carboxylate (Preparation 54, Step 1), instead of methyl 4-benzyl-3,4-dihydro-2H-benzo[b][1,4]thiazine-6-carboxylate (Preparation 40, Step 1). UPLC-MS m/z: 225 [M+H].

### Preparation 52: 1-(1-Benzylindolin-6-yl)-3-(1H-indol-6-yl)urea (Example 74)

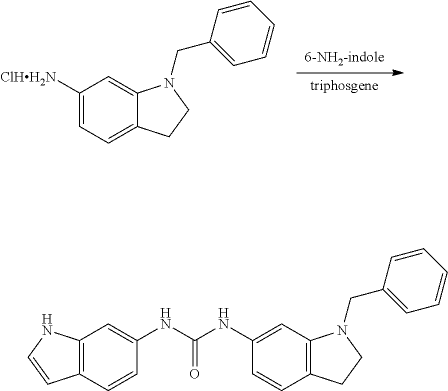

To a stirred solution of 1-benzylindolin-6-amine hydrochloride (Preparation 51, Step 2) (30 mg, 0.12 mmol) in THF (3 mL) was added TEA (0.016 ml, 0.12 mmol) at RT. After the addition was completed, the mixture was stirred at RT for 30 min. Triphosgene (13.66 mg, 0.05 mmol) was added and stirring was continued at RT for 1 h. 6-NH₂-indole (22.8 mg, 0.17 mmol) and TEA (0.032 ml, 0.24 mmol) were then added and the whole stirred at RT overnight. Progress of the reaction was monitored by UPLC-MS and after completion the mixture was evaporated in vacuo to give the crude product which was purified by prep-HPLC to afford the title compound (3 mg, 7% yield) as a white solid. Purity by UPLC: 96.11%; ¹H NMR (400 MHz; DMSO-d₆): δ 2.84 (t, J=8.16 Hz, 2H), 3.26-3.28 (m, 2H), 4.25 (s, 2H), 6.31 (s, 1H), 6.62-6.65 (m, 1H), 6.80-6.82 (m, 2H), 6.91-6.93 (m, 1H), 7.20 (t, J=2.64 Hz, 1H), 7.26-7.39 (m, 6H), 7.78 (s, 1H), 8.48 (s, 1H) 8.57 (s, 1H), 10.88 (s, 1H); UPLC-MS m/z: 383.11 [M+H].

### Example 76: 2-(6-(3-(1H-Indol-6-yl)ureido)-4-benzyl-3-oxo-3,4-dihydro-2H-benzo[b][1,4]thiazin-2-yl)acetamide

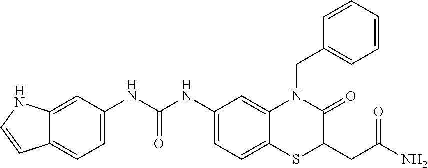 [SMILES: NC(=O)CC1Sc2ccc(NC(=O)Nc3ccc4cc[nH]c4c3)cc2N(Cc2ccccc2)C1=O]

Example 76 was prepared according to General Procedures 1-6, 26 and the methods described below.

### Preparation 55: 4-Benzyl-2-(cyanomethyl)-3-oxo-3,4-dihydro-2H-benzo[b][1,4]thiazine-6-carboxylic acid

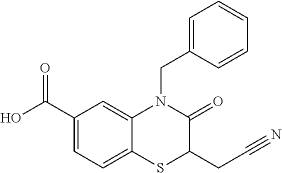 [SMILES: N#CCC1Sc2ccc(C(=O)O)cc2N(Cc2ccccc2)C1=O]

### Step 1: Methyl 4-benzyl-2-(cyanomethyl)-3-oxo-3,4-dihydro-2H-benzo[b][1,4]thiazine-6-carboxylate

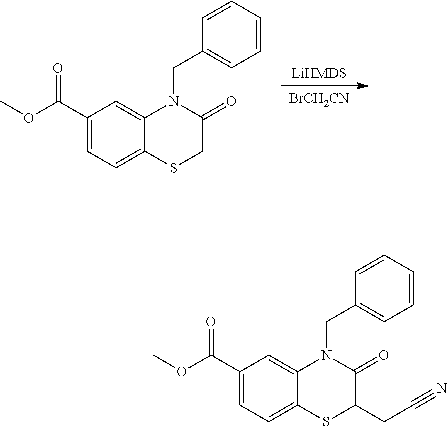

To a stirred solution of methyl 4-benzyl-3-oxo-3,4-dihydro-2H-benzo[b][1,4]thiazine-6-carboxylate (Preparation 2) (1.0 g, 3.2 mmol) in dry THF (20 mL) was added LiHMDS (3.6 mL, 4.8 mmol) at −78° C. under inert atmosphere and stirred for 5 min. then, bromoacetonitrile (270 μL, 3.85 mmol) was added to the reaction mixture and stirring continued for 30 min. at the same temperature. After this time the reaction mixture was brought to room temperature and stirred for 1 h. Completion of the reaction was monitored by TLC and UPLC-MS, after which the reaction mass was quenched with a saturated solution of ammonium chloride and extracted with EtOAc followed by a brine wash. The organic layer was dried over anhydrous Na₂SO₄, filtered and concentrated under reduced pressure to afford a crude viscous oil which was purified by Combi-flash on a 20 g column by eluting with 30% EtOAc/hexane as an eluent to afford the title compound (550 mg, 48% yield) as a pale yellow solid. UPLC-MS m/z: 353 [M+H].

### Step 2: 4-Benzyl-2-(cyanomethyl)-3-oxo-3,4-dihydro-2H-benzo[b][1,4]thiazine-6-carboxylic acid

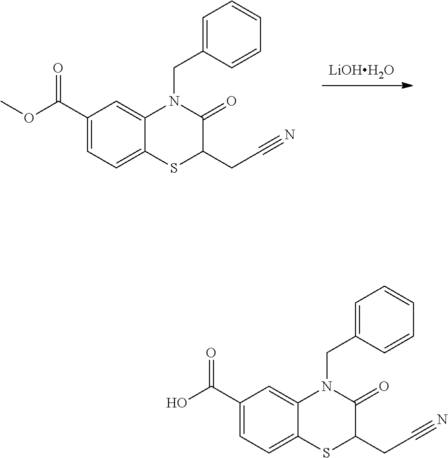 [SMILES: COC(=O)c1ccc2c(c1)N(Cc1ccccc1)C(=O)C(CC#N)S2 ; "LiOH·H2O" ; N#CCC1Sc2ccc(C(=O)O)cc2N(Cc2ccccc2)C1=O]

To a stirred solution of methyl 4-benzyl-2-(cyanomethyl)-3-oxo-3,4-dihydro-2H-benzo[b][1,4]thiazine-6-carboxylate (Preparation 55, Step 1) (0.6 g, 1.7 mmol) in a mixture of THF:MeOH:H₂O (12 mL, 2:1:1) was added LiOH.H₂O (0.29 g, 6.8 mmol) at RT and stirred for 2 h at the same temperature. When TLC and UPLC-MS showed complete consumption of the starting material with formation of the desired hydrolysed product, the solvents were evaporated under reduced pressure. The resulting residue was diluted with water and washed with MTBE. The aqueous layer was collected and acidified with 1N HCl to pH 5-6, then extracted with EtOAc and the organic layer was separated, washed with brine, dried over anhydrous Na₂SO₄, filtered and concentrated under reduced pressure to afford the title compound (550 mg, crude) as a pale yellow solid which was used in the next step without any further purification.

UPLC-MS m/z: 337 [M−H].

### Preparation 56: 1-(4-Benzyl-2-(cyanomethyl)-3-oxo-3,4-dihydro-2H-benzo[b][1,4]thiazin-6-yl-3-(1H-indol-6-yl)urea (Example 77)

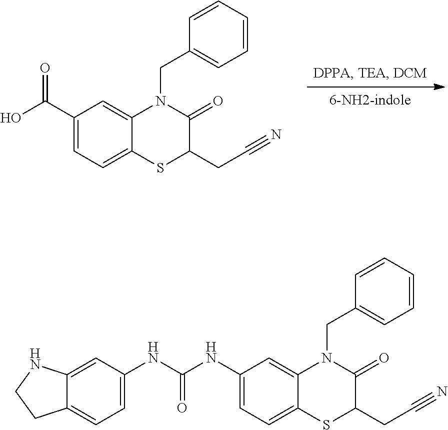 [SMILES: N#CCC1Sc2ccc(C(=O)O)cc2N(Cc2ccccc2)C1=O ; "6-NH2-indole" ; "DPPA, TEA, DCM" ; N#CCC1Sc2ccc(NC(=O)Nc3ccc4c(c3)NCC4)cc2N(Cc2ccccc2)C1=O]

To a stirred solution of 4-benzyl-2-(cyanomethyl)-3-oxo-3,4-dihydro-2H-benzo[b][1,4]thiazine-6-carboxylic acid (Preparation 55, Step 2) (0.10 g, 0.30 mmol) in DCM (5 mL) was added TEA (0.065 mL, 0.45 mmol) at 0-5° C. under an inert atmosphere followed by DPPA (0.095 mL, 0.45 mmol) and stirring continued for 5 min. at the same temperature. Then, the reaction mixture was brought slowly to RT and stirred overnight. Formation of the intermediate acyl azide was confirmed by TLC and UPLC-MS. Then, the reaction mixture was concentrated and toluene (5 mL) added followed by 6-amino-indole (60 mg, 0.45 mmol) and the whole was refluxed for 3 h. Completion of the reaction was confirmed by TLC and UPLC-MS, after which the solvents were removed on a rotary evaporator to give a crude material which was purified by prep-HPLC to afford the title compound (40 mg, 28% yield) as a black solid. Purity by UPLC: 97.92%; ¹H NMR (400 MHz; DMSO-d₆): δ 1.66-1.75 (m, 2H), 3.47-3.57 (m, 3H), 3.95-3.98 (dd, 1H, J1=1.88 Hz, J2=10.68 Hz), 4.17-4.20 (dd, 1H, J1=1.28 Hz, J2=10.68 Hz), 4.42-4.51 (m, 3H), 6.29 (s, 1H), 6.60-6.65 (m, 3H), 6.74-6.77 (dd, 1H, J1=1.68 Hz, J2=8.48 Hz), 7.18 (t, 1H, J=2.52 Hz), 7.24-7.26 (m, 1H), 7.30-7.36 (m, 5H), 7.73 (s, 1H), 8.19 (s, 1H), 8.38 (s, 1H), 10.84 (s, 1H); UPLC-MS m/z: 468.15 [M+H].

### Preparation 57: 2-(6-(3-(1H-Indol-6-yl)ureido)-4-benzyl-3-oxo-3,4-dihydro-2H-benzo[b][1,4]thiazin-2-yl)acetamide (Example 76)

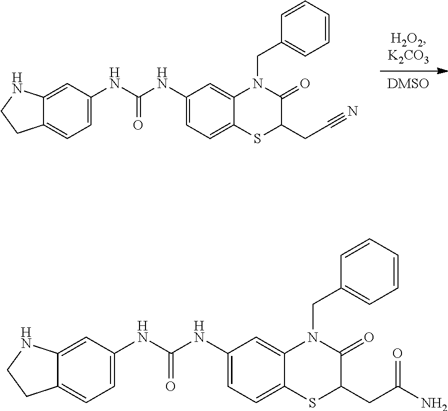

To a stirred solution of 1-(4-benzyl-2-(cyanomethyl)-3-oxo-3,4-dihydro-2H-benzo[b][1,4]thiazin-6-yl)-3-(1H-indol-6-yl)urea (Example 77) (100 mg, 0.21 mmol) in DMSO (1 mL) was added potassium carbonate (150 mg, 1.05 mmol) followed by hydrogen peroxide solution (1.5 mL) at RT and the combined mixture stirred for 1 h. Completion of the reaction was monitored by TLC and UPLC-MS. After completion of the reaction, the mixture was quenched with a saturated solution of sodium bisulphite and extracted with EtOAc followed by a brine wash. The separated organic layer was dried over anhydrous Na₂SO₄, filtered and concentrated under reduced pressure to give the crude product which was purified by prep-HPLC to afford the title compound (8 mg, 8% yield) as a pale yellow solid. Purity by UPLC: 95.05%; ¹H NMR (400 MHz; DMSO-d₆): δ 2.36-2.42 (m, 1H), 2.73-2.78 (m, 1H), 3.91-3.95 (s, 1H), 4.51-4.23 (m, 1H), 6.31 (s, 1H), 6.62-6.84 (m, 1H), 6.98 (s, 2H), 7.18-7.24 (m, 5H), 7.27-7.37 (m, 3H), 7.39-7.43 (m, 1H), 7.44-7.45 (m, 2H), 7.76 (s, 1H), 8.88 (s, 1H), 9.03 (s, 1H), 10.89 (s, 1H); UPLC-MS m/z: 484.15 [M−H]

### Example 78: 1-(3-Allyl-4-benzyl-3,4-dihydro-2H-benzo[b][1,4]oxazin-6-yl)-3-(1H-indol-6-yl)urea

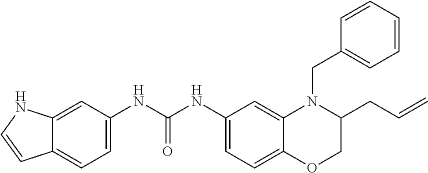 [SMILES: C=CCC1COc2ccc(NC(=O)Nc3ccc4cc[nH]c4c3)cc2N1Cc1ccccc1]

Example 78 was prepared according to General Procedures 1, 4, 6, 20-21 and the methods described below.

### Preparation 58: 3-Allyl-4-benzyl-3,4-dihydro-2H-benzo[b][1,4]oxazin-6-amine

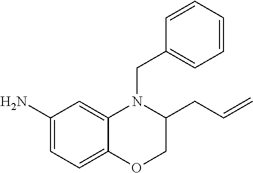 [SMILES: C=CCC1COc2ccc(N)cc2N1Cc1ccccc1]

### Step 1: 4-Benzyl-6-nitro-3-((trimethylsilyl)oxy)-3,4-dihydro-2H-benzo[b][1,4]oxazine

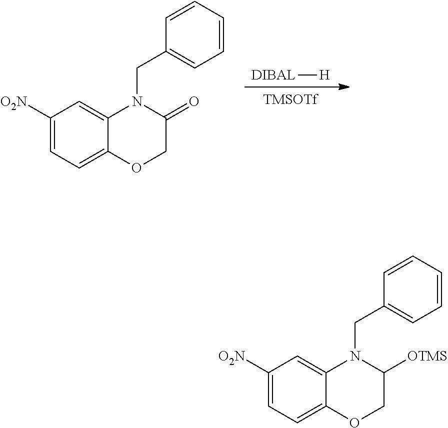

To a stirred solution of 4-benzyl-6-nitro-2H-benzo[b][1,4]oxazin-3(4H)-one (synthesized according to the method described in Preparation 2 from commercially available 6-nitro-2H-benzo[b][1,4]oxazin-3(4H)-one) (200 mg, 0.7 mmol) in DCM (7 mL) was added DIBAL-H (1 mL, 1.06 mmol) at −78° C. under a nitrogen atmosphere. The combined mixture was stirred for 2 h at the same temperature and then pyridine (0.33 mL, 2.46 mmol) and TMSOTf (0.38 mL, 2.11 mmol) were added to the reaction mixture. The temperature of the reaction was then slowly allowed to rise to 0-5° C. Progress of the reaction was monitored by TLC and after completion of the reaction, Et₂O (200 mL) was added and the mixture was filtered. The separated organic layer was then concentrated in vacuo to afford the title compound (240 mg, crude) as a yellow solid which was used in the next step without any further purification.

### Step 2: 3-Allyl-4-benzyl-6-nitro-3,4-dihydro-2H-benzo[b][1,4]oxazine

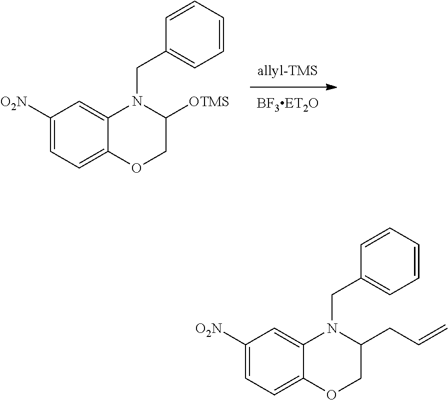

To a stirred solution of 4-benzyl-6-nitro-3-((trimethylsilyl)oxy)-3,4-dihydro-2H-benzo[b][1,4]oxazine (Preparation 58, Step 1) (240 mg, 0.67 mmol) in DCM (7 mL) was added allyl-TMS (0.42 mL, 2.68 mmol) and BF3.Et₂O (0.55 mL, 2.68 mmol) at −78° C. under nitrogen. The temperature was then slowly raised to 0-5° C. Progress of the reaction was checked by UPLC-MS and after completion the reaction was quenched with water (50 mL) and extracted with EtOAc. The separated organic layer was collected, dried over anhydrous Na₂SO₄, filtered and evaporated to dryness. The crude product was purified by column chromatography to afford the title compound (160 mg, 73% yield) as a yellow solid. UPLC-MS m/z: 311 [M+H].

### Step 3: 3-Allyl-4-benzyl-3,4-dihydro-2H-benzo[b][1,4]oxazin-6-amine

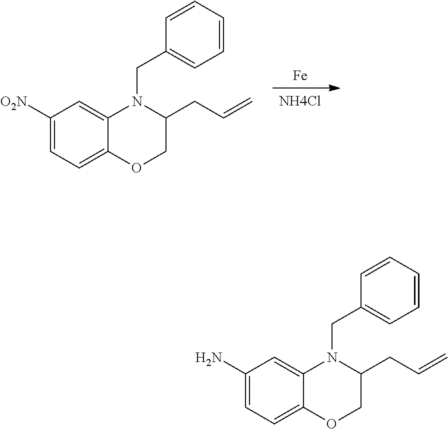

To a stirred solution of 3-allyl-4-benzyl-6-nitro-3,4-dihydro-2H-benzo[b][1,4]oxazine (Preparation 58, Step 2) (110 mg, 0.35 mmol) in EtOH (4 mL) was added Fe-powder (197.9 mg, 3-54 mmol) and NH₄Cl (4 mL) at RT. It was then heated to 90° C. for 1 h. Progress of the reaction was monitored by UPLC-MS. After completion of the reaction it was diluted with water and extracted with EtOAc. The separated organic layer was collected and filtered over a silica gel bed. The filtrate was collected, dried over anhydrous Na₂SO₄ and concentrated in vacuo to afford the title compound (150 mg, crude). The crude obtained was taken on to the next step. UPLC-MS m/z: 281 [M+H].

### Preparation 59: 1-(3-Allyl-4-benzyl-3,4-dihydro-2H-benzo[b][1,4]oxazin-6-yl)-3-(1H-indol-6-yl)urea (Example 78)

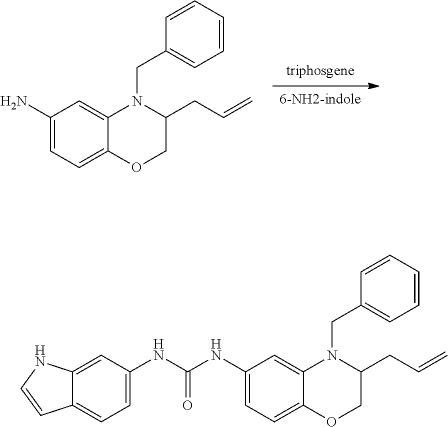 [SMILES: C=CCC1COc2ccc(N)cc2N1Cc1ccccc1 ; "6-NH2-indole" ; "triphosgene" ; C=CCC1COc2ccc(NC(=O)Nc3ccc4cc[nH]c4c3)cc2N1Cc1ccccc1]

To a stirred solution of 6-amino-indole (84.88 mg, 0.64 mmol) in THF (4 mL) was added triphosgene (55-58 mg, 0.19 mmol) at 0-5° C. under nitrogen. The stirring was continued at RT for 1 h, then 3-allyl-4-benzyl-3,4-dihydro-2H-benzo[b][1,4]oxazin-6-amine (Preparation 58, Step 3) (150 mg, 0.54 mmol) and TEA (0.18 mL, 1.34 mmol) were added and the combined mixture was further stirred at RT for 2 h. Completion of the reaction was confirmed by UPLC-MS and after completion the solvent was evaporated and the resulting residue was diluted with water and extracted with EtOAc. The organic layer was dried and concentrated in vacuo to give a crude material which was purified by Combi-flash followed by prep-HPLC to afford the title compound (27.2 mg, 76% yield) as a yellow solid. Purity by UPLC: 98.59%; ¹H NMR (400 MHz; DMSO-d₆): δ 2.31-2.38 (m, 1H), 2.36-2.39 (m, 1H), 3.50 (s, 1H), 3.96 (d, 1H, J=9.6 Hz), 4.15 (d, 1H, J=10.5 Hz), 4.52 (s, 2H), 5.09 (m, 2H), 5.86 (m, 1H), 6.31 (s, 1H), 6.65 (d, 3H), 6.76 (d, 1H, J=8.36 Hz), 7.19-7.37 (m, 7H), 7.75 (s, 1H), 8.23 (s, 1H), 8.41 (s, 1H), 10.87 (s, 1H); UPLC-MS m/z: 439 [M+H].

### Example 79: 1-(4-Benzyl-3-(2,3-dihydroxypropyl)-3,4-dihydro-2H-benzo[b][1,4]oxazin-6-yl)-3-(1H-indol-6-yl)urea

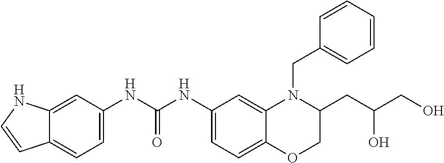 [SMILES: O=C(Nc1ccc2c(c1)N(Cc1ccccc1)C(CC(O)CO)CO2)Nc1ccc2cc[nH]c2c1]

Example 79 was prepared according to General Procedures 1, 4, 6, 20-22 and the methods described below.

### Preparation 60: 3-(4-Benzyl-6-nitro-3,4-dihydro-2H-benzo[b][1,4]oxazin-3-yl)propane-1,2-diol

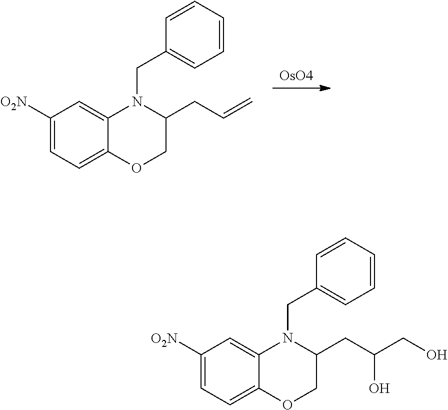 [SMILES: C=CCC1COc2ccc([N+](=O)[O-])cc2N1Cc1ccccc1 ; "OsO4" ; O=[N+]([O-])c1ccc2c(c1)N(Cc1ccccc1)C(CC(O)CO)CO2]

To a stirred solution of 3-allyl-4-benzyl-6-nitro-3,4-dihydro-2H-benzo[b][1,4]oxazine (Preparation 58, Step 2) (250 mg, 0.81 mmol) in tert-BuOH/H₂O solution (10 mL, 1:1) was added OsO₄ (20.48 mg 0.08 mmol) and NMO (188.7 mg, 1.61 mmol). The resulting reaction mixture was stirred at RT for 12 h. Progress of the reaction was checked by LCMS and after completion of the reaction it was further diluted with EtOAc. The organic layer was washed with 10% HCl, water and finally with brine. The organics were then dried over Na₂SO₄ and concentrated in vacuo to afford the title compound (240 mg, crude) as a brown solid. UPLC-MS m/z: 445 [M+H].

### Example 79: 1-(4-Benzyl-3-(2,3-dihydroxypropyl)-3,4-dihydro-2H-benzo[b][1,4]oxazin-6-yl)-3-(1H-indol-6-yl)urea

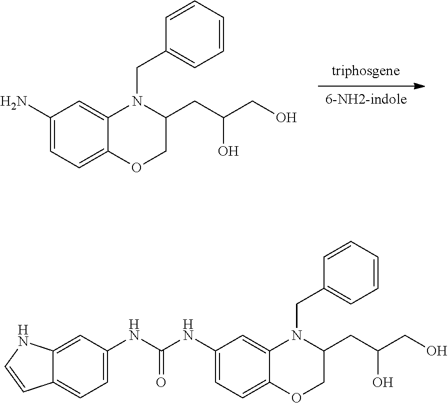 [SMILES: Nc1ccc2c(c1)N(Cc1ccccc1)C(CC(O)CO)CO2 ; "6-NH2-indole" ; "triphosgene" ; O=C(Nc1ccc2c(c1)N(Cc1ccccc1)C(CC(O)CO)CO2)Nc1ccc2cc[nH]c2c1]

To a stirred solution of 6-amino-indole (133.7 mg, 1.01 mmol) in THF (4 mL) was added triphosgene (120 mg, 0.4 mmol) at 0-5° C. under nitrogen. Stirring was continued at RT for 1 h, then 3-(6-amino-4-benzyl-3,4-dihydro-2H-benzo[b][1,4]oxazin-3-yl)propane-1,2-diol (prepared according to method described in Preparation 58, Step 3 from 3-(4-benzyl-6-nitro-3,4-dihydro-2H-benzo[b][1,4]oxazin-3-yl)propane-1,2-diol (Preparation 60)) (212 mg, 0.67 mmol) and TEA (340.6 mg, 3.37 mmol) were added and the combined mixture was further stirred at RT for 2 h. Completion of the reaction was confirmed by UPLC-MS after which the solvent was evaporated and the resulting residue was purified by prep-HPLC to afford the title compound (60 mg, 19% yield) as a grey solid. Purity by UPLC: 96.96%; ¹H NMR (400 MHz; DMSO-d₆): δ 1.35-1.46 (m, 1H), 1.73-1.80 (m, 1H), 3.20-3.30 (m, 1H), 3.57-3.59 (m, 2H), 4.0-4.01 (m, 1H), 4.17-4.24 (m, 1H), 4.42-4.50 (m 1H), 4.54-4.67 (m, 2H), 6.30 (bs, 1H), 6.59-6.65 (m, 3H), 6.73-6.77 (m, 1H), 7.18 (bs, 1H), 6.23-6.26 (m, 1H), 7.29-7.36 (m, 5H), 7.73 (s, 1H), 8.19 (s, 1H), 8.37-8.39 (m, 2H), (s, 1H), 10.85 (s, 1H); UPLC-MS m/z: 473 [M+H].

### Example 80: 1-(4-Benzyl-3-(2-hydroxyethyl)-3-,4-dihydro-2H-benzo[b][1,4]oxazin-6-yl)-3-(1H-indol-6-yl)urea

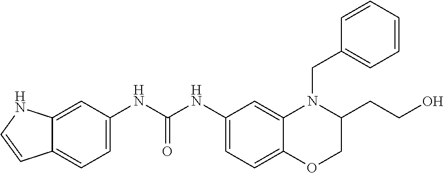 [SMILES: O=C(Nc1ccc2c(c1)N(Cc1ccccc1)C(CCO)CO2)Nc1ccc2cc[nH]c2c1]

Example 80 was prepared according to General Procedures 1, 4, 6, 20-23 and the methods described below.

### Preparation 61: 2-(4-Benzyl-6-nitro-3,4-dihydro-2H-benzo[b][1,4]oxazin-3-yl)ethan-1-ol

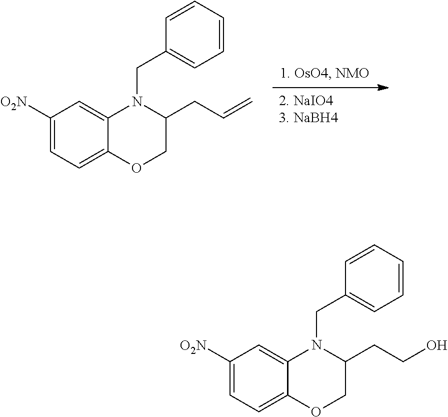

To a stirred solution of 3-allyl-4-benzyl-6-nitro-3,4-dihydro-2H-benzo[b][1,4]oxazine (Preparation 58, Step 2) (250 mg, 0.81 mmol) in tert-BuOH/H₂O solution (10 mL, 1:1) was added OsO₄ (20.48 mg, 0.08 mmol) and NMO (188.7 mg, 1.61 mmol). The resulting reaction mixture was stirred at RT for 12 h. Progress of the reaction was checked by LCMS and after completion the reaction was diluted with EtOAc and washed with 10% HCl, water and finally with brine. The organics were dried and concentrated in vacuo to afford the crude corresponding diol intermediate. The crude product was dissolved in tert-BuOH/H₂O solution (10 mL, 1:1) and NaIO4 (689.19 mg, 3.22 mmol) added at RT. The resulting reaction mixture was stirred at RT for 12 h. Progress of the reaction was checked by LCMS and after completion of the reaction it was diluted with water and extracted with EtOAc. The separated organic layer was dried and concentrated in vacuo to afford the crude corresponding aldehyde (200 mg, 0.64 mmol) which was dissolved in methanol (8 mL) and NaBH₄ (48.67 mg, 1.28 mmol) added at 0-5° C. Then the reaction mixture was further stirred at RT for 2 h. After completion of the reaction it was quenched with NH₄Cl solution (20 mL). The aqueous reaction mixture was extracted with EtOAc. The separated organic layers were dried over Na₂SO₄ and concentrated in vacuo to afford the title compound (200 mg, crude) which was taken on to the next step without any further purification. UPLC-MS m/z: 315 [M+H].

### Example 80: 1-(4-Benzyl-3-(2-hydroxyethyl)-3,4-dihydro-2H-benzo[b][1,4]oxazin-6-yl-3-(1H-indol-6-yl)urea

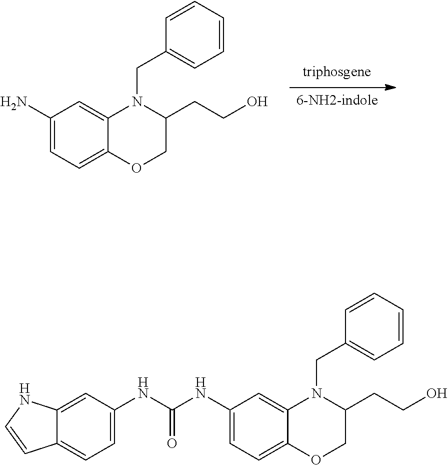 [SMILES: Nc1ccc2c(c1)N(Cc1ccccc1)C(CCO)CO2 ; "6-NH2-indole" ; "triphosgene" ; O=C(Nc1ccc2c(c1)N(Cc1ccccc1)C(CCO)CO2)Nc1ccc2cc[nH]c2c1]

To a stirred solution of 6-aminoindole (81.72 mg, 0.62 mmol) in THF (4 mL) was added triphosgene (66.81 mg, 0.23 mmol) at 0-5° C. under nitrogen. Stirring was continued at RT for 1 h, then 2-(6-amino-4-benzyl-3,4-dihydro-2H-benzo[b][1,4]oxazin-3-yl)ethan-1-ol (prepared according to method described in Preparation 58, Step 3 from 2-(4-benzyl-6-nitro-3,4-dihydro-2H-benzo[b][1,4]oxazin-3-yl)ethan-1-ol (Preparation 61)) (160 mg, 0.56 mmol) and TEA (0.17 ml, 1.24 mmol) were added and the whole further stirred at RT for 12 h. Completion of the reaction was confirmed by UPLC-MS and after completion the solvent was evaporated and the resulting residue was purified by prep-HPLC to afford the title compound (40 mg, 16% yield) as a grey solid. Purity by UPLC: 99.5%; ¹H NMR (400 MHz; DMSO-d₆): δ 1.66-1.75 (m, 2H), 3.47-3.57 (m, 3H), 3.95-3.98 (dd, 1H, J1=1.88 Hz, J2=10.68 Hz), 4.17-4.20 (dd, 1H, J1=1.28 Hz, J2=10.68 Hz), 4.42-4.51 (m, 3H), 6.29 (s, 1H), 6.60-6.65 (m, 3H), 6.74-6.77 (dd, 1H, J1=1.68 Hz, J2=8.48 Hz), 7.18 (t, 1H, J=2.52 Hz), 7.24-7.26 (m, 1H), 7.30-7.36 (m, 5H), 7.73 (s, 1H), 8.19 (s, 1H), 8.38 (s, 1H), 10.84 (s, 1H); UPLC-MS m/z: 443 [M+H].

### Example 81: 1-(4-Benzyl-3-cyano-3,4-dihydro-2H-benzo[b][1,4]oxazin-6-yl)-3-(1H-indol-6-yl)urea

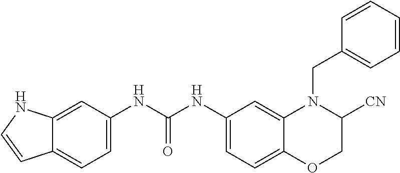 [SMILES: N#CC1COc2ccc(NC(=O)Nc3ccc4cc[nH]c4c3)cc2N1Cc1ccccc1]

Example 81 was prepared according to General Procedures 1, 4, 6, 20 and the methods described below.

### Preparation 62: 6-Amino-4-benzyl-3,4-dihydro-2H-benzo[b][1,4]oxazine-3-carbonitrile

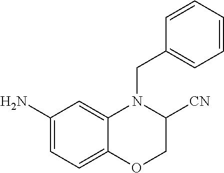 [SMILES: N#CC1COc2ccc(N)cc2N1Cc1ccccc1]

### Step 1: 4-Benzyl-6-nitro-3,4-dihydro-2H-benzo[b][1,4]oxazine-3-carbonitrile

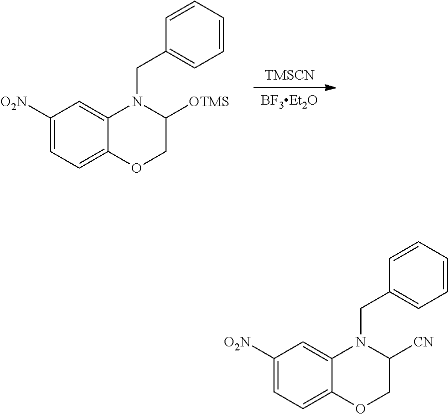

To a stirred solution of 4-benzyl-6-nitro-3-((trimethylsilyl)oxy)-3,4-dihydro-2H-benzo[b][1,4]oxazine (Preparation 58, Step 1) (355 mg, 0.99 mmol) in DCM (10 mL) was added TMSCN (0.49 mL, 3.96 mmol) and BF₃.Et₂O (0.81 mL, 3.96 mmol) at −78° C. under nitrogen. The temperature was then slowly raised to 0-5° C. Progress of the reaction was checked by UPLC and after 2 h formation of the desired product was confirmed. The reaction was quenched with water and then extracted with EtOAc. The combined organic layers were collected, dried over Na₂SO₄ and evaporated in vacuo to give the crude product which was purified by Combi-flash chromatography to afford the title compound (190 mg, 65% yield) as a yellow solid. UPLC-MS m/z: 296 [M+H].

### Step 2: 6-Amino-4-benzyl-3,4-dihydro-2H-benzo[b][1,4]oxazine-3-carbonitrile

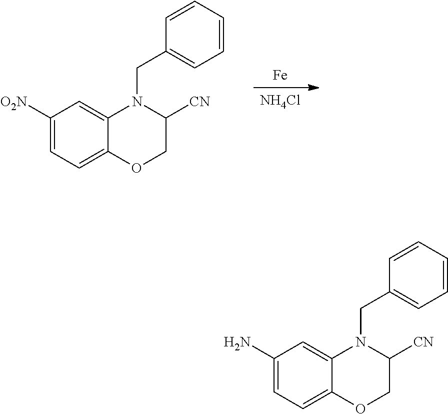

To a stirred solution of 4-benzyl-6-nitro-3,4-dihydro-2H-benzo[b][1,4]oxazine-3-carbonitrile (Preparation 62, Step 1) (0.180 g, 0.61 mmol) in ethanol (4 mL) was added Fe powder (0.33 mg, 6.1 mmol) and a saturated solution of NH₄Cl (4 mL) in ice-cold water. The mixture was kept at ice cold temperature for 5 min. after which time the mixture was refluxed for 1 h. Completion of the reaction was confirmed by TLC and LCMS. The reaction mixture was filtered through a celite pad and washed with ethanol. The ethanol mixture was evaporated under reduced pressure, diluted with water, extracted with EtOAc, dried with Na₂SO₄ and concentrated in vacuo to afford the title compound (160 mg, crude) as a brown oily crude which was used in the next step without any further purification. UPLC-MS m/z: 264.15 [M+H].

### Example 81: 1-(4-Benzyl-3-cyano-3,4-dihydro-2H-benzo[b][1,4]oxazin-6-yl)-3-(1H-indol-6-yl)urea

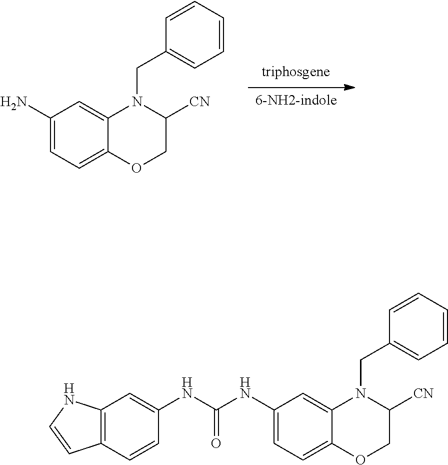 [SMILES: N#CC1COc2ccc(N)cc2N1Cc1ccccc1 ; "6-NH2-indole" ; "triphosgene" ; N#CC1COc2ccc(NC(=O)Nc3ccc4cc[nH]c4c3)cc2N1Cc1ccccc1]

To a stirring solution of 6-aminoindole (0.120 g, 0.9 mmol) in THF (3 mL) was added triphosgene (0.108 g, 0.39 mmol) at 0-5° C. and the mixture was stirred for five min. followed by 1 h at RT. Completion of the first stage of the reaction was confirmed by TLC and then 6-amino-4-benzyl-3,4-dihydro-2H-benzo[b][1,4]oxazine-3-carbonitrile (Preparation 62, Step 2) (0.160 g, 0.60 mmol) and TEA (0.500 mL, 0.6 mmol) were added into the reaction mixture at 0-5° C. The resulting reaction mixture was stirred at RT for 1 h. UPLC and TLC showed mass of the desired product. The reaction mixture was diluted with water and extracted with EtOAc. The combined organics were washed with 1 N NaOH solution followed by brine and dried over anhydrous Na₂SO₄, filtered and evaporated under reduced pressure to obtain a crude product which was purified by column chromatography using 2.5% MeOH in DCM as eluent to afford the title compound (180 mg, 72% yield) as a black solid. Purity by UPLC: 93.27%; ¹H NMR (400 MHz; DMSO-d₆): δ 3.34 (d, 2H, J=11.2 Hz), 4.56 (m, 2H), 4.89 (s, 1H), 6.31 (s, 1H), 6.76-6.86 (m, 3H), 7.21-7.75 (m, 8H), 7.75 (s, 1H), 8.27 (s, 1H), 8.38 (s, 1H), 10.89 (s, 1H); UPLC-MS m/z: 424.19 [M+H].

### Preparation 63: 1-(3-(Aminomethyl)-4-benzyl-3,4-dihydro-2H-benzo[b][1,4]oxazin-6-yl-3-(1H-indol-6-yl)urea (Example 82)

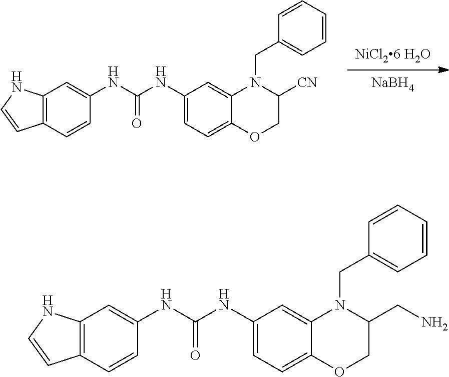 [SMILES: N#CC1COc2ccc(NC(=O)Nc3ccc4cc[nH]c4c3)cc2N1Cc1ccccc1 ; "NaBH4" ; "NiCl2·6 H2O" ; NCC1COc2ccc(NC(=O)Nc3ccc4cc[nH]c4c3)cc2N1Cc1ccccc1]

To a stirring solution of 1-(4-benzyl-3-cyano-3,4-dihydro-2H-benzo[b][1,4]oxazin-6-yl)-3-(1H-indol-6-yl)urea (Example 81) (80 mg, 0.189 mmol) in methanol (2 mL) was added NiCl₂.6H₂O (45 mg, 0.014 mmol) and NaBH₄ (4.2 mg, 0.11 mmol) at 0-5° C. The reaction mixture was stirred at RT for 30 min. and after completion of the reaction (monitored by LCMS & TLC) the reaction mixture was quenched with NH₄Cl solution. The methanol was evaporated under reduced pressure and the resulting residue was diluted with water, extracted with EtOAc, dried over anhydrous Na₂SO₄, and evaporated under reduced pressure to obtain the crude product which was purified by prep-HPLC to afford the title compound (10 mg, 12% yield) as a yellow solid. Purity by UPLC: 96.85%; ¹H NMR (400 MHz; DMSO-d₆): δ 2.56-2.61 (m, 1H), 2.68-2.72 (m, 1H), 3.10-3.25 (m, 2H), 3.86 (d, 1H, J=9.8 Hz), 4.41 (d, 1H, J=10.56 Hz) 4.54 (s, 2H), 6.29 (s, 1H), 6.58-6.64 (m, 3H), 6.77 (d, 1H, J=8.28 Hz), 7.17 (s, 1H), 7.24 (d, 1H, J=6.56 Hz), 7.30-7.34 (m, 5H), 7.74 (s, 1H), 8.48 (s, 1H), 8.65 (s, 1H), 10.83 (s, 1H); UPLC-MS m/z: 428.32 [M+H].

### Preparation 64: 6-3-(1H-Indol-6-yl)ureido)-4-benzyl-3,4-dihydro-2H-benzo[b][1,4]oxazine-3-carboxamide (Example 83)

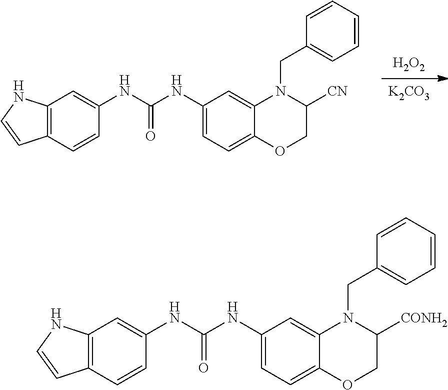

To a stirred solution of 1-(4-benzyl-3-cyano-3,4-dihydro-2H-benzo[b][1,4]oxazin-6-yl)-3-(1H-indol-6-yl)urea (Example 81) (100.0 mg, 0.24 mmol) in MeOH (8 mL) was added K₂CO₃ (163.18 mg, 1.18 mmol) at 0-5° C. and the whole was stirred for 5 min. Then H₂O₂ (0.6 mL, 30% aq.) was added at 0-5° C. and stirring continued for 2 h. The reaction was monitored by LCMS which showed formation of the desired product. The solvent was evaporated in vacuo to give the crude product which was purified by prep-HPLC to afford the title compound (12 mg, 12% yield) as a yellow solid. Purity by UPLC: 96.11%; ¹H NMR (400 MHz; DMSO-d₆): δ 4.00 (s, 2H), 4.32 (d, 1H, J=16.5 Hz), 4.50 (d, 1H, J=9 Hz), 4.72 (d, 1H, J=16.44 Hz), 6.30 (s, 1H), 6.59-6.80 (m, 4H), 7.18-7.34 (m, 9H), 7.97 (s, 1H), 8.31 (s, 1H), 8.44 (s, 1H), 10.85 (s, 1H); UPLC-MS m/z: 442.31 [M+H].

### Examples 186-188

The examples in the table below were prepared according to the above methods used to make Examples 78-83 as described in General Procedures 1-6 using the appropriate amine. Purification was as stated in the aforementioned methods.

| | | IUPAC | | LCMS | Purity |
|---|---|---|---|---|---|
| Ex | Structure | Name | 1H-NMR | [M + H] | (%) |
| 186 | 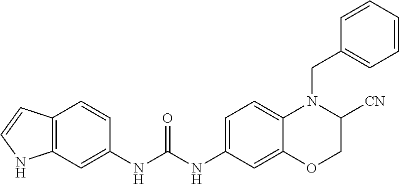 [SMILES: N#CC1COc2cc(NC(=O)Nc3ccc4cc[nH]c4c3)ccc2N1Cc1ccccc1] | 1-(4-benzyl- 3-cyano-3,4- dihydro-2H- benzo[b][1,4] oxazin-7-yl)- 3-(1H-indol- 6-yl)urea | (400 MHz; DMSO- d₆): δ 4.35 (m, 2H), 4.56 (m, 2H), 4.77 (s, 1H), 6.31 (s, 1H), 6.67 (d, 1H, J = 8.76 Hz), 6.80 (m, 2 H), 7.13 (s, 1H), 7.19 (s, 1H), 7-39-7-30 (m, 6H), 7.77 (s, 1H), 8.37 (s, 1H), 8.46 (s, 1 H), 10.89 (s, 1H). | 424.13 | 99.04 |
| 187 | 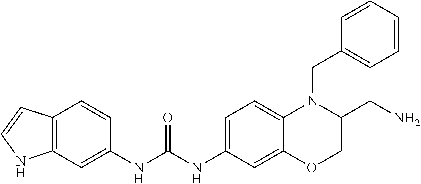 [SMILES: NCC1COc2cc(NC(=O)Nc3ccc4cc[nH]c4c3)ccc2N1Cc1ccccc1] | 1-(3- (aminomethyl)- 4-benzyl- 3,4-dihydro- 2H- benzo[b][1,4] oxazin-7-yl)- 3-(1H-indol- 6-yl)urea | (400 MHz; DMSO- d₆): δ 2.59 (m, 2H), 2.99 (m, 2H), 3.94 (d, 1H, J = 10.2 Hz), 4.53-441 (m, 3H) 6.30 (s, 1H), 6.42 (d, 1H, J = 8.28 Hz), 6.69 (d, 1H, J = 7.28 Hz), 6.82 (m, 1H), 6.99 (s, 1H), 7.18- 7.37 (m, 7H), 7.77 (s, 1H), 8.44 (s, 1H), | 428.16 | 96.08 |
| | | | 8.64 (s, 1H), 10.86 | | |
| | | | (s, 1H). | | |
| 188 | 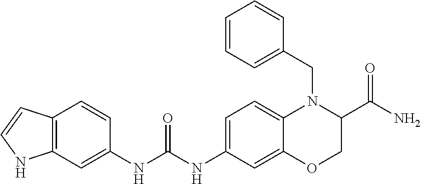 [SMILES: NC(=O)C1COc2cc(NC(=O)Nc3ccc4cc[nH]c4c3)ccc2N1Cc1ccccc1] | 7-(3-(1H- indol-6- yl)ureido)-4- benzyl-3,4- dihydro-2H- benzo[b][1,4] oxazine-3- carboxamide | (400 MHz; DMSO- d₆): δ 3.94 (s, 1H)), 3-99 (s, 1H), 4.32 (d, 1H, J = 14.2 Hz), 4.50 (s, 1H), 4.66 (d, 1H, J = 15 Hz), 6.30 (s, 1H), 6.62 (s, 1H), 6.75-7-34 (m, 9H), 7.78 s, 1H), 8.47 (s, 1H), 8.63 (s, 1H), 10.85 (s, 1H). | 442.09 | 97.28 |

### Example 84: 2-(6-(3-(1H-Indol-6-yl)ureido)-2,3-dihydro-4H-benzo[b][1,4]oxazin-4-yl)-2-phenylacetamide

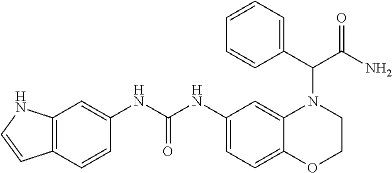 [SMILES: NC(=O)C(c1ccccc1)N1CCOc2ccc(NC(=O)Nc3ccc4cc[nH]c4c3)cc21]

Example 84 was prepared according to General Procedures 1, 3-4, 6, 27 and the methods described below.

### Preparation 65: 2-(6-Amino-2,3-dihydro-4H-benzo[b][1,4]oxazin-4-yl-2-phenylacetamide

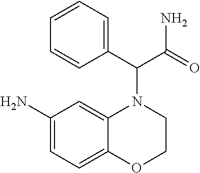 [SMILES: NC(=O)C(c1ccccc1)N1CCOc2ccc(N)cc21]

### Step 1: Methyl 2-(6-nitro-2,3-dihydro-4H-benzo[b][1,4]oxazin-4-yl)-2-phenylacetate

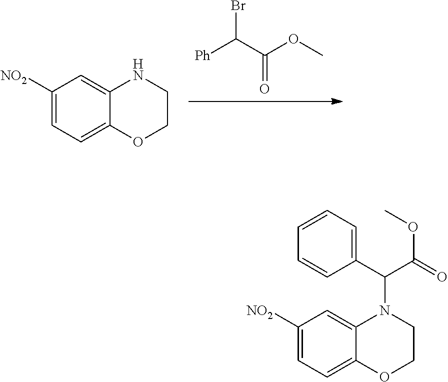 [SMILES: O=[N+]([O-])c1ccc2c(c1)NCCO2 ; COC(=O)C(Br)c1ccccc1 ; COC(=O)C(c1ccccc1)N1CCOc2ccc([N+](=O)[O-])cc21]

To a stirred solution of commercially available 6-nitro-3,4-dihydro-2H-benzo[b][1,4]oxazine (2 g, 11.1 mmol) in ACN (40.0 mL) was added methyl 2-bromo-2-phenylacetate (5.23 mL, 33.3 mmol) and the reaction mixture was stirred at 100° C. in a sealed tube for 16 h. The excess solvent was concentrated under reduced pressure and the reaction mixture was quenched with Na₂CO₃ solution and the organics extracted with EtOAc (3×50 mL). The combined organic layers were washed with brine solution (1×30 mL), dried over Na₂SO₄ and filtered. The filtrate was concentrated under reduced pressure to obtain the crude product. The crude was purified by silica gel column chromatography (5-10% EtOAc-hexane) to afford the title compound (1.8 g, 49% yield) as a yellow sticky solid. LCMS m/z: 329.1 [M+H].

### Step 2: 2-(6-Nitro-2,3-dihydro-4H-benzo[b][1,4]oxazin-4-yl)-2-phenylacetic acid

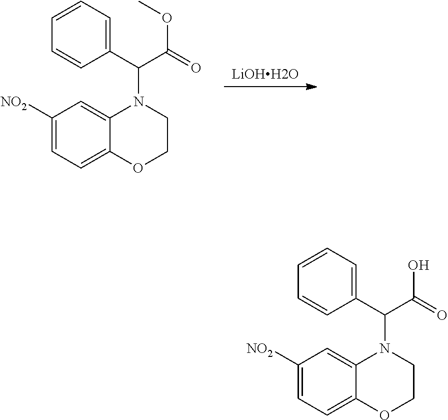 [SMILES: COC(=O)C(c1ccccc1)N1CCOc2ccc([N+](=O)[O-])cc21 ; "LiOH·H2O" ; O=C(O)C(c1ccccc1)N1CCOc2ccc([N+](=O)[O-])cc21]

To a stirred solution of methyl 2-(6-nitro-2,3-dihydro-4H-benzo[b][1,4]oxazin-4-yl)-2-phenylacetate (Preparation 65, Step 1) (0.200 g, 0.609 mmol) in THF:MeOH:water (10 mL, 2:1:1, v/v/v) was added LiOH.H₂O (0.102 g, 2.437 mmol) at 0-5° C. and the reaction mixture was stirred at room temperature for 3 h. After complete consumption of the starting material (confirmed by LCMS), water was added and the reaction mixture was acidified with 1N HCl and extracted with EtOAc (3×50 mL). The combined organic layers were washed with brine, dried over anhydrous Na₂SO₄ and evaporated under reduced pressure to afford the title compound (172 mg, 90% yield) as a yellow solid. LCMS m/z: 315.2 [M+H].

### Step 3: 2-(6-Nitro-2,3-dihydro-4H-benzo[b][1,4]oxazin-4-yl-2-phenylacetamide

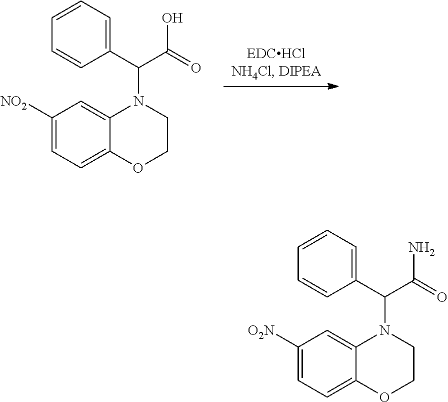 [SMILES: O=C(O)C(c1ccccc1)N1CCOc2ccc([N+](=O)[O-])cc21 ; "EDC·HClNH4Cl, DIPEA" ; NC(=O)C(c1ccccc1)N1CCOc2ccc([N+](=O)[O-])cc21]

To a stirred solution of 2-(6-nitro-2,3-dihydro-4H-benzo[b][1,4]oxazin-4-yl)-2-phenylacetic acid (Preparation 65, Step 2) (0.172 g, 0.55 mmol) in DMF (3 mL) was added EDC-HCl (0.157 g, 0.82 mmol) and DIPEA (0.21 mL, 1.64 mmol) at 0-5° C. and the reaction mixture was stirred for 10 min. keeping the temperature at 0-5° C., then NH₄Cl (0.150 g, 2.74 mmol) was added and the reaction mixture was stirred at RT for 16 h. After completion of the reaction (monitored by TLC), the solvent was evaporated under reduced pressure, extracted with EtOAc, dried over anhydrous Na₂SO₄ and evaporated under reduced pressure to afford the crude product which was purified by column chromatography using 40% EtOAc in hexane as eluent to afford the title compound (120 mg, 70% yield) as an off-white solid. LCMS m/z: 314.1 [M+H].

### Step 4: 2-(6-Amino-2,3-dihydro-4H-benzo[b][1,4]oxazin-4-yl)-2-phenylacetamide

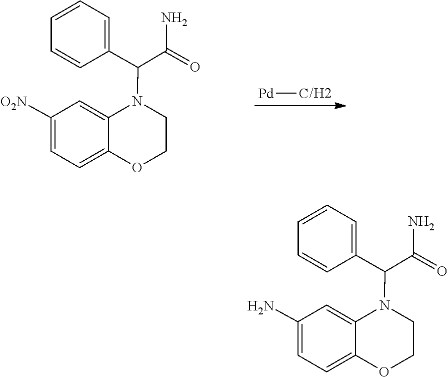

To a stirred solution of 2-(6-nitro-2,3-dihydro-4H-benzo[b][1,4]oxazin-4-yl)-2-phenylacetamide (Preparation 65, Step 3) (0.200 g, 0.638 mmol) in MeOH (6 mL), was added Pd—C(0.05 g, 10% w/w) and the reaction mixture was stirred under hydrogen balloon pressure for 3 h. After completion of the reaction (monitored by TLC), the reaction mixture was filtered through a celite pad and washed with MeOH. The filtrate was evaporated under reduced pressure to afford the crude material which was purified by column chromatography using 40% EtOAc in hexane as eluent to afford the title compound (100 mg, crude) as a gummy solid. LCMS m/z: 284.2 [M+H].

### Preparation 66: 2-(6-3-(1H-Indol-6-yl)ureido)-2,3-dihydro-4H-benzo[b][1,4]oxazin-4-yl)-2-phenylacetamide (Example 84)

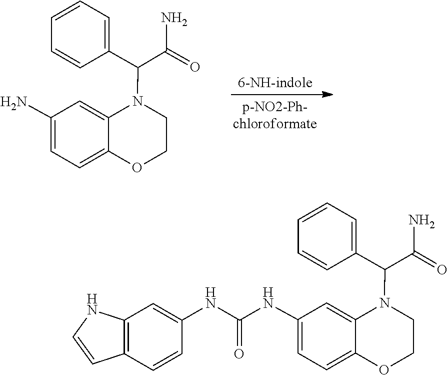 [SMILES: NC(=O)C(c1ccccc1)N1CCOc2ccc(N)cc21 ; NC(=O)C(c1ccccc1)N1CCOc2ccc(NC(=O)Nc3ccc4cc[nH]c4c3)cc21]

To a stirred solution of 1H-indol-6-amine (51.3 mg, 0.39 mmol) in THF (3 mL) was added p-nitrophenyl chloroformate (107 mg, 0.53 mmol) at 0-5° C. and the whole stirred at room temperature for 3 h. Then to the reaction mixture was added TEA (0.2 mL, 1.41 mmol) and 2-(6-amino-2, 3-dihydro-4H-benzo[b][1,4]oxazin-4-yl)-2-phenylacetamide (Preparation 65, Step 4) (100 mg, 0.35 mmol) at the same temperature and the combined mixture was stirred for another 2 h. The reaction was monitored by LCMS. After completion the solvent was evaporated to obtain the crude product. The crude was purified by reverse phase prep-HPLC to afford the title compound (14 mg, 9% yield) as an off white solid. Purity by UPLC: 99.36%; ¹H NMR (400 MHz; DMSO-d₆): δ 2.81-2.85 (m, 1H), 3.33-3.38 (m, 1H), 3.88 (t, 1H, J=7.56 Hz), 4.12 (m, 1H), 5.35 (s, 1H), 6.30 (s, 1H), 6.60 (d, 1H, J=8.44 Hz), 6.80-6.71 (m, 2H), 6.83 (s, 1H), 7.17 (s, 1H), 7.32-7.41 (m, 7H), 7.76 (s, 2H), 8.16 (s, 1H), 8.42 (s, 1H), 10.85 (s, 1H); LCMS m/z: 442.2 [M+H].

### Example 81: 1-(4-(2-Hydroxy-1-phenylethyl)-3,4-dihydro-2H-benzo[b][1,4]oxazin-6-yl)-3-(1H-indol-6-yl)urea

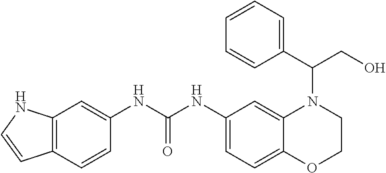 [SMILES: O=C(Nc1ccc2c(c1)N(C(CO)c1ccccc1)CCO2)Nc1ccc2cc[nH]c2c1]

Example 85 was prepared according to General Procedures 1, 3-4, 6 and the methods described below.

### Preparation 67: Methyl 2-(6-amino-2,3-dihydro-4H-benzo[b][1,4]oxazin-4-yl-2-phenylacetate

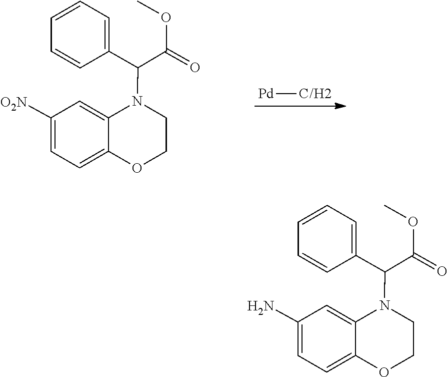

To a stirred and degassed solution of methyl 2-(6-nitro-2,3-dihydro-4H-benzo[b][1,4]oxazin-4-yl)-2-phenylacetate (Preparation 65, Step 1) (0.530 g, 1.614 mmol) in MeOH (20 mL) was added Pd—C(0.055 g, 10% w/w). The reaction mixture was then stirred at RT in the presence of hydrogen gas for 4 h. After completion of the reaction (monitored by TLC), the reaction mixture was filtered through a celite pad and washed thrice with MeOH. The solvent was evaporated under reduced pressure to obtain the crude product which was purified by column chromatography using 30% EtOAc in hexane as eluent to afford the title compound (0.4 g, 90% yield) as a yellow gummy solid. LCMS m/z: 299.25 [M+H].

### Preparation 68: Methyl 2-(6-(3-(1H-indol-6-yl)ureido)-2,3-dihydro-4H-benzo[b][1,4]oxazin-4-yl-2-phenylacetate (Example 86)

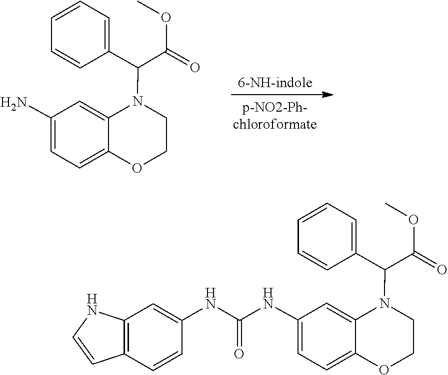 [SMILES: COC(=O)C(c1ccccc1)N1CCOc2ccc(N)cc21 ; COC(=O)C(c1ccccc1)N1CCOc2ccc(NC(=O)Nc3ccc4cc[nH]c4c3)cc21]

To a stirred solution of 1H-indol-6-amine (0.05 g, 0.37 mmol) in THF (2.5 mL) was added Et₃N (0.14 mL, 1.01 mmol) and p-nitrophenyl chloroformate (0.10 g, 0.50 mmol) at 0-5° C. and the resulting reaction mixture was stirred at 0-5° C. for 1 h. To the reaction mixture was added methyl 2-(6-amino-2,3-dihydro-4H-benzo[b][1,4]oxazin-4-yl)-2-phenylacetate (Preparation 67) (0.10 g, 0.34 mmol) in THF (1.5 mL) at 0-5° C. and the reaction mixture was stirred at RT for 16 h. After completion of the reaction (monitored by TLC, 5% acetone in DCM), the solvent was evaporated under reduced pressure and extracted with EtOAc (2×30 mL). The combined organic layers were dried over anhydrous Na₂SO₄ and evaporated under reduced pressure to afford the crude product which was purified by column chromatography using 2% acetone in DCM as eluent followed by trituration with pentane to give the title compound (0.05 g, 35% yield) as an off white solid. Purity by UPLC: 97.85%; ¹H NMR (400 MHz; DMSO-d₆): δ 2.83 (d, 1H, J=12.44 Hz), 3.41-3.48 (m, 1H), 3.75 (s, 3H), 3.92 (t, 1H, J=9.24 Hz), 4.13 (t, 1H, J=94 Hz), 5.71 (s, 1H), 6.31 (s, 1H), 6.63 (d, 1H, J=8.68 Hz), 6.73-6.81 (m, 2H), 6.97 (s, 1H), 7.19 (s, 1H), 7.33-7.45 (m, 6H), 7.79 (s, 1H), 8.25 (s, 1H), 8.42 (s, 1H), 10.87 (s, 1H); LCMS m/z: 457.36 [M+H].

### Preparation 69: 1-(4-(2-Hydroxy-1-phenylethyl)-3,4-dihydro-2H-benzo[b][1,4]oxazin-6-yl-3-(1H-indol-6-yl)urea (Example 85)

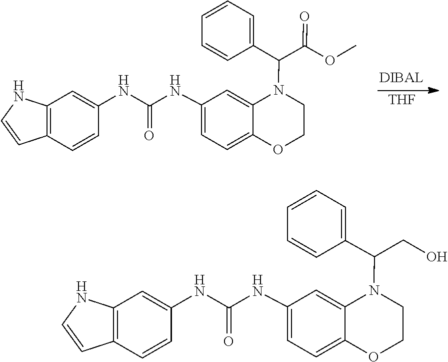 [SMILES: COC(=O)C(c1ccccc1)N1CCOc2ccc(NC(=O)Nc3ccc4cc[nH]c4c3)cc21 ; O=C(Nc1ccc2c(c1)N(C(CO)c1ccccc1)CCO2)Nc1ccc2cc[nH]c2c1 ; "THF"]

To a stirred solution of methyl 2-(6-(3-(1H-indol-6-yl)ureido)-2,3-dihydro-4H-benzo[b][1,4]oxazin-4-yl)-2-phenylacetate (Example 86) (100 mg, 0.22 mmol) in THF (3 mL) was added DIBAL-H (0.66 mL, 0.66 mmol, 1M in toluene) dropwise at 0-5° C. The mixture was then stirred at the same temperature for 2 h. The reaction mixture was quenched by dropwise addition of a saturated solution of Rochelle salt at RT and the resulting solution was stirred at RT for 1 h. The reaction mass was filtered through a celite bed. The celite bed was washed with EtOAc, the organics were separated and the aqueous layer was extracted with EtOAc (2×20 mL). The combined organic layers were washed with brine (1×20 mL), dried over Na₂SO₄ and concentrated under reduced pressure to obtain the crude product. This crude was purified by reverse phase prep-HPLC to afford the title compound (16 mg, 17% yield) as a yellow solid. Purity by UPLC: 98.85%; ¹H NMR (400 MHz; DMSO-d₆): δ 3.31 (s, 1H), 3.51 (s, 1H), 3.91 (t, 2H, J=5.35 Hz), 4.05 (d, 1H, J=6.24 Hz), 4.12 (d, 1H, J=5.12 Hz), 4.84 (t, 1H, J=6.74 Hz), 4.99 (t, 1H, J=6.74 Hz) 6.31 (s, 1H), 6.55 (m, 2H), 6.78 (s, 2H), 6.90 (s, 1H), 7.19-7.38 (m, 6H), 7.77 (s, 1H), 8.14 (s, 1H), 8.32 (s, 1H), 10.85 (s, 1H); LCMS m/z: 429.2 [M+H].

### Examples 189-190

The examples in the table below were prepared according to the above methods used to make Example 84-86 as described in General Procedures 1, 3-4, 6, 27 using the appropriate amine. Purification was as stated in the aforementioned methods.

| | | IUPAC | | LCMS | Purity |
|---|---|---|---|---|---|
| Ex | Structure | Name | 1H-NMR | [M+H] | (%) |
| 189 | 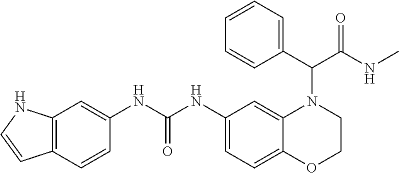 [SMILES: CNC(=O)C(c1ccccc1)N1CCOc2ccc(NC(=O)Nc3ccc4cc[nH]c4c3)cc21] | 2-(6-(3- (1H-indol- 6- yl)ureido)- 2,3- dihydro- 4H- benzo[b][1, 4]oxazin-4- yl)-N- methyl-2- | (400 MHz; DMSO- d₆): δ 2.67 (s, 3H), 2.83-2.86 (m, 1H), 3.32 (d, 1H, J = 4.12 Hz), 3.91 (t, 1H, J = 7.28 Hz)), 4.11-4.15 (m, 1H), 5.37 (s, 1H), 6.31 (s, 1H), 6.63 (d, 1H, J = 8.4 Hz), 6.84- 6.71 (m, 3H), 7.19 (t, 1H, J = 2.48 Hz), 7.29- | 456.44 | 98.67 |
| | | phenylacetamide | 7.42 (m, 5H), 7.78 (s, | | |
| | | | 1H), 8.18 (s, 1H), 8.27 | | |
| | | | (d, 1H, J = 4.64 Hz), | | |
| | | | 8.42 (s, 1H), 10.88 (s, | | |
| | | | 1H). | | |
| 190 | 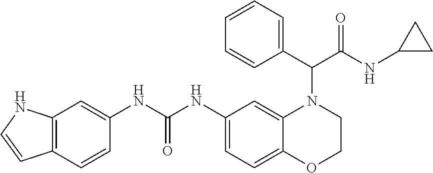 [SMILES: O=C(Nc1ccc2c(c1)N(C(C(=O)NC1CC1)c1ccccc1)CCO2)Nc1ccc2cc[nH]c2c1] | 2-(6-(3- (1H-indol- 6- yl)ureido)- 2,3- dihydro- 4H- benzo[b][1, 4]oxazin-4- yl)-N- cyclopropyl-2- | (400 MHz; DMSO- d₆): δ 0.47 (s, 2H), 0.64 (s, 2H), 2.70 (d, 1H, J = 11 Hz), 2.88 (d, 1H, J = 12.2 Hz)), 3.90 (s, 1H), 4.09 (S, 1H), 5-34 (s, 1H), 6.31 (s, 1H), 6.60-6.87 (m, 4H), 7.19-7.40 (m, 7H), 7.76 (s, 1H), 8.17 (s, 1H), 8.27 (d, 1H, J = | 482.4 | 99.16 |
| | | phenylacetamide | 4.64 Hz), 8.45 (s, | | |
| | | | 2H), 10.87 (s, 1H). | | |

### Example 191: 6-(4-(1H-Indol-6-yl)piperazin-1-yl)-4-benzyl-2H-benzo[b][1,4]thiazin-3(4H)-one

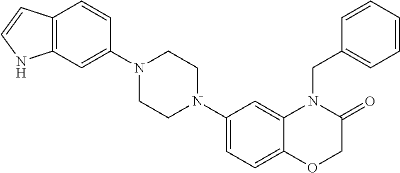 [SMILES: O=C1COc2ccc(N3CCN(c4ccc5cc[nH]c5c4)CC3)cc2N1Cc1ccccc1]

Example 191 was prepared according to General Procedures 4, 25 and the methods described below.

### Preparation 70: 4-Benzyl-6-bromo-2H-benzo[b][1,4]thiazin-3(4H)-one

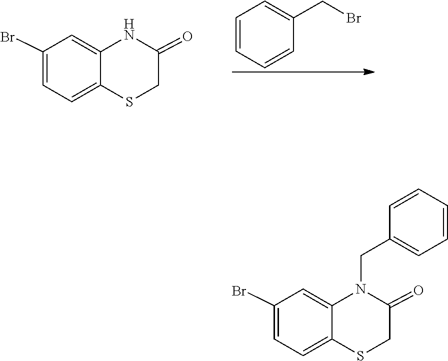 [SMILES: O=C1CSc2ccc(Br)cc2N1 ; BrCc1ccccc1 ; O=C1CSc2ccc(Br)cc2N1Cc1ccccc1]

K₂CO₃ (627 mg, 4.54 mmol) was added to a solution of commercially available 6-bromo-2H-benzo[b][1,4]thiazin-3(4H)-one (500 mg, 3.026 mmol) in DMF (3 mL) at RT. After stirring the mixture for 2-3 min., benzyl bromide (0.395 mL, 3.33 mmol) was added to the mixture and the whole was heated at 80° C. for 12 h. Progress of the reaction was monitored by LCMS and after completion the reaction mass was quenched with ice-water. The product was extracted with EtOAc. The combined organic layers were washed with water, brine solution, dried over anhydrous Na₂SO₄ and evaporated under reduced pressure to obtain the crude product. The crude product was purified by silica gel column chromatography to afford the title compound (450 mg, 44.5% yield) as a white solid.

### Preparation 71: 6-(Piperazin-1-yl)-1H-indole

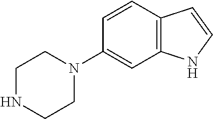 [SMILES: c1cc2ccc(N3CCNCC3)cc2[nH]1]

### Step 1: tert-Butyl 4-(1H-indol-6-yl)piperazine-1-carboxylate

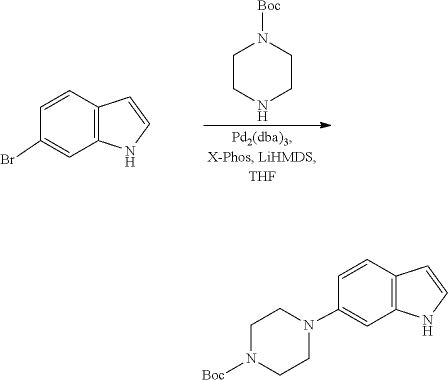 [SMILES: Brc1ccc2cc[nH]c2c1 ; CC(C)(C)OC(=O)N1CCNCC1 ; CC(C)(C)OC(=O)N1CCN(c2ccc3cc[nH]c3c2)CC1]

LiHMDS (1.12 mL, 1.12 mmol) was added to a degassed mixture of commercially available 6-bromo-1H-indole (100 mg, 0.51 mmol), tert-butyl piperazine-1-carboxylate (114 mg, 0.61 mmol), Pd₂(dba)₃ (4.6 mg, 0.005 mmol) and X-Phos (7.3 mg, 0.015 mmol) in THF (2 mL) in a sealed tube at RT. The tube was again purged with argon and then sealed. The mixture was stirred for 1-2 min. at RT and then heated at 65° C. for 24 h. Progress of the reaction was monitored by LCMS and after completion the reaction mixture was quenched with a saturated NH₄Cl solution and extracted with EtOAc. The combined organic layers were washed with brine, dried over anhydrous Na₂SO₄ and concentrated under vacuum to obtain the crude product which was purified by silica gel column chromatography to afford the title compound (100 mg, 65% yield) as a white solid.

### Step 2: 6-(Piperazin-1-yl)-1H-indole

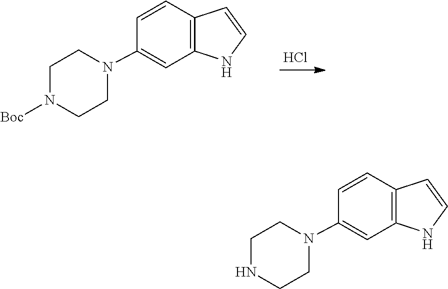 [SMILES: CC(C)(C)OC(=O)N1CCN(c2ccc3cc[nH]c3c2)CC1 ; Cl ; c1cc2ccc(N3CCNCC3)cc2[nH]1]

4M HCl in 1,4-Dioxane (3 mL) was added to a solution of tert-butyl 4-(1H-indol-6-yl) piperazine-1-carboxylate (Preparation 71, Step 1) (600 mg, 1.99 mmol) in dioxane at 0-5° C. Thereafter, the reaction was stirred at RT for 3 h. The reaction mass was quenched with saturated aqueous NaHCO₃ solution and the product was extracted with EtOAc. The combined organic layers were washed with brine solution, dried over anhydrous Na₂SO₄ and evaporated under reduced pressure to obtain the crude product. The crude product was purified by silica gel column chromatography to afford the title compound (210 mg, 52.4% yield) as a brown solid. LCMS m/z: 202 [M+H].

### Preparation 72: 6-(4-(1H-indol-6-yl)piperazin-1-yl)-4-benzyl-2H-benzo[b][1,4]thiazin-3(4H)-one (Example 191)

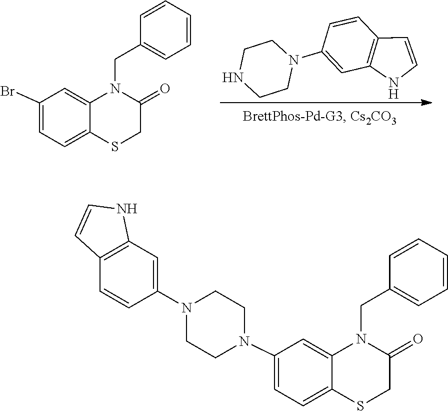 [SMILES: O=C1CSc2ccc(Br)cc2N1Cc1ccccc1 ; c1cc2ccc(N3CCNCC3)cc2[nH]1 ; O=C1CSc2ccc(N3CCN(c4ccc5cc[nH]c5c4)CC3)cc2N1Cc1ccccc1]

A mixture of 6-(piperazin-1-yl)-1H-indole (Preparation 71, Step 2) (60 mg, 0.29 mmol), 4-benzyl-6-bromo-3,4-dihydro-2H-1,4-benzothiazin-3-one (Preparation 70) (149 mg, 0.45 mmol), BrettPhos-Pd-G3 (27 mg, 0.03 mmol) and Cs₂CO₃ (291 mg, 0.894 mmol) in dioxane (4 mL) was heated at 100° C. for 24 h. Progress of the reaction was monitored by LCMS and after completion the solvents were evaporated under reduced pressure to give a crude compound which was purified by Prep-HPLC to afford the title compound (15 mg, 11% yield) as an off white sticky solid. Purity by HPLC 93.36%; ¹H NMR (400 MHz; DMSO-d₆): δ 2.85 (s, 4H), 2.88 (s, 4H), 3.77 (s, 2H), 5.30 (s, 2H), 6.51 (d, 1H, J=2.96 Hz), 6.77 (s, 1H), 6.85 (d, 1H, J=8.8 Hz), 7.21-7.43 (m, 9H), 7.61 (d, 1H, J=8.32 Hz); LCMS m/z: 455.33 [M+H].

### Example 192: 1-(4-Benzyl-R-oxo-3,4-dihydro-2H-benzo[b][1,4]thiazin-6-yl)-2-cyano-3-(1H-indol-6-yl)guanidine

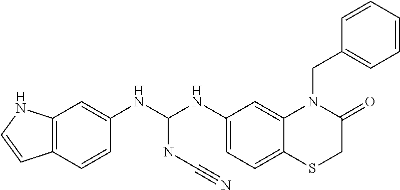 [SMILES: N#CNC(Nc1ccc2c(c1)N(Cc1ccccc1)C(=O)CS2)Nc1ccc2cc[nH]c2c1]

Example 192 was prepared according to General Procedures 2-4 and the methods described below.

### Preparation 73: 6-Isothiocyanato-1H-indole

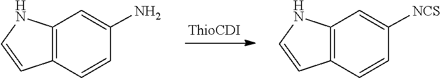

To a stirred solution of 6-amino indole (200 mg, 1.52 mmol) in DMF (10 mL) was added thio-CDI (297 mg, 1.67 mmol) in DMF (2 mL) dropwise at 0-5° C. The reaction was stirred at RT for 2 h. After completion of the reaction (checked by LCMS), it was quenched with ice-cold water (20 mL) and extracted with EtOAc. The combined organic layers were washed with water, brine, dried over anhydrous Na₂SO₄ and concentrated under reduced pressure to obtain the title compound (40 mg, 20% yield) as a dark brown solid. The crude was used in the next step without any further purification.

### Preparation 74: 1-(4-Benzyl-3-oxo-3,4-dihydro-2H-benzo[b][1,4]thiazin-6-yl-3-(1H-indol-6-yl)thiourea

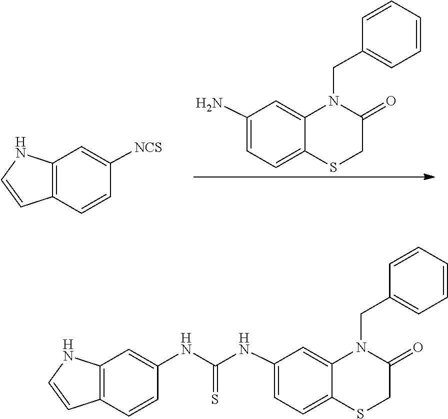

To a stirred solution of 6-isothiocyanato-1H-indole (Preparation 73) (40 mg, 0.23 mmol) in DCM (2.0 mL) was added 6-amino-4-benzyl-1,4-benzothiazin-3-one (Preparation 5) (62.1 mg, 0.23 mmol) at RT and the reaction mixture was stirred at the same temperature for 16 h. After completion of the reaction (monitored by LCMS) the reaction mass was evaporated to dryness to afford the title compound (80 mg, 78% yield) as a brown solid. The crude was used in the next step without any further purification. LCMS m/z: 445.41 [M+H].

### Preparation 7.: Methyl-N′-(4-benzyl-3-oxo-3,4-dihydro-2H-benzo[b][1,4]thiazin-6-yl)-N-(1H-indol-6-yl)carbamimidothioate

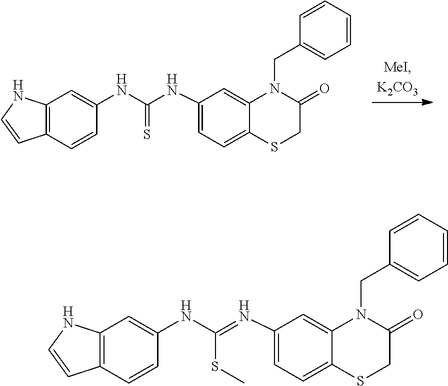 [SMILES: O=C1CSc2ccc(NC(=S)Nc3ccc4cc[nH]c4c3)cc2N1Cc1ccccc1 ; CS/C(=N\c1ccc2c(c1)N(Cc1ccccc1)C(=O)CS2)Nc1ccc2cc[nH]c2c1]

To a solution of 1-(4-benzyl-3-oxo-1,4-benzothiazin-6-yl)-3-(1H-indol-6-yl)thiourea (Preparation 74) (80 mg, 0.18 mmol) in acetone (2 mL) was added K₂CO₃ (62.3 mg, 0.45 mmol) and Mel (0.03 mL, 0.45 mmol) at RT. The reaction mixture was stirred at RT for 4 h. Progress of the reaction was monitored by LCMS and after completion the solvent was evaporated under vacuum. The residue was taken up in EtOAc and washed with water. The organic layer was dried over anhydrous Na₂SO₄ and concentrated under reduced pressure to give the title compound (50 mg, 61% yield) as a brown solid.

The crude was used in the next step without any further purification. LCMS m/z: 459.17 [M+H].

### Preparation 76: 1-(4-Benzyl-3-oxo-3,4-dihydro-2H-benzo[b][1,4]thiazin-6-yl-2-cyano-3-(1H-indol-6-yl)guanidine (Example 192)

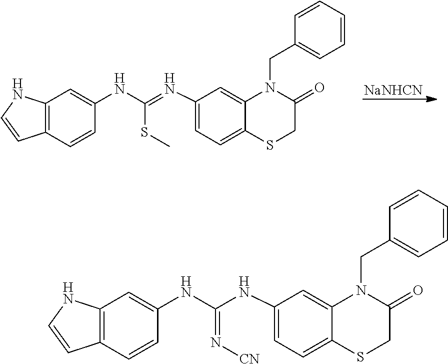

A stirred solution of 3-(4-benzyl-3-oxo-1,4-benzothiazin-6-yl)-1-(1H-indol-6-yl)-2-methyl-isothiourea (Preparation 75) (50 mg, 0.11 mmol) in 2-propanol (1 mL) was treated with sodium hydrogencyanamide (8.74 mg, 0.14 mmol) and heated in a microwave at 80° C. for 1 h. After completion of the reaction (monitored by LCMS) the solvent was evaporated to obtain the crude product which was purified by prep-HPLC to afford the title compound (6 mg, 12.2% yield) as an off white sticky solid. Purity by HPLC 99.26%; ¹H NMR (400 MHz; DMSO-d₆): δ 3.63 (s, 2H), 5.12 (s, 2H), 6.39 (s, 1H), 6.85 (d, 1H, J=7.92 Hz), 7.00 (d, 1H, J=7.96 Hz), 7.19-7.47 (m, 10H), 9.15 (s, 1H), 9.38 (s, 1H), 11.08 (s, 1H); LCMS m/z: 453.29 [M+H].

Biological Assay

Reporter Gene Expression Assay in THP-1 Cells

THP1-Dual™ cells (Invivogen) were derived from the human THP-1 monocyte cell line by stable integration of two inducible reporter constructs. As a result, THP1-Dual™ cells allow the simultaneous study of the IRF pathway, by assessing the activity of a secreted luciferase (Lucia) and the NF-κB pathway, by monitoring the activity of secreted SEAP. 5×10⁴ THP1-Dual™ cells were seeded in 384-well plates in growth medium and preincubated with novel compounds for 10 minutes followed by stimulation with 5 μM 2′,3′-cGAMP. After 20 hr of stimulation the supernatant was removed and the IRF pathway reporter protein was readily measured in the cell culture supernatant using QUANTI-Luc™ (Invivogen), a luciferase detection reagent on a Spectramax i3X luminometer.

In the tables below, IC₅₀ value ranges for exemplary compounds are given. The IC₅₀ ranges are indicated as “A” for values less than or equal to 1 μM, “B” for values greater than 1 μM and less than or equal to 10 μM, and “C” for values greater than 10 μM.

Activity data

| | THP-1 (HAQ) |
|---|---|
| Ex. No. | Activity |
| 1 | B |
| 2 | B |
| 3 | B |
| 4 | B |
| 6 | B |
| 9 | B |
| 12 | C |
| 16 | C |
| 17 | C |
| 18 | A |
| 19 | B |
| 22 | A |
| 23 | B |
| 24 | B |
| 25 | B |
| 26 | B |
| 27 | C |
| 28 | B |
| 29 | B |
| 30 | B |
| 31 | B |
| 32 | B |
| 33 | B |
| 38 | C |
| 39 | B |
| 40 | B |
| 41 | B |
| 42 | B |
| 43 | B |
| 44 | B |
| 45 | B |
| 46 | C |
| 47 | B |
| 48 | C |
| 49 | C |
| 50 | A |
| 51 | B |
| 52 | B |
| 53 | C |
| 55 | B |
| 56 | B |
| 57 | C |
| 59 | B |
| 60 | B |
| 62 | C |
| 63 | C |
| 64 | B |
| 65 | A |
| 66 | A |
| 67 | A |
| 68 | B |
| 69 | A |
| 72 | A |
| 73 | B |
| 74 | B |
| 75 | B |
| 76 | B |
| 77 | B |
| 78 | A |
| 79 | B |
| 80 | B |
| 81 | B |
| 82 | C |
| 83 | B |
| 84 | B |
| 85 | B |
| 86 | B |
| 87 | B |
| 88 | C |
| 89 | A |
| 90 | B |
| 91 | C |
| 92 | B |
| 93 | B |
| 94 | A |
| 95 | A |
| 96 | C |
| 97 | A |
| 98 | B |
| 99 | A |
| 100 | B |
| 101 | A |
| 102 | A |
| 103 | A |
| 104 | A |
| 105 | A |
| 106 | A |
| 107 | A |
| 108 | A |
| 109 | A |
| 110 | B |
| 111 | B |
| 112 | A |
| 113 | B |
| 114 | A |
| 115 | B |
| 116 | B |
| 117 | A |
| 118 | C |
| 119 | B |
| 120 | B |
| 121 | B |
| 122 | B |
| 123 | B |
| 124 | A |
| 125 | A |
| 126 | A |
| 127 | B |
| 128 | C |
| 129 | A |
| 130 | C |
| 131 | C |
| 132 | B |
| 133 | B |
| 134 | B |
| 135 | B |
| 136 | B |
| 137 | B |
| 138 | C |
| 139 | B |
| 140 | B |
| 141 | B |
| 142 | B |
| 143 | C |
| 144 | B |
| 145 | B |
| 146 | B |
| 147 | B |
| 148 | B |
| 149 | B |
| 150 | B |
| 151 | C |
| 152 | B |
| 153 | C |
| 154 | C |
| 155 | C |
| 156 | B |
| 157 | B |
| 158 | C |
| 159 | B |
| 160 | B |
| 161 | C |
| 162 | C |
| 163 | C |
| 164 | B |
| 165 | B |
| 166 | B |
| 167 | C |
| 168 | C |
| 169 | A |
| 170 | B |
| 171 | A |
| 172 | B |
| 173 | B |
| 174 | A |
| 175 | B |
| 176 | B |
| 177 | B |
| 178 | B |
| 179 | B |
| 180 | B |
| 181 | A |
| 182 | A |
| 183 | C |
| 184 | B |
| 185 | A |
| 186 | B |
| 187 | B |
| 188 | B |
| 189 | B |
| 190 | B |
| 191 | B |
| 192 | C |
| 193 | C |
| 194 | C |
| 195 | C |

1. A compound of formula (I): 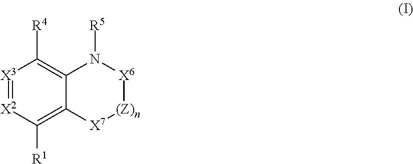
wherein X² is CR² or N;
X³ is CR³ or N;
X⁶ is C═O, or CR⁷R⁸;
the or each Z is independently CR⁹R¹⁰ or NR⁹;
X⁷ is S, SO, SO₂, O, NR¹¹ or CR¹¹R¹²;
n is 0, 1 or 2;
wherein, when n is 1 and Z is CR⁹R¹⁰; then X⁷ is S, SO, SO₂, O or NR¹¹; and
when n is 1 and Z is NR⁹ then X⁷ is CR¹¹R¹²;
R¹, R⁴, and R⁸ are each independently selected from the group consisting of H, halogen, OH, CN, COOR¹³, CONR¹³R¹⁴, NR¹³R¹⁴, NR¹³COR¹⁴, optionally substituted C₁-C₆ alkyl, optionally substituted C₁-C₆ alkylsulfonyl, optionally substituted mono or bicyclic C₃-C₆ cycloalkyl, optionally substituted C₂-C₆ alkenyl, optionally substituted C₂-C₆ alkynyl, optionally substituted C₁-C₆ alkoxy, optionally substituted C₁-C₆ alkoxycarbonyl group, mono or bicyclic optionally substituted C₆-C₁₂ aryl, mono or bicyclic optionally substituted 5 to 10 membered heteroaryl, optionally substituted mono or bicyclic 3 to 8 membered heterocycle, optionally substituted aryloxy, optionally substituted heteroaryloxy and optionally substituted heterocyclyloxy;
R⁹, R¹⁰, R¹¹ and R¹² are each independently selected from the group consisting of H, halogen, OH, CN, COOR¹³, CONR¹³R¹⁴, NR¹³R¹⁴, NR¹³COR¹⁴, optionally substituted C₁-C₆ alkyl, optionally substituted C₂-C₆ alkenyl and optionally substituted C₂-C₆ alkynyl;
one of R² and R³ is -L¹-L²-L³-L⁴-R¹⁵ and, when X² is CR² and X³ is CR³, the other of R² and R³ is selected from the group consisting of H, halogen, OH, CN, COOR¹³, CONR¹³R¹⁴, NR¹³R¹⁴, NR¹³COR¹⁴, optionally substituted C₁-C₆ alkyl, optionally substituted C₁-C₆ alkylsulfonyl, optionally substituted mono or bicyclic C₃-C₆ cycloalkyl, optionally substituted C₂-C₆ alkenyl, optionally substituted C₂-C₆ alkynyl, optionally substituted C₁-C₆ alkoxy, optionally substituted C₁-C₆ alkoxycarbonyl group, mono or bicyclic optionally substituted C₆-C₁₂ aryl, mono or bicyclic optionally substituted 5 to 10 membered heteroaryl, optionally substituted mono or bicyclic 3 to 8 membered heterocycle, optionally substituted aryloxy and optionally substituted heteroaryloxy, optionally substituted heterocyclyloxy;
R⁵ and R⁷ are selected from the group consisting of H, halogen, OH, CN, COOR¹³, CONR¹³R¹⁴, NR¹³R¹⁴, NR¹³COR¹⁴, optionally substituted C₁-C₆ alkyl, optionally substituted C₁-C₆ alkylsulfonyl, optionally substituted mono or bicyclic C₃-C₆ cycloalkyl, optionally substituted C₂-C₆ alkenyl, optionally substituted C₂-C₆ alkynyl, optionally substituted C₁-C₆ alkoxy, optionally substituted C₁-C₆ alkoxycarbonyl group, mono or bicyclic optionally substituted C₆-C₁₂ aryl, mono or bicyclic optionally substituted 5 to 10 membered heteroaryl, optionally substituted mono or bicyclic 3 to 8 membered heterocycle, optionally substituted aryloxy, optionally substituted heteroaryloxy, optionally substituted heterocyclyloxy and L⁵-L⁶-R¹⁶; wherein a maximum of one of R⁵ and R⁷ is -L⁵-L⁶-R¹⁶;
R¹³ and R¹⁴ are each independently selected from the group consisting of H, halogen, OH, CN, COOH, CONH₂, NH₂, NHCOH, optionally substituted C₁-C₆ alkyl, optionally substituted C₁-C₆ alkylsulfonyl, optionally substituted mono or bicyclic C₃-C₆ cycloalkyl, optionally substituted C₂-C₆ alkenyl, optionally substituted C₂-C₆ alkynyl, optionally substituted C₁-C₆ alkoxy, optionally substituted C₁-C₆ alkoxycarbonyl group, mono or bicyclic optionally substituted C₆-C₁₂ aryl, mono or bicyclic optionally substituted 5 to 10 membered heteroaryl, optionally substituted mono or bicyclic 3 to 8 membered heterocycle, optionally substituted aryloxy, optionally substituted heteroaryloxy and optionally substituted heterocyclyloxy;
a) L¹ is NR⁷ or O, L² is C═O, C═S, C═NR¹⁹ or SO₂, and L³ is absent, NR¹⁸ or O;
b) L¹ is absent, L² is C═O, C═S, C═NR¹⁹ or SO₂, and L³ is NR¹⁸ or O and when L² is C═O, L³ is not NR¹⁸;
c) L¹ is absent or is a C₁-C₃ alkylene; L² is absent; and L³ is O; or
d) L¹ is a C₅-C₆ cycloalkylene, a C₆ arylene, a 5 membered heteroarylene or a 5 to 6 membered heterocyclylene; L² is absent and L³ is absent;
L⁴ is absent or is an optionally substituted C₁-C₆ alkylene, an optionally substituted C₂-C₆ alkenylene, an optionally substituted C₂-C₆ alkynylene, an optionally substituted C₃-C₆ cycloalkylene, an optionally substituted C₆-C₁₂ arylene, an optionally substituted 5 to 10 membered heteroarylene or an optionally substituted 3 to 8 membered heterocyclylene;
L⁵ is absent or an optionally substituted C₁-C₆ alkylene, an optionally substituted C₂-C₆ alkenylene, an optionally substituted C₂-C₆ alkynylene, O, S, S═O, SO₂ or NR¹⁹;
L⁶ is absent or an optionally substituted C₁-C₆ alkylene, an optionally substituted C₂-C₆ alkenylene, an optionally substituted C₂-C₆ alkynylene, O, S, S═O, SO₂ or NR¹⁹;
R¹⁵ is H, optionally substituted C₁-C₆ alkyl, optionally substituted C₂-C₆ alkenyl, optionally substituted C₂-C₆ alkynyl, optionally substituted mono or bicyclic C₃-C₆ cycloalkyl, mono or bicyclic optionally substituted C₆-C₁₂ aryl, mono or bicyclic optionally substituted 5 to 10 membered heteroaryl or optionally substituted mono or bicyclic 3 to 8 membered heterocycle, wherein when R¹⁵ is an optionally substituted aryl the aryl is unsubstituted or substituted with one or more substituents selected from the group consisting of optionally substituted C₁-C₆ alkyl, halogen, OH, oxo, OP(O)(OR²⁰)(OR²¹), optionally substituted C₁-C₆ alkoxy, NR²⁰R²¹, CONR²⁰R²¹, CN, C(O)R²⁰, COOR²⁰, NO₂, azido, SO₂R²⁰, C(O)R²⁰ and NR²⁰COR²¹ and when R¹⁵ is an optionally substituted heteroaryl, an optionally cycloalkyl or an optionally substituted heterocycle the heteroaryl, cycloalkyl or heterocycle is unsubstituted or substituted with one or more substituents selected from the group consisting of optionally substituted C₁-C₆ alkyl, halogen, OH, oxo, OP(O)(OR²⁰)(OR²¹), optionally substituted C₁-C₆ alkoxy, NR²⁰R²¹, CONR²⁰R²¹, CN, C(O)R²⁰, COOR²⁰, NO₂, azido, SO₂R²⁰, C(O)R²⁰ and NR²⁰COR²¹;
R¹⁶ is H, optionally substituted C₂-C₆ alkenyl, optionally substituted C₂-C₆ alkynyl, optionally substituted mono or bicyclic C₃-C₆ cycloalkyl, mono or bicyclic optionally substituted C₆-C₁₂ aryl, mono or bicyclic optionally substituted 5 to 10 membered heteroaryl or optionally substituted mono or bicyclic 3 to 8 membered heterocycle; and
R¹⁷ to R¹⁹ are independently H, an optionally substituted C₁-C₆ alkyl, an optionally substituted C₂-C₆ alkenyl, an optionally substituted C₂-C₆ alkynyl or CN;
or a pharmaceutically acceptable complex, salt, solvate, tautomeric form or polymorphic form thereof.

2. (canceled)

3. The compound of claim 1, wherein R¹ and R⁴ are independently H, halogen, OH, CN, optionally substituted C₁-C₆ alkyl, optionally substituted C₂-C₆ alkenyl or optionally substituted C₂-C₆ alkynyl.

4. The compound of claim 1, wherein X² is CR² and X³ is CR³, and one of R² and R³ is -L¹-L²-L³-L⁴-R¹⁵ and the other of R² and R³ is H, halogen, OH, CN, optionally substituted C₁-C₆ alkyl, optionally substituted C₂-C₆ alkenyl or optionally substituted C₂-C₆ alkynyl.

5. The compound of claim 1, wherein;
L¹ is NR¹⁷, L² is C═O, C═S, C═NR¹⁹ or SO₂ and L³ is absent or is NR¹⁸; or
L¹ is absent, L² is C═S, C═NR¹⁹ or SO₂ and L³ is NR¹⁸; or
L¹ is absent or a C₁-C₃ alkylene, L² is absent and L³ is O; or
L¹ is an optionally substituted C₅-C₆ cycloalkylene, an optionally substituted C₆ arylene, an optionally substituted 5 membered heteroarylene or an optionally substituted 5 or 6 membered heterocyclylene, L² is absent and L³ is absent.

6. (canceled)

7. (canceled)

8. The compound of claim 1, wherein -L¹-L²-L³- are 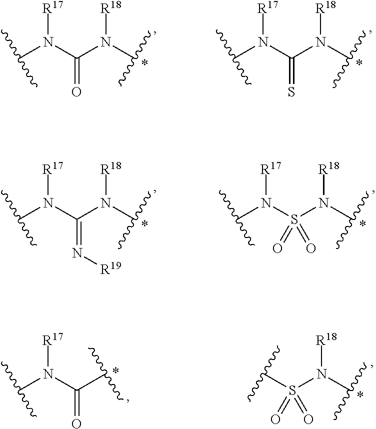
—O—*, —CH₂O—*, 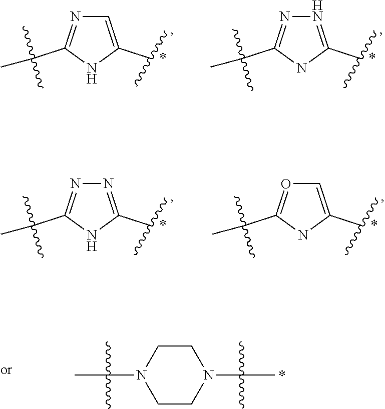
where an asterisk indicates the point of bonding to L⁴ or, when L⁴ is absent, R¹⁵.

9. The compound of claim 1, wherein L⁴ is:
absent, an optionally substituted C₁-C₆ alkylene, an optionally substituted C₂-C₆ alkenylene or an optionally substituted C₂-C₆ alkynylene; or
an optionally substituted C₃-C₆ cycloalkylene, an optionally substituted C₆-C₁₂ arylene, an optionally substituted 5 to 10 membered heteroarylene or an optionally substituted 3 to 8 membered heterocyclylene.

10. (canceled)

11. The compound of claim 1, wherein -L¹-L²-L³-L⁴- is —OCH₂CH₂—*, —CH₂OCH₂—* , 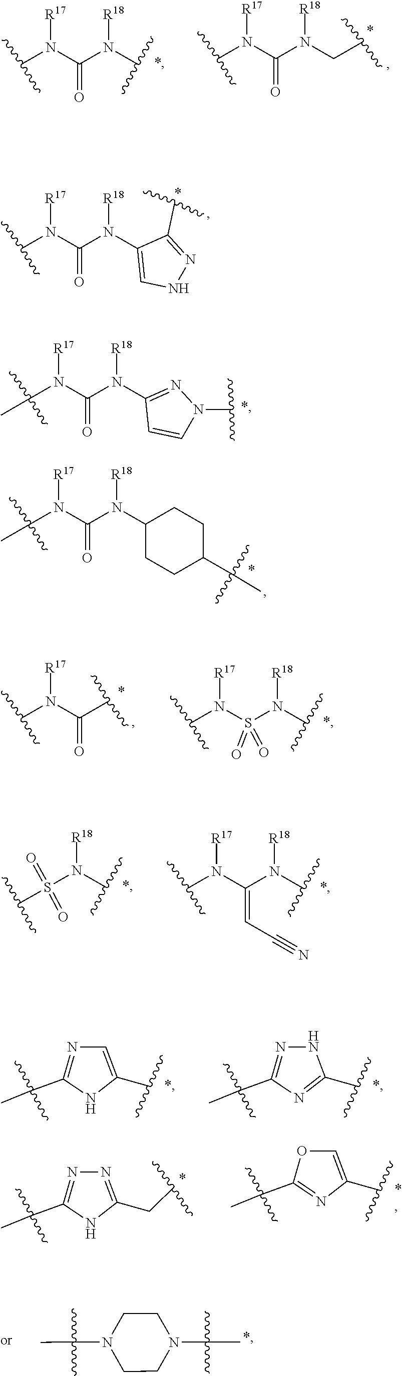
where an asterisk indicates the point of bonding to R¹⁵.

12. The compound of claim 1, wherein R¹⁷ and R¹⁸ are independently H, optionally substituted C₁-C₆ alkyl, optionally substituted C₂-C₆ alkenyl or optionally substituted C₂-C₆ alkynyl and R¹⁹ is H, C₁-C₃ alkyl, C₂-C₃ alkenyl or C₂-C₃ alkynyl.

13. The compound of claim 1, wherein R¹⁵ is:
a mono or bicyclic C₆-C₁₂ aryl and the aryl is unsubstituted or substituted with one or more substituents selected from the group consisting of optionally substituted C₁-C₆ alkyl, halogen, OH, oxo, OP(O)(OR²⁰)(OR²¹), optionally substituted C₁-C₆ alkoxy, NR²⁰R²¹, CONR²⁰R²¹, CN, C(O)R²⁰, COOR²⁰, NO₂, azido, SO₂R²⁰, C(O)R²⁰ and NR²⁰COR²¹;
a mono or bicyclic optionally substituted 5 to 10 membered heteroaryl, an optionally substituted C₃-C₆ cycloalkyl or an optionally substituted 3 to 8 membered heterocycle, and the heteroaryl, cycloalkyl or heterocycle is unsubstituted or substituted with one or more substituents selected from the group consisting of optionally substituted C₁-C₆ alkyl, halogen, OH, oxo, OP(O)(OR²⁰)(OR²¹), optionally substituted C₁-C₆ alkoxy, NR²⁰R²¹, CONR²⁰R²¹, CN, C(O)R²⁰, COOR²⁰, NO₂, azido, SO₂R²⁰, C(O)R²⁰ and NR²⁰COR²¹; or
phenyl,

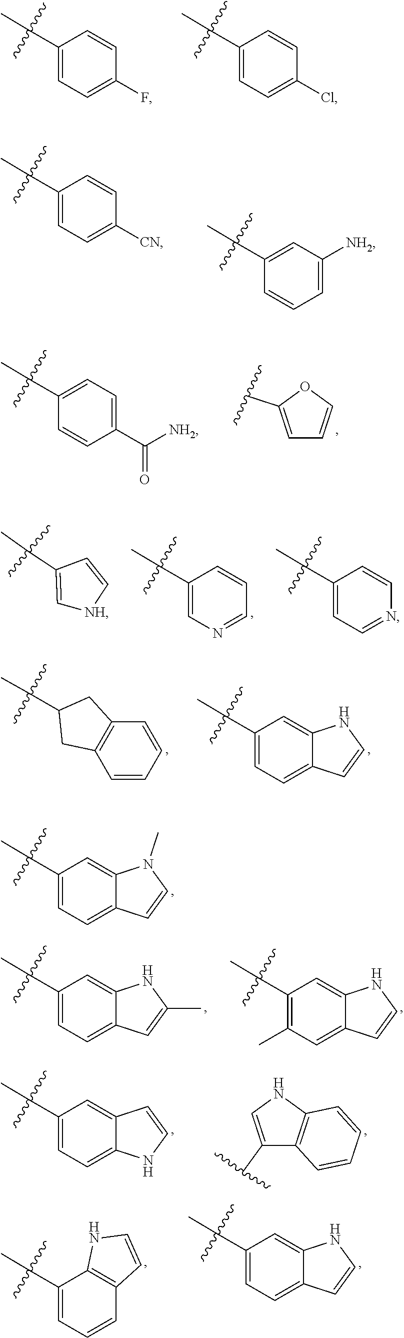

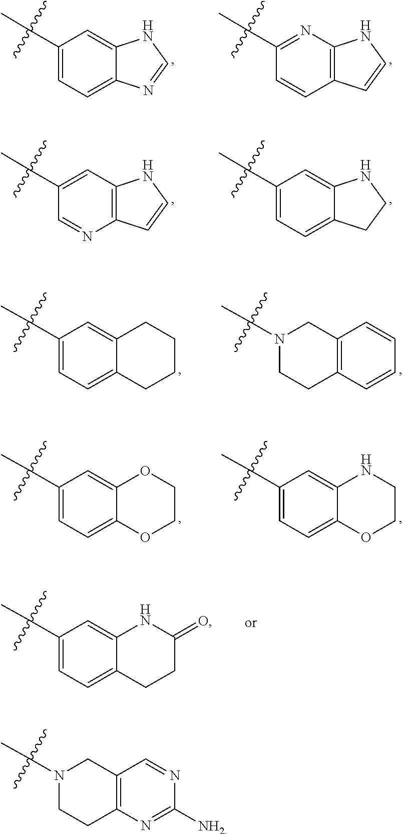

14. (canceled)

15. (canceled)

16. The compound of claim 1, wherein R⁵ is -L⁵-L⁶-R¹⁶.

17. The compound of claim 16, wherein L⁵ is absent, an optionally substituted C₁-C₃ alkylene, an optionally substituted C₂-C₃ alkenylene or an optionally substituted C₂-C₃ alkynylene, optionally wherein L⁵ is CH₂, CH₂CH₂, CO, 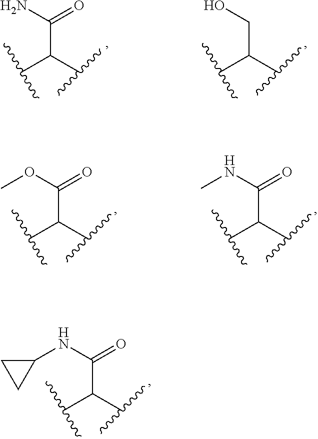
or absent.

18. (canceled)

19. The compound of claim 16, wherein L⁶ is absent, O, S, S═O, SO₂ or NR¹⁹.

20. The compound of claim 16, wherein R¹⁶ is:
optionally substituted mono or bicyclic C₃-C₆ cycloalkyl, mono or bicyclic optionally substituted C₆-C₁₂ aryl, mono or bicyclic optionally substituted 5 to 10 membered heteroaryl or optionally substituted mono or bicyclic 3 to 8 membered heterocycle, optionally wherein the cycloalkyl, aryl, heteroaryl or heterocycle is unsubstituted or substituted with one or more substituents selected from the group consisting of optionally substituted C₁-C₆ alkyl, optionally substituted C₂-C₆ alkenyl, optionally substituted C₂-C₆ alkynyl, optionally substituted C₁-C₆ alkoxy, halogen, OH, CN, oxo, C(O)R²⁰, COOR²⁰, OC(O)R²⁰, CONR²⁰R²¹, NR²⁰R²¹, NR²⁰C(O)R²¹, ═NOR²⁰, SR²⁰, SO₂R²⁰, OSO₂R²⁰, SO₂NR²⁰R²¹, OP(O)(OR²⁰)(OR²¹), optionally substituted C₆-C₁₂ aryl, optionally substituted 5 to 10 membered heteroaryl, optionally substituted C₃-C₆ cycloalkyl and optionally substituted 3 to 8 membered heterocycle; or
cyclopropyl, cyclopentyl, phenyl,

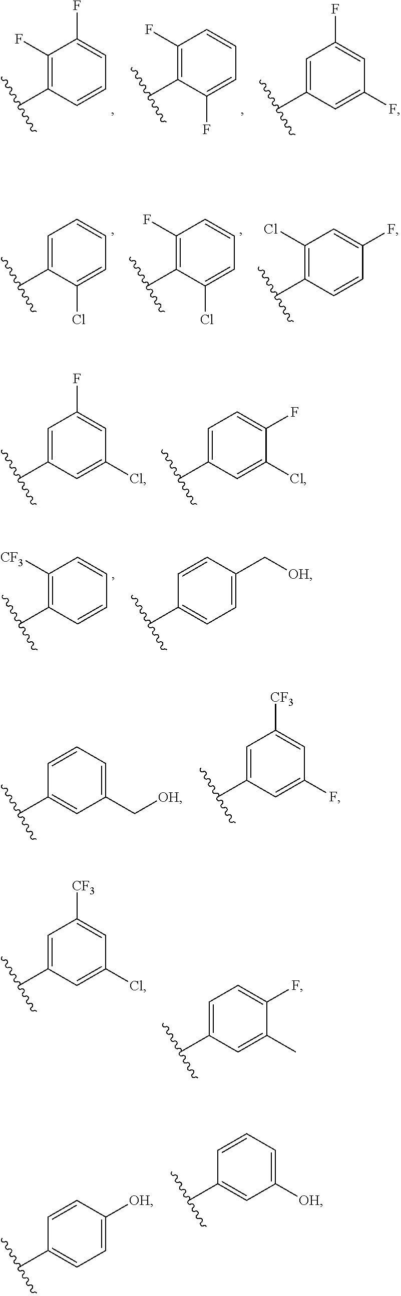

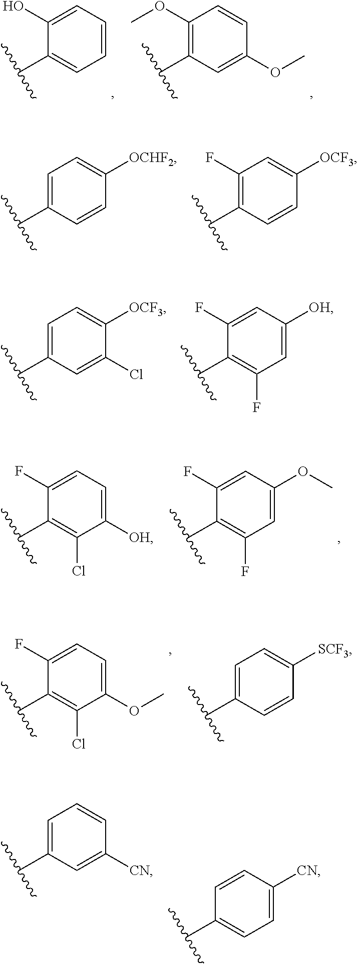

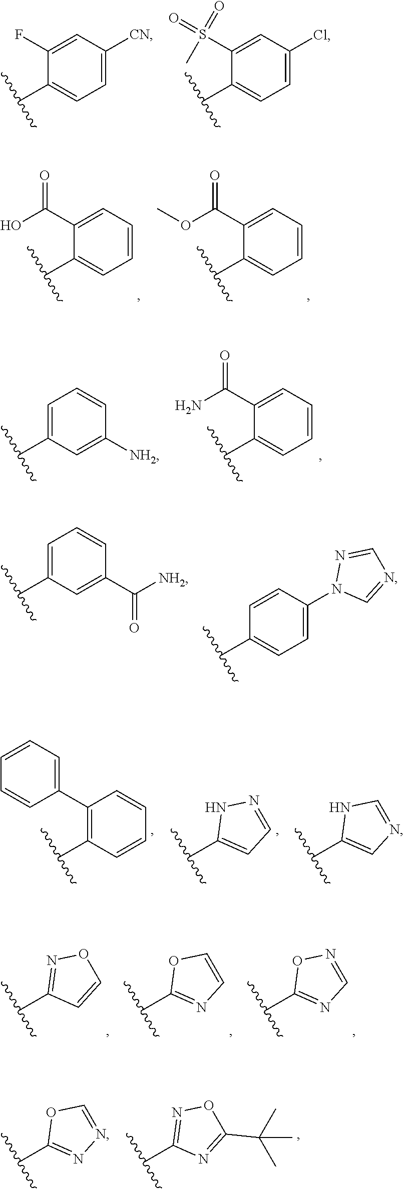

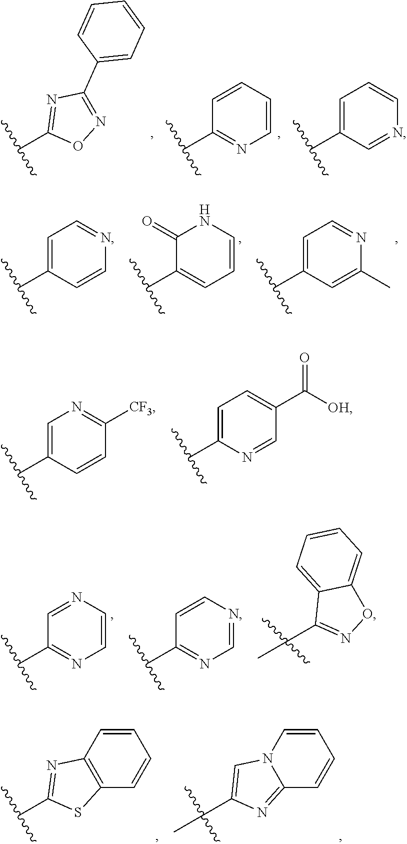

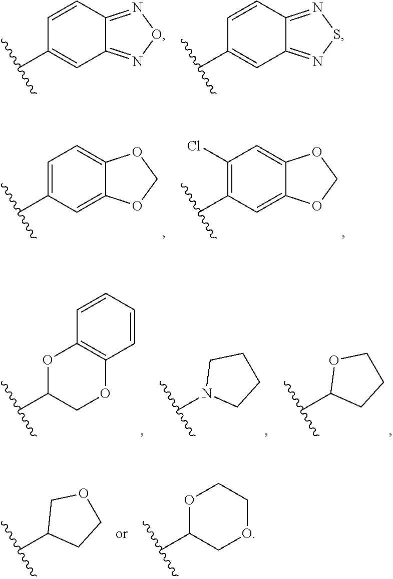

21. (canceled)

22. (canceled)

23. The compound of claim 1, wherein R⁵ is H, optionally substituted C₁-C₆ alkyl, optionally substituted C₂-C₆ alkenyl or optionally substituted C₂-C₆ alkynyl and/or R⁷ and R¹ are independently H, halogen, OH, CN, COOR¹³, CONR¹³R¹⁴, NR¹³R¹⁴, NR¹³COR⁴, optionally substituted C₁-C₆ alkyl, optionally substituted C₂-C₆ alkenyl or optionally substituted C₂-C₆ alkynyl.

24.-25. (canceled)

26. The compound of claim 1, wherein n is 1.

27. The compound of claim 25, wherein Z is CR⁹R¹⁰ and X⁷ is S, SO, SO₂, O or NR¹¹, optionally wherein R⁹ and R¹⁰ are H, halogen, OH, CN, COOR¹³, CONR¹³R¹⁴, NR¹³R¹⁴, NR¹³COR⁴, optionally substituted C₁-C₆ alkyl, optionally substituted C₂-C₆ alkenyl or optionally substituted C₂-C₆ alkynyl and R¹³ and R⁴ are H, optionally substituted C₁-C₃ alkyl, optionally substituted C₂-C₃ alkenyl or optionally substituted C₂-C₃ alkynyl and/or X⁷ is S or O.

28. (canceled)

29. (canceled)

30. The compound of claim 25, wherein Z is NR⁹ and X⁷ is CR¹¹R¹² optionally wherein R⁹ is H, optionally substituted C₁-C₆ alkyl, optionally substituted C₂-C₆ alkenyl or optionally substituted C₂-C₆ alkynyl and R¹¹ and R¹² are independently H, halogen, OH, CN, optionally substituted C₁-C₆ alkyl, optionally substituted C₂-C₆ alkenyl or optionally substituted C₂-C₆ alkynyl.

31. (canceled)

32. The compound of claim 1, wherein n is 0.

33. The compound of claim 1, wherein the compound is a compound of formula (II) or (III): 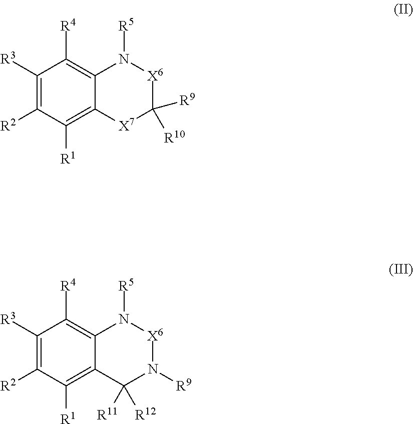
optionally wherein the compound is a compound of formula (IIa), (IIb), (IIIa) or (IIb):

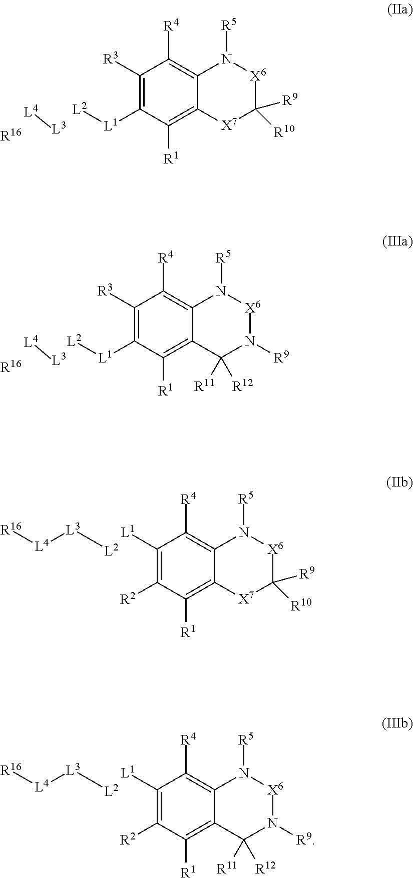

34. (canceled)

35. The compound of claim 1, wherein the compound is:
1-(4-Benzyl-3-oxo-3,4-dihydro-2H-benzo[b][1,4]thiazin-6-yl)-3-(1H-indol-6-yl)urea;
1-(4-Benzyl-3-oxo-3,4-dihydro-2H-benzo[b][1,4]thiazin-6-yl)-3-phenylurea;
1-(4-Benzyl-3-oxo-3,4-dihydro-2H-benzo[b][1,4]thiazin-6-yl)-3-(4-fluorophenyl)urea;
1-(4-Benzyl-3-oxo-3,4-dihydro-2H-benzo[b][1,4]thiazin-6-yl)-3-(pyridin-3-yl)urea;
1-(4-Benzyl-3-oxo-3,4-dihydro-2H-benzo[b][1,4]thiazin-6-yl)-3-(pyridin-4-yl)urea;
1-(4-Benzyl-3-oxo-3,4-dihydro-2H-benzo[b][1,4]thiazin-6-yl)-3-(1H-indol-5-yl)urea;
1-(4-Benzyl-3-oxo-3,4-dihydro-2H-benzo[b][1,4]thiazin-6-yl)-3-(4-cyanobenzyl)urea;
1-(4-Fluorophenyl)-3-(3-oxo-4-(pyridin-3-ylmethyl)-3,4-dihydro-2H-benzo[b][1,4]thiazin-6-yl)urea;
1-(4-Fluorophenyl)-3-(3-oxo-4-(pyridin-4-ylmethyl)-3,4-dihydro-2H-benzo[b][1,4]thiazin-6-yl)urea;
1-(4-Benzyl-3-oxo-3,4-dihydro-2H-benzo[b][1,4]thiazin-6-yl)-3-(1H-indol-3-yl)urea;
1-(4-Benzyl-3-oxo-3,4-dihydro-2H-benzo[b][1,4]thiazin-6-yl)-3-(1H-indol-7-yl)urea;
1-(4-Benzyl-3-oxo-3,4-dihydro-2H-benzo[b][1,4]thiazin-7-yl)-3-(1H-indol-6-yl)urea;
1-(4-Benzyl-3-oxo-3,4-dihydro-2H-benzo[b][1,4]thiazin-6-yl)-3-(2,3-dihydrobenzo[b][1,4]dioxin-6-yl)urea;
1-(4-(Benzo[d]isoxazol-3-ylmethyl)-3-oxo-3,4-dihydro-2H-benzo[b][1,4]thiazin-6-yl)-3-(1H-indol-6-yl)urea;
1-(3-Aminophenyl)-3-(4-benzyl-3-oxo-3,4-dihydro-2H-benzo[b][1,4]thiazin-6-yl)urea;
1-(4-Fluorophenyl)-3-(4-(imidazo[1,2-a]pyridin-2-ylmethyl)-3-oxo-3,4-dihydro-2H-benzo[b][1,4]thiazin-6-yl)urea;
1-(4-Benzyl-3-oxo-3,4-dihydro-2H-benzo[b][1,4]thiazin-6-yl)-3-(1-methyl-1H-indol-6-yl)urea;
1-(4-Benzyl-3-oxo-3,4-dihydro-2H-benzo[b][1,4]thiazin-6-yl)-3-(1H-indazol-6-yl)urea;
1-(4-Benzyl-3-oxo-3,4-dihydro-2H-benzo[b][1,4]thiazin-6-yl)-3-(2-oxo-1,2,3,4-tetrahydroquinolin-7-yl)urea;
1-(1H-Benzo[d]imidazol-6-yl)-3-(4-benzyl-3-oxo-3,4-dihydro-2H-benzo[b][1,4]thiazin-6-yl)urea;
1-(4-Benzyl-3-oxo-3,4-dihydro-2H-benzo[b][1,4]thiazin-6-yl)-3-(1H-indol-6-yl)-1-methylurea;
1-(4-Benzyl-2-methyl-3-oxo-3,4-dihydro-2H-benzo[b][1,4]thiazin-6-yl)-3-phenylurea;
4-(3-(4-Benzyl-3-oxo-3,4-dihydro-2H-benzo[b][1,4]thiazin-6-yl)ureido)benzamide;
(S)-1-(1-Benzyl-3,4-dimethyl-2-oxo-1,2,3,4-tetrahydroquinazolin-7-yl)-3-(4-fluorophenyl)urea;
1-(1-Benzyl-3-methyl-2-oxo-1,2,3,4-tetrahydroquinazolin-7-yl)-3-(4-fluorophenyl)urea;
1-(1-Benzyl-3-methyl-2-oxo-1,2,3,4-tetrahydroquinazolin-7-yl)-3-(1H-indol-6-yl)urea ex 40;
4-(2-Chloro-6-fluorobenzyl)-6-(4-fluorophenethoxy)-2H-benzo[b][1,4]thiazin-3(4H)-one;
4-(2-Chloro-6-fluorobenzyl)-6-(((4-fluorobenzyl)oxy)methyl)-2H-benzo[b][1,4]thiazin-3(4H)-one;
4-(2-Chloro-6-fluorobenzyl)-6-(5-phenyl-1H-imidazol-2-yl)-2H-benzo[b][1,4]thiazin-3(4H)-one;
4-(2-Chloro-6-fluorobenzyl)-6-(5-phenyl-1H-1,2,4-triazol-3-yl)-2H-benzo[b][1,4]thiazin-3(4H)-one;
6-(5-Benzyl-4H-1,2,4-triazol-3-yl)-4-(2-chloro-6-fluorobenzyl)-2H-benzo[b][1,4]thiazin-3(4H)-one;
4-(2-Chloro-6-fluorobenzyl)-6-(4-phenyloxazol-2-yl)-2H-benzo[b][1,4]thiazin-3(4H)-one;
N-(4-Benzyl-3-oxo-3,4-dihydro-2H-benzo[b][1,4]thiazin-6-yl)-2-(1H-indol-6-yl)acetamide;
N-(4-Benzyl-3-oxo-3,4-dihydro-2H-benzo[b][1,4]thiazin-6-yl)-2-(furan-2-yl)acetamide;
4-Benzoyl-N-(furan-2-ylmethyl)-3,4-dihydro-2H-benzo[b][1,4]thiazine-6-sulfonamide;
1-(4-Benzyl-3,4-dihydro-2H-benzo[b][1,4]thiazin-6-yl)-3-(1H-indol-6-yl)urea;
1-(4-Benzyl-3-oxo-3,4-dihydro-2H-benzo[b][1,4]oxazin-7-yl)-3-(1H-indol-6-yl)urea;
1-(4-benzyl-3-oxo-3,4-dihydro-2H-benzo[b][1,4]thiazin-6-yl)-3-(3,4-dihydro-2H-benzo[b][1,4]oxazin-6-yl)urea;
1-(4-benzyl-3-oxo-3,4-dihydro-2H-benzo[b][1,4]thiazin-7-yl)-3-(4-fluorophenyl)urea;
1-(4-(3-aminobenzyl)-3-oxo-3,4-dihydro-2H-benzo[b][1,4]thiazin-6-yl)-3-(1H-indol-6-yl)urea;
3-(4-benzyl-3-oxo-3,4-dihydro-2H-benzo[b][1,4]thiazin-6-yl)-1-(1H-indol-6-yl)-1-methylurea;
1-(1H-indol-6-yl)-3-(4-((2-methylpyridin-4-yl)methyl)-3-oxo-3,4-dihydro-2H-benzo[b][1,4]thiazin-6-yl)urea;
1-(1H-indol-6-yl)-3-(4-methyl-3-oxo-3,4-dihydro-2H-benzo[b][1,4]thiazin-6-yl)urea;
1-(1H-indol-6-yl)-3-(3-oxo-3,4-dihydro-2H-benzo[b][1,4]oxazin-7-yl)urea;
1-(4-(2-chloro-6-fluorobenzyl)-3-oxo-3,4-dihydro-2H-benzo[b][1,4]thiazin-7-yl)-3-(1H-indol-6-yl)urea;
1-(4-benzyl-2,2-dimethyl-3-oxo-3,4-dihydro-2H-benzo[b][1,4]oxazin-7-yl)-3-(1H-indol-6-yl)urea;
1-(4-benzyl-3-oxo-3,4-dihydro-2H-benzo[b][1,4]oxazin-7-yl)-3-(5-methyl-1H-indol-6-yl)urea;
1-(4-benzyl-3-oxo-3,4-dihydro-2H-benzo[b][1,4]oxazin-7-yl)-3-(2-methyl-1H-indol-6-yl)urea;
1-(4-(2-chloro-6-fluorobenzyl)-3-oxo-3,4-dihydro-2H-benzo[b][1,4]oxazin-7-yl)-3-(1H-indol-6-yl)urea;
1-(4-(2-chloro-4-fluorobenzyl)-3-oxo-3,4-dihydro-2H-benzo[b][1,4]oxazin-7-yl)-3-(1H-indol-6-yl)urea;
1-(4-(2,3-difluorobenzyl)-3-oxo-3,4-dihydro-2H-benzo[b][1,4]oxazin-7-yl)-3-(1H-indol-6-yl)urea;
1-(4-(2,6-difluorobenzyl)-3-oxo-3,4-dihydro-2H-benzo[b][1,4]oxazin-7-yl)-3-(1H-indol-6-yl)urea;
1-(4-((2,3-dihydrobenzo[b][1,4]dioxin-2-yl)methyl)-3-oxo-3,4-dihydro-2H-benzo[b][1,4]oxazin-7-yl)-3-(1H-indol-6-yl)urea;
3-((7-(3-(1H-indol-6-yl)ureido)-3-oxo-2,3-dihydro-4H-benzo[b][1,4]oxazin-4-yl)methyl)benzamide;
1-(4-(3-chloro-5-(trifluoromethyl)benzyl)-3-oxo-3,4-dihydro-2H-benzo[b][1,4]oxazin-7-yl)-3-(1H-indol-6-yl)urea;
1-(4-(2-(2-chlorophenoxy)ethyl)-3-oxo-3,4-dihydro-2H-benzo[b][1,4]oxazin-7-yl)-3-(1H-indol-6-yl)urea;
1-(4-([1,1′-biphenyl]-2-ylmethyl)-3-oxo-3,4-dihydro-2H-benzo[b][1,4]oxazin-7-yl)-3-(1H-indol-6-yl)urea;
1-(4-(3-chloro-5-fluorobenzyl)-3-oxo-3,4-dihydro-2H-benzo[b][1,4]oxazin-7-yl)-3-(1H-indol-6-yl)urea;
1-(4-((6-chlorobenzo[d][1,3]dioxol-5-yl)methyl)-3-oxo-3,4-dihydro-2H-benzo[b][1,4]oxazin-7-yl)-3-(1H-indol-6-yl)urea;
1-(1H-indol-6-yl)-3-(3-oxo-4-((6-(trifluoromethyl)pyridin-3-yl)methyl)-3,4-dihydro-2H-benzo[b][1,4]oxazin-7-yl)urea;
1-(4-(3,5-difluorobenzyl)-3-oxo-3,4-dihydro-2H-benzo[b][1,4]oxazin-7-yl)-3-(1H-indol-6-yl)urea;
1-(1H-indol-6-yl)-3-(3-oxo-4-(4-((trifluoromethyl)thio)benzyl)-3,4-dihydro-2H-benzo[b][1,4]oxazin-7-yl)urea;
1-(4-(3-chloro-4-(trifluoromethoxy)benzyl)-3-oxo-3,4-dihydro-2H-benzo[b][1,4]oxazin-7-yl)-3-(1H-indol-6-yl)urea;
1-(4-(4-fluoro-3-methylbenzyl)-3-oxo-3,4-dihydro-2H-benzo[b][1,4]oxazin-7-yl)-3-(1H-indol-6-yl)urea;
1-(4-(4-(difluoromethoxy)benzyl)-3-oxo-3,4-dihydro-2H-benzo[b][1,4]oxazin-7-yl)-3-(1H-indol-6-yl)urea;
1-(4-(2-chlorophenethyl)-3-oxo-3,4-dihydro-2H-benzo[b][1,4]oxazin-7-yl)-3-(1H-indol-6-yl)urea;
1-(4-(3-chloro-4-fluorobenzyl)-3-oxo-3,4-dihydro-2H-benzo[b][1,4]oxazin-7-yl)-3-(1H-indol-6-yl)urea;
1-(4-(3-fluoro-5-(trifluoromethyl)benzyl)-3-oxo-3,4-dihydro-2H-benzo[b][1,4]oxazin-7-yl)-3-(1H-indol-6-yl)urea;
1-(1H-indol-6-yl)-3-(3-oxo-4-(2-(trifluoromethyl)benzyl)-3,4-dihydro-2H-benzo[b][1,4]oxazin-7-yl)urea;
1-(4-(benzo[d][1,3]dioxol-5-ylmethyl)-3-oxo-3,4-dihydro-2H-benzo[b][1,4]oxazin-7-yl)-3-(1H-indol-6-yl)urea;
1-(4-(2,5-dimethoxybenzyl)-3-oxo-3,4-dihydro-2H-benzo[b][1,4]oxazin-7-yl)-3-(1H-indol-6-yl)urea;
1-(4-(3-cyanobenzyl)-3-oxo-3,4-dihydro-2H-benzo[b][1,4]oxazin-7-yl)-3-(1H-indol-6-yl)urea;
1-(4-(benzo[d]thiazol-2-ylmethyl)-3-oxo-3,4-dihydro-2H-benzo[b][1,4]oxazin-7-yl)-3-(1H-indol-6-yl)urea;
6-((7-(3-(1H-indol-6-yl)ureido)-3-oxo-2,3-dihydro-4H-benzo[b][1,4]oxazin-4-yl)methyl)nicotinic acid;
1-(4-(benzo[c][1,2,5]thiadiazol-5-ylmethyl)-3-oxo-3,4-dihydro-2H-benzo[b][1,4]oxazin-7-yl)-3-(1H-indol-6-yl)urea;
1-(4-(4-cyano-2-fluorobenzyl)-3-oxo-3,4-dihydro-2H-benzo[b][1,4]oxazin-7-yl)-3-(1H-indol-6-yl)urea;
1-(1H-indol-6-yl)-3-(3-oxo-4-((3-phenyl-1,2,4-oxadiazol-5-yl)methyl)-3,4-dihydro-2H-benzo[b][1,4]oxazin-7-yl)urea;
1-(4-(4-chloro-2-(methylsulfonyl)benzyl)-3-oxo-3,4-dihydro-2H-benzo[b][1,4]oxazin-7-yl)-3-(1H-indol-6-yl)urea;
1-(4-(4-cyanobenzyl)-3-oxo-3,4-dihydro-2H-benzo[b][1,4]oxazin-7-yl)-3-(1H-indol-6-yl)urea;
1-(4-(2-chloro-6-fluoro-3-methoxybenzyl)-3-oxo-3,4-dihydro-2H-benzo[b][1,4]thiazin-7-yl)-3-(1H-indol-6-yl)urea;
1-(4-(2-chloro-6-fluoro-3-hydroxybenzyl)-3-oxo-3,4-dihydro-2H-benzo[b][1,4]thiazin-7-yl)-3-(1H-indol-6-yl)urea;
1-(4-(2,6-difluoro-4-methoxybenzyl)-3-oxo-3,4-dihydro-2H-benzo[b][1,4]thiazin-7-yl)-3-(1H-indol-6-yl)urea;
1-(4-(2,6-difluoro-4-hydroxybenzyl)-3-oxo-3,4-dihydro-2H-benzo[b][1,4]thiazin-7-yl)-3-(1H-indol-6-yl)urea;
1-(4-(4-(1H-1,2,4-triazol-1-yl)benzyl)-3-oxo-3,4-dihydro-2H-benzo[b][1,4]oxazin-7-yl)-3-(1H-indol-6-yl)urea;
1-(4-(benzo[c][1,2,5]oxadiazol-5-ylmethyl)-3-oxo-3,4-dihydro-2H-benzo[b][1,4]oxazin-7-yl)-3-(1H-indol-6-yl)urea;
1-(4-benzyl-3-oxo-3,4-dihydro-2H-benzo[b][1,4]oxazin-7-yl)-3-(furan-2-ylmethyl)urea;
1-(4-benzyl-3-oxo-3,4-dihydro-2H-benzo[b][1,4]oxazin-7-yl)-3-(3-(4-chlorophenyl)-1H-pyrazol-4-yl)urea;
1-((1H-pyrrol-3-yl)methyl)-3-(4-benzyl-3-oxo-3,4-dihydro-2H-benzo[b][1,4]oxazin-7-yl)urea;
1-(4-benzyl-3-oxo-3,4-dihydro-2H-benzo[b][1,4]oxazin-7-yl)-3-(5,6,7,8-tetrahydronaphthalen-2-yl)urea;
1-(4-((5-(tert-butyl)-1,2,4-oxadiazol-3-yl)methyl)-3-oxo-3,4-dihydro-2H-benzo[b][1,4]oxazin-7-yl)-3-(1H-indol-6-yl)urea;
1-(4-benzyl-3-oxo-3,4-dihydro-2H-benzo[b][1,4]oxazin-7-yl)-3-(1,2,3,4-tetrahydronaphthalen-2-yl)urea;
1-(4-benzyl-3-oxo-3,4-dihydro-2H-benzo[b][1,4]oxazin-7-yl)-3-(2,3-dihydro-1H-inden-2-yl)urea;
1-(4-benzyl-3-oxo-3,4-dihydro-2H-benzo[b][1,4]oxazin-7-yl)-3-(4-phenylcyclohexyl)urea;
1-(4-benzyl-3-oxo-3,4-dihydro-2H-benzo[b][1,4]oxazin-7-yl)-3-(1-phenyl-1H-pyrazol-3-yl)urea;
1-(4-(4-hydroxybenzyl)-3-oxo-3,4-dihydro-2H-benzo[b][1,4]oxazin-7-yl)-3-(1H-indol-6-yl)urea;
methyl 2-((7-(3-(1H-indol-6-yl)ureido)-3-oxo-2,3-dihydro-4H-benzo[b][1,4]oxazin-4-yl)methyl)benzoate;
N-(4-benzyl-3-oxo-3,4-dihydro-2H-benzo[b][1,4]oxazin-7-yl)-3,4-dihydroisoquinoline-2(1H)-carboxamide;
1-(4-benzyl-3-oxo-3,4-dihydro-2H-benzo[b][1,4]oxazin-7-yl)-3-(indolin-6-yl)urea;
2-((7-(3-(1H-indol-6-yl)ureido)-3-oxo-2,3-dihydro-4H-benzo[b][1,4]oxazin-4-yl)methyl)benzoic acid;
2-((7-(3-(1H-indol-6-yl)ureido)-3-oxo-2,3-dihydro-4H-benzo[b][1,4]oxazin-4-yl)methyl)benzamide;
1-(4-((1,4-dioxan-2-yl)methyl)-3-oxo-3,4-dihydro-2H-benzo[b][1,4]oxazin-7-yl)-3-(1H-indol-6-yl)urea;
1-(1H-indol-6-yl)-3-(3-oxo-4-((tetrahydrofuran-2-yl)methyl)-3,4-dihydro-2H-benzo[b][1,4]oxazin-7-yl)urea;
1-(1H-indol-6-yl)-3-(3-oxo-4-(pyridin-4-ylmethyl)-3,4-dihydro-2H-benzo[b][1,4]oxazin-7-yl)urea;
1-(1H-indol-6-yl)-3-(3-oxo-4-(pyridin-3-ylmethyl)-3,4-dihydro-2H-benzo[b][1,4]oxazin-7-yl)urea;
1-(1H-indol-6-yl)-3-(3-oxo-4-(pyridin-2-ylmethyl)-3,4-dihydro-2H-benzo[b][1,4]oxazin-7-yl)urea;
1-(1H-indol-6-yl)-3-(3-oxo-4-((tetrahydrofuran-3-yl)methyl)-3,4-dihydro-2H-benzo[b][1,4]oxazin-7-yl)urea;
1-(4-((1,3,4-oxadiazol-2-yl)methyl)-3-oxo-3,4-dihydro-2H-benzo[b][1,4]oxazin-7-yl)-3-(1H-indol-6-yl)urea;
1-(4-(3-hydroxybenzyl)-3-oxo-3,4-dihydro-2H-benzo[b][1,4]oxazin-7-yl)-3-(1H-indol-6-yl)urea;
1-(4-(4-(hydroxymethyl)benzyl)-3-oxo-3,4-dihydro-2H-benzo[b][1,4]oxazin-7-yl)-3-(1H-indol-6-yl)urea;
1-(1H-indol-6-yl)-3-(3-oxo-4-(pyrazin-2-ylmethyl)-3,4-dihydro-2H-benzo[b][1,4]oxazin-7-yl)urea;
3-((7-(3-(1H-indol-6-yl)ureido)-3-oxo-2,3-dihydro-4H-benzo[b][1,4]oxazin-4-yl)methyl)benzamide;
1-(4-(3-(hydroxymethyl)benzyl)-3-oxo-3,4-dihydro-2H-benzo[b][1,4]oxazin-7-yl)-3-(1H-indol-6-yl)urea;
1-(4-(cyanomethyl)-3-oxo-3,4-dihydro-2H-benzo[b][1,4]oxazin-7-yl)-3-(1H-indol-6-yl)urea;
1-(1H-indol-6-yl)-3-(3-oxo-4-((2-oxo-1,2-dihydropyridin-3-yl)methyl)-3,4-dihydro-2H-benzo[b][1,4]oxazin-7-yl)urea;
1-(4-(2-hydroxybenzyl)-3-oxo-3,4-dihydro-2H-benzo[b][1,4]oxazin-7-yl)-3-(1H-indol-6-yl)urea;
1-(1H-indol-6-yl)-3-(3-oxo-4-(pyrimidin-4-ylmethyl)-3,4-dihydro-2H-benzo[b][1,4]oxazin-7-yl)urea;
1-(4-((1H-pyrazol-5-yl)methyl)-3-oxo-3,4-dihydro-2H-benzo[b][1,4]oxazin-7-yl)-3-(1H-indol-6-yl)urea;
1-(4-((1H-imidazol-5-yl)methyl)-3-oxo-3,4-dihydro-2H-benzo[b][1,4]oxazin-7-yl)-3-(1H-indol-6-yl)urea;
1-(4-((1,2,4-oxadiazol-5-yl)methyl)-3-oxo-3,4-dihydro-2H-benzo[b][1,4]oxazin-7-yl)-3-(1H-indol-6-yl)urea;
1-(1H-indol-6-yl)-3-(4-(isoxazol-3-ylmethyl)-3-oxo-3,4-dihydro-2H-benzo[b][1,4]oxazin-7-yl)urea;
7-(3-(1H-indol-6-yl)ureido)-4-benzyl-3-oxo-3,4-dihydro-2H-benzo[b][1,4]oxazine-6-carboxamide;
1-(4-benzyl-6-bromo-3-oxo-3,4-dihydro-2H-benzo[b][1,4]oxazin-7-yl)-3-(1H-indol-6-yl)urea;
1-(4-fluorophenyl)-3-(4-methyl-3-oxo-3,4-dihydro-2H-benzo[b][1,4]thiazin-6-yl)urea;
2-amino-N-(4-benzyl-3-oxo-3,4-dihydro-2H-benzo[b][1,4]thiazin-6-yl)-7,8-dihydropyrido[4,3-d]pyrimidine-6(5H)-carboxamide;
1-(1H-indol-6-yl)-3-(3-oxo-3,4-dihydro-2H-benzo[b][1,4]thiazin-6-yl)urea;
N-(4-benzyl-3-oxo-3,4-dihydro-2H-benzo[b][1,4]thiazin-6-yl)-1H-indole-6-carboxamide;
1-(4-Benzyl-3,4-dihydro-2H-benzo[b][1,4]oxazin-7-yl)-3-(1H-indol-6-yl)urea;
1-(4-benzyl-3,4-dihydro-2H-benzo[b][1,4]oxazin-6-yl)-3-(1H-indol-6-yl)urea;
1-(4-benzoyl-3,4-dihydro-2H-benzo[b][1,4]thiazin-6-yl)-3-(1H-indol-6-yl)urea;
1-(4-benzyl-3,4-dihydro-2H-benzo[b][1,4]thiazin-7-yl)-3-(1H-indol-6-yl)urea;
1-(4-benzyl-3,4-dihydro-2H-benzo[b][1,4]thiazin-6-yl)-3-(1H-indol-3-yl)urea;
1-(4-(cyclopropylmethyl)-3,4-dihydro-2H-benzo[b][1,4]thiazin-6-yl)-3-(1H-indol-6-yl)urea;
1-(1H-indol-6-yl)-3-(4-(pyridin-4-ylmethyl)-3,4-dihydro-2H-benzo[b][1,4]thiazin-6-yl)urea;
1-(4-(cyclopentylmethyl)-3,4-dihydro-2H-benzo[b][1,4]thiazin-6-yl)-3-(1H-indol-6-yl)urea;
1-(1H-indol-6-yl)-3-(4-(2-(pyrrolidin-1-yl)ethyl)-3,4-dihydro-2H-benzo[b][1,4]thiazin-6-yl)urea;
1-(4-benzyl-1-oxido-3,4-dihydro-2H-benzo[b][1,4]thiazin-6-yl)-3-(1H-indol-6-yl)urea;
1-(1H-indol-6-yl)-3-(4-methyl-3,4-dihydro-2H-benzo[b][1,4]thiazin-6-yl)urea;
1-(4-(2-chloro-6-fluorobenzyl)-3,4-dihydro-2H-benzo[b][1,4]oxazin-6-yl)-3-(1H-indol-6-yl)urea;
1-(4-(2-chloro-6-fluorobenzyl)-3,4-dihydro-2H-benzo[b][1,4]oxazin-7-yl)-3-(1H-indol-6-yl)urea;
1-(4-(2-chloro-6-fluorobenzyl)-3,4-dihydro-2H-benzo[b][1,4]thiazin-7-yl)-3-(1H-indol-6-yl)urea;
1-(4-benzyl-3,4-dihydro-2H-benzo[b][1,4]oxazin-6-yl)-3-(1H-pyrrolo[2,3-b]pyridin-6-yl)urea;
1-(4-benzyl-3,4-dihydro-2H-benzo[b][1,4]thiazin-6-yl)-3-(4-fluorophenyl)urea;
1-(4-(2-fluoro-4-(trifluoromethoxy)benzyl)-3,4-dihydro-2H-benzo[b][1,4]oxazin-6-yl)-3-(1H-indol-6-yl)urea;
4-benzyl-N-(1H-indol-6-ylsulfamoyl)-2,3-dihydro-1,4-benzoxazin-6-amine 1-(4-benzyl-3,4-dihydro-2H-benzo[b][1,4]oxazin-6-yl)-3-(1H-pyrrolo[3,2-b]pyridin-6-yl)urea;
1-(4-benzyl-3,4-dihydro-2H-benzo[b][1,4]oxazin-6-yl)-3-(1H-indol-6-yl)-1-methylurea;
6-(3-(1H-indol-6-yl)ureido)-4-benzyl-3,4-dihydro-2H-benzo[b][1,4]oxazine-7-carboxamide;
1-(4-benzyl-7-bromo-3,4-dihydro-2H-benzo[b][1,4]oxazin-6-yl)-3-(1H-indol-6-yl)urea;
1-(1H-Indol-6-yl)-3-(4-phenyl-3,4-dihydro-2H-benzo[b][1,4]thiazin-6-yl)urea;
1-(1H-Indol-6-yl)-3-(3-oxo-4-phenyl-3,4-dihydro-2H-benzo[b][1,4]oxazin-7-yl)urea;
N-1H-indol-6-yl-N′-(4-phenyl-3,4-dihydro-2H-1,4-benzoxazin-7-yl)sulfuric diamide
1-(1H-indol-6-yl)-3-(4-phenyl-3,4-dihydro-2H-benzo[b][1,4]oxazin-7-yl)urea;
1-(1H-indol-6-yl)-3-(4-phenyl-3,4-dihydro-2H-benzo[b][1,4]thiazin-7-yl)urea;
1-(1H-indol-6-yl)-3-(3-oxo-4-phenyl-3,4-dihydro-2H-benzo[b][1,4]oxazin-6-yl)urea;
1-(1H-indol-6-yl)-3-(4-phenyl-3,4-dihydro-2H-benzo[b][1,4]oxazin-6-yl)urea;
1-(1H-indol-6-yl)-3-(4-(oxazol-2-yl)-3,4-dihydro-2H-benzo[b][1,4]oxazin-7-yl)urea;
1-(4-Benzyl-1-methyl-1,2,3,4-tetrahydroquinoxalin-6-yl)-3-(1H-indol-6-yl)urea;
1-(1-Benzylindolin-6-yl)-3-(1H-indol-6-yl)urea;
2-(6-(3-(1H-Indol-6-yl)ureido)-4-benzyl-3-oxo-3,4-dihydro-2H-benzo[b][1,4]thiazin-2-yl)acetamide;
1-(3-Allyl-4-benzyl-3,4-dihydro-2H-benzo[b][1,4]oxazin-6-yl)-3-(1H-indol-6-yl)urea;
1-(4-Benzyl-3-(2,3-dihydroxypropyl)-3,4-dihydro-2H-benzo[b][1,4]oxazin-6-yl)-3-(1H-indol-6-yl)urea;
1-(4-Benzyl-3-(2-hydroxyethyl)-3,4-dihydro-2H-benzo[b][1,4]oxazin-6-yl)-3-(1H-indol-6-yl)urea;
1-(4-Benzyl-3-cyano-3,4-dihydro-2H-benzo[b][1,4]oxazin-6-yl)-3-(1H-indol-6-yl)urea;
1-(3-(Aminomethyl)-4-benzyl-3,4-dihydro-2H-benzo[b][1,4]oxazin-6-yl)-3-(1H-indol-6-yl)urea;
6-(3-(1H-Indol-6-yl)ureido)-4-benzyl-3,4-dihydro-2H-benzo[b][1,4]oxazine-3-carboxamide;
1-(4-benzyl-3-cyano-3,4-dihydro-2H-benzo[b][1,4]oxazin-7-yl)-3-(1H-indol-6-yl)urea;
1-(3-(aminomethyl)-4-benzyl-3,4-dihydro-2H-benzo[b][1,4]oxazin-7-yl)-3-(1H-indol-6-yl)urea;
7-(3-(1H-indol-6-yl)ureido)-4-benzyl-3,4-dihydro-2H-benzo[b][1,4]oxazine-3-carboxamide;
2-(6-(3-(1H-Indol-6-yl)ureido)-2,3-dihydro-4H-benzo[b][1,4]oxazin-4-yl)-2-phenylacetamide;
1-(4-(2-Hydroxy-1-phenylethyl)-3,4-dihydro-2H-benzo[b][1,4]oxazin-6-yl)-3-(1H-indol-6-yl)urea;
Methyl 2-(6-(3-(1H-indol-6-yl)ureido)-2,3-dihydro-4H-benzo[b][1,4]oxazin-4-yl)-2-phenylacetate;
2-(6-(3-(1H-indol-6-yl)ureido)-2,3-dihydro-4H-benzo[b][1,4]oxazin-4-yl)-N-methyl-2-phenylacetamide;
2-(6-(3-(1H-indol-6-yl)ureido)-2,3-dihydro-4H-benzo[b][1,4]oxazin-4-yl)-N-cyclopropyl-2-phenylacetamide;
6-(4-(1H-Indol-6-yl)piperazin-1-yl)-4-benzyl-2H-benzo[b][1,4]thiazin-3(4H)-one;
1-(4-Benzyl-3-oxo-3,4-dihydro-2H-benzo[b][1,4]thiazin-6-yl)-2-cyano-3-(1H-indol-6-yl)guanidine; or
1-(4-Benzyl-2-(cyanomethyl)-3-oxo-3,4-dihydro-2H-benzo[b][1,4]thiazin-6-yl)-3-(1H-indol-6-yl)urea.

36. A pharmaceutical composition comprising a compound according to claim 1, or a pharmaceutically acceptable salt, solvate, tautomeric form or polymorphic form thereof, and a pharmaceutically acceptable vehicle.

37.-41. (canceled)

42. A method of modulating the STING protein in a subject, the method comprising administering, to a subject in need of such treatment, a therapeutically effective amount of a compound of formula (I), as defined by claim 1, or a pharmaceutically acceptable complex, salt, solvate, tautomeric form or polymorphic form thereof.

43. A method of treating, ameliorating or preventing a disease selected from liver fibrosis, fatty liver disease, non-alcoholic steatohepatitis (NASH), pulmonary fibrosis, lupus, sepsis, rheumatoid arthritis (RA), type I diabetes, STING-associated vasculopathy with onset in infancy (SAVI), Aicardi-Goutieres syndrome (AGS), familial chilblain lupus (FCL), systemic lupus erythematosus (SLE), retinal vasculopathy, neuroinflammation, systemic inflammatory response syndrome, pancreatitis, cardiovascular disease, renal fibrosis, stroke and age-related macular degeneration (AMD); the method comprising administering, to a subject in need of such treatment, a therapeutically effective amount of a compound of formula (I), as defined by claim 1, or a pharmaceutically acceptable complex, salt, solvate, tautomeric form or polymorphic form thereof.

44. The method of claim 42, wherein the disease is fibrosis or fatty liver disease, and the fibrosis is selected from the group consisting of liver fibrosis, pulmonary fibrosis or renal fibrosis, and the fatty liver disease is non-alcoholic (or simple) fatty liver or non-alcoholic steatohepatitis (NASH).
